SEQUENCE LISTING (17 sequences)

SEQ ID NO: 1 - DNA, 23 bp, Artificial Sequence
  Description of Artificial Sequence Synthetic primer
cattggagcaagtgttggatctt

SEQ ID NO: 2 - DNA, 22 bp, Artificial Sequence
  Description of Artificial Sequence Synthetic primer
gagctaatgcatgccattctca

SEQ ID NO: 3 - DNA, 18 bp, Artificial Sequence
  Description of Artificial Sequence Synthetic primer
gggggcaccagaggcagt

SEQ ID NO: 4 - DNA, 19 bp, Artificial Sequence
  Description of Artificial Sequence Synthetic primer
ggttgtggcgggggcagtt

SEQ ID NO: 5 - DNA, 23 bp, Artificial Sequence
  Description of Artificial Sequence Synthetic primer
gggcttgaagctgcttacgaatt

SEQ ID NO: 6 - DNA, 25 bp, Artificial Sequence
  Description of Artificial Sequence Synthetic primer
cagcatcgatatgcttcacagttct

SEQ ID NO: 7 - DNA, 20 bp, Artificial Sequence
  Description of Artificial Sequence Synthetic primer
tggacctgacctgccgtcta

SEQ ID NO: 8 - DNA, 22 bp, Artificial Sequence
  Description of Artificial Sequence Synthetic primer
ccctgttgctgtagccaaattc

SEQ ID NO: 9 - DNA, 23 bp, Artificial Sequence
  Description of Artificial Sequence Synthetic primer
ggctacttgggctattgtaaagg

SEQ ID NO: 10 - DNA, 23 bp, Artificial Sequence
  Description of Artificial Sequence Synthetic primer
cagtttctccgacaactttctct

SEQ ID NO: 11 - DNA, 20 bp, Artificial Sequence
  Description of Artificial Sequence Synthetic primer
gaccagtcaacaggggacat

SEQ ID NO: 12 - DNA, 22 bp, Artificial Sequence
  Description of Artificial Sequence Synthetic primer
aacacttcgtggggtccttttc

SEQ ID NO: 13 - protein, 28 aa, Artificial Sequence
  Description of Artificial Sequence Synthetic polypeptide
MFVFLVLLPLVSSQCVNLTTRTQLPPAY

SEQ ID NO: 14 - protein, 78 aa, Artificial Sequence
  Description of Artificial Sequence Synthetic polypeptide
RTFLLKYNENGTITDAVDCALDPLSETKCTLKSFTVEKGIYQTSNFRVQPTESIVRFPNITNLCPFGEVFNATRFASV

SEQ ID NO: 15 - protein, 90 aa, Artificial Sequence
  Description of Artificial Sequence Synthetic polypeptide
SNCVADYSVLYNSASFSTFKCYGVSPTKLNDLCFTNVYADSFVIRGDEVRQIAPGQTGKIADYNYKLPDDFTGCVIAWNSNNLDSKVGGN

SEQ ID NO: 16 - protein, 196 aa, Artificial Sequence
  Description of Artificial Sequence Synthetic polypeptide
ELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNS

SEQ ID NO: 17 - protein, 56 aa, Artificial Sequence
  Description of Artificial Sequence Synthetic polypeptide
LGFIAGLIAIVMVTIMLCCMTSCCSCLKGCCSCGSCCKFDEDDSEPVLKGVKLHYT

(19) United States
(12) Patent Application Publication
(10) Pub. No.: US 2024/0041813 A1
(43) Pub. Date: Feb. 8, 2024

(54) VERY-LONG-CHAIN POLYUNSATURATED FATTY ACIDS, ELOVANOID HYDROXYLATED DERIVATIVES, AND METHODS OF USE

(71) Applicant: Boards of Supervisors of Louisiana State University and Agricultural and Mechanical College, Baton Rouge, LA (US)

(72) Inventor: Nicolas G. BAZAN, New Orleans, LA (US)

(21) Appl. No.: 18/025,604

(22) PCT Filed: Sep. 10, 2021
(86) PCT No.: PCT/US2021/049946

§ 371 Date: Mar. 9, 2023

Related U.S. Application Data
(60) Provisional application No. 63/076,900, filed on Sep. 10, 2020.
(60) Provisional application No. 63/076,911, filed on Sep. 10, 2020.
(60) Provisional application No. 63/092,937, filed on Oct. 16, 2020.
(60) Provisional application No. 63/125,521, filed on Dec. 15, 2020.

(51) Int. Cl. A61K 31/202; A61P 25/28

(52) U.S. Cl. CPC A61K 31/202; A61P 25/28

(57) ABSTRACT

This disclosure is directed is directed to compositions and methods for preventing, treating, or ameliorating the symptoms of a viral infection.

This application is an International Application which claims priority from U.S. Provisional Patent Application No. 63/076,900, filed on 10 Sep. 2020, U.S. Provisional Patent Application No. 63/092,937, filed on 16 Oct. 2020, 63/076,911, filed on 10 Sep. 2020, and U.S. Provisional Patent Application No. 63/125,521 filed on 15 Dec. 2020, the contents of each of which are incorporated herein by reference in their entireties.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

This invention was made with government support under Grant No. R01 EY019465 awarded by the National Institutes of Health and Grant No. EY005121 awarded by the National Institutes of Health. The government has certain rights in the invention.

### FIELD OF THE DISCLOSURE

This disclosure is directed is directed to compositions and methods for preventing, treating, or ameliorating the symptoms of a viral infection.

### BACKGROUND

Infection by a virus can lead to a viral disease or viral induced inflammatory response or immune dysfunction. These diseases and inflammatory/immune responses can be harmful or deadly to an organism. In December 2019, a new infectious respiratory disease (Coronavirus disease 2019, naming the coronavirus disease (COVID-19) caused by severe acute respiratory syndrome coronavirus 2 (SARS-CoV-2) emerged, quickly becoming a pandemic and a global threat to public health. The virus has a single-stranded RNA with a 30 kb genome, which encodes the spike (S) protein that expresses a receptor-binding domain (RBD) for the angiotensin-converting enzyme 2 (ACE2) receptor. In addition, S protein contains cleavage sites for cell proteases FURIN and transmembrane serine protease 2 (TMPRSS2) that allow viral cell entrance.

### SUMMARY OF THE DISCLOSURE

In an embodiment, the VLC-PUFA is provided as a pharmaceutical composition. In a further embodiment, the pharmaceutical composition comprises a composition for topical administration, a composition for intranasal administration, a composition for oral administration, or a composition for parenteral administration. In a further embodiment, the composition for intranasal administration comprises an inhalant. In an embodiment, the pharmaceutical composition further comprises one or more additional active agents such as anti-oxidants, PAF-receptor antagonists, or antivirals.

In an embodiment, the viral inflammatory response or viral disease is indicated by increased production of pro-inflammatory cytokines and chemokines by a cell. In a further embodiment, the pro-inflammatory cytokines and chemokines comprise at least one of IL-6, IL-1β, IL-8/CXCL8, CCL2/MCP-1, CXCL1/KC/GRO, VEGF, ICAM1(CD54). In an embodiment, the VLC-PUFA abrogates the production of pro-inflammatory cytokines and chemokines. In an embodiment, the cell comprises an epithelial cell. In a further embodiment, the epithelial cell comprises a respiratory epithelial cell. In a further embodiment, the respiratory epithelial cells comprise bronchioles cells and alveoli cells.

In an embodiment, the VLC-PUFA is administered topically, orally, intranasally, or parenterally.

In an embodiment, the therapeutically effective amount comprises about 500 nM concentration, greater than about 500 nM concentration, or less than about 500 nM concentration.

In an embodiment the VLC-PUFA is administered prior to exposure to a virus, at about the same time as exposure to a virus, or after exposure to a virus.

In an embodiment, the viral inflammatory response or viral disease comprises a coronavirus infection. In a further embodiment, the coronavirus infection comprises SARS-CoV-2 infection.

Aspects of the invention are drawn to a method of treating or preventing a viral infection or symptom thereof in a subject. In embodiments the method comprises administering to the subject a therapeutically effective amount of a very long chain polyunsaturated fatty acid (VLC-PUFA), arachidonic acid, docosahexaenoic acid, or a derivative thereof.

In embodiments, the treating or preventing comprises reducing an immune response. For example, the immune response comprises tissue inflammation. For example, the tissue comprises ocular tissue, brain tissue, gastrointestinal tissue, skin tissue, or heart tissue.

In embodiments, the VLC-PUFA comprises a compound of A or B:

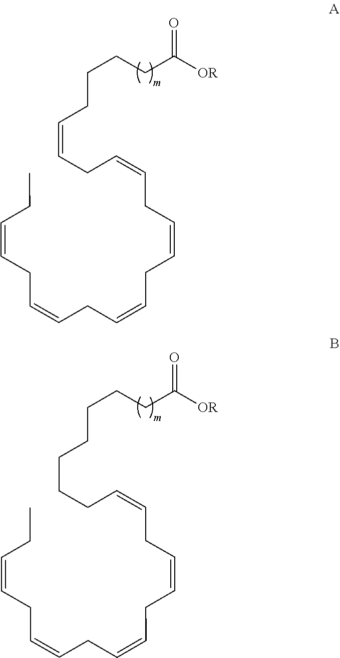

In embodiments, R comprises —H, —OH, methyl, ethyl, propyl, or an alkyl group; wherein m comprises 0-19; or any combination thereof.

In embodiments, the compound comprises (14Z,17Z,20Z,23Z,26Z,29Z)-dotriaconta-14,17,20,23,26,29-hexaenoic acid) or (16Z,19Z,22Z,25Z,28Z,31Z)-tetratriaconta-16,19,22,25,28,31-hexaenoic acid).

In embodiments, the compound comprises a compound of A1 or B1:

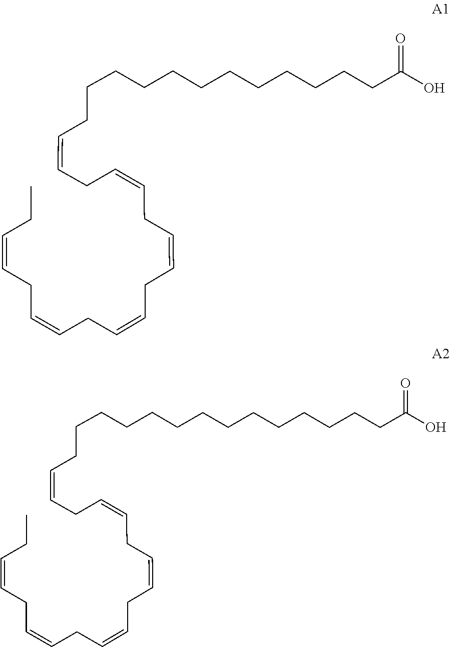 [SMILES: CC/C=C\C/C=C\C/C=C\C/C=C\C/C=C\C/C=C\CCCCCCCCCCCCC(=O)O ; CC/C=C\C/C=C\C/C=C\C/C=C\C/C=C\C/C=C\CCCCCCCCCCCCCCC(=O)O]

In embodiments, the VLC-PUFA derivative comprises:

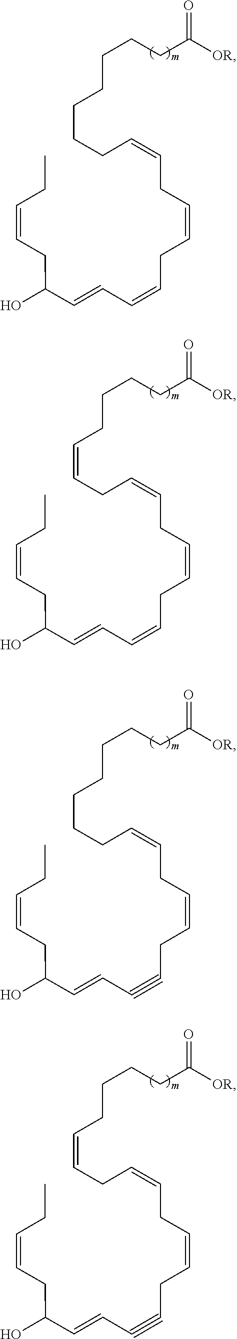

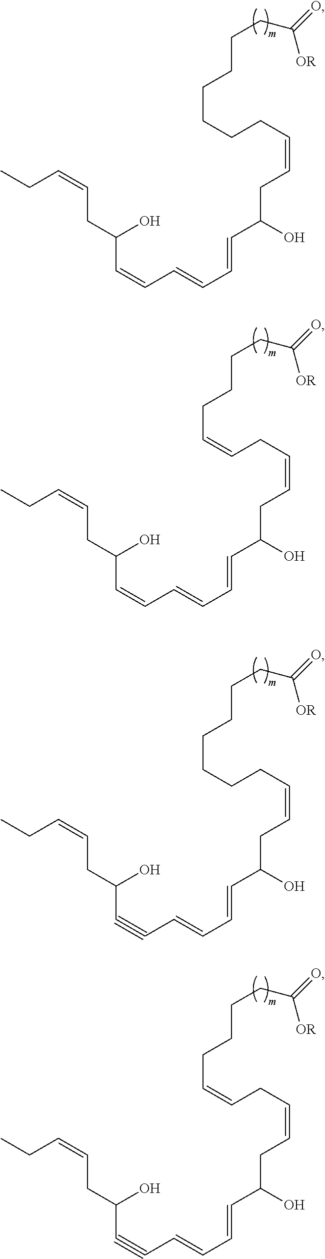

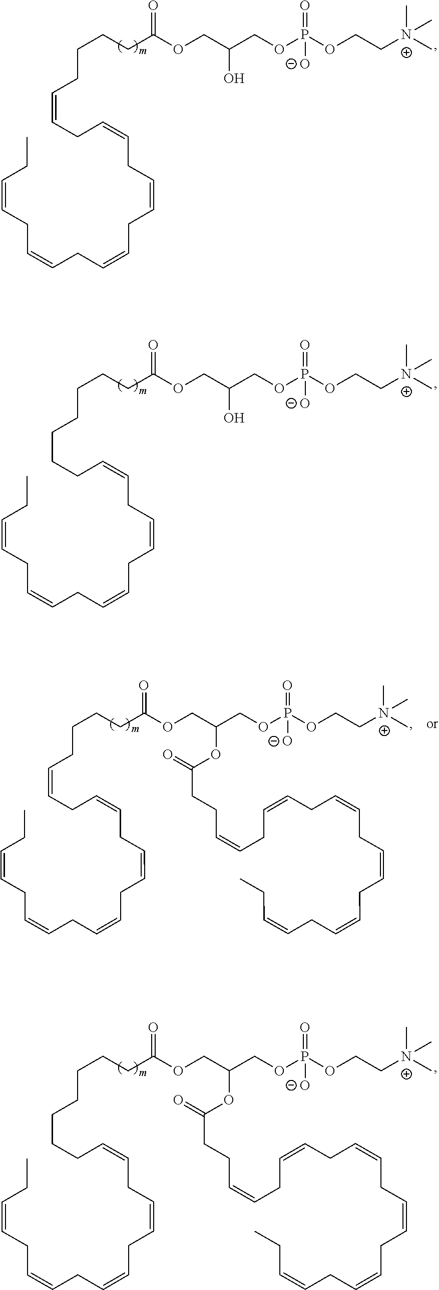

wherein m is selected from a group consisting of 0 to 19; and
wherein —COOR is a carboxylic acid group, a pharmaceutically acceptable carboxylic ester,
or a pharmaceutically acceptable salt thereof.

In embodiments, if —COOR is a carboxylic acid salt, the R group is a cation selected from a group consisting of an ammonium cation, an iminium cation, or a metal cation selected from a group consisting of sodium, potassium, magnesium, zinc, or calcium cation.

In embodiments, if —COOR is a carboxylic ester, the R group is selected from a group consisting of methyl, ethyl, alkyl, a part of a phospholipid, or a derivative thereof.

In embodiments, the arachidonic acid derivative comprises a lipoxin compound:

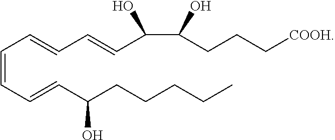 [SMILES: CCCCC[C@@H](O)/C=C/C=C\C=C\C=C\[C@@H](O)[C@@H](O)CCCC(=O)O]

In embodiments, the docosahexaenoic acid derivative comprises a resolving compound:

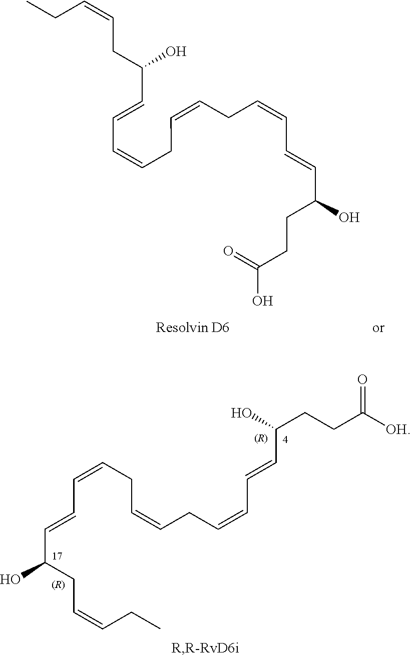

In embodiments, the VLC-PUFA derivative comprises:

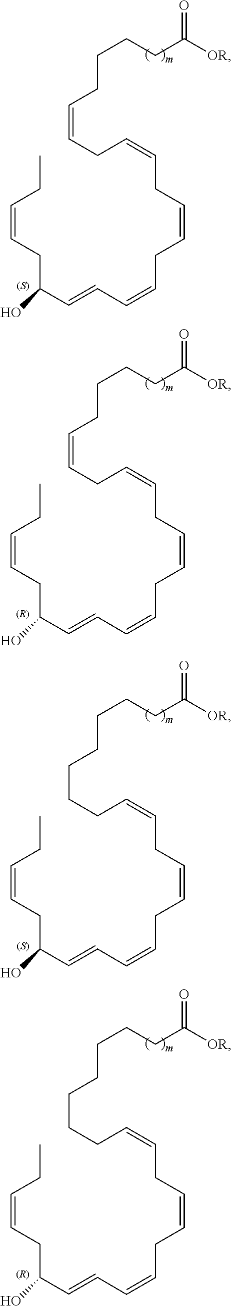

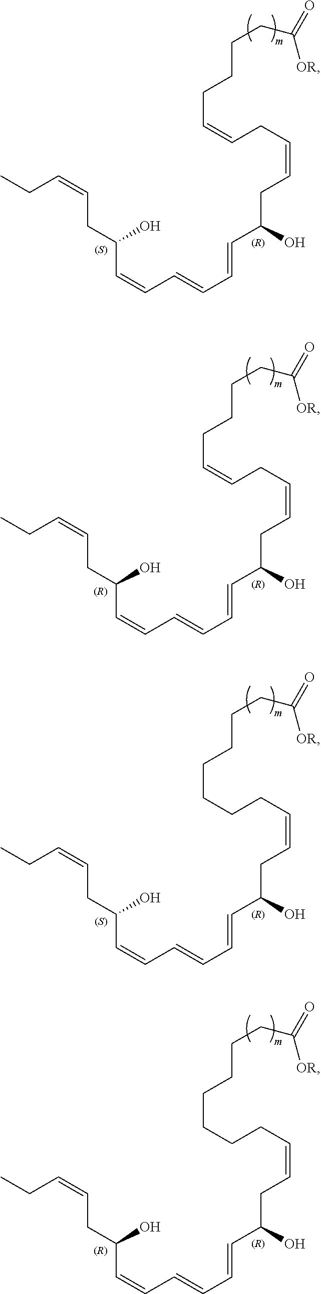

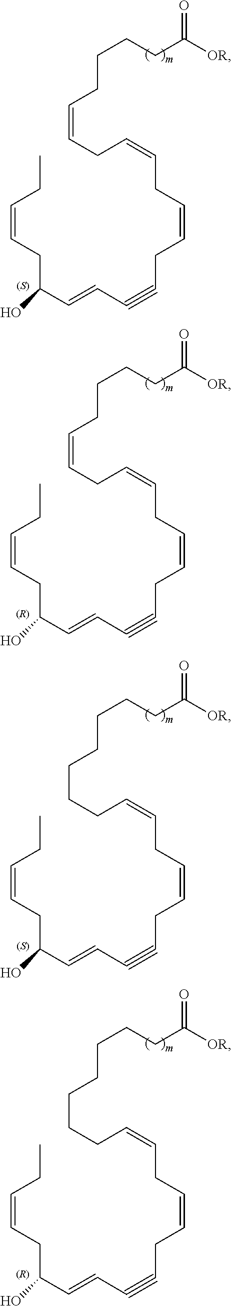

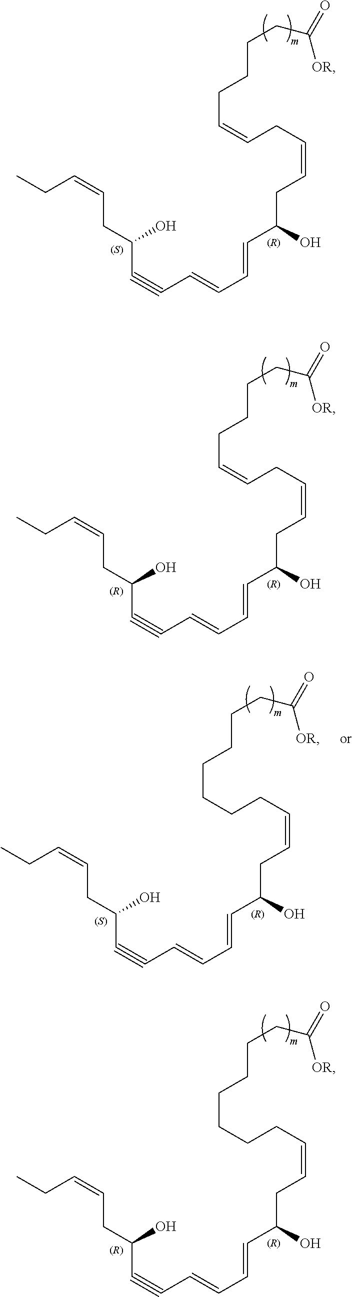

wherein m is selected from a group consisting of 0 to 19; and
wherein —COOR is a carboxylic acid group, a pharmaceutically acceptable carboxylic ester,
or a pharmaceutically acceptable salt thereof.

In embodiments, if —COOR is a carboxylic acid salt, the R group is a cation selected from a group consisting of an ammonium cation, an iminium cation, or a metal cation selected from a group consisting of sodium, potassium, magnesium, zinc, or calcium cation.

In embodiments, if —COOR is a carboxylic ester, the R group is selected from a group consisting of methyl, ethyl, alkyl, a part of a phospholipid, or a derivative thereof.

In embodiments, the VLC-PUFA derivative comprises:

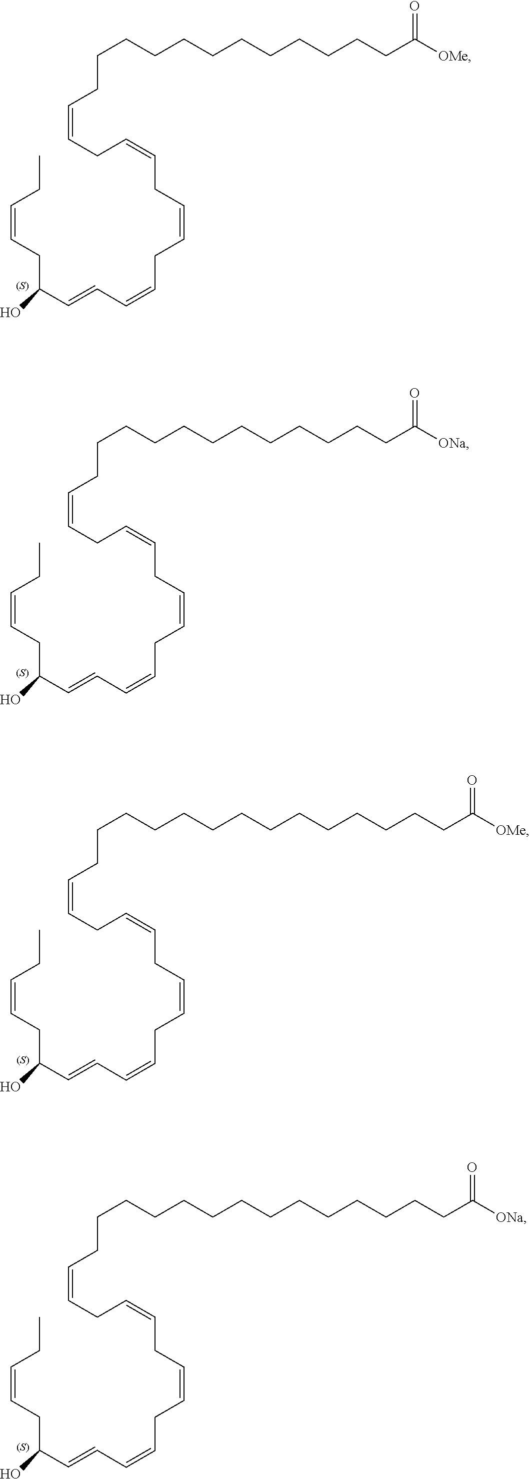

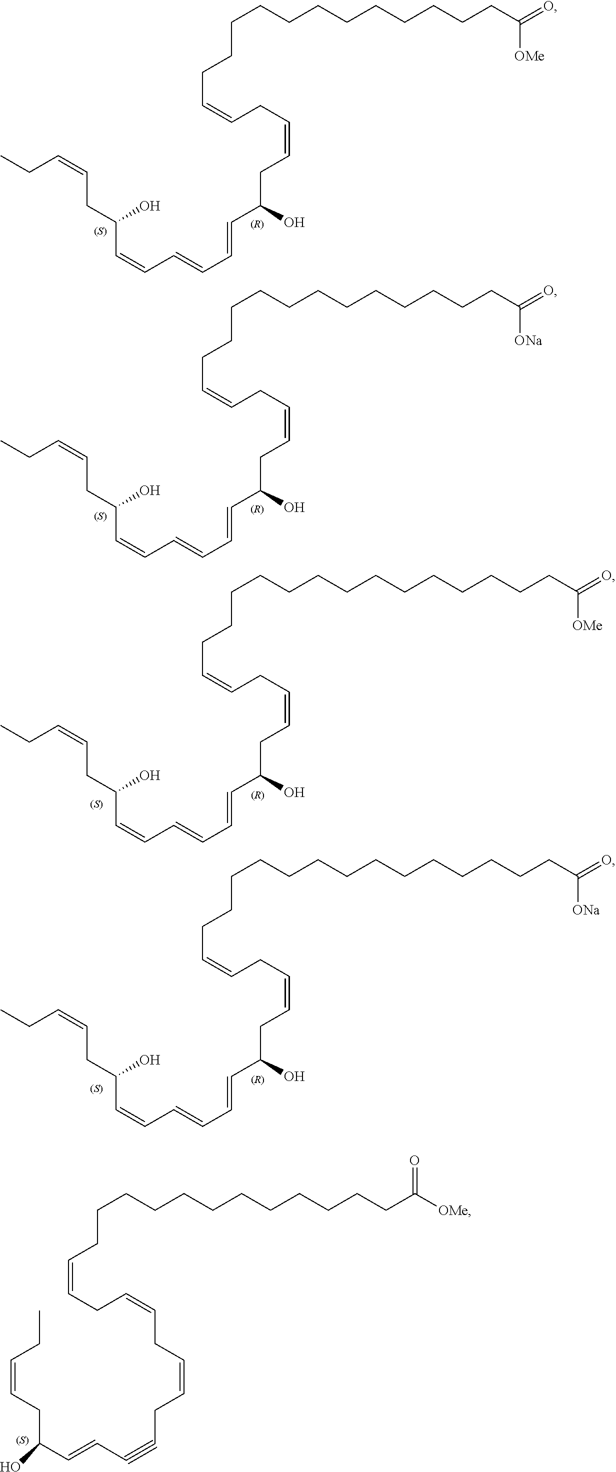

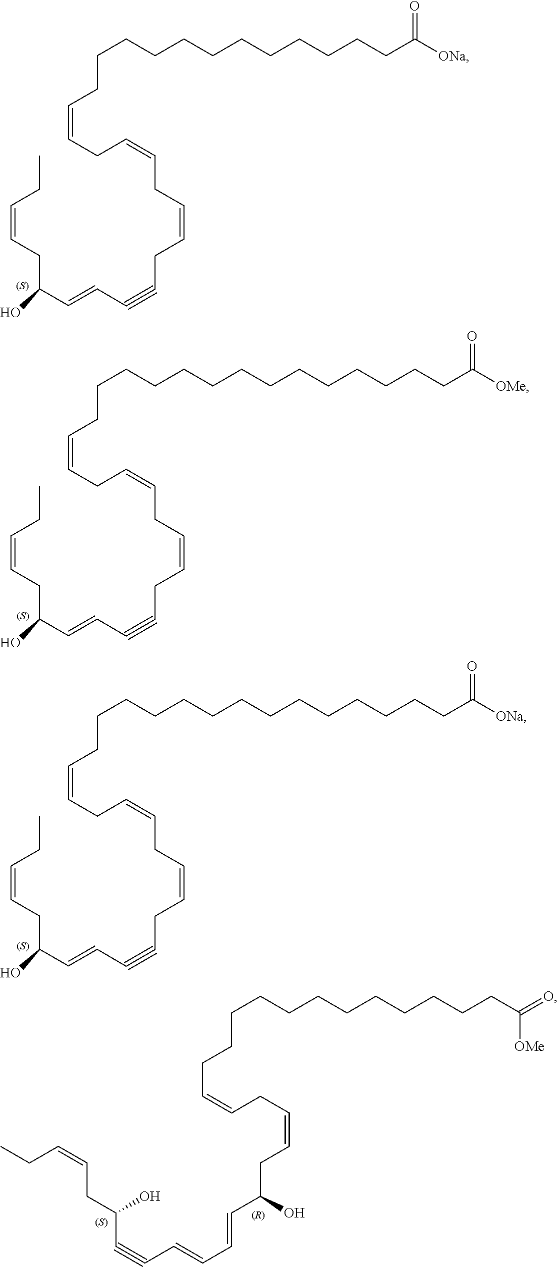

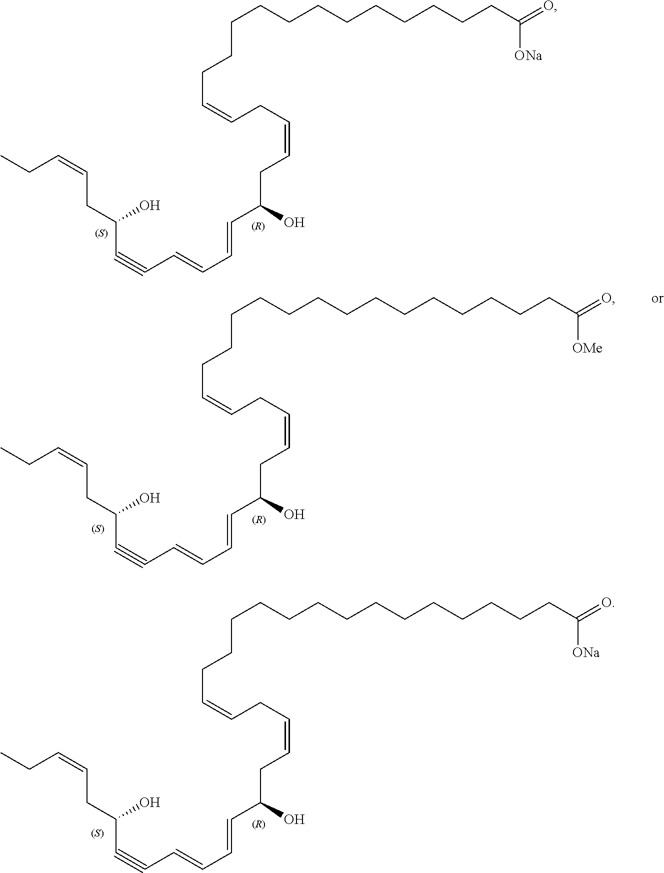

In embodiments, the compound is administered as a pharmaceutical composition.

In embodiments, the pharmaceutical composition is administered topically, intranasally, orally, ocularly, parenterally, or nebulized.

In embodiments, the nebulized pharmaceutical comprises an aerosol or spray.

In embodiments, the pharmaceutical composition administered ocularly comprises an eye drop.

In embodiments, the method further comprises administering to the subject one or more active agents. For example, the agent comprises an anti-inflammatory, a pain reliever, an antioxidant, a PAF-receptor antagonist, or an antiviral.

In embodiments, the VLC-PUFA, arachidonic acid, docosahexaenoic acid, or derivative thereof is administered prior to, subsequent to, or concurrently with onset of viral infection.

In embodiments, the immune response is indicated by increased production of pro-inflammatory cytokines, chemokines, or a combination thereof. For example, the pro-inflammatory cytokines and chemokines comprise IL-6, IL-β, IL-8/CXCL8, CCL2/MCP-1, CXCL1/KC/GRO, VEGF, or ICAM1(CD54).

In embodiments, the therapeutically effective amount comprises a concentration of about 500 nM to about 700 nM.

In embodiments, the viral infection comprises a coronavirus infection, an influenza virus infection, or an adenovirus infection.

The method of claims 28, wherein the coronavirus comprises SARS-CoV-2.

The disclosure is focused on compounds, compositions and methods for applications to alleviate a symptom of, preventing, or treating a viral infection, viral disease, or viral induced inflammatory/immune response in a subject. Further aspects of the disclosure will be readily appreciated upon review of the detailed description of its various embodiments, described below, when taken in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a scheme illustrating the postulated biosynthesis of elovanoids (ELV) from omega-3 (n-3 or n3) very long chain polyunsaturated fatty acids (n3 VLC-PUFA).

FIG. 2 is a scheme illustrating the biosynthesis of n3 VLC-PUFA.

FIGS. 3A-3K illustrate the generation and structural characterization of elovanoids ELV-N32 and ELV-N34 from cultured primary human retinal pigment epithelial cells (RPE).

FIG. 3A is a scheme illustrating ELV-N32 and ELV-N34 synthesis from the intermediates (1, 2, and 3), each of which was prepared in stereochemically-pure form. The stereochemistry of intermediates 2 and 3 was pre-defined by using enantiomerically-pure epoxide starting materials. The final ELVs (4) were assembled via iterative couplings of intermediates 1, 2, and 3, and were isolated as the methyl esters (Me) or sodium salts (Na).

FIG. 3B illustrates the elution profile of C32:6n3, endogenous mono-hydroxy-C32:6n3, and ELV-N32 shown with ELV-N32 standard. MRM of ELV-N32 shows two large peaks eluted earlier than the peak when standard ELV-N32 is eluted, displaying the same fragmentation patterns (shown in the insert spectra), suggesting that they are isomers.

FIG. 3C illustrates the chromatogram for full daughter scans for ELV-N32 and ELV-N34.

FIG. 3D illustrates the fragmentation pattern of ELV-N32.

FIG. 3E illustrates the elution profile of C34:6n3, ELV-N34, 29 monohydroxy-34:6, and endogenous ELV-N34 and isomers.

FIG. 3F illustrates the UV spectrum of endogenous ELV-N34 showing triene features, with λmax at 275 nm and shoulders at 268 and 285 nm.

FIG. 3G illustrates the fragmentation pattern of ELV-N34.

FIG. 3H illustrates the full fragmentation spectra of endogenous ELV.

FIG. 3I illustrates the ELV standard shows that all major peaks from standard match to the endogenous peaks. However, endogenous ELV has more fragments that don't show up in the standard, suggesting that it includes different isomers.

FIG. 3J illustrates the full fragmentation spectra of endogenous ELV-N34 peaks match to standard ELV-N34.

FIG. 3K illustrates the existence of ELV-N34 isomers.

FIGS. 4A-4K illustrate the structural characterization of elovanoids ELV-N32 and ELV-N34 from neuronal cell cultures. Cerebral cortical mixed neuronal cells were incubated with 32:6n3 and 34:6n3 10 μM each under OGD conditions.

FIG. 4A is a scheme illustrating ELV-N32 and ELV-N34 synthesis from the intermediates (a, b, and c), each of which was prepared in stereochemically-pure form. The stereochemistry of intermediates b and c was pre-defined by using enantiomerically-pure epoxide starting materials. The final ELVs (d) were assembled via iterative couplings of intermediates a, b, and c, and were isolated as the methyl esters (Me) or sodium salts (Na).

FIG. 4B shows retention times of the 32:6n3, endogenous mono-hydroxy-32:6, ELV-N32, and ELV-N32. MRM of ELV-N32 shows two large peaks eluted earlier than the peak when standard ELV-N32 is eluted, but they show the same fragmentation patterns, indicating that they are isomers.

FIG. 4C illustrates the same features as in FIG. 4A, were shown in 34:6n3 and ELV-N34.

FIG. 4D illustrates the UV spectrum of endogenous ELV-N32 shows triene features.

FIG. 4E illustrates the full fragmentation spectra of endogenous ELV-N32.

FIG. 4F illustrates the UV spectrum of endogenous ELV-N34 showing triene features, with λₘₐₓ at 275 nm and shoulders at 268 and 285 nm.

FIG. 4G illustrates the fragmentation pattern of endogenous ELV-N34.

FIG. 411 illustrates the full fragmentation pattern of endogenous ELV-N32.

FIG. 41 illustrates that the ELV-N34 standard shows major peaks from the standard match to the endogenous peaks; endogenous ELV-N34 has more fragments that do not show up in the standard. Without wishing to be bound by theory, this indicates that it can contain isomers.

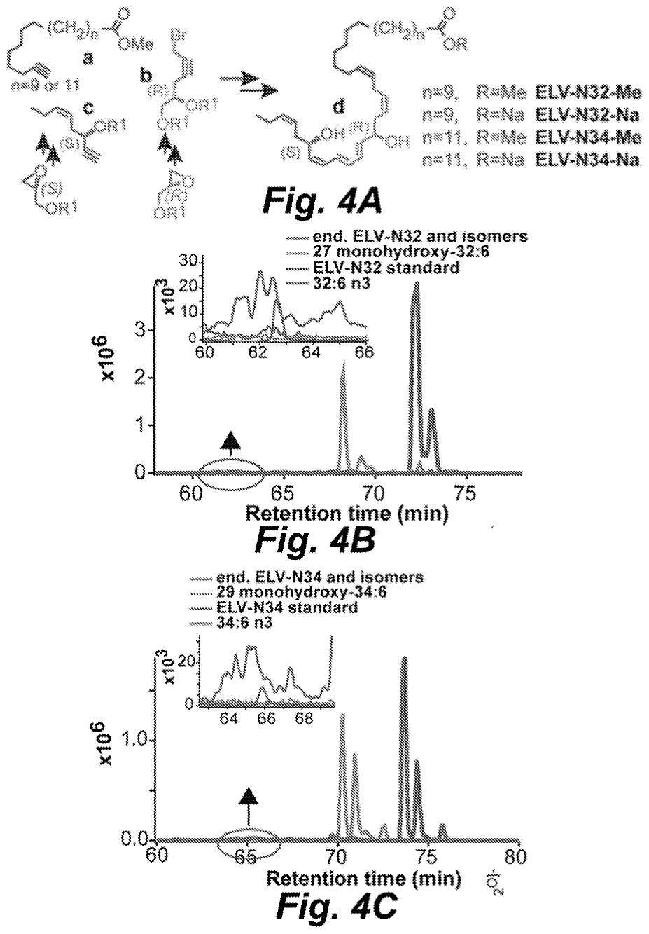

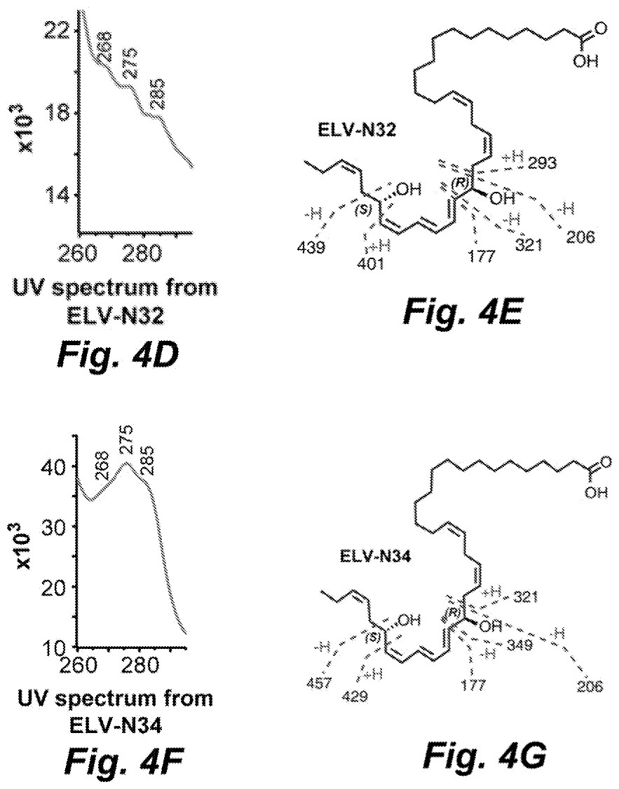

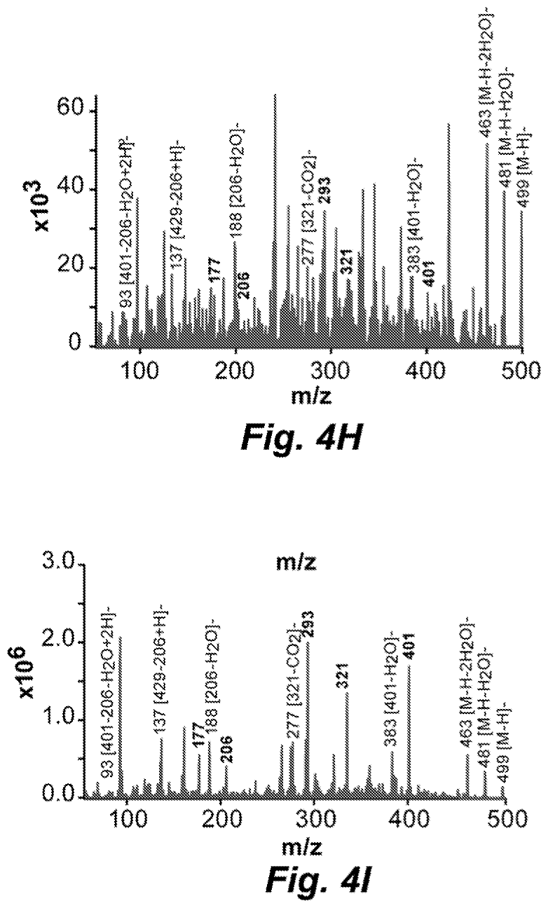

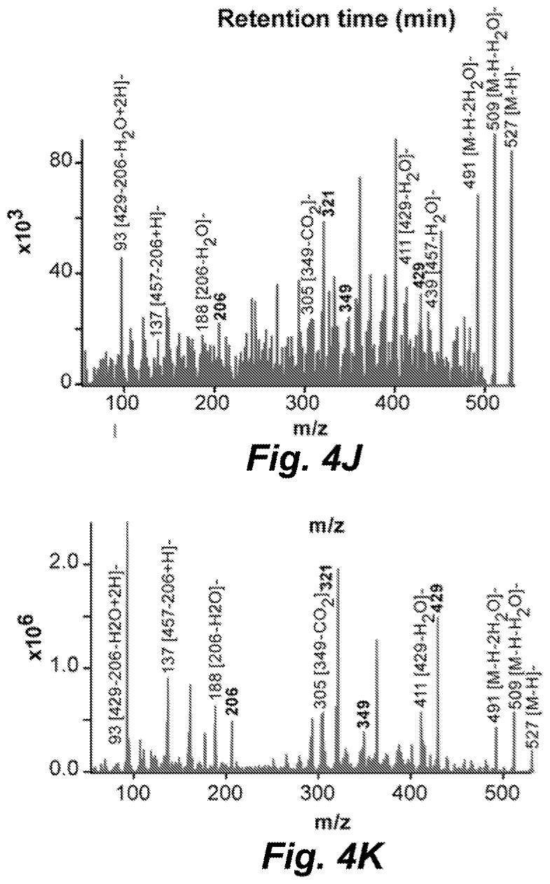

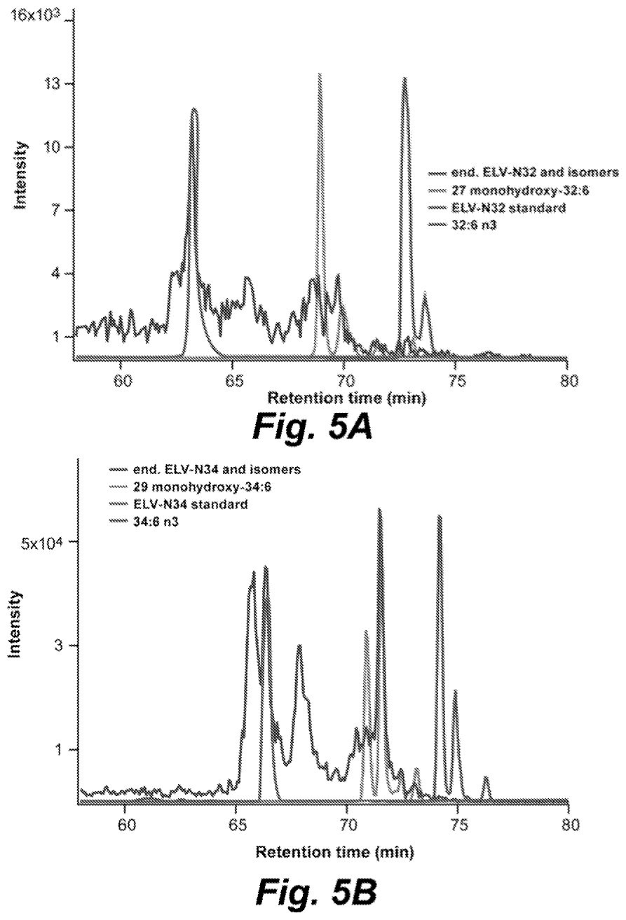

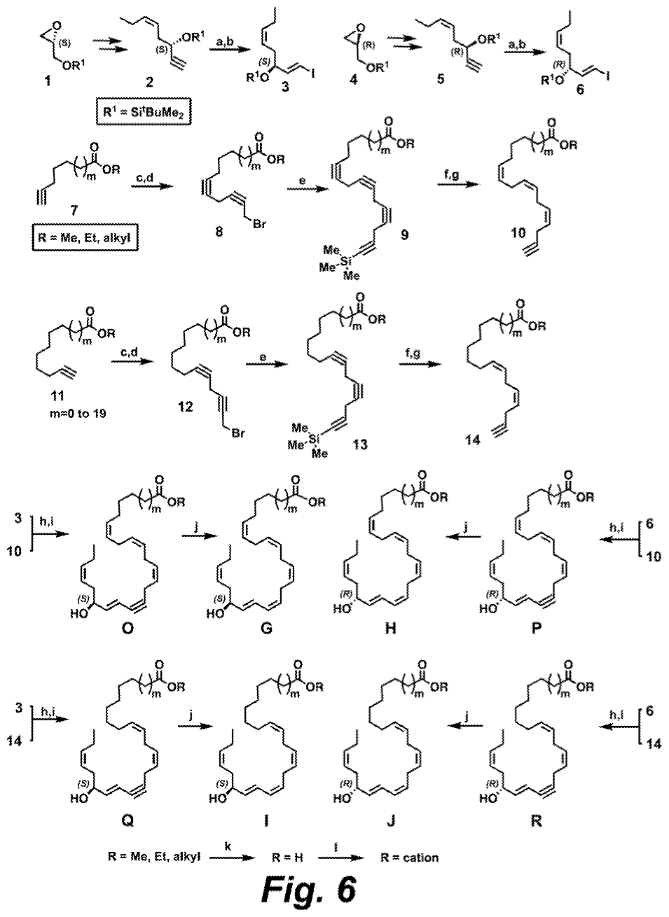

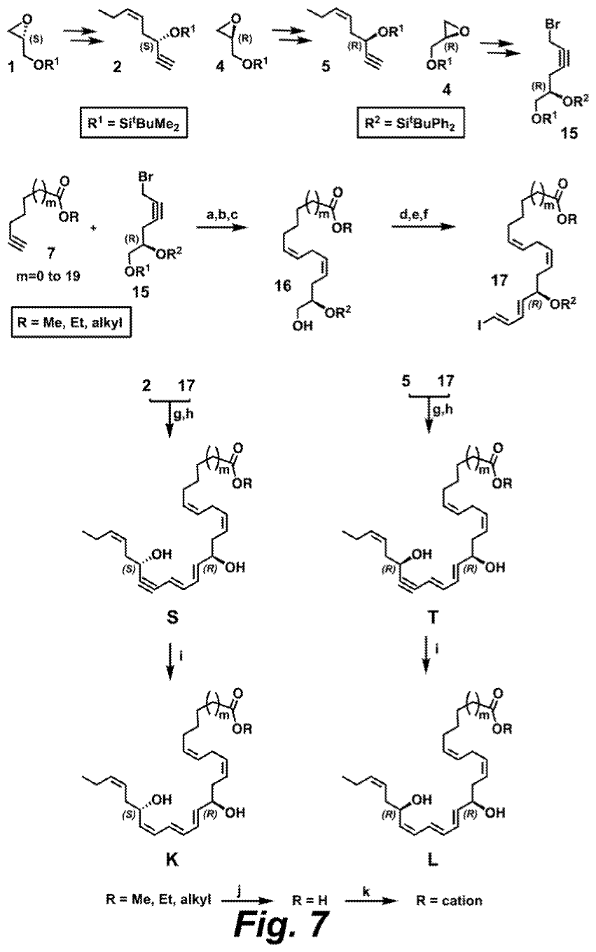

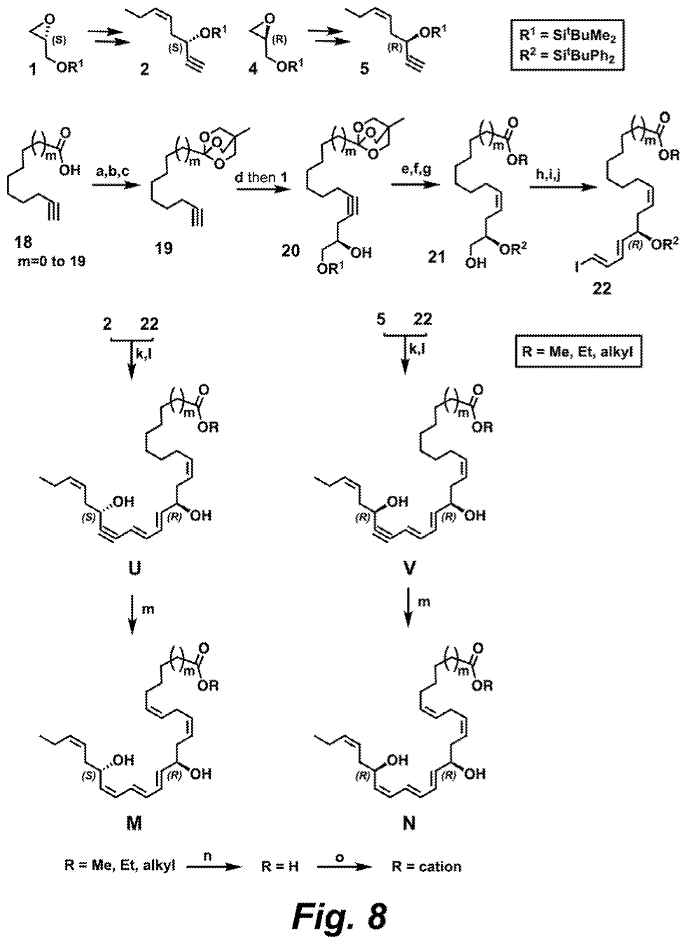

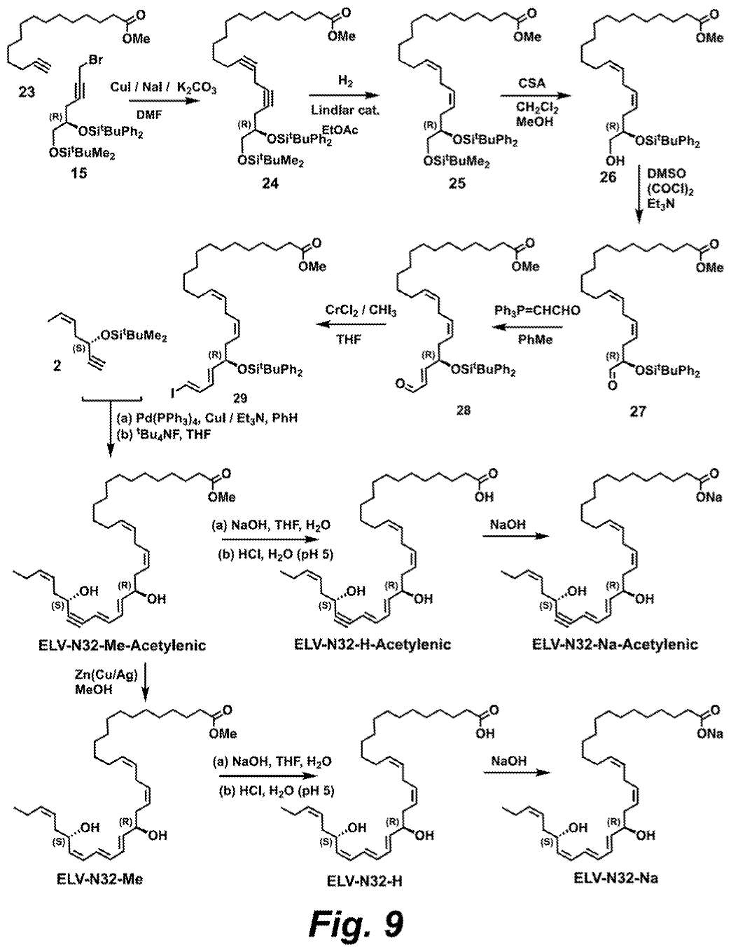

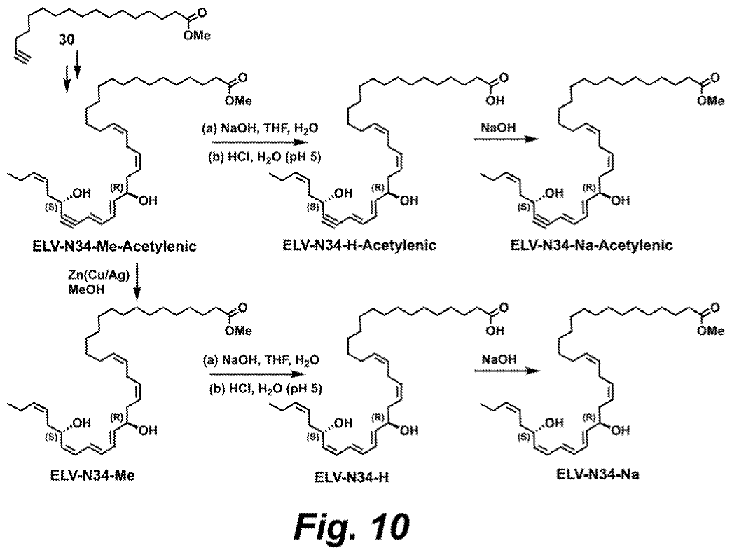

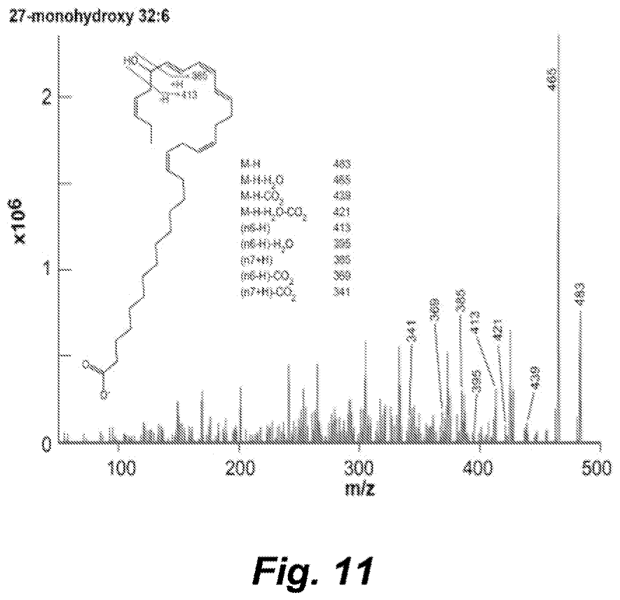

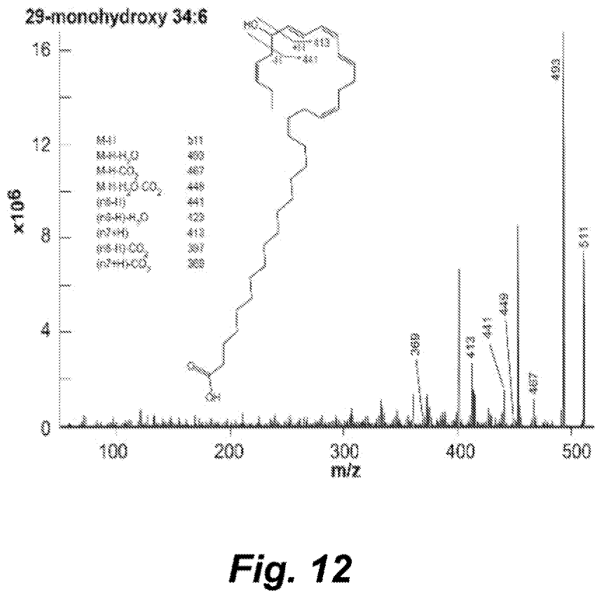

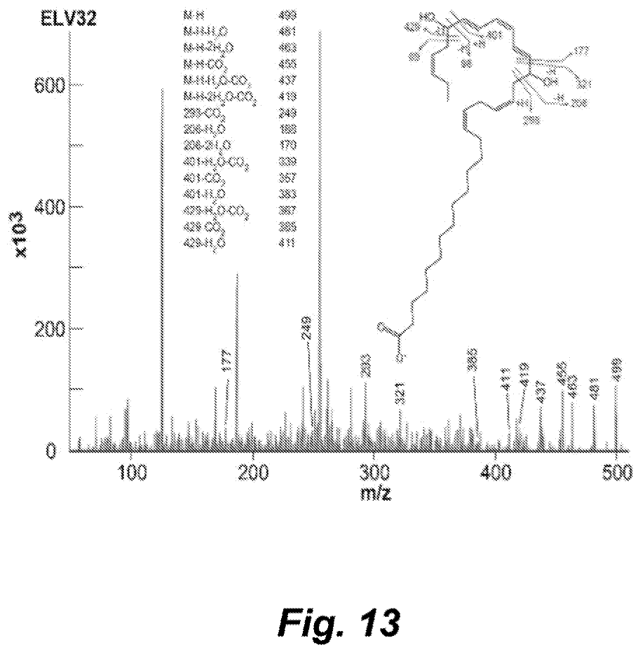

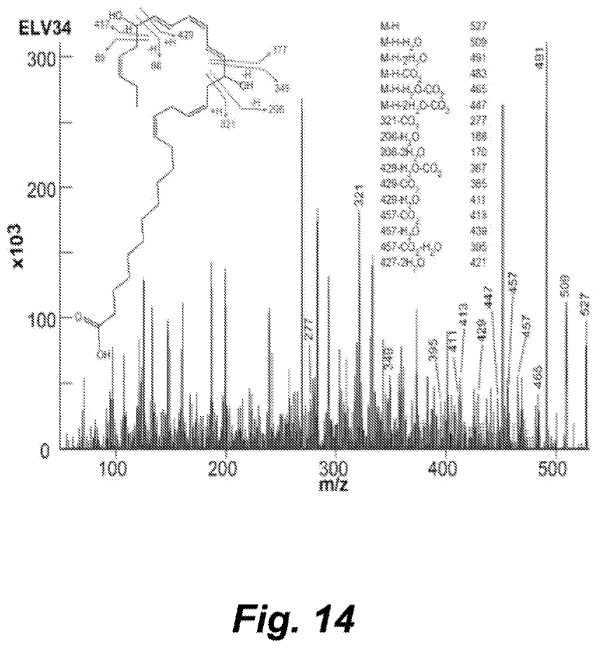

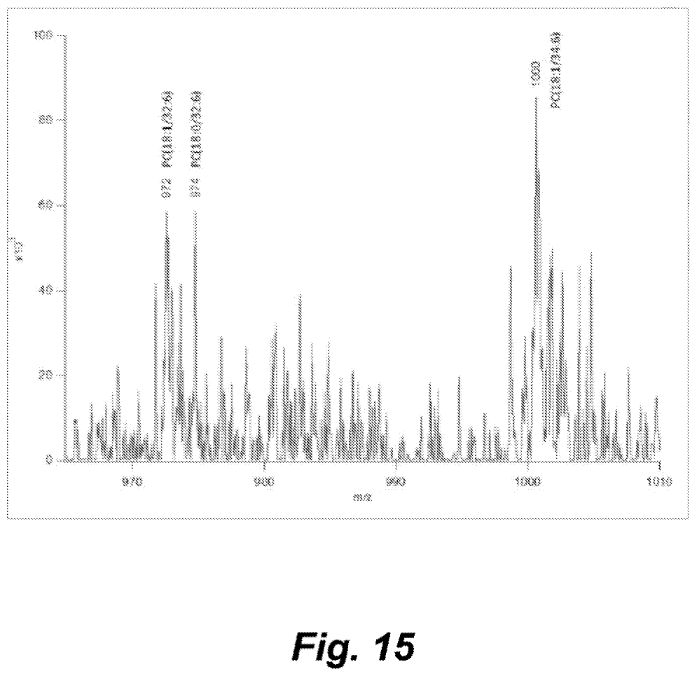

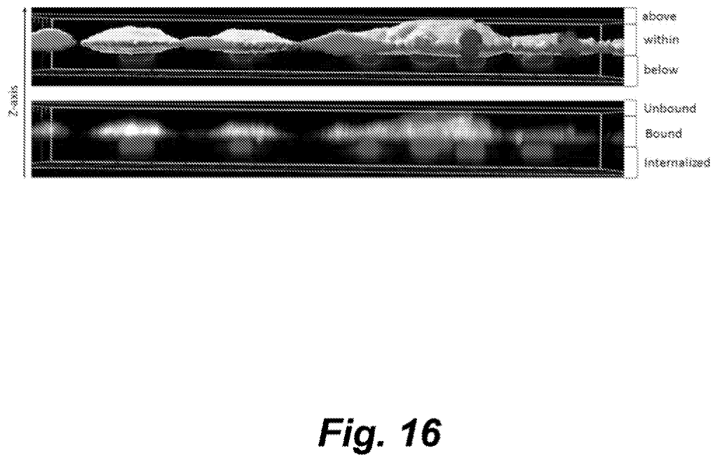

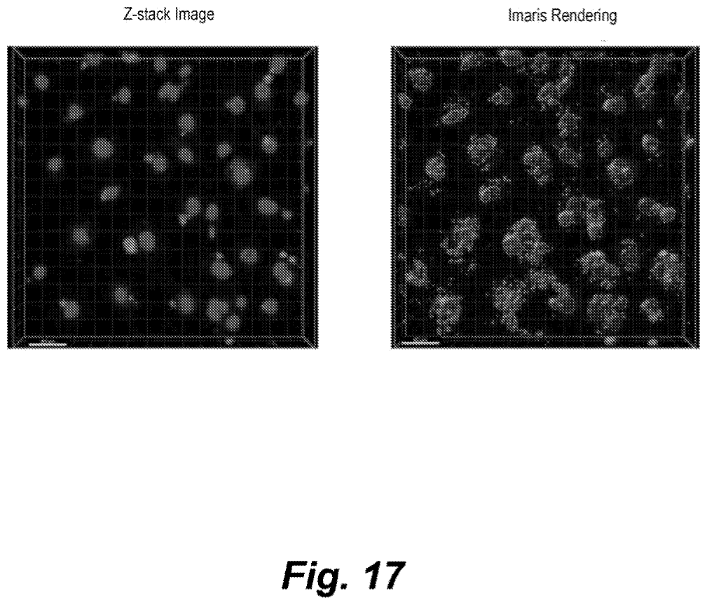

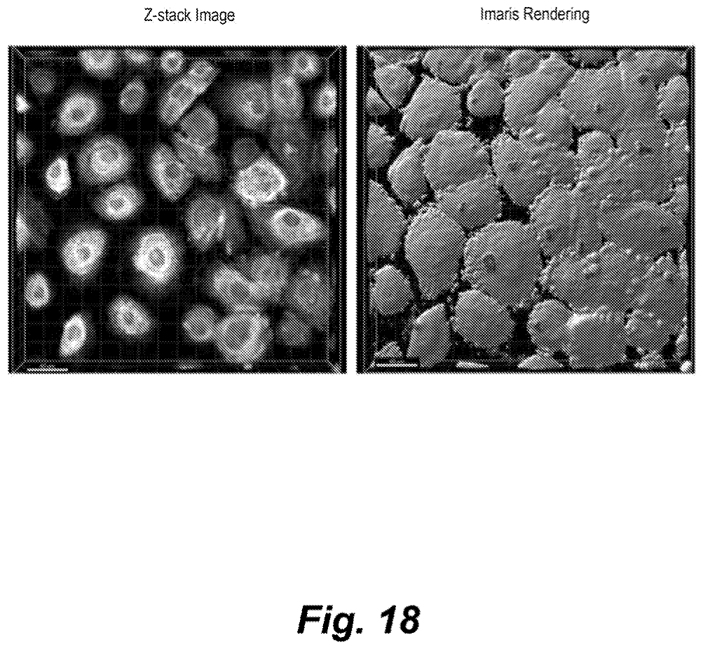

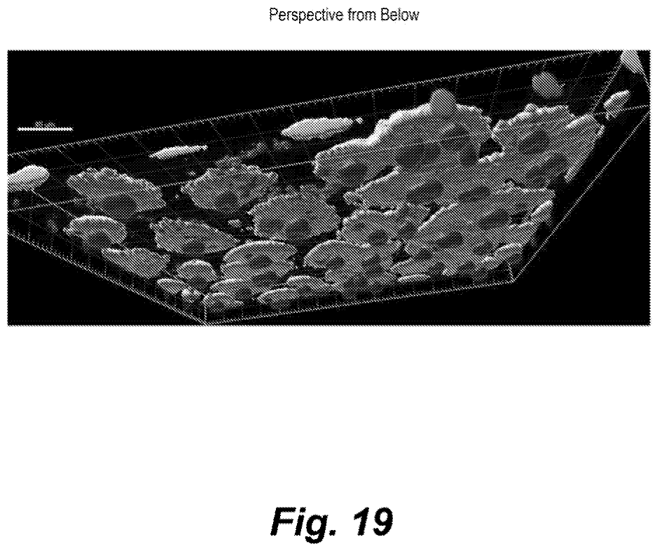

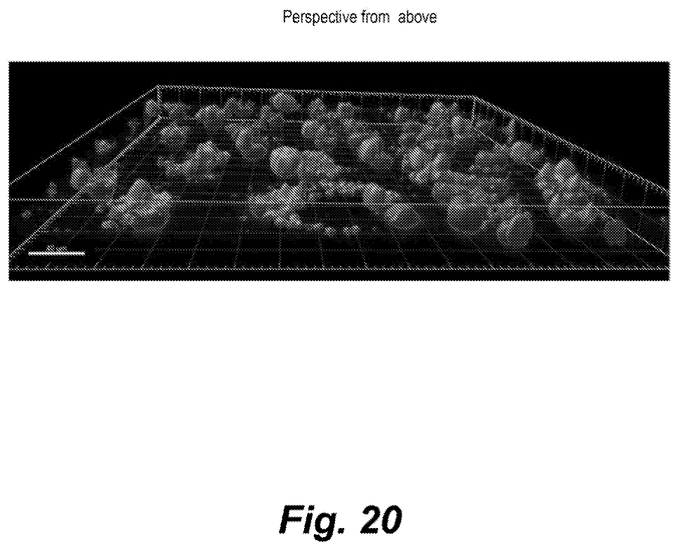

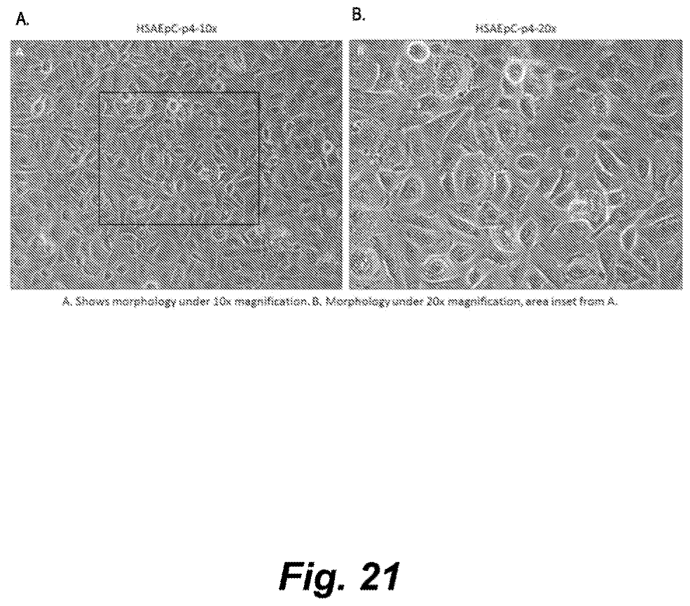

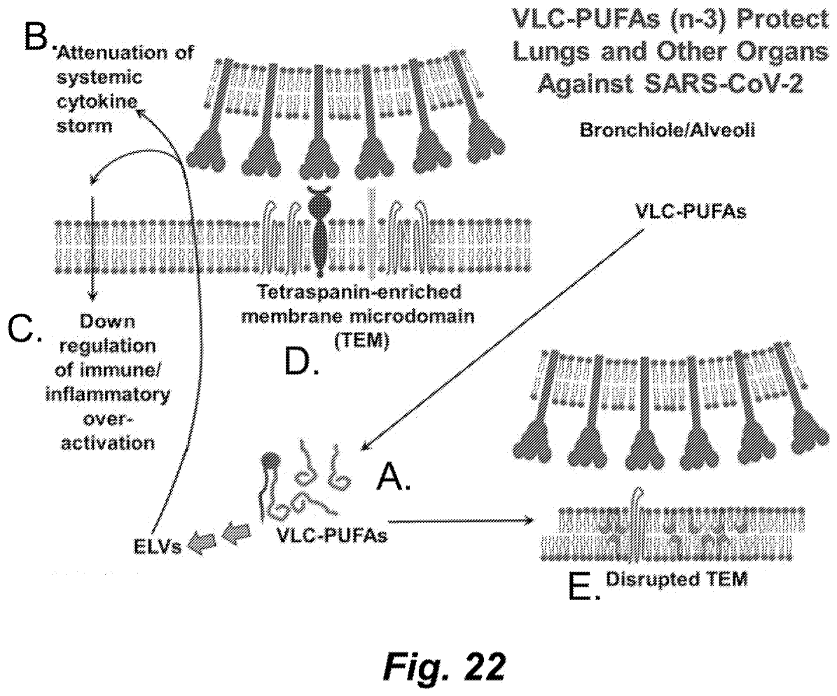

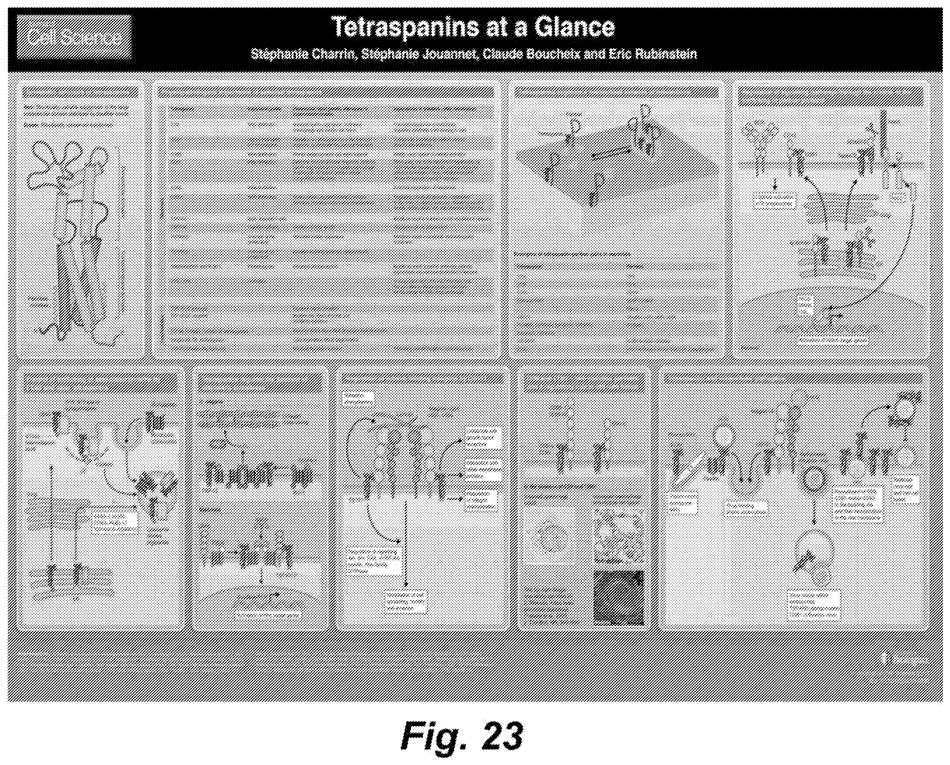

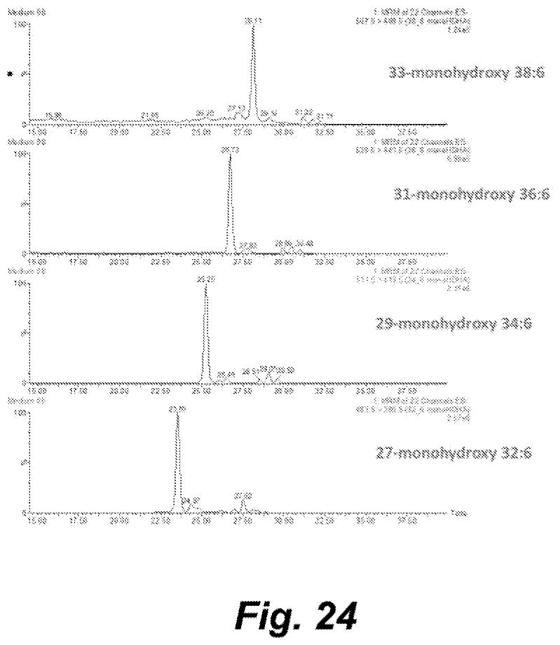

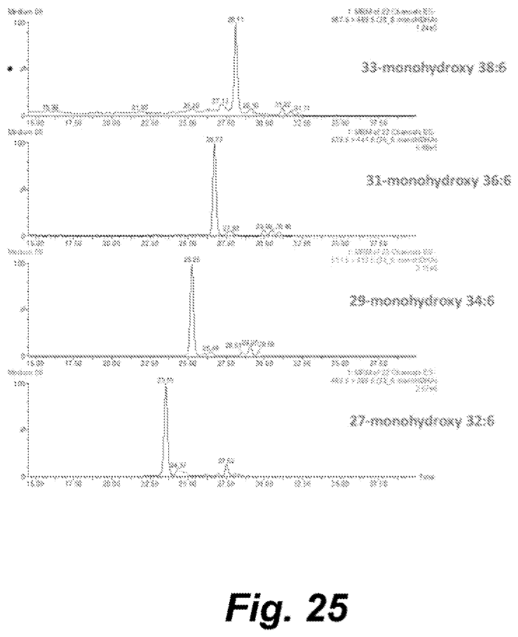

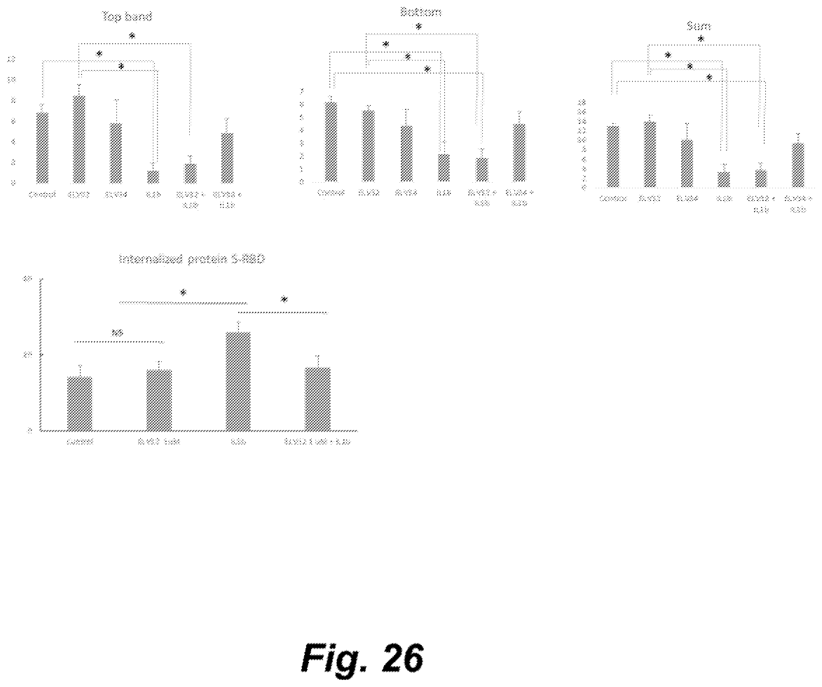

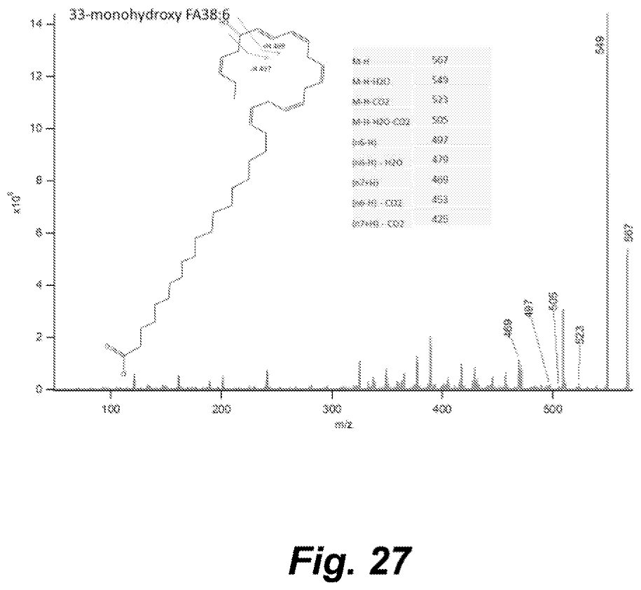

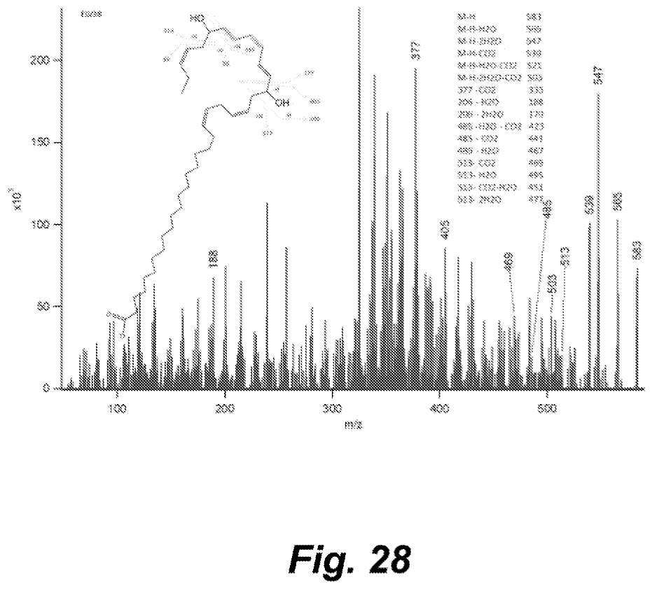

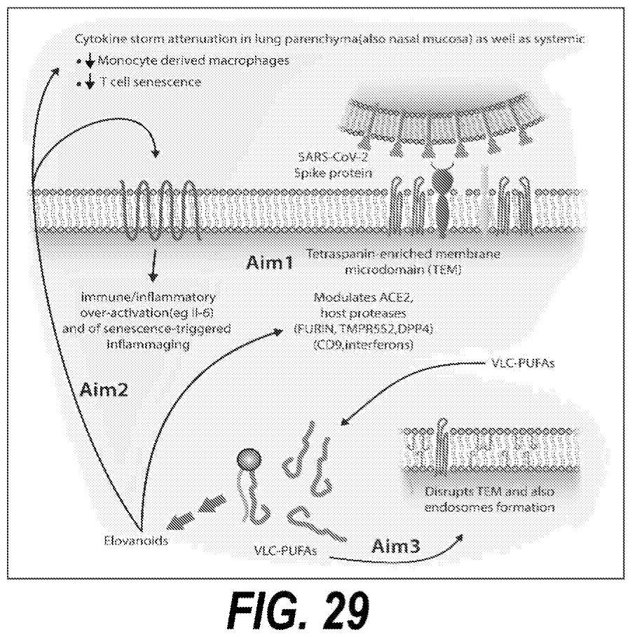

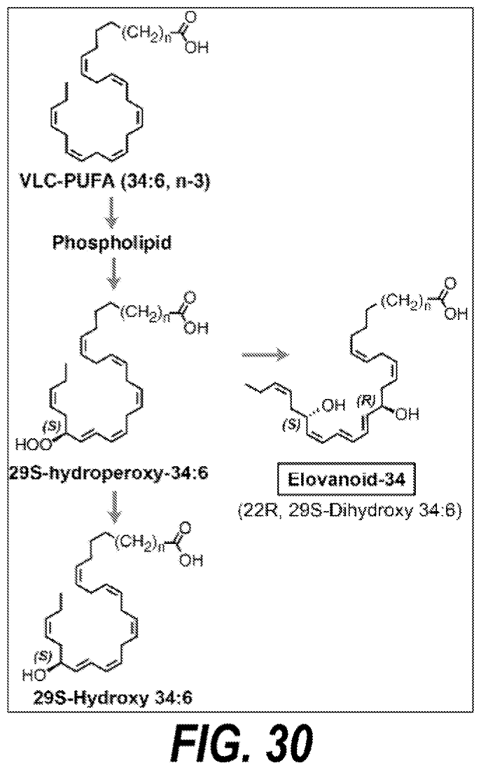

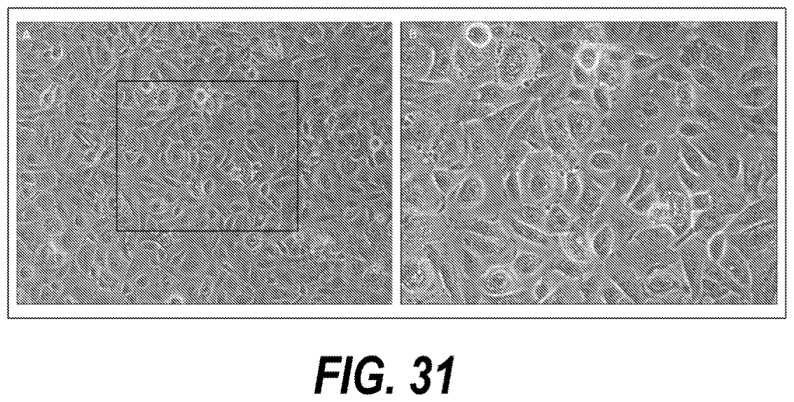

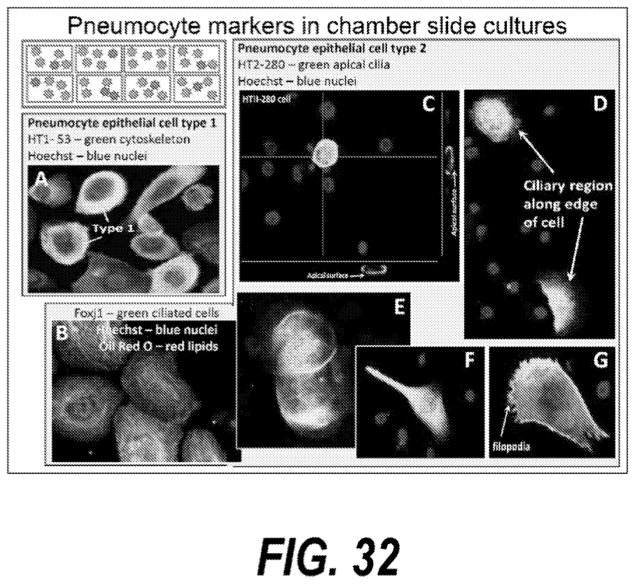

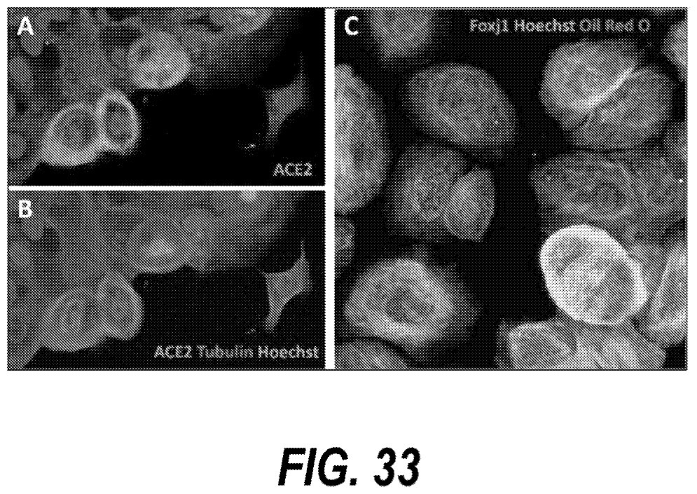

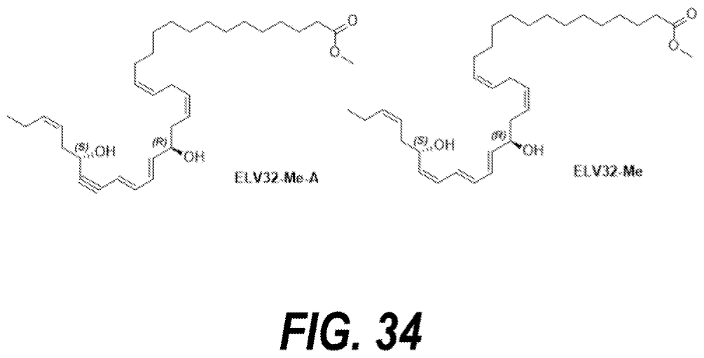

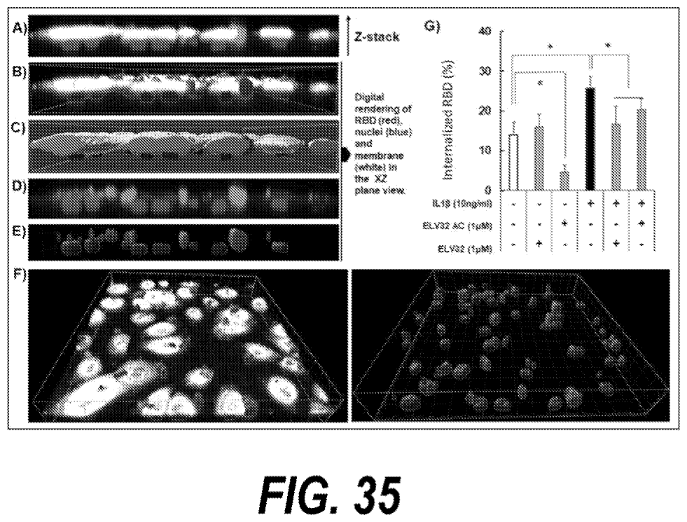

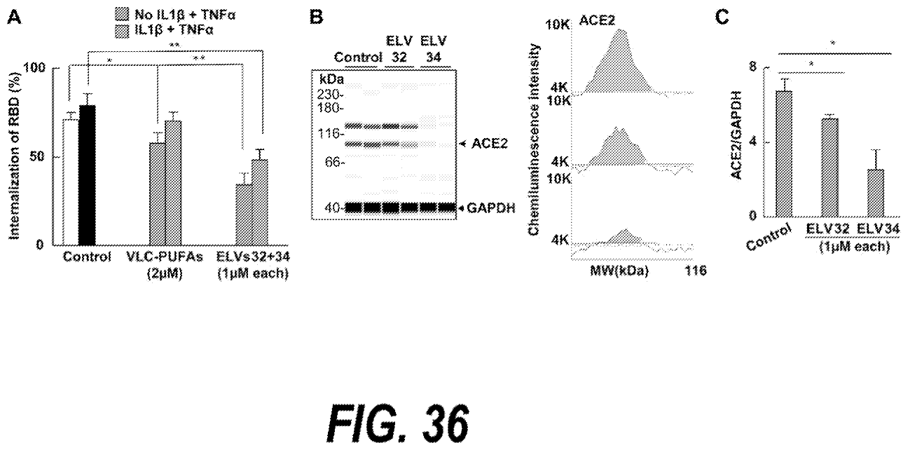

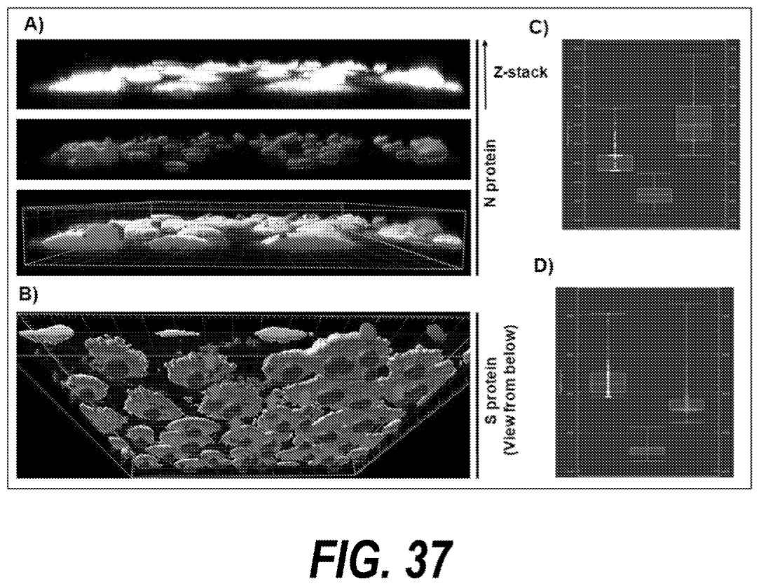

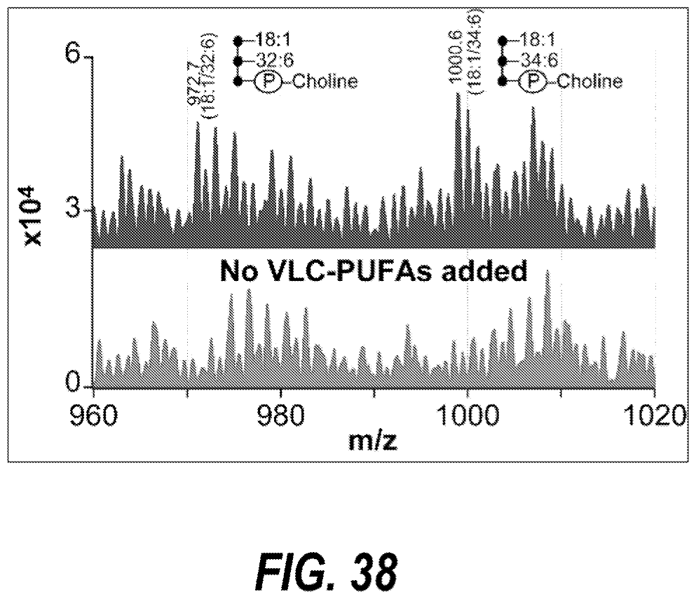

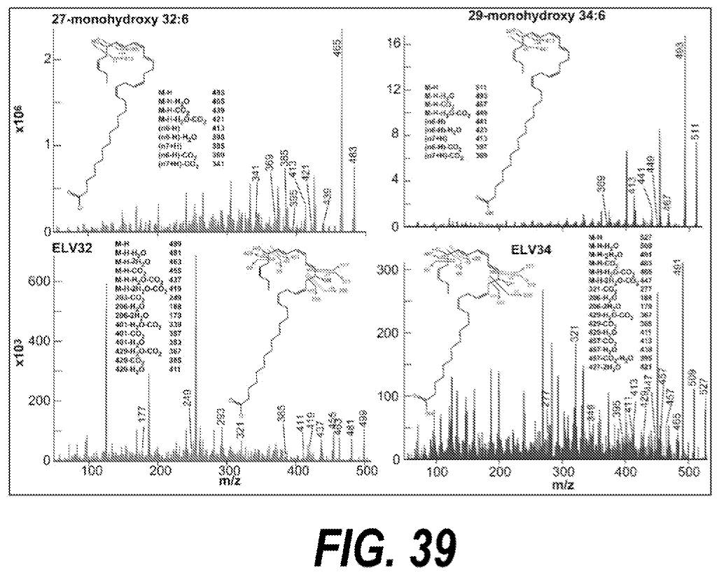

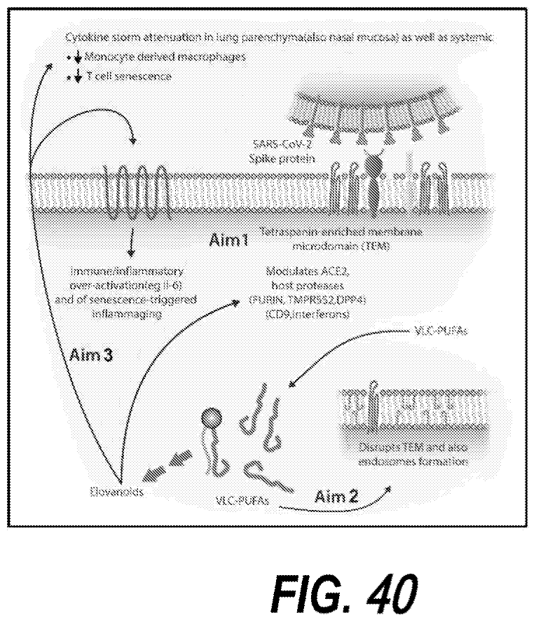

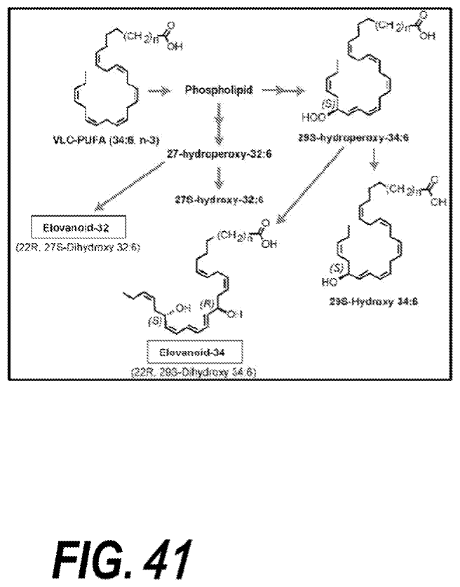

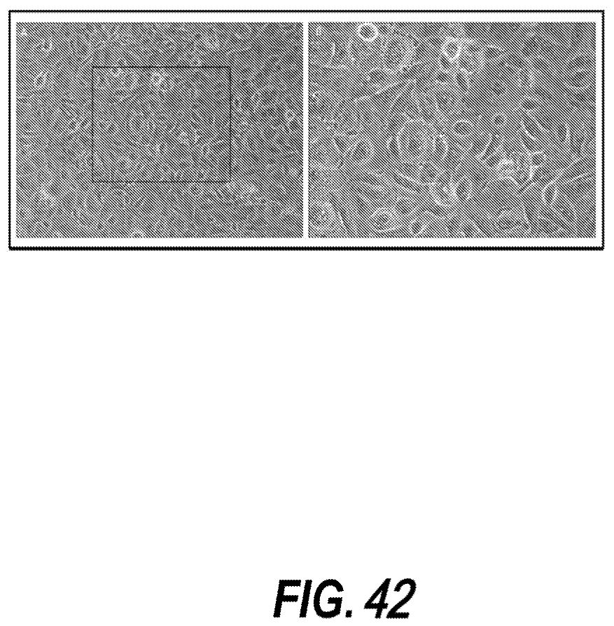

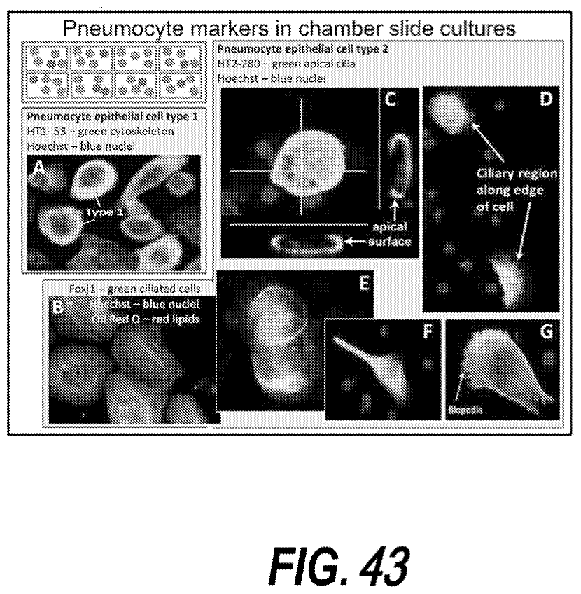

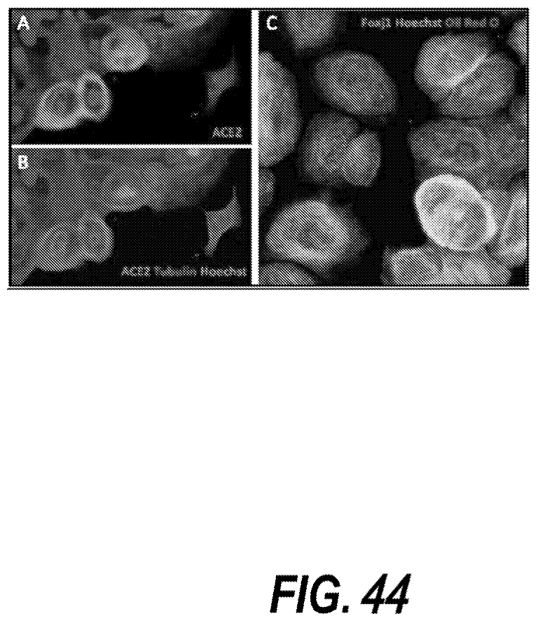

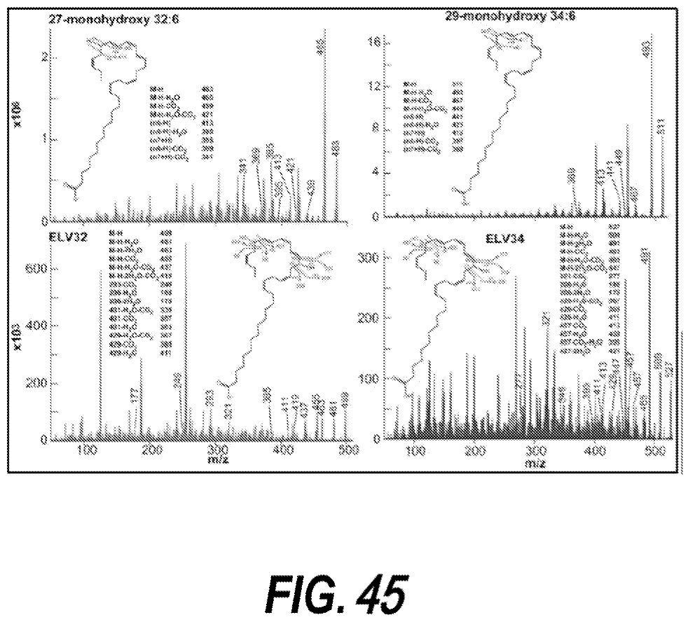

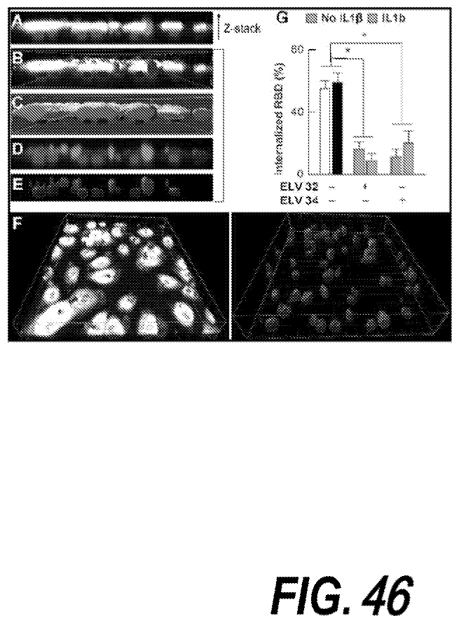

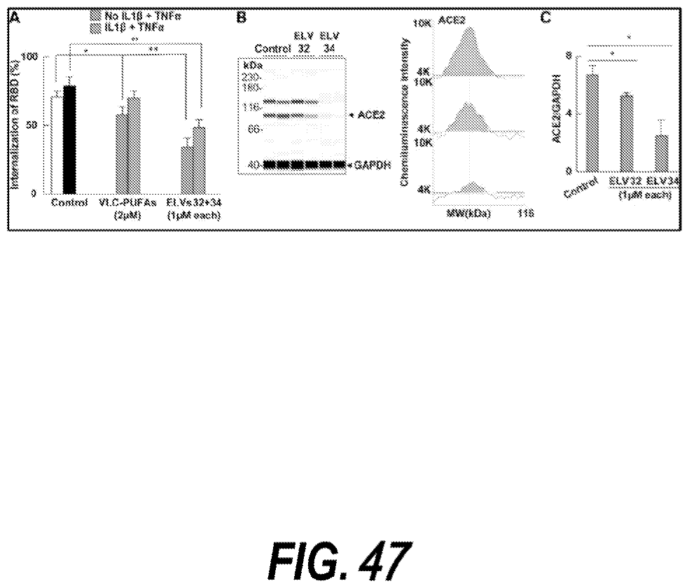

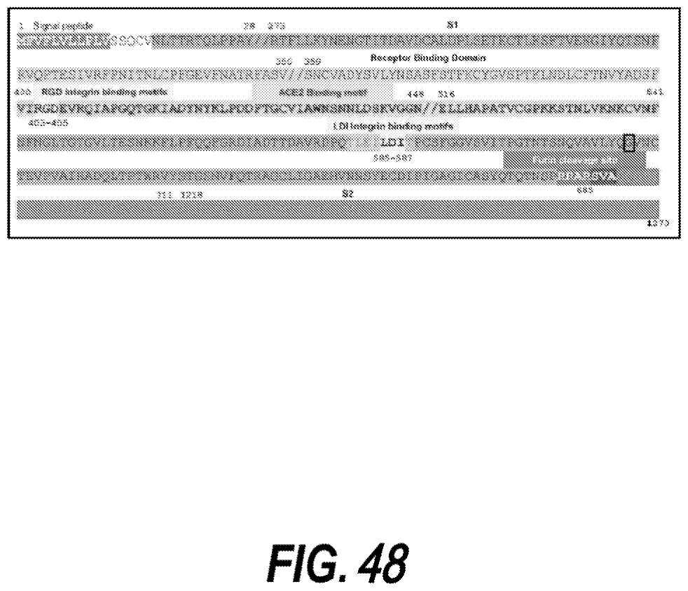

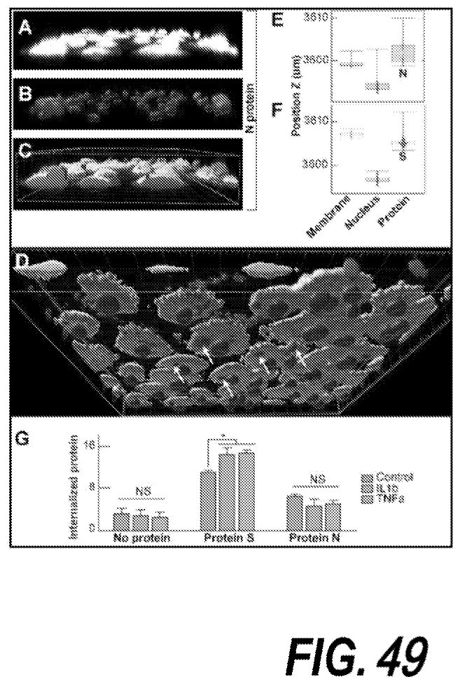

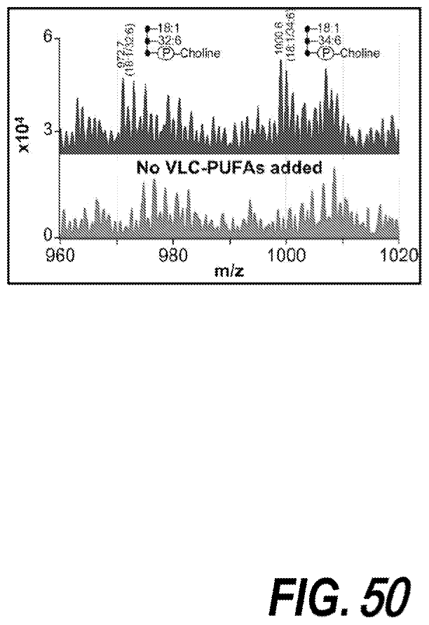

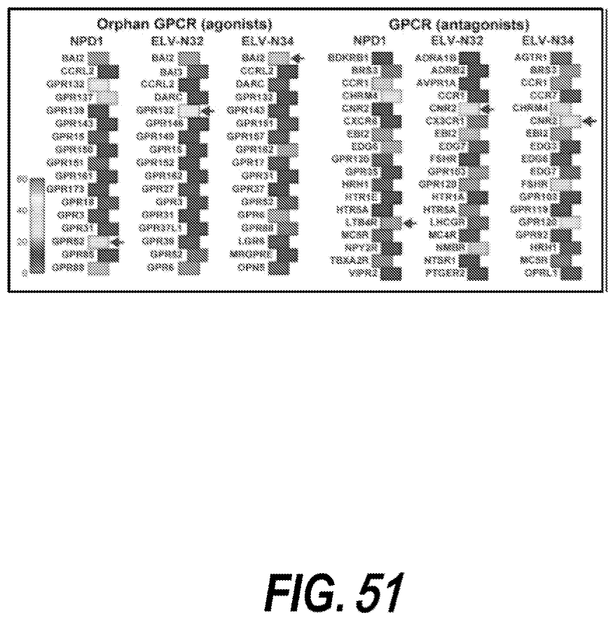

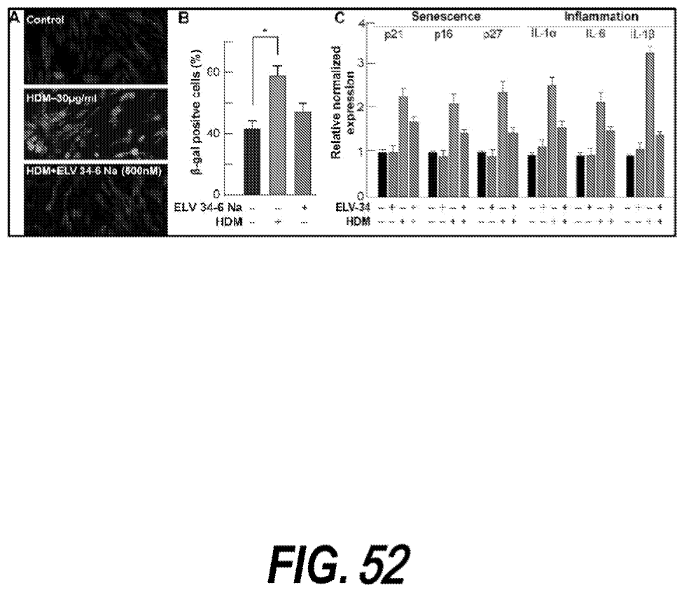

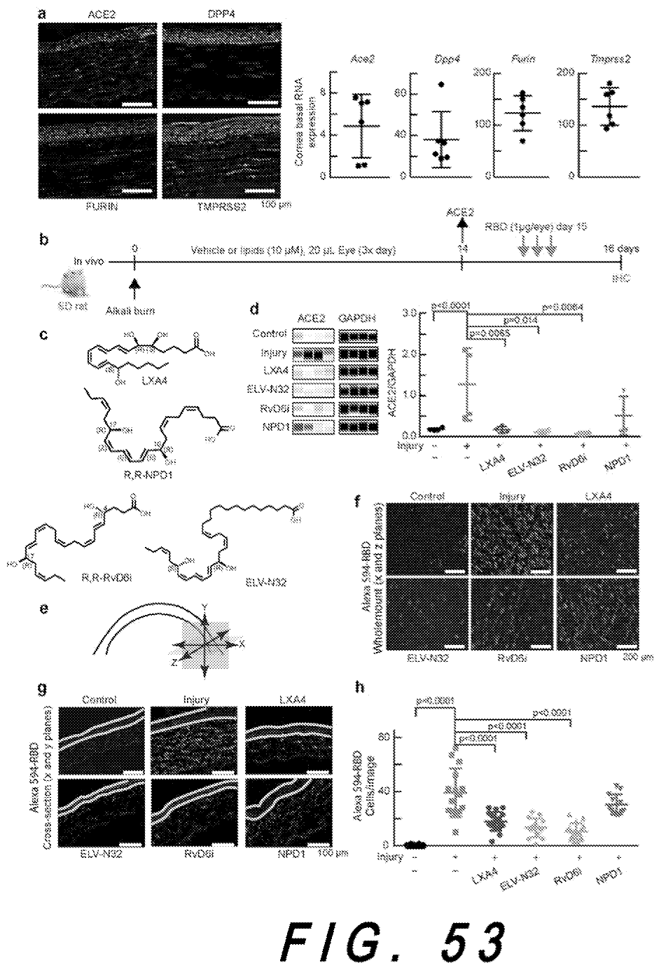

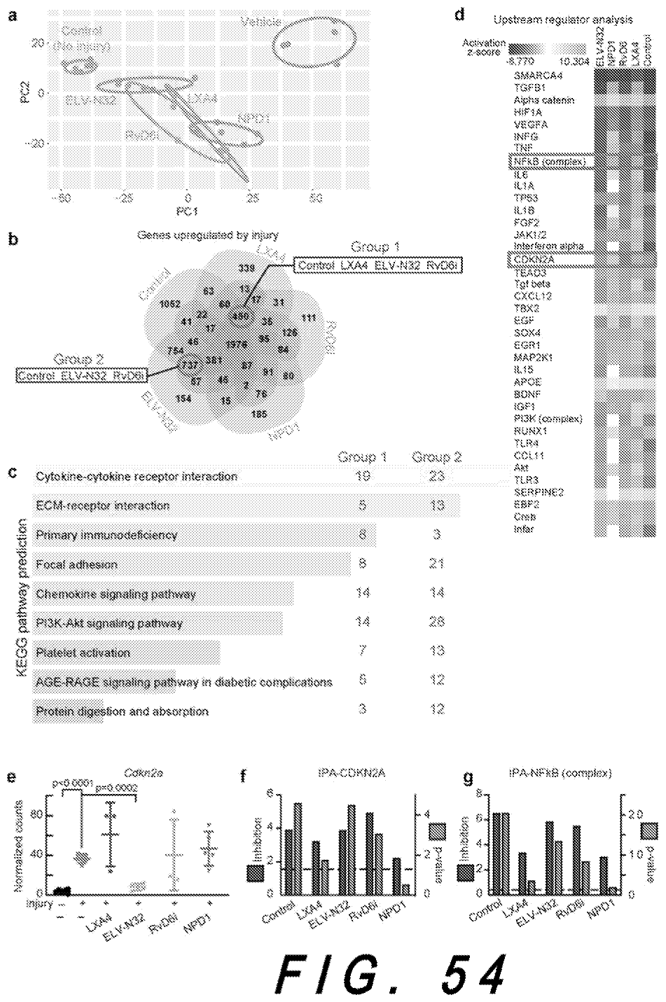

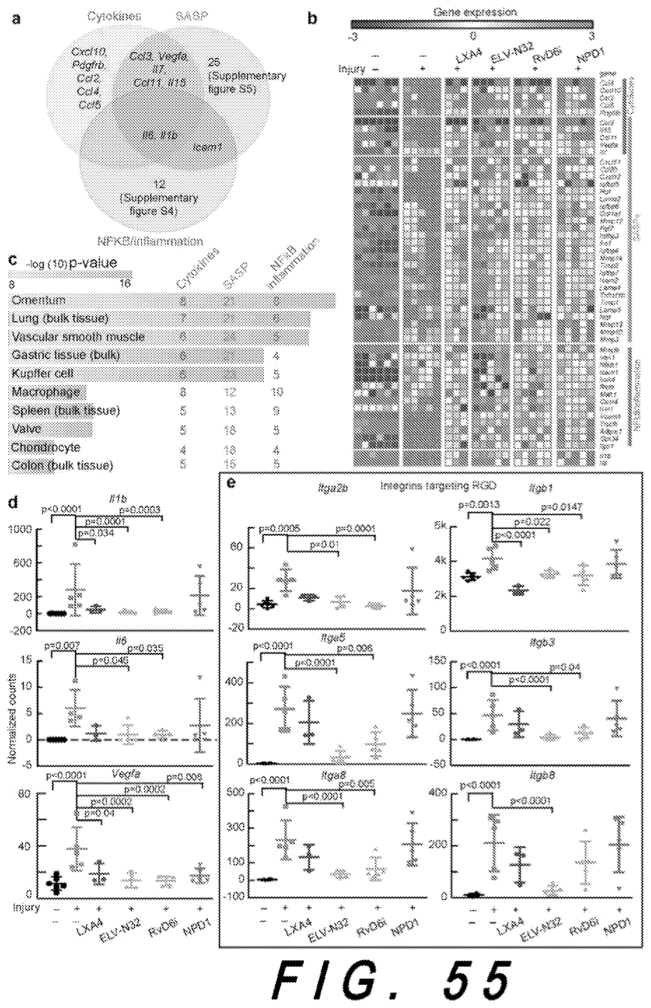

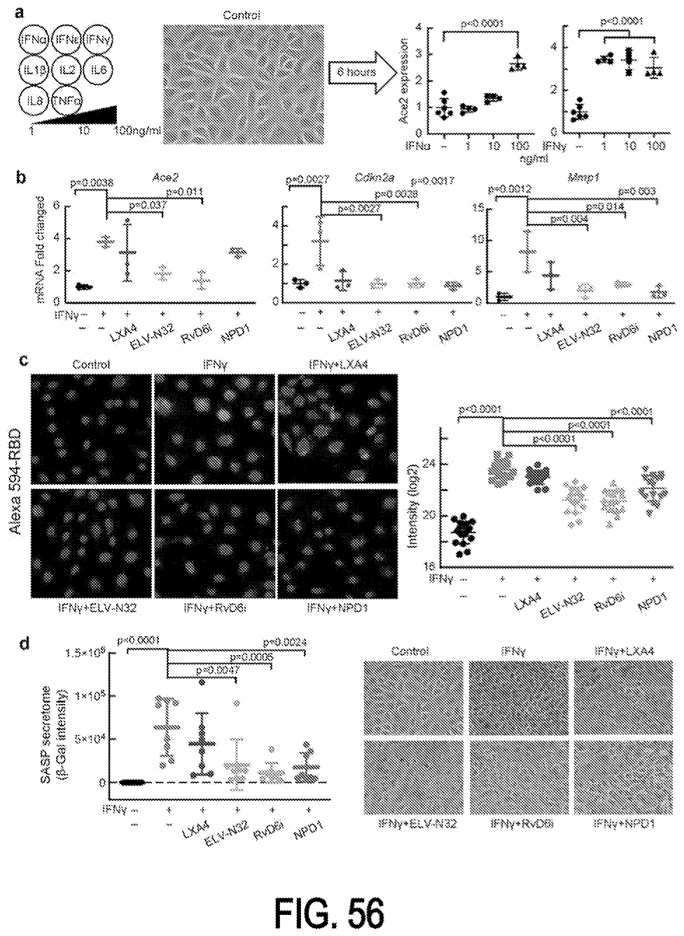

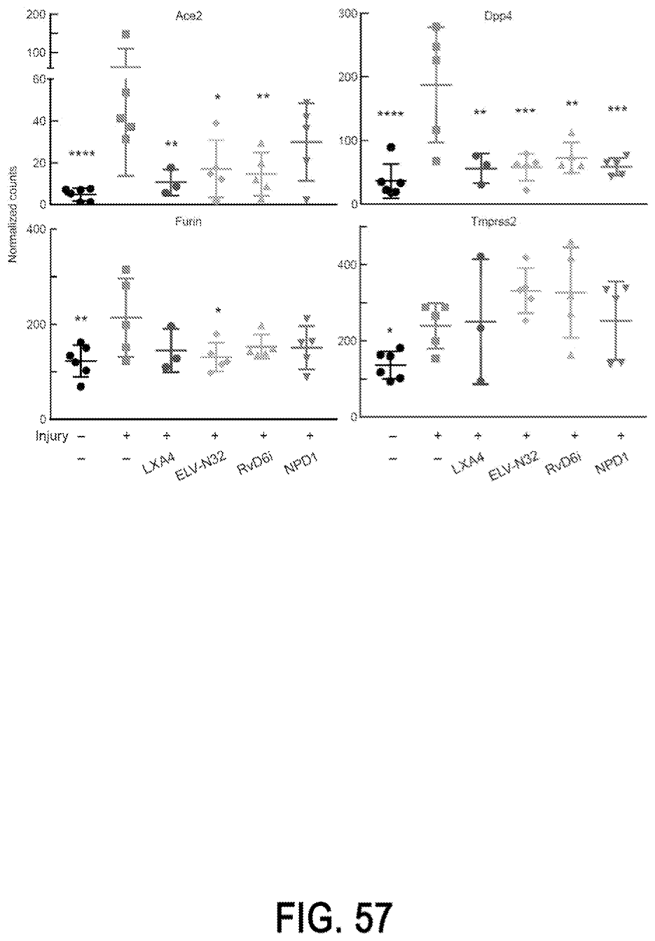

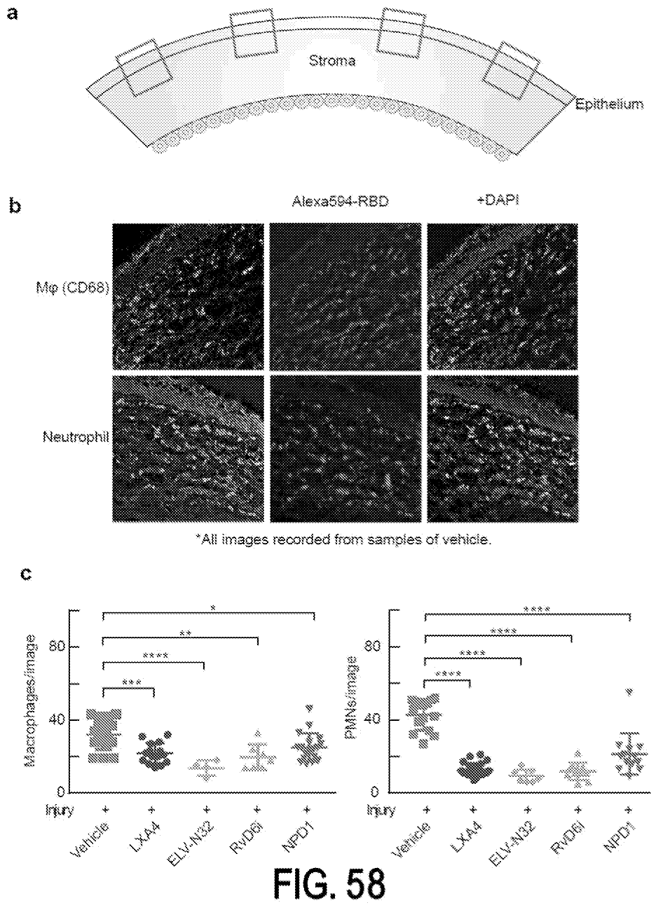

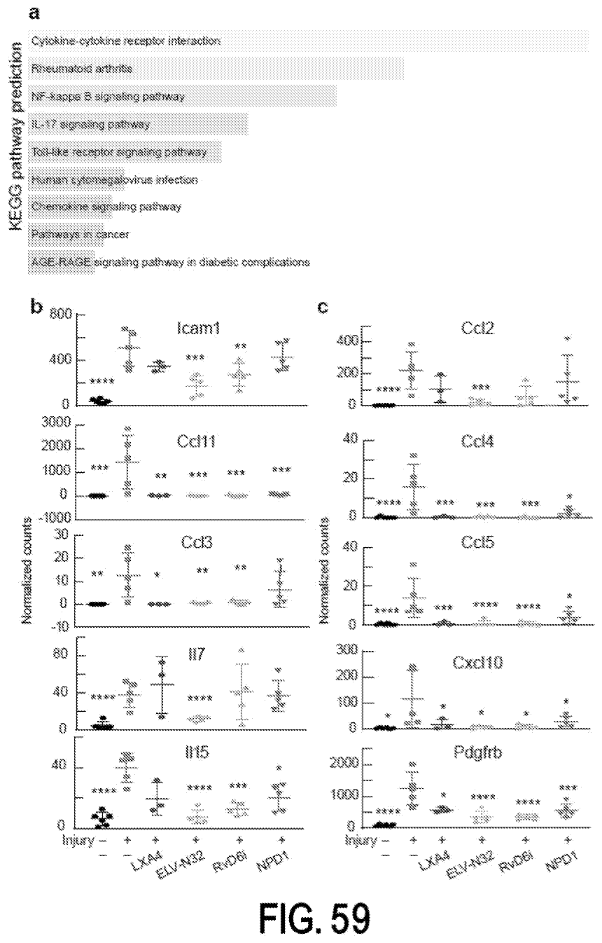

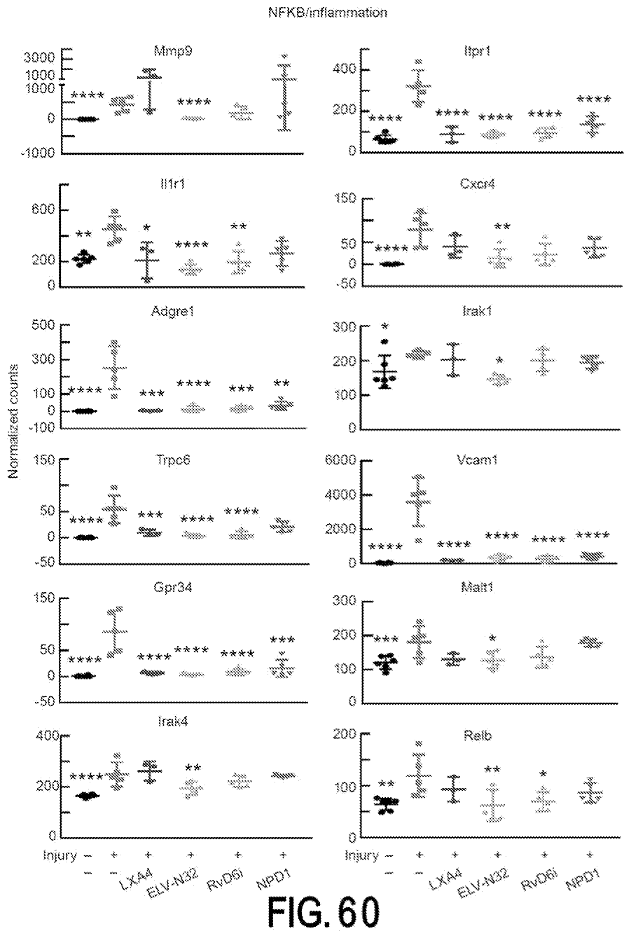

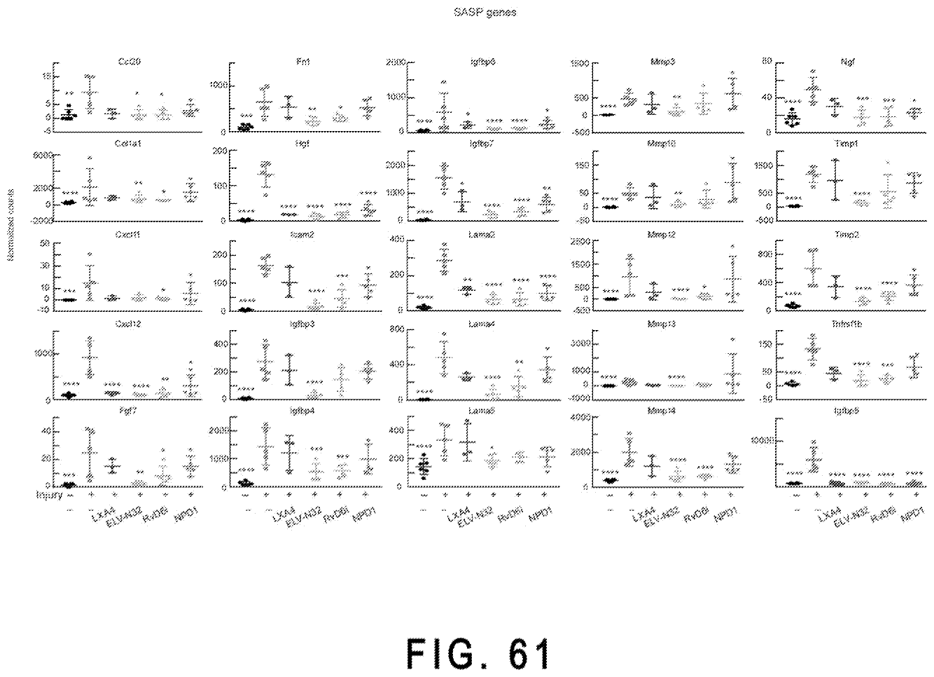

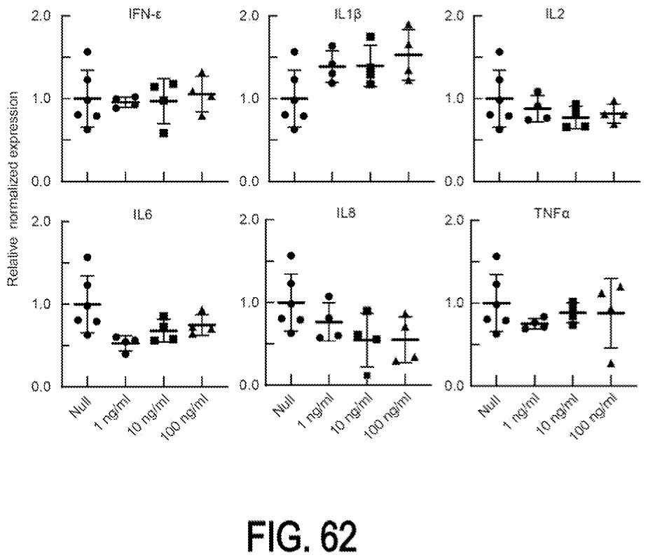

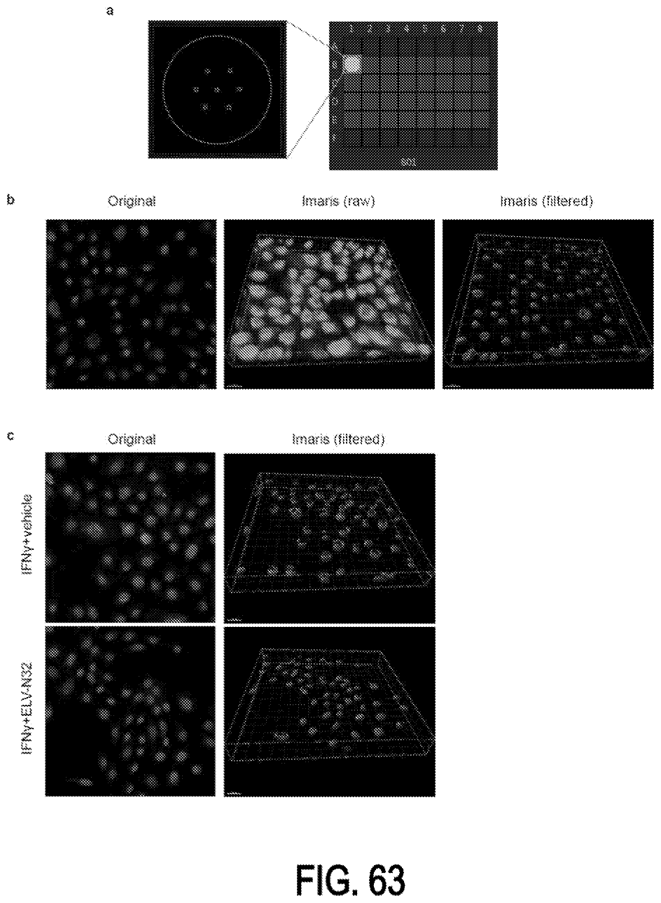

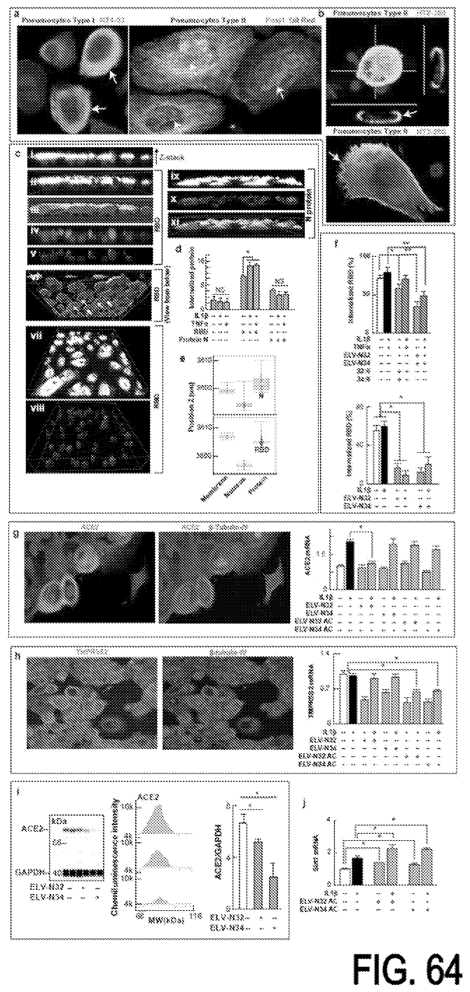

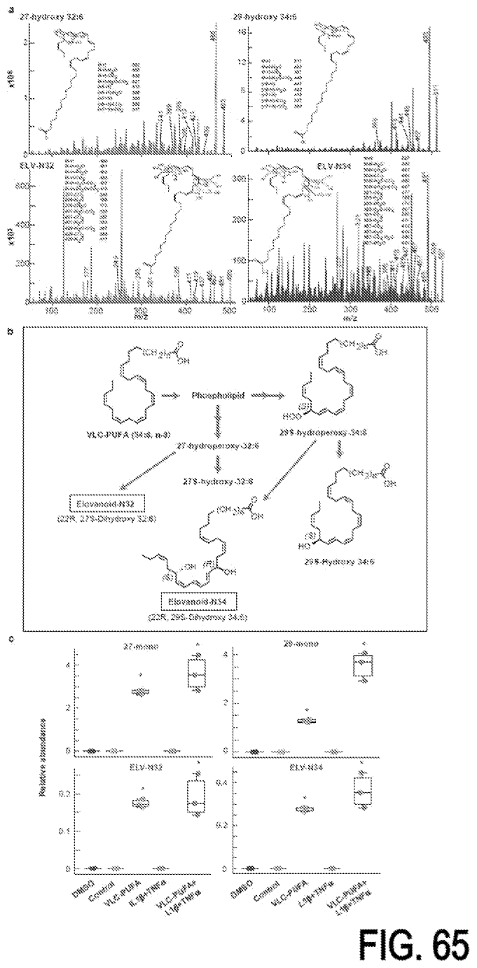

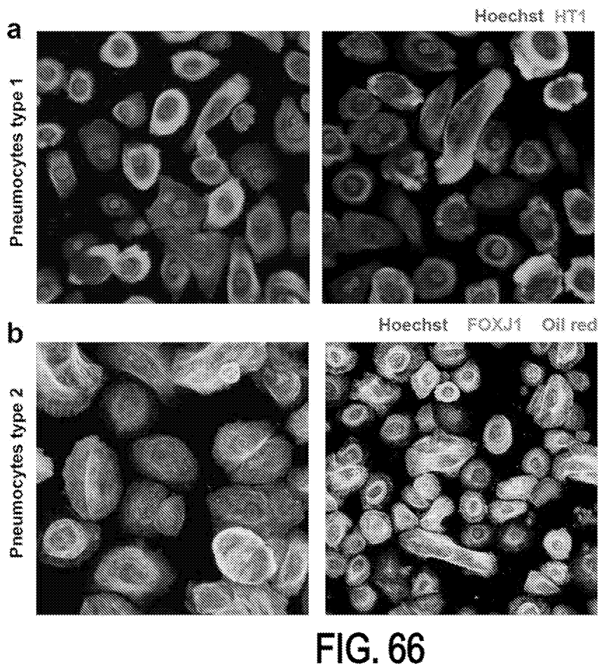

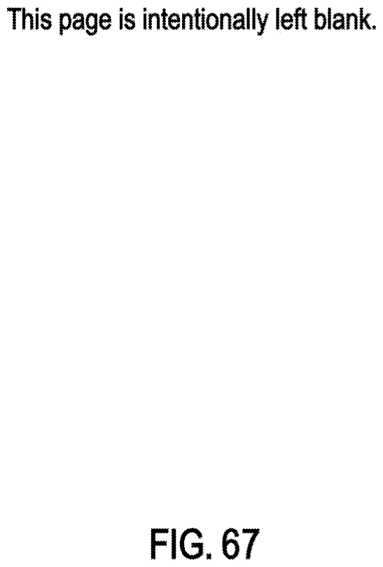

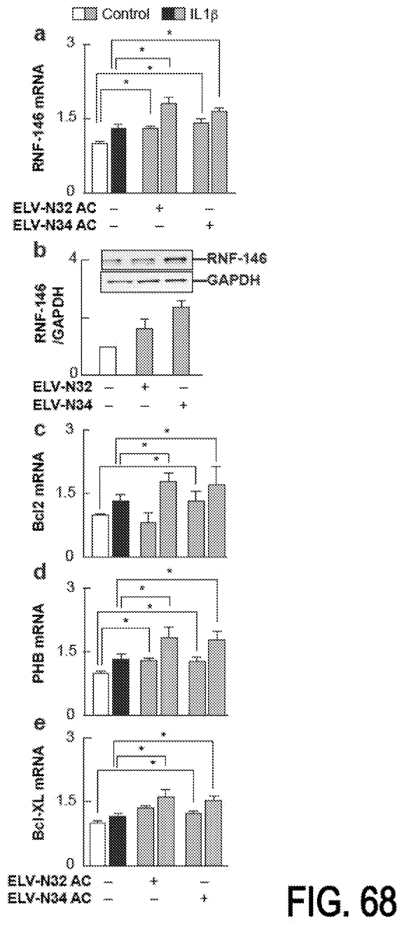

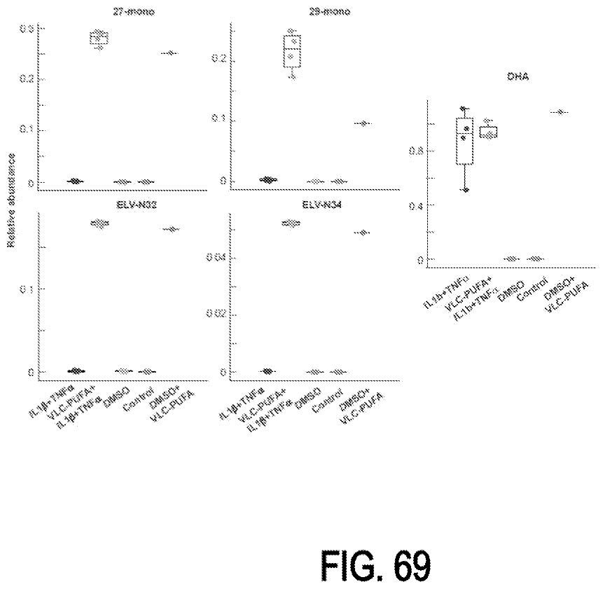

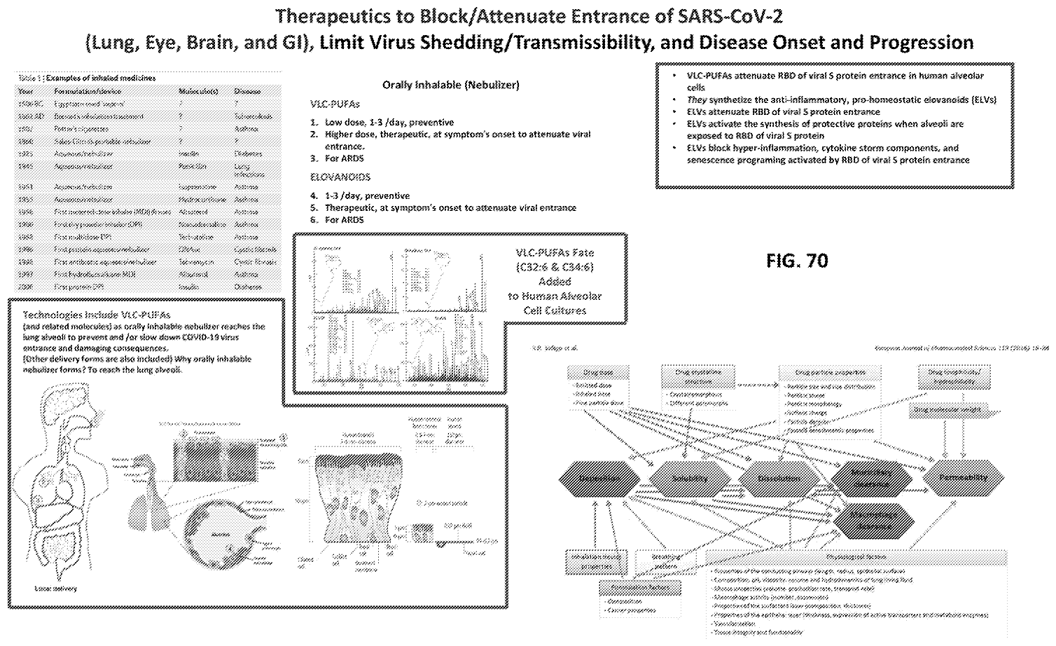

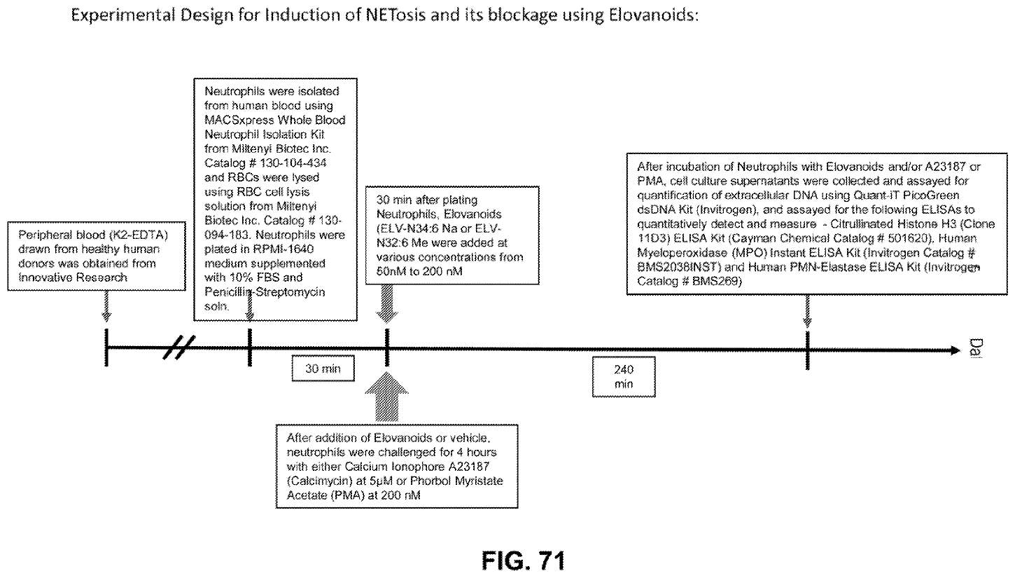

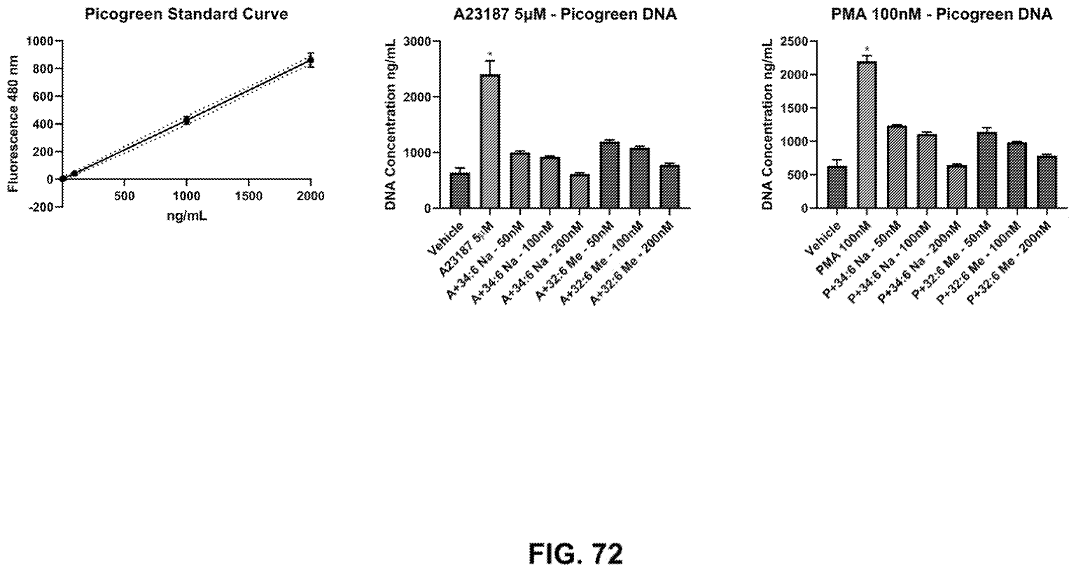

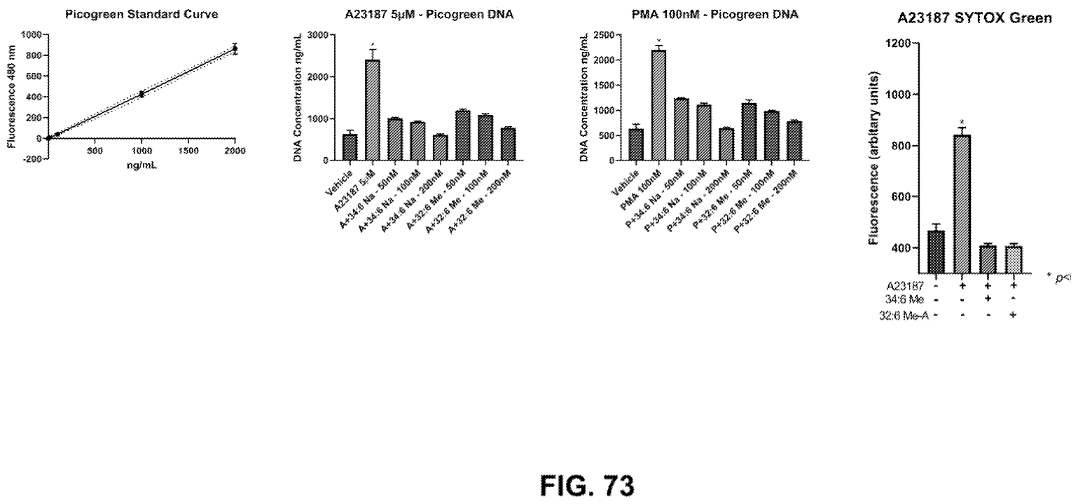

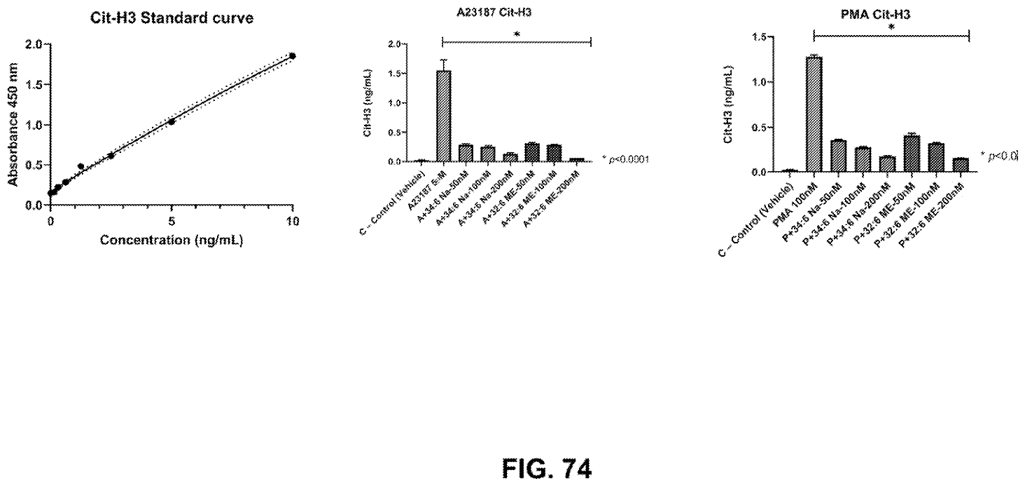

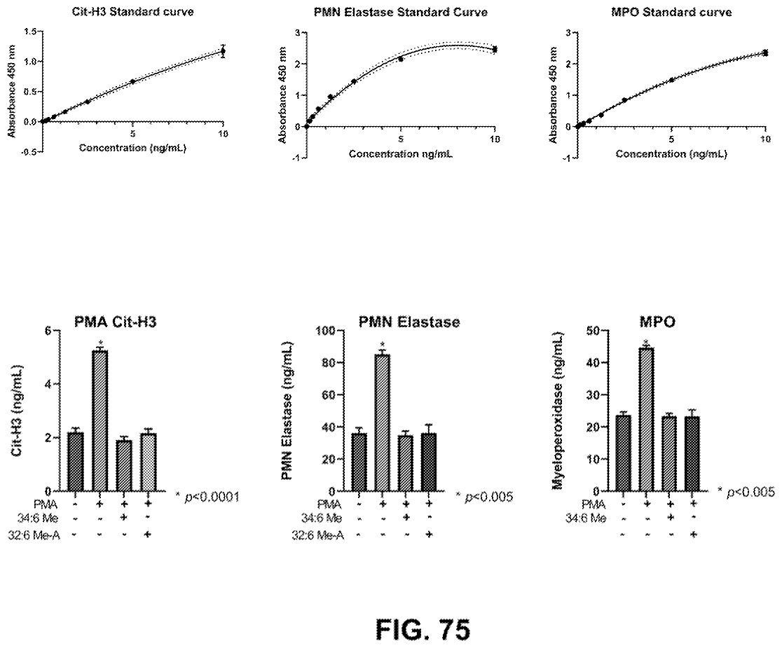

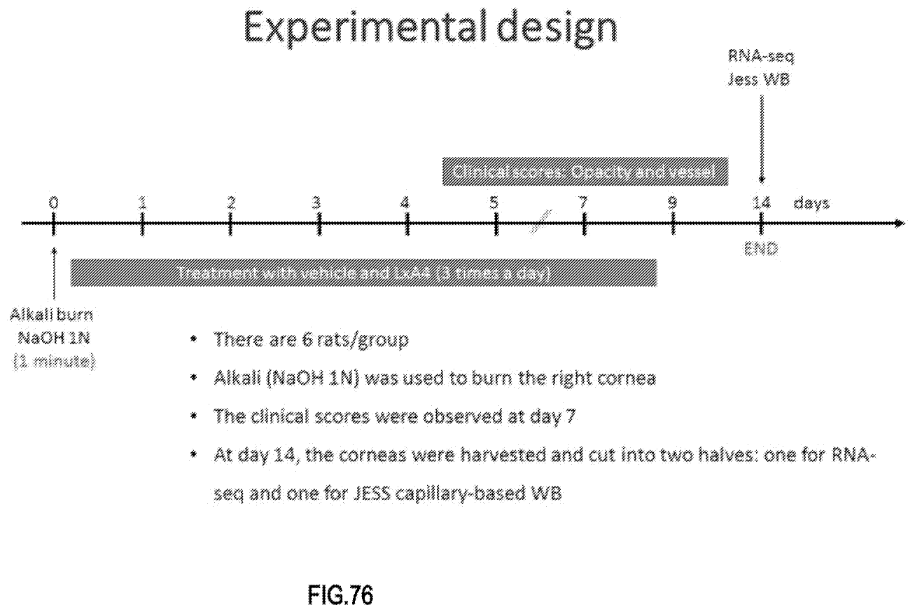

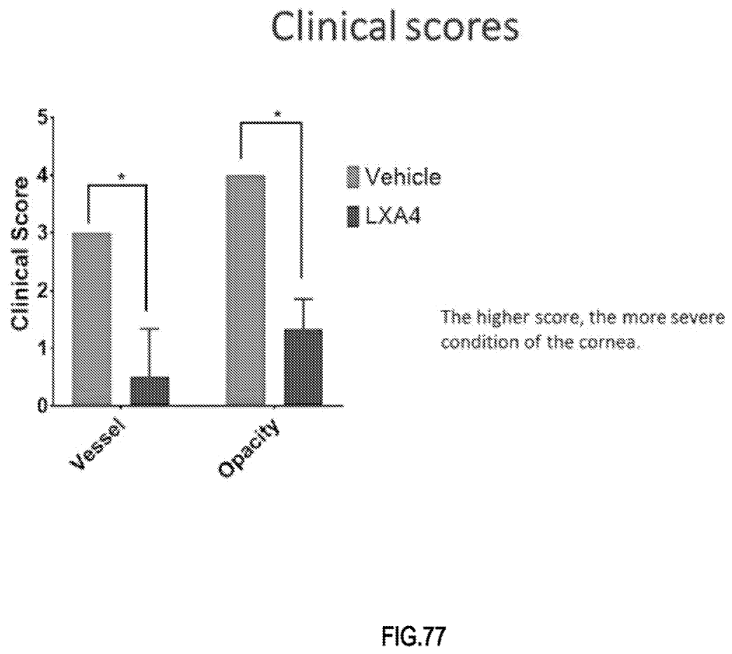

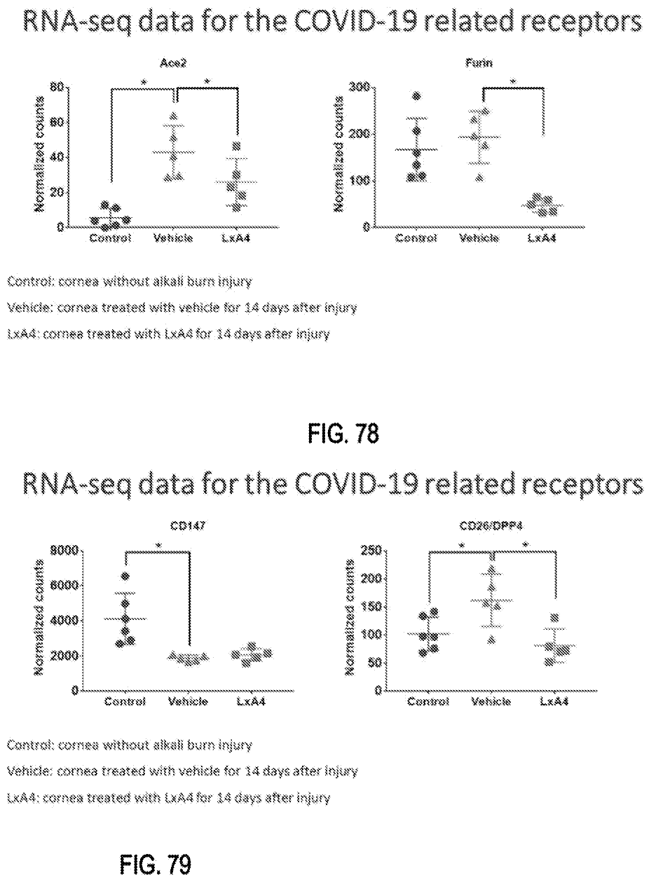

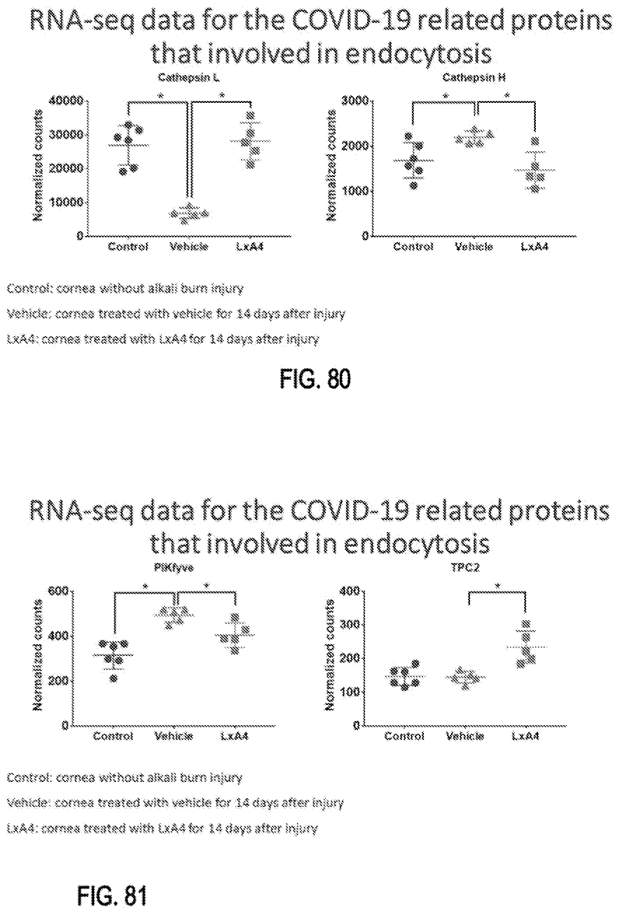

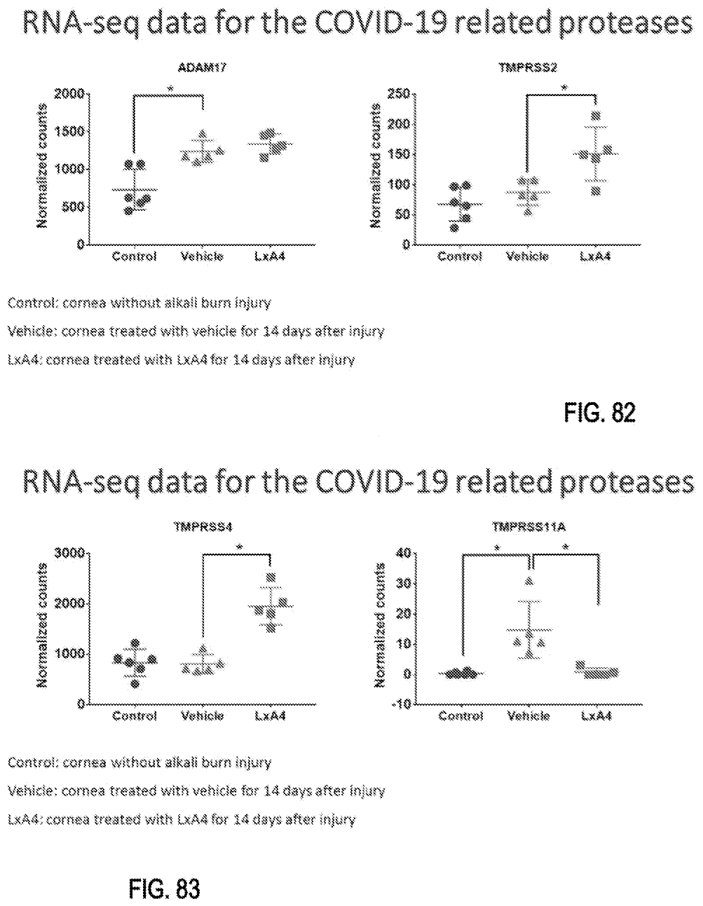

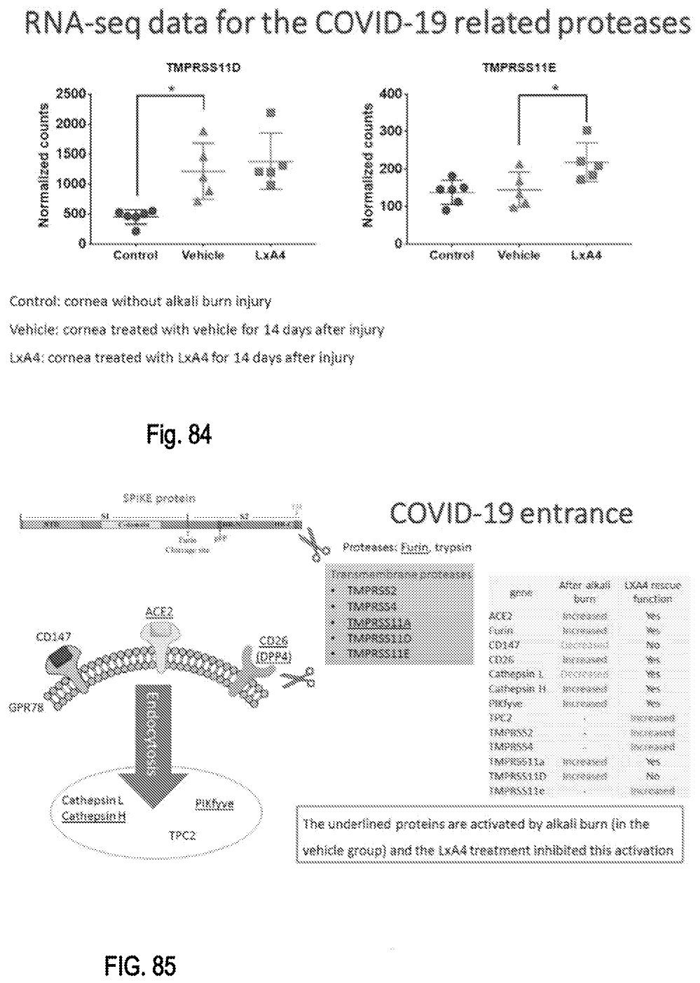

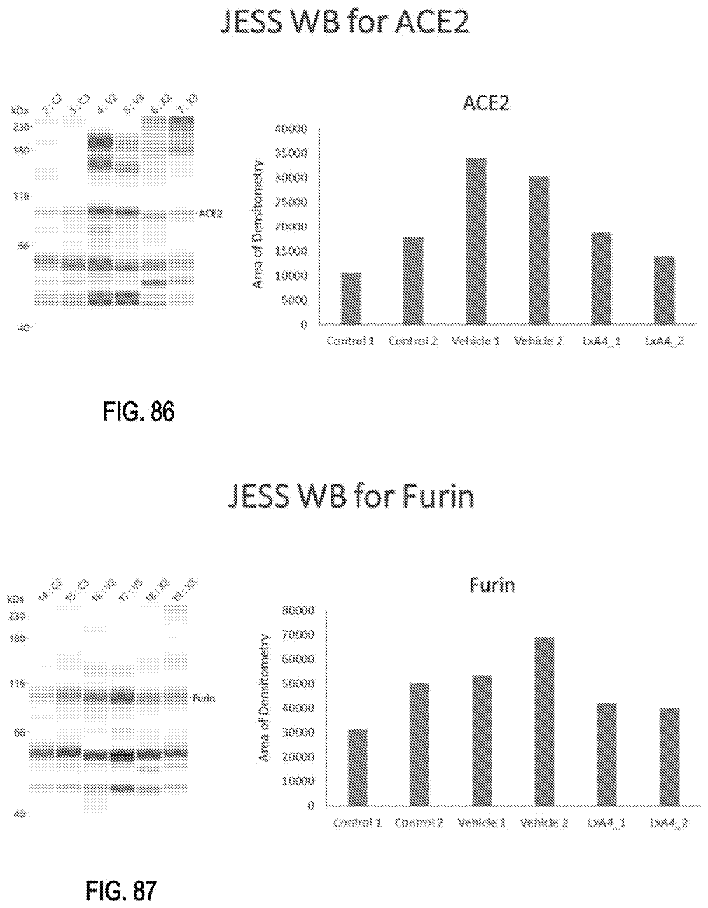

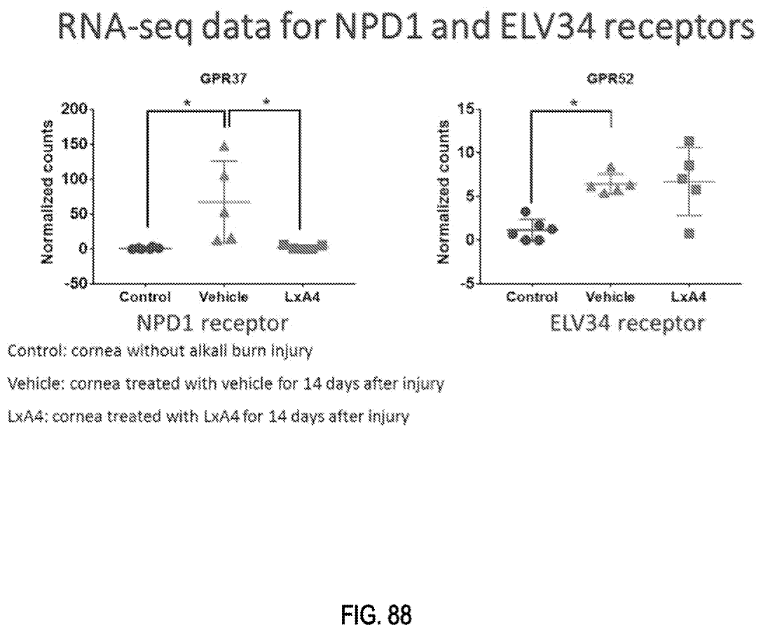

FIG. 4J illustrates the ELV-N34 full fragmentation spectra; the endogenous ELV-N34 peaks match to the standard ELV-N34.

FIG. 4K illustrates fragmentation of what can be ELV-N34 isomers.

FIGS. 5A and 5B illustrate the detection of ELV-N32 and ELV-N34 in neuronal cell cultures. Cells were incubated with C32:6n3 and C34:6n3 5 μM each, under OGD conditions.

FIG. 5A illustrates the VLC-PUFA C32:6n3, endogenous 27-hydroxy-32:6n3, endogenous 27,33-dihydroxy-32:6n3 (ELV-N32), and synthetic ELV-N32 prepared in stereochemical pure form via stereocontrolled total organic synthesis. MRM of endogenous ELV-N32 matches well with the MRM of the synthetic ELV-N32 standard.

FIG. 5B illustrates the same features as in FIG. 5A were shown in C34:6n3 and ELV-N34, with more peaks in ELV-N34 MRMs, which indicates isomers.

FIG. 6 illustrates Scheme 1 for the total synthesis of mono-hydroxylated elovanoids G, H, I, J, O, P, Q, R. Reagents & Conditions: (a) Catechol borane, heat; (b)N-iodo-succinimide, MeCN; (c) 4-chlorobut-2-yn-1-ol, Cs₂CO₃, NaI, CuI, DMF; (d) CBr₄, PPh₃, CH₂Cl₂, 0° C.; (e) ethynyl-trimethylsilane, CuI, NaI, K₂CO₃, DMF; (f) Lindlar cat., H₂, EtOAc; (g) Na₂CO₃, MeOH; (h) Pd(PPh₃)₄, CUT, Et₃N: (i) ^{t}Bu₄NF, THF; (j) Lindlar cat., H₂, EtOAc or Zn(Cu/Ag), MeOH; (k) NaOH, THF, H₂O, then acidification with HCl/H₂O; (1) NaOH, KOH, or the like. or amine, imine, etc.

FIG. 7 illustrates Scheme 2 for the total synthesis of di-hydroxylated elovanoids K, L, S, and T. Reagents & Conditions: (a) CuI, NaI, K₂CO₃, DMF; (b) camphorsulfonic acid (CSA), CH₂Cl₂, MeOH, rt; (c) Lindlar cat., H₂, EtOAc; (d) DMSO, (COCl)₂, Et₃N, −78° C.; (e) Ph₃P═CHCHO, PhMe, reflux; (f) CHIS, CrCl₂, THF, 0° C.; (g) cat. Pd(Ph₃)₄, CuI, Phil, rt; (h) ^{t}Bu₄NF, THF, rt; (i) Zn(Cu/Ag), MeOH, 40° C.; (j) NaOH, THF, H₂O, then acidification with HCl/H₂O; (k) NaOH, KOH, etc. or amine, imine, etc.

FIG. 8 illustrates Scheme 3 for the total synthesis of di-hydroxylated elovanoids M, N, U, and V. Reagents & Conditions: (a) cyanuric chloride, Et₃N, acetone, rt; (b) (3-methyloxetan-3-yl)methanol, pyridine, CH₂Cl₂, 0° C.; (c) BF₃, OEt₂, CH₂Cl₂; (d) nBuLi, BF₃, OEt₂, THF, −78° C., then 1; (e) ^{t}BuPh₂SiCl, imidazole, DMAP, CH₂Cl₂, rt; (f) camphorsulfonic acid, CH₂Cl₂, ROH, rt; (g) Lindlar cat., H₂, EtOAc; (h) DMSO, (COCl)₂, Et₃N, −78° C.; (i) Ph₃P═CHCHO, PhMe, reflux; (j) CHI₃, CrCl₂, THF, 0° C.; (k) cat. Pd(Ph₃)₄, CuI, PhH, rt; (l) ^{t}Bu₄NF, THF, rt; (m) Zn(Cu/Ag), MeOH, 40° C.; (n) NaOH, THF, H₂O, then acidification with HCl/H₂O; (o) NaOH, KOH, etc. or amine, imine, etc.

FIG. 9 illustrates Scheme 4 for the total synthesis of 32-carbon di-hydroxylated elovanoids.

FIG. 10 illustrates Scheme 5 for the total synthesis of 34-carbon di-hydroxylated elovanoids.

FIG. 11 shows upon adding VLC-PUFA (FA32:6) to the incubation medium of cell cultures of human bronchiole and alveoli, 27-mono-hydroxyl-32:6, stable precursors of ELV32 were identified. Full fragmentation of these endogenous molecules shows good matches to their theoretical peaks. The insert shows the structure along with the product ions when they are cleaved at the given bonds. 27-mono-hydroxy 32:6 (deprotonated) shown below

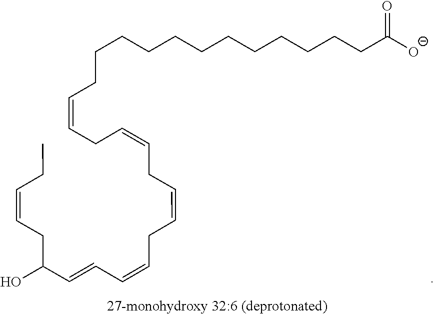 [SMILES: CC/C=C\CC(O)/C=C/C=C\C/C=C\C/C=C\C/C=C\CCCCCCCCCCCCC(=O)[O-]]

FIG. 12 shows upon adding VLC-PUFA (FA34:6) to the incubation medium of cell cultures of human bronchiole and alveoli, 29-mono-hydroxy-34:6, stable precursors of ELV34 were identified. Full fragmentation of these endogenous molecules shows good matches to their theoretical peaks. The insert shows the structure along with the product ions when they are cleaved at the given bonds. 29-mono-hydroxy-34:6 as shown below

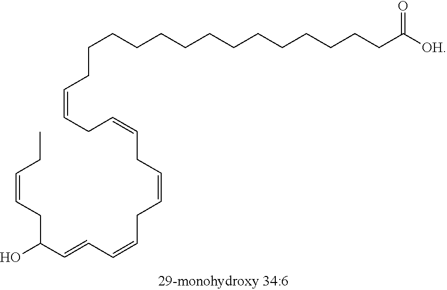 [SMILES: CC/C=C\CC(O)/C=C/C=C\C/C=C\C/C=C\C/C=C\CCCCCCCCCCCCCCC(=O)O]

FIG. 13 shows ELV32 synthesized from VLC-PUFA (FA32:6) in cultures of human bronchiole and alveoli. Full fragmentation patterns form the mass spectrometry of endogenous ELV32 show good matches to their standards. The insert shows the structure along with the product ions when they are cleaved at the given bonds. ELV32 (deprotonated) as shown below

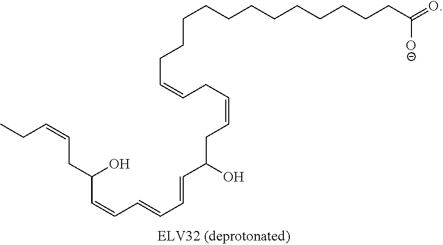 [SMILES: CC/C=C\CC(O)\C=C/C=C/C=C/C(O)C/C=C\C/C=C\CCCCCCCCCCCCC(=O)[O-]]

FIG. 14 shows ELV34 synthesized from VLC-PUFAs (FA34:6) in cultures of human bronchiole and alveoli. Full fragmentation patterns from the mass spectrometry of endogenous ELV34 shows good matches to their standards. The insert shows the structure along with the product ions when they are cleaved at the given bonds. ELV34 as shown below

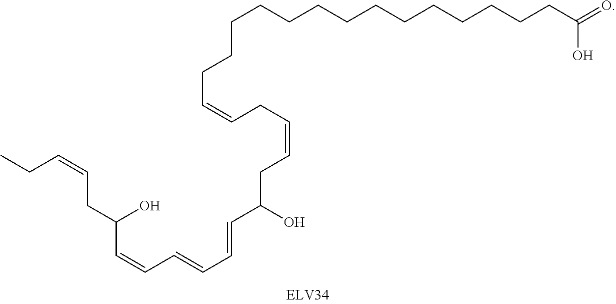 [SMILES: CC/C=C\CC(O)\C=C/C=C/C=C/C(O)C/C=C\C/C=C\CCCCCCCCCCCCCCC(=O)O]

FIG. 15 shows a mass spectrum. Human lung cell cultures with VLC-PUFAs (FA32:6 and FA34:6) were added to the medium show that the VLC PUFAs have been incorporated in the phosphatidylcholine molecular species. They are paired with FA18:1 or FA18:0.

FIG. 16 shows transversal cut of the image and Imaris reconstruction showing different layers. Transversal plane in the image (bottom panel) and its rendering (top panel). The membrane staining performed using Cell mask deep red is further depicted. The nuclei are shown in blue (stained with Hoechst 33342) and the RBD domain from S protein of SARS-CoV-2 conjugated with Alexa Fluor 546 is shown in red.

FIG. 17 shows a rendition of Imaris to the nuclei (blue) and S protein RBD domain (red) alone, viewed from the top.

FIG. 18 shows image (left) and the rendition (right) of Imaris from the FIG. 17 plus the membrane staining.

FIG. 19 show renderings depicting the amount of protein that is bound or take-up by the cell. Perspective from below.

FIG. 20 shows renderings depicting the amount of protein that is bound or take-up by the cell. From above.

FIG. 21 shows Human Small Airway Epithelial cells isolated from 78-year-old Caucasian male morphology day 14. Panel A Shows morphology under 10× magnification. Panel B Shows Morphology under 20 magnification, area inset from Panel A.

FIG. 22 provides a schematic of embodiments described herein. Without wishing to be bound by theory, VLC-PUFAs (n-3) and elovanoids (ELVs) protect lungs and cells of other barrier organs (for example, nasal mucosa, cornea of the eye, and GI enterocytes) against SARS-CoV-2. Panel A illustrates the arrival of VLC-PUFAs (e.g. after oral or nasal inhalation) and its uptake in cells of the bronchiole/alveoli or nasal mucosa where ELVs are biosynthesized using the VLC-PUFAs as a starting point. ELVs, then became paracrine of autocrine effectors′; Panel B illustrates ELVs as paracrine mediators attenuate the cytokine storm in the lung parenchyma (or nasal mucosa) as well as systemic cytokine storm, in addition they inhibit monocyte derived macrophage formation and inhibit T cell senescence; Panel C illustrates ELVs autocrine mediators through membrane receptors downregulate the overactivation of the immune/inflammatory response (that include inflammasome formation, Interleukin 6 synthesis, for example) and of senescence-triggered inflammation; Panel D illustrates ELVs also modulates elements of the tetraspanin membrane microdomains (TEM) essential for the interaction between virus and host that include latching the receptor binding domain of the spike glycoprotein of SARS-CoV-2 to ACE2 for cell attachment; without wishing to be bound by theory, ELVs will modulate ACE2 expression, its shedding and counteract ensuing dysfunctions of the renin-angiotensin system. Dysfunctions of this system leads to induction of damaging inflammation in the lung. ELVs in addition modulate expression of host proteases (such as FURIN, TMPR5S2, DPP4) necessary for cleavage of the viral protein to allow a conformational change for fusion/entrance of the virus into the cell. ELVs also regulate expression of other molecules, such as CD-9 and interferons; Panel E illustrates VLC-PUFAs incorporate into phospholipids and in turn disrupts TEM and also endosomes formation, key in the virus life cycle, and in addition, includes downregulation of the ACE2 receptor, and/or modifies the membrane microdomain where host receptor is located, and/or the T1VIPRSS2 protease for post fusion.

FIG. 23 shows tetraspanins at a glance. Adapted from Charrin, et al (2014).

FIG. 24 shows chromatograms of 33-monohydroxy 38:6, 31-monohydroxy 36:6, 29-monohydroxy 34:6, and 27-monohydroxy 32:6.

FIG. 25 shows chromatograms of 33-monohydroxy 38:6, 31-monohydroxy 36:6, 29-monohydroxy 34:6, and 27-monohydroxy 32:6.

FIG. 26 shows (top) that ELVs downregulate the expression of the ACE-2 receptor analyzed by the Jess system and (bottom) that in the presence of Interleukin1-beta ELV-32 diminishes receptor-binding domain (RBD) of the SARS-CoV-2 spike entrance in human alveoli culture.

FIG. 27 shows the structure and full fragmentation spectrum of 33-monohydroxy 38:6 n-3 detected in the lung (alveoli) cell culture incubated with FA 38:6 and shows a good match to the theoretical full fragmentation of 33-monohydroxy 38:6 n-3. 33-monohydroxy FA38:6 (deprotonated) as shown below

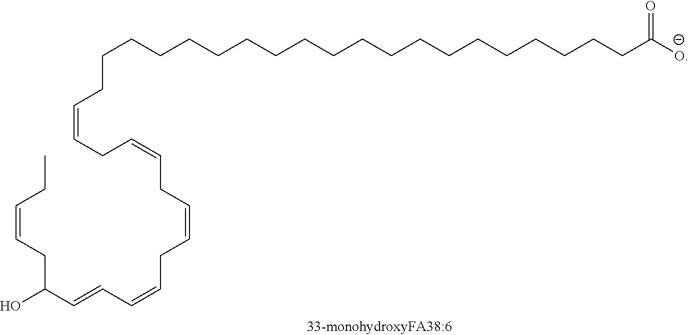

FIG. 28 shows the structure and full fragmentation spectrum of (26, 33)-dihydroxy 38:6 n-3 (ELV38) matches well to the full fragmentation spectrum of m/z of 583, which was detected from lung (alveoli) cell culture incubated with FA 38:6. ELV 38 (deprotonated) as shown below

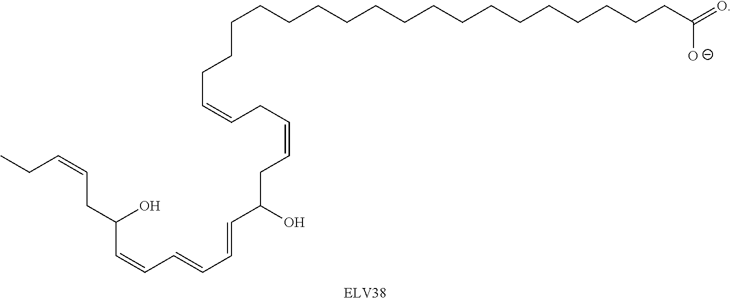 [SMILES: CC/C=C\CC(O)\C=C/C=C/C=C/C(O)C/C=C\C/C=C\CCCCCCCCCCCCCCCCCCC(=O)[O-]]

FIG. 29 shows VLC-PUFAs (n-3) induce lipidome remodeling and elovanoid (ELVs) synthesis in human alveoli in cell cultures. Without wishing to be bound by theory, ELVs downregulate availability of a) ACE2, thus hindering cell surface virus binding and b) key host proteases (type II serine protease TMPRSS2, furin, and DPP4) that mediate S protein activation and initial viral cell entry. Without wishing to be bound by theory, VLC-PUFAs (n-3) curtail inflammation and attenuates cytokine storm by fostering the synthesis of ELVs that, in turn, would target lung and nasal parenchyma tissues. In addition, ELVs will elicit intracellular protective events through a G protein. Without wishing to be bound by theory, VLC-PUFAs induce lipidome remodeling by activating the synthesis of lung atypical phospholipids and disrupt tetraspanin-enriched membrane microdomains (they are not lipid rafts), contributing to blocking SARS-CoV-2 virus cell binding and entrance, and in addition perturb endosome formation, hinder virus replication, and limit virus shedding.

FIG. 30 shows metabolic fate of 34:6n-3 very long chain-polyunsaturated fatty acid (VLC-PUFA) in human alveoli cells in primary culture. The VLC-PUFA is incorporated in atypical lung molecular species of phospholipids and then lead to the formation of a short-lived lipoxygenase metabolite, 29S-hydroperoxy-34:6, which in turn forms the stable 29S-hydroxy-34:6. We clearly showed that elovanoid-34 is subsequently synthetized. Similar metabolism was found when 32:6n-3 was incubated with these cells that resulted in the formation of elovanoid-32.

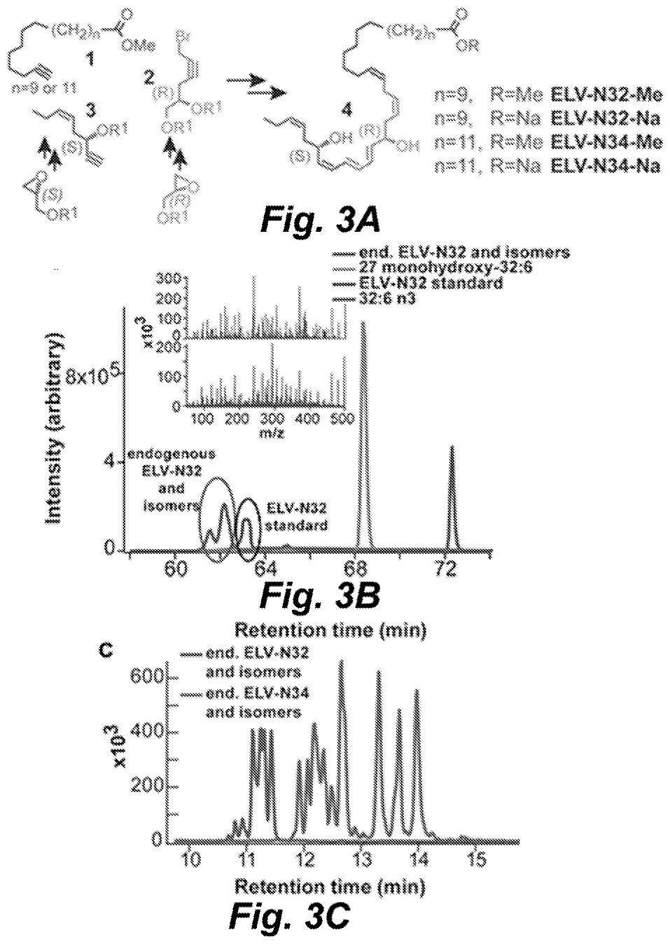

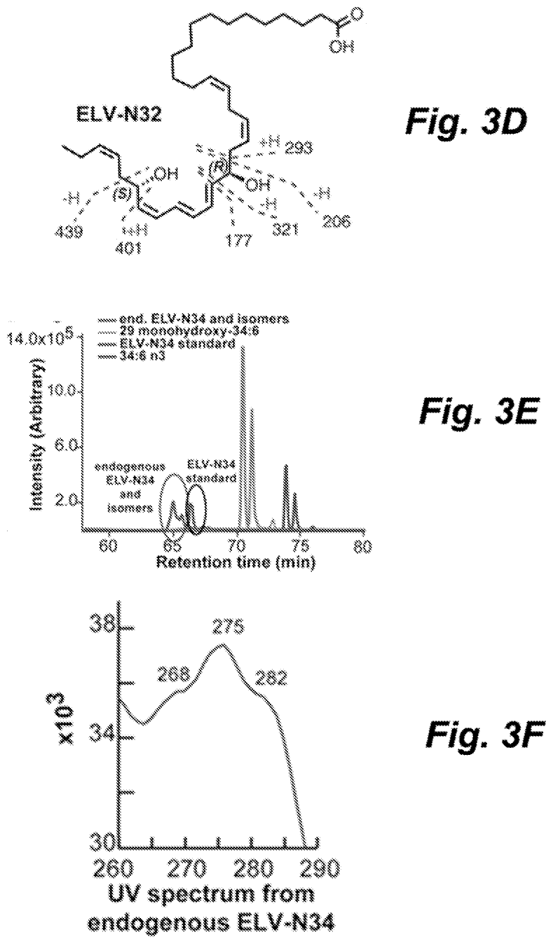

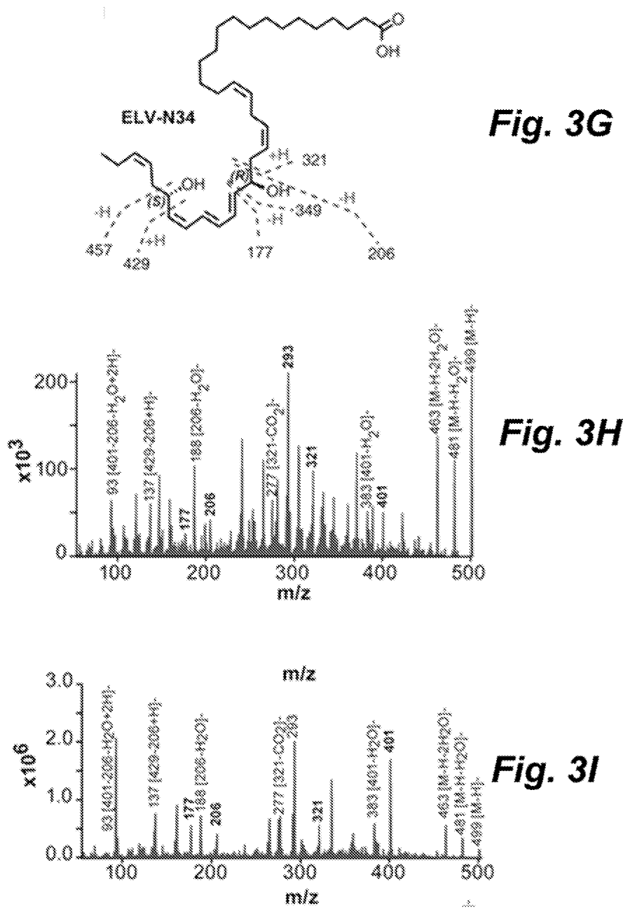

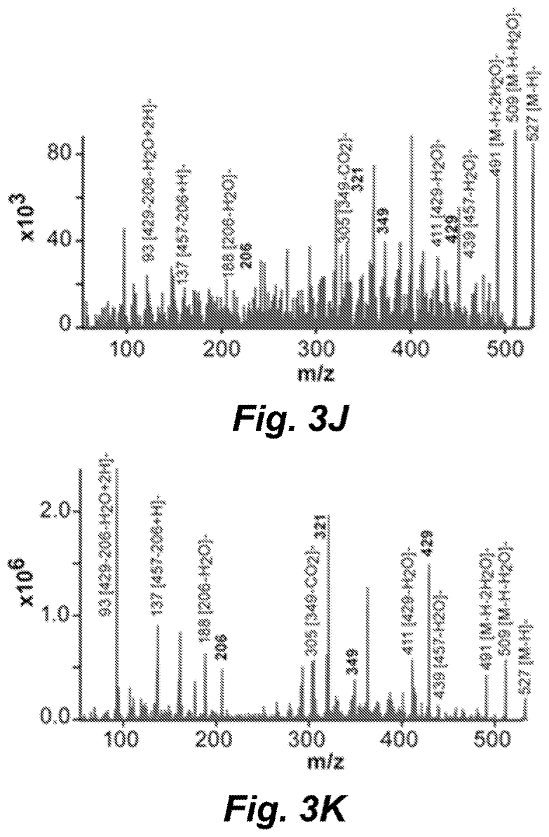

FIG. 3I shows human airway epithelial cells from a 78-year-old normal Caucasian male—morphology day 14 in culture. (Panel A) 10× magnification. (Panel B) 20× magnification from area inset from Panel A.

FIG. 32 shows pneumocyte markers in chamber slide cultures. (Panel A) Type I lung epithelial (alveolar) cells labeled (green) with the specific marker HT1-53. (Panel B) Type II lung epithelial (alveolar) cells labeled with a ciliated cell marker Foxj1 (green), which is required for cilia formation and is an early marker of epithelial cell differentiation, recovery, and function, and Oil Red O(red), a marker for the type II lipid/lamellar bodies. (Panels C-F) HT2-280 labeled (green) type II human lung cells. (Panel C) A single type II cell is viewed from the x, y, and z axis, showing the position of the labeled cilia. (Panel D) Additional ciliated cells are shown. (Panel E) Type II cells can undergo cell division and (Panels F-G) are motile; note the double nuclei here. Hoechst-stain label nuclei are shown in blue.

FIG. 33 shows type II pneumocytes. ACE2 label (green) in a human lung cell culture (Panel A), combined with the type II marker, f3-tubulin IV (red) (Panel B). Nuclei are blue. Foxj1, a marker of ciliated alveolar cells, labels type II cells (green), and Oil Red O(red) indicates the formation of the lipid-containing lamellar bodies unique to type II cells (Panel C). Nuclei are blue.

FIG. 34 shows chemical structures of the ELVs in FIG. 35G. Panel A shows ELV32-A is the methyl ester of an acetylenic compound with a triple bond between C25 and C26. Panel B shows ELV32 methyl ester.

FIG. 35 shows internalization of the receptor-binding domain (RBD/Alexa 546) of Spike protein of SARS-CoV-2 in human alveoli in culture is exacerbated by IL1β and downregulated by ELVs. (Panel A) XZ plane of a Z-stack image, in blue nuclei, in red RBD and in white membrane (upper panel), (Panel B) digitalization of nuclei and RBD signal not membrane, (Panel C) complete digitalized image, (Panel D) Nuclei and RBD signal, (Panel E) digitalized nuclei and RBD. (Panel F) Superior view of digitalized nuclei and S-Protein with (left panel) and without membrane (right panel). (Panel G) Quantification of the RBD internalized protein. The whole RBD signal was matched to 100% and using the Z position reference of the membrane, the % of above (free RBD), within (bound RBD) and below (internalized RBD) where determined. *p<0.05, ANOVA p=0.0005 in N=24 images for ELV32 methyl ester- and ELV 32(AC) acetylenic methyl ester-treated cells and 48 images for Controls and IL1f3. ELV structures in FIG. 34.

FIG. 36 shows RBD protein internalization decreased by ELVs 32 and 34 and their precursors, VLC-PUFAs 32:6 and 34:6. (Panel A) Added VLC-PUFAs induced ELV synthesis in human primary alveolar cells and prompted decreased RBD domain of spike protein internalization. Bars: mean of RBD quantification located below the membrane line (FIGS. 35, 37). N=24 *p<0.05; **p<0.001. (Panel B) JESS electrophoretic ACE2 separation (left), densitometry of bands (right). (Panel C) Quantification of ACE2 standardized by GAPDH. Bars are mean plus standard deviation *p<0.05.

FIG. 37 shows specificity of RBD internalization in human alveoli in cell culture. Viral Nucleocapsid N protein tagged with Alexa Fluor-594 was used in parallel to RBD to determine specific internalization of RBD. (Panel A) XZ plane of a Z-stack image of alveoli culture after 24 h exposure to N protein. (Panel B) View from below of a digitalized image of alveoli cells exposed to RBD tagged with Alexa Fluor-594 for 24 h. Red arrows show the position of internalized RBD. (Panels C & D) The plots show digitalized elements position in the Z axis from 2 representative images (Panel C) of N protein exposed and (Panel D) from RBD exposed alveoli cells. The membrane thickness was taken as reference in each picture to determine % of proteins above (free protein), within (bound protein) and below (internalized protein). N protein did not internalize and was found partially bound to the membrane while most remained free.

FIG. 38 shows VLC-PUFAs 32:6 and 34:6 are added to the lung cell cultures and found incorporated into phosphatidylcholine molecular species to form PC(18:1/32:6) and PC(18:1/34:6).

FIG. 39 shows the fate of VLC-PUFAs (C32:6 and C34:6, 2 μM each) added to human lung cell cultures towards ELV synthesis. 27-monohydroxy-32:6, 29-monohydroxy-34:6, as well as ELVs are formed. Full fragmentation of 27-monohydroxy-32:6 and 29-monohydroxy-34:6 show matches to their theoretical peaks. The inserts depicts the structures of 27-monohydroxy-32:6 and 29-monohydroxy-34:6 along with the product ions as they are cleaved at a given bonds. The complete structures of ELV-32 and ELV-34 were confirmed to be: ELV-32: (14Z,17Z,20R,21E,23E,25Z,27S,29Z)-20,27-dihydroxydo-triaconta-14,17,21,23,25,29-hexaenoic acid; ELV-34: (16Z,19Z,22R,23E,25E,27Z,29S,31Z)-22,29-dihydroxytetra-triaconta-16,19,23,25,27,31-hexaenoic acid. The structures of the compounds described are

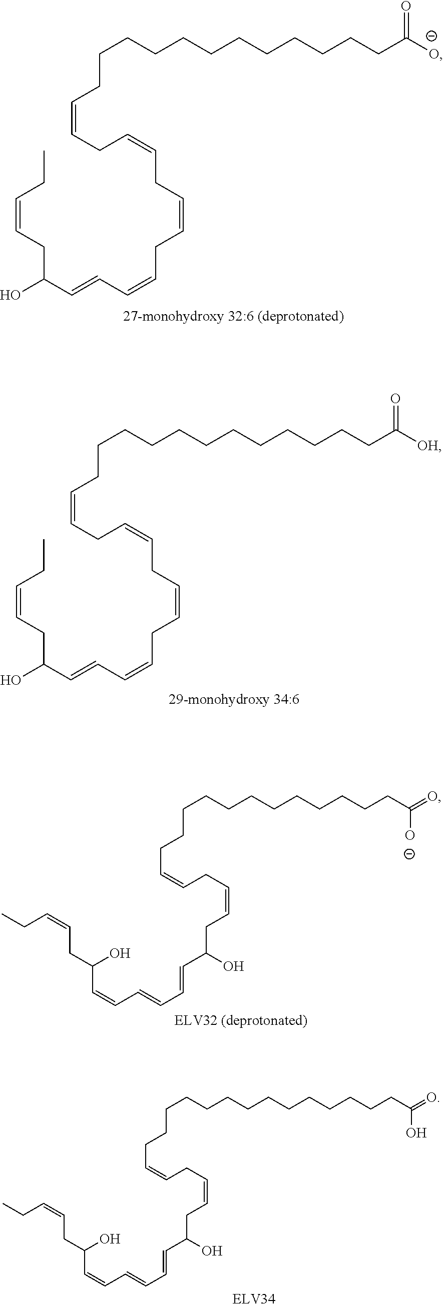

FIG. 40 shows: VLC-PUFAs (n-3) induce lipidome remodeling and synthesis of elovanoids (ELVs) in human alveoli and nasal mucosa: ELVs downregulate availability of a) ACE2, thus hindering cell surface virus binding (FIG. 46,47) and b) key host proteases furin, type II serine protease TMPRSS2, and DPP4 that mediate S protein activation and viral entry. VLC-PUFAs induce lipidome remodeling by the synthesis of lung atypical phospholipids (FIG. 50) and thus disrupt tetraspanin-membrane microdomains to contribute blocking SARS-CoV-2 virus cell binding and entrance, and in addition perturb endosome formation hindering virus replication. ELVs curtail inflammation and attenuate cytokine storm by autocrine (G protein mediated FIG. 51) and paracrine signaling.

FIG. 41 shows metabolic fate of 32:6n-3 and of 34:6n-3 VLC-PUFA in human alveolar cells in primary culture. They are incorporated in atypical lung phospholipids (FIG. 50) and lead to the formation of short-lived lipoxygenase metabolites, 27S-hydroperoxy-32:6 or 29S-hydroperoxy-34:6, respectively which in turn forms the stable 27S/OH-34:6 or 29S/OH-34:6 (FIG. 45). We showed that elovanoid-32 and 34 are subsequently synthetized (FIG. 45).

FIG. 42 shows human alveoli cells in primary culture from a 78-year-old normal Caucasian male (HSAEpC)—morphology day 14 in culture. (Panel A) 10× (Panel B) 20×, inset from A.

FIG. 43 shows pneumocyte markers in chamber slide cultures. (Panel A) Type I pneumocytes (green) with the specific marker HT1-53. (Panel B)Type II lung pneumocytes labeled with a ciliated cell marker Foxj1 (green), which is required for cilia formation and early marker of epithelial cell differentiation and function, and Oil Red O(red), a marker for type II lipid/lamellar bodies. (Panels C-F) HT2-280 labeled (green) type II human lung cells. (Panel C) A single type II cell is viewed from the X, Y, and Z axis, showing the position of the labeled cilia. (Panel D) Additional ciliated cells are shown. (Panel E) Type II cells can undergo cell division and (Panels F-G) are motile; note the double nuclei here. Hoechst-stain label nuclei are shown in blue.

FIG. 44 shows type II pneumocytes. ACE2 (green) in a culture of human alveolar cells (Panel A), combined with type II marker, β-tubulin IV (red) (Panel B). Nuclei (blue). Foxj1, a marker of ciliated alveolar cells, labels type II cells (green), and Oil Red O(red) shows lipid-containing lamellar bodies unique to type II cells (Panel C). Nuclei are blue.

FIG. 45 shows fate of VLC-PUFAs (C32:6 and C34:6, 2 μM each) added to human alveolar cell cultures to ELV synthesis (as in FIG. 41). Full fragmentation of 27/OH-32:6 and 29/OH-34:6 show matches to theoretical peaks. The inserts depict structures of 27/OH-32:6 and 29/OH-34:6 along with the product ions as they are cleaved at a given bond. The complete structures of ELV-32 and ELV-34 were confirmed to be: ELV32: (14Z,17Z,20R,21E,23E,25Z,27S,29Z)-20,27-dihydroxydo-triaconta-14,17,21,23,25,29-hexaenoic acid; ELV34: (16Z,19Z,22R,23E,25E,27Z,29S,31Z)-22,29-dihydroxytetra-triaconta-16,19,23,25,27,31-hexaenoic acid. The structures of the compounds described are

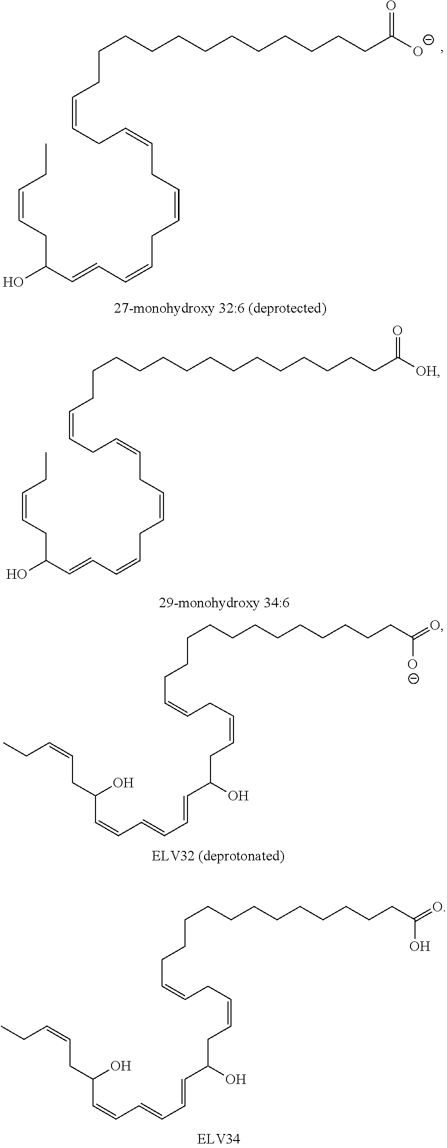 [SMILES: CC/C=C\CC(O)/C=C/C=C\C/C=C\C/C=C\C/C=C\CCCCCCCCCCCCC(=O)[O-] ; CC/C=C\CC(O)/C=C/C=C\C/C=C\C/C=C\C/C=C\CCCCCCCCCCCCCCC(=O)O ; CC/C=C\CC(O)\C=C/C=C/C=C/C(O)C/C=C\C/C=C\CCCCCCCCCCCCC(=O)[O-] ; CC/C=C\CC(O)\C=C/C=C/C=C/C(O)C/C=C\C/C=C\CCCCCCCCCCCCC(=O)O]

FIG. 46 shows internalization of receptor-binding domain (RBD/Alexa 546) of S protein in human alveoli in culture is increase by IL1β and downregulated by ELVs. (Panel A) XZ plane of a Z-stack image, in blue nuclei, in red RBD, and in white membrane (upper panel), (Panel B) digitalization of nuclei and RBD signal not membrane, (Panel C) complete digitalized image, (Panel D) Nuclei and RBD signal, (Panel E) digitalized nuclei and RBD. (Panel F) Superior view of digitalized nuclei and RBD with (left panel) and without membrane (right panel). (Panel G) Quantification of RBD internalized protein. The whole RBD signal was matched to 100% and using the Z position reference of the membrane, the % of above (free RBD), within (bound RBD) and below (internalized RBD) where determined. *p<0.05, ANOVA p=0.0005 in N=24 images for ELVs-treated cells and 48 images for Controls and

FIG. 47 shows RBD protein internalization decreased by ELVs 32 and 34 and their precursors, VLC-PUFAs 32:6 and 34:6. (Panel A) Added VLC-PUFAs induced ELV synthesis in alveolar cells (FIG. 41,45) and decreased RBD domain internalization. Bars: mean of RBD quantification located below the membrane line (FIG. 46). N=24 *p<0.05; **p<0.001. (Panel B) JESS WB ACE2 separation (left), bands densitometry (right). (Panel C) Quantification of ACE2. Bars are mean plus standard deviation *p<0.05.

FIG. 48 shows open reading frame cloned into pEFla-mCherry translated into the Spike protein sequence (using ORFinder NCBI). Signal peptide (membrane insertion consensus sequence, green), t receptor binding domain (RBD, light grey), RGD and LDI motifs for integrin binding (yellow), 51 portion (dark grey), ACE2 binding motif (aqua), Furin cleavage site (indigo) and S2 portion (pink) are indicated in the protein sequence. The ORF correspond to the new dominant variant of Spike with the re placement D614G, red (Korber et al. 2020).

FIG. 49 shows RBD internalization specificity in cultured human alveolar cells. Viral Nucleocapsid N protein tagged with Alexa Fluor-594 used in parallel to RBD to determine specific internalization of RBD. (Panel A) XZ plane of a Z-stack image of alveoli culture after 24 h exposure to N protein. (Panels B & C) Digitalized picture. (Panel D) View from below of digitalized image of alveoli cells exposed to RBD tagged with Alexa Fluor-594 for 24 h. White arrows show position of internalized RBD. (Panels E & F) The plots show digitalized elements position in the Z-axis from 2 representative images (Panel E) of N protein exposed, and (Panel F) from RBD exposed alveoli cells. The membrane thickness was taken as reference in each picture to determine % of proteins above (free protein), within (bound protein), and below (internalized protein). N protein did not internalize and was found partially bound to membrane while most remained free. (Panel G) Quantification of internalized signal when no protein, N and S were added to alveolar cells. Bars are mean of N=24 pictures +/−standard Error of the mean.

FIG. 50 shows VLC-PUFAs 32:6 and 34:6 (added to alveolar cells in culture), incorporated to phosphatidylcholine molecular species (18:1/32:6) and (18:1/34:6).

FIG. 51 shows heat maps of 168 GPCR and 73 Orphan GPRCs (antagonists/partial agonists). Screened by PathHunter 0-arrestin complementation vs. orphanMAX Panel (DiscoverX, Eurofins, CA). The GPCR targets (blue arrows) are active above threshold. NPD1, ELV32 or ELV34 (5 μM), or vehicle incubated with cells expressing GPCR panels. The GPCRs were blind screening (twice). Rainbow colored Heat maps (GraphPad Prism 8.2) shows % of activity (agonists) and % Inhibition (antagonists): smallest value 0 and largest value 60. Only GPCR that had high cutoff values (green to red) are considered putative candidates (blue arrows).

FIG. 52 shows ELVs inhibit house mite (HDM)-induced senescence in human nasal epithelial cells. (Panel A) Spider 0-gal for SASP, control and cells ELV34 and HDM. (Panel B) Quantification of 0-gal (N=12). (Panel C) RT-PCR of mRNA senescence genes; p21, p16 and p27 and inflammation: ILla, IL6 and IL10 (N=6). Bars: mean +/−standard error. *p<0.05.

FIG. 53 shows selective lipid mediators reduce cornea injury-induced expression of ACE2 and binding of Alexa 594-RBD. Panel a, Expression of Ace2, Dpp4, furin and Tmprss2 in the uninjured rat cornea. Left: representative immunofluorescence imaging. DAPI stains nuclei (blue). Immunofluorescence shows ACE2 expressed in the epithelium and stroma. Right: RNA-seq data. Panel b, Experimental design. After alkali burn, rats received eye drops of lipid mediators or vehicle 20 μl/eye, 3 times/day for 14 days (double-blinded). ACE2 expression was assayed at day 14 after injury +/−lipids treatment. At day 15, rats were treated with Alexa 594-RBD (1 μg/eye, 3 times) and corneas examined a day later. Panel c, Lipid mediators studied. The chirality in all figures of RvD6i and NPD1 used in this study had the R,R stereochemistry. Panel d, ACE2 abundance before and after injury +/−lipids using Jess capillary-based Western Blot system (Protein Simple). ACE2 densitometry normalized to GAPDH in the same capillary to minimize errors. Data is from one rat cornea for each data point (N=4). The p-values of ANOVA-post hoc Dunnett's multiple comparisons test with vehicle as reference are shown. Mean and SD are depicted as the lines. Panel e, Illustration showing corneal analysis by wholemount (x and z planes—orange color) and cross-section (x and y planes—blue color). Panel f, Wholemount images of binding of Alexa 594-RBD in corneas after injury and treatments. The control cornea (no-injury) has very low Alexa 594-RBD signal, while the injured cornea shows intense fluorescence. LXA4, ELV-N32, and RvD6i decrease Alexa 594-RBD binding while NPD1 fails. Panel g, Cross-section images of the same corneas shown in panel f. The green lines were added to separate the epithelium from the stroma. Most of Alexa 594-RBD signal was found in the stroma. Panel h, Quantification of Alexa 594-RBD positive cells. Each data point represents number of cells/cross-section image. Values are means±SD and p-values calculated by ANOVA-post hoc Dunnett's multiple comparisons test with vehicle as reference (4 images/cornea and 4 rat corneas/condition). The map of image capture is shown in FIG. 57.

FIG. 54 shows selective lipid mediators disrupt ACE2 upregulation and injury-mediated hyper-inflammation, senescence, and cytokine storm components. Panel a, PCA plot of RNA-seq data. Rat corneas were analyzed at day 14 after injury +/−treatments (FIG. 53 panel b). Each data point represents one animal (N=5/group, except LXA4 with N=3 and control with N=6). The eclipse of 95% confidence interval was used to group data points from the same set of treatment. Panel b, Venn diagram of significant genes (FDR <0.05) upregulated by the vehicle treatment of injured corneas (RNA-seq data set was analyzed using DEseq2 with vehicle injured corneas as reference). The negative log 2 fold change genes (upregulated by vehicle) with FDR <0.05 were used. We excluded NPD1 because it failed to decrease Ace2 expression upon injury (FIG. 53 panel d). The groups of shared genes between control-LXA4-ELV-N32-RvD6i and control-ELV-N32-RvD6i are depicted. Panel c, The KEGG-pathway enrichment networks of selected genes from panel b. Bars were sorted by p-value. The length of the bar represents the significance of the pathway, while the lighter the color, the higher the significance. The number shows amount of genes from denoted group that are enriched in each pathway. Panel d, IPA upstream regulator analysis of significant genes vs. vehicle (injury) group. There are proteins with negative activation z-score compared to vehicle group (blue color). Among those are CDKN2A and NFkB (complex). Panel e, RNA-seq normalized counts of Cdkn2a gene that encodes the senescence key-marker p 16INK4a; ELV-N32 decrease its expression. Data correspond to one cornea for each data point and is presented as mean±SD. The p-values were analyzed by ANOVA-post hoc Dunnett's multiple comparisons test with vehicle as reference. The normalized counts were used for analysis. IPA scores for CDKN2A (panel f) and NFkB (complex) (panel g) upstream regulators. The left y-axis is the inhibition z-score, while the right y-axis e is -log 10 of p-value. The cutoff line for p-value is <0.05.

FIG. 55 shows lipid mediators down-regulate injury-induced gene expression of NFkB/inflammation, senescence-associated secretory phenotype, and cytokine storm markers after cornea injury. Panel a, Venn diagram of cytokines, SASP, and NFkB inflammatory genes upregulated by injury. Panel b, Heatmap of normalized counts data. Each small square represents data from one cornea. There are 51 genes increased by injury, and most are inhibited by ELV-N32 and RvD6i treatment. Panel c, The ArchS4 human tissue analysis prediction for the 51 genes. The length of the bar represents the significance of the gene set in the tissues, while the lighter the color, the higher the significance. The number shows the amount of genes from the denoted group enriched in each pathway. Panel d, Scatter plots of IIIb, II6, and Vegfa genes. Panel e, Scatter plots of genes that encode proteins that target RGD. The p-value of ANOVA-post hoc Dunnett's multiple comparisons test with vehicle as reference are shown. Mean and SD are depicted as the lines. The normalized counts were used for analysis.

FIG. 56 shows lipid mediators attenuate IFNγ-induced ACE2 expression, senescence programming, and binding of Alexa 594-RBD in human corneal epithelial cells (HCEC). Panel a, Among several cytokines tested, IFNγ and α induces ACE2 expression in HCEC (6 hours after stimulation, analyzed by dd-PCR). Panel b, Effect of lipid mediators on gene expression of Ace2, Cdkn2a, and Mmp1 of HCEC after adding IFNγ (100 ng/ml). AACT normalized fold change was used. p-values of statistical t-test analysis in comparison to vehicle group are shown. Mean and SD are shown as the lines. Panel c, Alexa 594-RBD binding in HCEC. IFNγ (100 ng/ml) and lipid mediators (200 nM) were added to the HCEC for 12 h. Alexa 594-RBD (0.5 ng/well) was then added and images taken 24 h after. Fifteen images/condition analyzed. Representative images are shown (left side), and the Imaris based calculation was plotted (right-hand side). Data are presented as single image/each data point. The p-value of ANOVA-post hoc Dunnett's multiple comparisons test with vehicle as reference. Mean and SD are shown as the lines. Panel d, SASP Secretome ((3-Gal staining) of HCEC 24 h after IFN-γ challenge and +/−lipid mediators. Each point represents one image. The p-value of ANOVA-post hoc Dunnett's multiple comparisons test with vehicle as reference are shown. Mean and SD are shown as the lines. Representative images for each condition are in the right panel.

FIG. 57 shows lipid mediators on gene expression of Ace2, Dpp4, Furin, and Tmprss2 after cornea injury. RNA-seq data normalized counts, (mean and SD) analyzed by ANOVA-post hoc Dunnett's multiple comparisons test with vehicle as reference. *, p<0.05, **, p<***, p<0.001, and ****, p<0.0001.

FIG. 58 shows panel a, Illustration of unbiased microscopy analysis of the rat cornea. Four images were taken (red boxes) for cornea sections. Panel b, Representative images stained with CD68 and Neutrophil antibodies and with Alexa 594-RBD of a rat injured cornea. DAPI used to label the nuclei. Panel c, Quantification of macrophage (+CD68 cells) and neutrophil. Each data point represents number of cells/cross-section image. Values are means ±SD and p-values calculated by ANOVA-post hoc Dunnett's multiple comparisons test with vehicle as reference. *, p<0.05, **, p<0.01, ***, p<0.001, and ****, p<0.0001.

FIG. 59 shows lipid mediators attenuate expression of cytokine-storm related genes and SASP after cornea injury. Panel a, KEGG-pathway enrichment of 51 genes depicted in FIG. 66 panel b. Bars were sorted by p-value. The length of the bar represents the significance of the pathway, while the lighter the color, the more significant. The number shows the amount of genes from the denoted group that are enriched in each pathway. Panel b, RNA-seq gene expression share between cytokine-storm markers and SASP. Panel c, RNA-seq gene expression of cytokine storm-related genes. Normalized counts, (mean and SD) analyzed by ANOVA-post hoc Dunnett's multiple comparisons test with vehicle as reference. *, p<0.05, **, p<0.01, ***, p<0.001, and ****, p<0.0001.

FIG. 60 shows effect of lipid mediators on expression of NFkB inflammatory genes after cornea injury. Normalized counts, (mean and SD) analyzed by ANOVA-post hoc Dunnett's multiple comparisons test with vehicle as reference *, p<0.05, **, p<0.01, ***, p <0.001, and ****, p<0.0001.

FIG. 61 shows differential effect of lipid meditators on senescence programming gene expression after cornea injury, RNA-seq gene expression. Normalized counts, (mean and SD) analyzed by ANOVA-post hoc Dunnett's multiple comparisons test with vehicle as reference. *, p<0.05, **, p<0.01, ***, p<0.001, and ****, p<0.0001.

FIG. 62 shows dd-PCR gene expression analysis of Ace2 in HCEC after stimulation with 1, 10, and 100 ng/mL IFNε, IL 1β, IL2, IL6, IL8, and TNFα. There was no significant increase with any of the cytokines.

FIG. 63 shows unbiased imaging analysis for RBD binding in HCEC. Panel a, Images were taken in the Multi Area Time Lapse mode with an Olympus FV3000 confocal microscope. For each well, 7 designed areas were taken with the same parameters and Z-section range. Panel b, Images of a normal cornea showing the Imaris auto-fluorescence and the filtered image. Images were converted and inputted in the Imaris software, and the threshold for the control images (HCEC without Alexa 594-RBD) was defined. Then, the batch image processing was used to analyze all images with the defined threshold. The total sum intensity for each image was employed to evaluate binding efficiency. Panel c, Representative images of Alexa 594-RBD for vehicle and ELV-N32 treated HCEC from the microscopy (left) and after Imaris threshold-filtration (right).

FIG. 64 shows specific internalization of Spike's protein RBD is reduced by Elovanoids and their precursors in human primary alveolar cells. Panel a, Characterization of Pneumocytes type I (left panel) and type II (right panel) in the alveolar culture. Pneumocytes type I are reactive to HT1-53 (green), and pneumocytes type II are positive for oil red (red), a marker for the type II lipid/lamellar bodies and for ciliated cell marker Foxj1 (green) which is required for cilia formation and is an early marker of epithelial cell differentiation, recovery, and function. Panel b, Pneumocytes type II stained positive to HT2-280 labeled (green) type II human lung cells. Panels c-e, Differential entrance of S protein vs. N in human alveolar cells in culture. Z-stack shows signal distribution (upper panel), and in mobile cells undergoing cell division (lower panel). Panel c, XZ planes of a Z-stack showing white: membrane, red: RBD tagged with Alexa Fluor 594 and blue nuclei. i through v depict S protein view from XZ plane digitalized of the IMAMS image; vi to viii view from below, above and digitalized showing the internalized protein (white arrows); ix to xi XZ plane depicting the labeled N protein that remains in the surface or within the plasma membrane. Viral Nucleocapsid N protein was tagged with Alexa Fluor-594. XZ plane of a Z-stack image of alveoli culture after 24 h exposure to N protein. d, Quantification of internalized signal when no protein, N and S in the presence or absence of IL1β or TNFα. Panel e, position of the protein signal in the Z-axes. The plots show digitalized elements position in the Z-axis from 2 representative images of N protein (top panel), and for RBD (bottom panel). The membrane thickness was taken as reference in each picture to determine % of proteins above (free protein), within (bound protein), and below (internalized protein) where green dots are membrane signal, blue dots are nuclei, and red dots are protein. The Y-axis denotes the μm thickness of the image or Z. The boxes showed the median (middle), and the upper and lower lines depict the Quartile 1 and 3. The whiskers showed the maximum and minimum position adopted in the Z-axes. Panel f, RBD protein internalization decreased by ELV-N32, 34, and their precursors, VLC-PUFAs 32:6 and 34:6. The addition of ELV-N32 and ELV-N34, 204 of the acetylene form together (top panel) or 1 μM separated methyl ester form (bottom panel), and their precursors 32:6 and 34:6 (204) showed a decrease in the Alexa-Fluor 594-RBD internalization. Panels g-i, Expression of ACE2 and TMPRSS2 in alveolar cells. Immunocytochemistry of alveolar cells shows signal of ACE2 (Panel g, left panels) and TMPRSS2 (Panel h, left panels) in pneumocytes type II expressing β-tubulin-IV and the mRNA expression for both genes by Taqman qPCR (Panels g-h). Panel i, ACE2 protein quantification (by Jess-capillary Wester Blots-Protein Simple: left panel depicts ACE2 bands at 90 KDa for ACE2 and 40KDa for GAPDH; in the middle panel is shown the area below the pic intensity used to quantified the ACE2 band and the plot shows the quantitation of N=2 samples of alveolar cells +/−ELV-N32 and N34. Panel j, The expression of Sirtl mRNA, an ELV-N32 and N34 target. Bars depict the mean of N=24 pictures plus/minus standard error. P<0.05. Membrane (white), S or N Protein (red), and nuclei (blue). Boxes color code all the histograms in the figure.

FIG. 65 shows fate of VLC-PUFAs (C32:6 and C34:6, 2 μM each) added to human primary alveolar cells cultures towards ELV synthesis. Panel a, 27-monohydroxy-32:6, 29-monohydroxy-34:6, and ELVs are formed. Full fragmentation of 27-monohydroxy-32:6 and 29-monohydroxy-34:6 show matches to their theoretical peaks (top panels). The inserts depict the structures of 27-monohydroxy-32:6 and 29-monohydroxy-34:6 along with the product ions as they are cleaved at a given bond. The complete structures of ELV-N32 and ELV-N34 were confirmed to be: ELV-N32:(14Z,17Z,20R,21E,23E,25Z,27S,29Z)-20,27-dihydroxydo-triaconta 14,17,21,23,25,29-hexaenoic acid; ELV-N34: (16Z,19Z,22R,23E,25E,27Z,29S,31Z)-22,29-dihydroxytetra-triaconta-16,19,23,25,27, 31-hexaenoic acid (bottom panels). Panel b, Synthesis Pathways for ELV-N32 and ELV-N34 from VLC-PUFAs 34:6, n-3. Panel c, Relative abundance of the elovanoids N32 and N34 (bottom panels) and their intermediates 27 mono-hydroxy and 29 mon-hydroxy (top panels) when the alveolar cells are exposed to the precursors VLC-PUFAs: 32:6 and 34:6 in the presence or absence of 10 ng/ml IL1β and 10 ng/ml TNFα for 24 hours. The plot shows the box upper limit 3^{rd} Quartile, bottom side 1^{st} quartile, middle line: the median and the whiskers denote the maximum and minimum observations. *p<0.05 in t-test comparisons with the respective control. The compounds described in panel a are as follows:

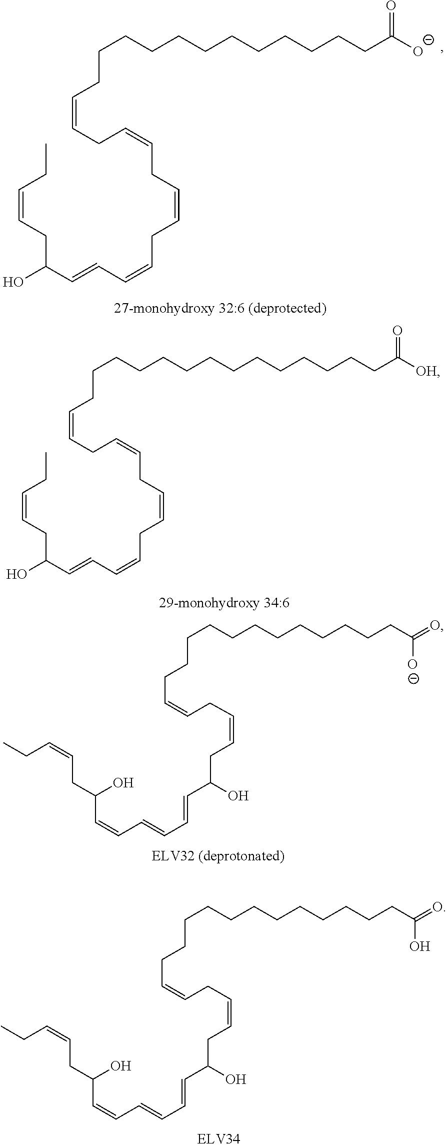 [SMILES: CC/C=C\CC(O)/C=C/C=C\C/C=C\C/C=C\C/C=C\CCCCCCCCCCCCC(=O)[O-] ; CC/C=C\CC(O)/C=C/C=C\C/C=C\C/C=C\C/C=C\CCCCCCCCCCCCCCC(=O)O ; CC/C=C\CC(O)\C=C/C=C/C=C/C(O)C/C=C\C/C=C\CCCCCCCCCCCCC(=O)[O-] ; CC/C=C\CC(O)\C=C/C=C/C=C/C(O)C/C=C\C/C=C\CCCCCCCCCCCCC(=O)O]

FIG. 66 shows pneumocyte markers in chamber slide cultures. Panel a shows Type I pneumocytes labeled (green) with the specific marker HT1-53 (upper panels). Panel b shows Type II lung pneumocytes labeled with a ciliated cell marker Foxj1 (green), which is required for cilia formation and is an early marker of epithelial cell differentiation, recovery, and function, and Oil Red O(red), a marker for the type II lipid/lamellar bodies (bottom panels). Nuclei were labeled with Hoechst (blue).

FIG. 67 shows chemical structures of the elovanoids N32 (top) and N34 (second). ELV-N32, R denotes the substituent in the carbon located is H=hydrogen or Me=methyl-ester. The third and fourth molecule from top to bottom shows the triple bond that make the molecules acetylenic.

FIG. 68, panels a, c, d, and e, shows semi-quantitative real time PCR quantitation of target of elovanoids N32 and N34, RNF-146 (alias Iduna), Prohibitin (PHB), Bcl2 and Bcl-XL (Primers in Table 1), in alveolar cells exposed to the acetylene form of the ELVs for 24 hours in the presence or absence of 10 ng/ml IL1β. FIG. 68, Panel b, shows Western blot assay on alveolar cells exposed for 24 h to Elovanoids N32 and N34 with the substituent R=methyl ester.

FIG. 69 shows Relative abundance of the elovanoids N32 and N34 (bottom panels) and their intermediates 27 mono-hydroxy and 29 mon-hydroxy (top panels) when the alveolar cells are exposed to the precursors VLC-PUFAs: 32:6 and 34:6 in the presence or absence of 10 ng/ml IL1β and 10 ng/ml TNFα for 24 hours. DHA abundance was analyzed as a specificity control. The plot shows the box upper limit 3^{rd} Quartile, bottom side 1^{st} quartile, middle line: the median and the whiskers denote the maximum and minimum observations. *p<0.05 in t-test comparisons with the respective control. Repetition of the experiment of FIG. 76 in 24 well plates.

FIG. 70 shows schematics validating therapeutics to block/attenuate entrance of SARS-CoV-2

FIG. 71 shows a graphic of a non-limiting example of an experimental design for induction of NETosis and its blockage using Elovanoids.

FIG. 72 shows graphs of a picogreen standard curve, A23187 5 μM-Picogreen DNA, and PMA 100 nM-Picogreen DNA.

FIG. 73 shows graphs of picogreen standard curve, A23187 5 μM-Picogreen DNA, PMA 100 nM-Picogreen DNA, and A23187 SYTOX Green.

FIG. 74 shows graphs of Cit-H3 Standard curve, A32187 Cit-H3, and PMA Cit-H3.

FIG. 75 shows graphs of Cit-H3 Standard curve, PMN Elastase Standard Curve, MPO Standard Curve, PMA Cit-H3, PMN Elastase, and MPO.

FIG. 76 shows an exemplary, non-limiting experimental design.

FIG. 77 shows exemplary, non-limiting clinical scores. The higher the score, the more severe the condition of the cornea.

FIG. 78 shows RNA seq data for the COVID-19 related receptors.

FIG. 79 shows the RNA-seq data for the COVID-19 related receptors.

FIG. 80 shows the RNA-seq data for the COVID-19 related proteins that are involved in endocytosis.

FIG. 81 shows the RNA-seq data for the COVID-19 related proteins that are involved in endocytosis.

FIG. 82 shows the RNA-seq data for the COVID-19 related proteases.

FIG. 83 shows the RNA-seq data for the COVID-19 related proteases.

FIG. 84 shows the RNA-seq data for the COVID-19 related proteases.

FIG. 85 shows COVID-19 entrance.

FIG. 86 shows the JESS WB for ACE2.

FIG. 87 shows the JESS WB for furin.

FIG. 88 shows the RNA-seq data for NPD1 and ELV34 receptors.

### DETAILED DESCRIPTION OF THE DISCLOSURE

The present invention is drawn towards compositions and methods of treating a viral infection, viral disease, or viral inflammatory response or immune dysfunction associated therewith by administering omega-3 very-long-chain polyunsaturated fatty acids (n-3 VLC-PUFA) to induce the biosynthesis of their hydroxylated derivatives (e.g., elovanoids), administering elovanoids and isomers thereof, administering Lipoxins (e.g. Lipoxin A4), administering Resolvins (e.g. Resolvin D6, R,R-RvD6i, an isomer thereof), or a combination thereof. Without wishing to be bound by theory, the biomolecules referenced herein can curtail virus-associated inflammation and reduce the cytokine storm by downregulating pro-inflammatory signaling and by modulating the expression of ACE2, TMPRSS2, furin and/or DPP4. For example, the inflammation and/or cytokine storm can be associated with a coronavirus, such as the SARS-CoV-2. These therapies could be deployed in many formats as described herein, such as a new oral inhalable lung surfactant, to attenuate or prevent virus cell entrance and prevent or limit virus shedding/transmissibility, and in doing so, attenuate viral disease onset and progression. As described herein, embodiments can be administered as a nasal formulation, such as an inhalable composition or nasal spray, to target the nasal mucosa.

In embodiments, the composition described herein comprises a biomolecule, such as Lipoxin A4, Resolvin D6, Resolving D6i, elovanoids, elovanoid-N32, isomers thereof, or variants thereof. The terms “elovanoid-N32” and “elovanoid N-32” can be used interchangeably. The terms “elovanoid N-34” and “elovanoid-N34” can be used interchangeably. The term “biomolecule” can refer any molecule of biological origin, composite, or fragmentary form thereof, or derivative thereof.

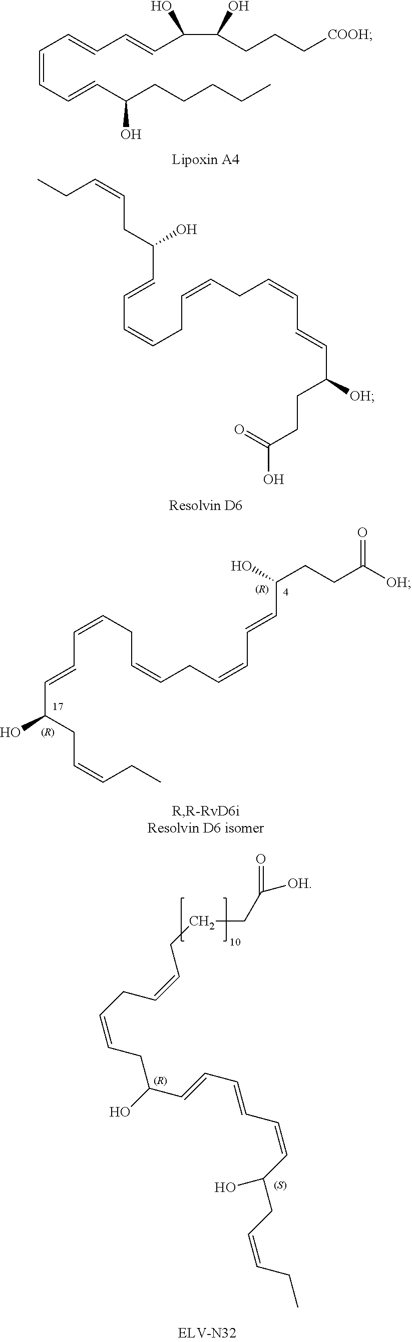

Before the present disclosure is described in greater detail, it is to be understood that this disclosure is not limited to any one embodiment described, and as such can, of course, vary. It is also to be understood that the terminology used herein is for the purpose of describing embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges can independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as understood by one of ordinary skill in the art to which this disclosure belongs. Although any methods and materials similar or equivalent to those described herein can also be used in the practice or testing of the present disclosure, the advantageous methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that can need to be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present disclosure. Any recited method can be carried out in the order of events recited or in any other reasonable order.

Embodiments of the disclosure will employ, unless otherwise indicated, techniques of medicine, organic chemistry, biochemistry, molecular biology, pharmacology, toxicology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that can have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which can contain additional structural groups, composition components or method steps (or analogs or derivatives thereof as discussed above). Such additional structural groups, composition components or method steps, etc., however, do not materially affect the characteristic(s) of the compositions or methods, compared to those of the corresponding compositions or methods disclosed herein.

Prior to describing the various embodiments, the following exemplary descriptions are provided.

As used herein, the nomenclature alkyl, alkoxy, carbonyl, etc. is used as is understood by those of skill in the chemical art. As used in this specification, alkyl groups can include straight-chained, branched and cyclic alkyl radicals containing up to about 20 carbons, or 1 to 16 carbons, and are straight or branched. Alkyl groups herein include, but are not limited to, methyl, ethyl, propyl, isopropyl, isobutyl, n-butyl, sec-butyl, tert-butyl, isopentyl, neopentyl, tert-pentyl and isohexyl.

As used herein, lower alkyl can refer to carbon chains having from about 1 or about 2 carbons up to about 6 carbons. Suitable alkyl groups can be saturated or unsaturated. Further, an alkyl can also be substituted one or more times on one or more carbons with substituents selected from a group consisting of C1-C15 alkyl, allyl, allenyl, alkenyl, C3-C7 heterocycle, aryl, halo, hydroxy, amino, cyano, oxo, thio, alkoxy, formyl, carboxy, carboxamido, phosphoryl, phosphonate, phosphonamido, sulfonyl, alkylsulfonate, arylsulfonate, and sulfonamide. Additionally, an alkyl group can contain up to 10 heteroatoms, in certain embodiments, 1, 2, 3, 4, 5, 6, 7, 8 or 9 heteroatom substituents. Suitable heteroatoms include nitrogen, oxygen, sulfur and phosphorous.

As used herein, “cycloalkyl” refers to a mono- or multicyclic ring system, in certain embodiments of 3 to 10 carbon atoms, in other embodiments of 3 to 6 carbon atoms. The ring systems of the cycloalkyl group can be composed of one ring or two or more rings which can be joined together in a fused, bridged or spiro-connected fashion.

As used herein, “aryl” refers to aromatic monocyclic or multicyclic groups containing from 3 to 16 carbon atoms. As used in this specification, aryl groups are aryl radicals, which can contain up to 10 heteroatoms, in certain embodiments, 1, 2, 3 or 4 heteroatoms. An aryl group can also be substituted one or more times, in certain embodiments, 1 to 3 or 4 times with an aryl group or a lower alkyl group and it can be also fused to other aryl or cycloalkyl rings. Suitable aryl groups include, for example, phenyl, naphthyl, tolyl, imidazolyl, pyridyl, pyrroyl, thienyl, pyrimidyl, thiazolyl and furyl groups.

As used in this specification, a ring can have up to 20 atoms that can include one or more nitrogen, oxygen, sulfur or phosphorous atoms, provided that the ring can have one or more substituents selected from the group consisting of hydrogen, alkyl, allyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, phosphonate, phosphonamido, and sulfonyl, and further provided that the ring can also contain one or more fused rings, including carbocyclic, heterocyclic, aryl or heteroaryl rings.

As used herein, alkenyl and alkynyl carbon chains, if not specified, contain from 2 to carbons, or 2 to 16 carbons, and are straight or branched. Alkenyl carbon chains of from 2 to 20 carbons, in certain embodiments, contain 1 to 8 double bonds, and the alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds.

As used herein, “heteroaryl” can refer to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group can be fused to a benzene ring. Heteroaryl groups include, but are not limited to, furyl, imidazolyl, pyrrolidinyl, pyrimidinyl, tetrazolyl, thienyl, pyridyl, pyrrolyl, N-methylpyrrolyl, quinolinyl and isoquinolinyl.

As used herein, “heterocyclyl” can refer to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen can be quaternized to form an ammonium group where the substituents are selected as above.

As used herein, “aralkyl” can refer to an alkyl group in which one of the hydrogen atoms of the alkyl is replaced by an aryl group.

As used herein, “halo”, “halogen” or “halide” can refer to F, Cl, Br or I.

As used herein, “haloalkyl” can refer to an alkyl group in which one or more of the hydrogen atoms are replaced by halogen. Such groups include, but are not limited to, chloromethyl and trifluoromethyl.

As used herein, “aryloxy” can refer to RO—, in which R is aryl, including lower aryl, such as phenyl.

As used herein, “acyl” can refer to a —COR group, including for example alkylcarbonyl, cycloalkylcarbonyl, arylcarbonyl, or heteroarylcarbonyls, all of which can be substituted.

As used herein, “n-3” or “n3”, “n-6” or “n6”, for example, can refer to the customary nomenclature of polyunsaturated fatty acids or their derivatives, wherein the position of a double bond (C═C) is at the carbon atom counted from the end of the carbon chain (methyl end) of the fatty acid or fatty acid derivative. For example, “n-3” can refer to the third carbon atom from the end of the carbon chain of the fatty acid or fatty acid derivative. Similarly, “n-3” or “n3”, “n-6” or “n6”, etc. also can refer to the position of a substituent such as a hydroxyl group (OH) located at a carbon atom of the fatty acid or fatty acid derivative, wherein the number (e.g. 3, 6, 9, 12 for example) is counted from the end of the carbon chain of the fatty acid or fatty acid derivative.

Abbreviations for any protective groups and other compounds can be in accord with their common usage, recognized abbreviations, or the IUPAC-IUB Commission on Biochemical Nomenclature (see, (1972) Biochem. 11:942-944).

As used herein, wherein in chemical structures of the compounds of the disclosure are shown having a terminal carboxyl group “—COOR,” the “R” can be a group covalently bonded to the carboxyl such as an alkyl group. In embodiments, the carboxyl group can further have a negative charge as “—COO” and R is a cation including a metal cation, an ammonium cation and the like.

The term “subject” or “patient” can refer to any organism to which aspects of the invention can be administered, e.g., for experimental, diagnostic, prophylactic, and/or therapeutic purposes. For example, subjects to which compounds of the disclosure can be administered include animals, such as mammals. Non-limiting examples of mammals include primates, such as humans. For veterinary applications, a wide variety of subjects will be suitable, e.g., livestock such as cattle, sheep, goats, cows, swine, and the like; poultry such as chickens, ducks, geese, turkeys, and the like; and domesticated animals for example pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject. As used herein, “pharmaceutically acceptable derivatives” of a compound can include salts, esters, enol ethers, enol esters, acetals, ketals, orthoesters, hemiacetals, hemiketals, acids, bases, solvates, hydrates or prodrugs thereof. Such derivatives can be readily prepared by those of skill in this art using known methods for such derivatization. The compounds produced can be administered to animals or humans without substantial toxic effects and either are pharmaceutically active or are prodrugs.

Pharmaceutically acceptable salts can include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl) aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

Compounds of those described herein, for example, can exist in the form of salts, for example acid addition salts or, in certain cases salts of organic and inorganic bases such as phenolate, carboxylate, sulphonate and phosphate salts. All such salts are within the scope of this disclosure.

The salts of the disclosure can be synthesized from the parent compound that contains a basic or acidic moiety by conventional chemical methods such as methods described in Pharmaceutical Salts: Properties. Selection, and Use, P. Heinrich Stahl (ed), Camille G. Wermuth (ed), ISBN:3-90639-026-8, Hardcover, 388 pages, August 2002. In embodiments, such salts can be prepared by reacting the free acid or base forms of these compounds with the appropriate base or acid in water or in an organic solvent, or in a mixture of the two; for example, non-aqueous media such as ether, ethyl acetate, ethanol, isopropanol, or acetonitrile are used.

Pharmaceutically acceptable esters can include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

Pharmaceutically acceptable enol ethers can include, but are not limited to, derivatives of formula C═C(OR) where R is hydrogen, alkyl, alkenyl, alkynyl, aryl, heteroaryl, aralkyl, heteroaralkyl, cycloalkyl, or heterocyclyl. Pharmaceutically acceptable enol esters can include, but are not limited to, derivatives of formula C═C(OC(O)R) where R is hydrogen, alkyl, alkenyl, alkynyl, aryl, heteroaryl, aralkyl, heteroaralkyl, cycloalkyl ar heterocyclyl.

Pharmaceutically acceptable solvates and hydrates are complexes of a compound with one or more solvent or water molecules, or 1 to about 100, or 1 to about 10, or one to about 2, 3 or 4, solvent or water molecules.

A reference to a compound of the disclosure and sub-groups thereof also includes ionic forms, salts, solvates, isomers, tautomers, esters, prodrugs, isotopes and protected forms thereof; such as, the salts or tautomers or isomers or solvates thereof; and more advantageously, the salts or tautomers or solvates thereof. As used herein, the term “isomer” can refer to molecules or polyamtoic ions with identical molecular formulas, but distinct arrangements of atoms in space. For example, constitutional isomers and stereoisomers of compounds described herein are also embodiments of the invention. For example, the enantiomers and diastereomers of compounds described herein can be aspects of the invention. For example, if (R,S) of a compound is described herein, (R, R), (S, R), and (S, S) can also be aspects of the invention. As used herein, the term “enantiomer” can refer to molecules which are nonsuperimposable mirror images of each other. As used herein, the term “diastereomer” can refer to a stereoisomer of a compound having two or more chiral centers that is not a mirror image of another stereoisomer of the same compound.

“Formulation” as used herein can refer to any collection of components of a compound, mixture, or solution selected to provide optimal properties for a specified end use, including product specifications and/or service conditions. The term formulation can include liquids, semi-liquids, colloidal solutions, dispersions, emulsions, microemulsions, and nanoemulsions, including oil-in-water emulsions and water-in-oil emulsions, pastes, powders, and suspensions. The formulations can also be included, or packaged, with other non-toxic compounds, such as cosmetic carriers, excipients, binders and fillers, and the like. For example, the acceptable cosmetic carriers, excipients, binders, and fillers for use in the practice of the invention are those which render the compounds amenable to oral delivery and/or provide stability such that the formulations of the present invention exhibit a commercially acceptable storage shelf life.

As used herein, the term “administering” can refer to introducing a substance, such as a VLC-PUFA, elovanoid, Lipoxin, Resolvin, derivatives thereof, isomers thereof, or a combination thereof into a subject. Any route of administration can be utilized including, for example, intranasal, topical, oral, parenteral, intravitreal, intraocular, ocular, subretinal, intrathecal, intravenous, subcutaneous, transcutaneous, intracutaneous, intracranial and the like administration. In embodiments, “administering” can also refer to providing a therapeutically effective amount of a formulation or pharmaceutical composition to a subject. The formulation or pharmaceutical compound can be administered alone, but can be administered with other compounds, excipients, fillers, binders, carriers or other vehicles selected based upon the chosen route of administration and standard pharmaceutical practice. Administration can be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

In embodiments a therapeutically effective amount of a composition described herein can comprise less than about 0.1 mg/kg, about 0.1 mg/kg, about 0.5 mg/kg, about 1.0 mg/kg, about 2.5 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 120 mg/kg, about 135 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 525 mg/kg, about 550 mg/kg, about 575 mg/kg, about 600 mg/kg, about 625 mg/kg, about 650 mg/kg, about 675 mg/kg, about 700 mg/kg, about 725 mg/kg, about 750 mg/kg, about 775 mg/kg, about 800 mg/kg, about 825 mg/kg, about 850 mg/kg, about 875 mg/kg, about 900 mg/kg, about 1.0 g/kg, about 1.5 g/kg, about 2.0 g/kg, about 2.5 g/kg, about 5 g/kg, about 10 g/kg, about 25 g/kg, about 50 g/kg, or more than 50 g/kg of compound per body weight of a subject.

In embodiments a therapeutically effective amount of a composition described herein can comprise a concentration of about 1 nM, about 10 nM, about 25 nM, about 50 nM, about nM, about 100 nM, about 200 nM, about 250 nM, about 300 nM, about 400 nM, about 500 nM, about 600 nM, about 700 nM, about 800 nM, about 900 nM, and about 1000 nM. For example, the concentration can be about 500 nM. For example, the concentration can be about 700 nM.

The formulations or pharmaceutical composition can also be included, or packaged, with other non-toxic compounds, such as pharmaceutically acceptable carriers, excipients, binders and fillers including, but not limited to, glucose, lactose, gum acacia, gelatin, mannitol, xanthan gum, locust bean gum, galactose, oligosaccharides and/or polysaccharides, starch paste, magnesium trisilicate, talc, corn starch, starch fragments, keratin, colloidal silica, potato starch, urea, dextrans, dextrins, and the like. For example, the pharmaceutically acceptable carriers, excipients, binders, and fillers for use in the practice of the present invention are those which render the compounds of the invention amenable to intranasal delivery, oral delivery, parenteral delivery, intravitreal delivery, intraocular delivery, ocular delivery, subretinal delivery, intrathecal delivery, intravenous delivery, subcutaneous delivery, transcutaneous delivery, intracutaneous delivery, intracranial delivery, topical delivery and the like. Moreover, the packaging material can be biologically inert or lack bioactivity, such as plastic polymers or silicone, and can be processed internally by the subject without affecting the effectiveness of the composition/formulation packaged and/or delivered therewith.

“Parenteral administration” can refer to administration via injection or infusion. Parenteral administration includes, but is not limited to, subcutaneous administration, intravenous administration, intramuscular administration.

For oral preparations, the composition or pharmaceutical composition can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Embodiments of the composition or pharmaceutical composition can be formulated into preparations for injection by dissolving, suspending, or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the composition or pharmaceutical composition can be utilized in aerosol formulation to be administered via inhalation. Embodiments of the composition or pharmaceutical composition can be formulated into pressurized acceptable propellants such as dichiorodifluoromethane, propane, nitrogen and the like.

Unit dosage forms for oral administration, such as syrups, elixirs, and suspensions, can be provided wherein each dosage unit, for example, teaspoonful, tablespoonful, tablet or suppository, contains a predetermined amount of the composition containing one or more compositions. Similarly, unit dosage forms for injection or intravenous administration may comprise the composition or pharmaceutical composition in a composition as a solution in sterile water, normal saline or another pharmaceutically acceptable carrier.

Embodiments of the composition or pharmaceutical composition can be formulated in an injectable composition in accordance with the disclosure. For example, injectable compositions are prepared as liquid solutions or suspensions; solid forms suitable for solution in, or suspension in, liquid vehicles prior to injection can also be prepared. The preparation can also be emulsified or the active ingredient (triamino-pyridine derivative and/or the labeled triamino-pyridine derivative) encapsulated in liposome vehicles in accordance with the disclosure.

In an embodiment, the composition or pharmaceutical composition can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Embodiments of the composition or pharmaceutical composition can be administered to a subject in one or more doses. Those of skill will readily appreciate that dose levels can vary as a function of the specific composition or pharmaceutical composition administered, the severity of the symptoms and the susceptibility of the subject to side effects. Dosages for a given compound are readily determinable by those of skill in the art by a variety of means.

In an embodiment, multiple doses of the composition or pharmaceutical composition are administered. The frequency of administration of the composition or pharmaceutical composition can vary depending on any of a variety of factors, e.g., severity of the symptoms, and the like. For example, in an embodiment, the composition or pharmaceutical composition can be administered once per month, twice per month, three times per month, every other week (qow), once per week (qw), twice per week (biw), three times per week (tiw), four times per week, five times per week, six times per week, every other day (qod), daily (ad), twice a day (qid), three times a day (tid), or four times a day. As discussed above, in an embodiment, the composition or pharmaceutical composition is administered 1 to 4 times a day over a 1 to 10-day time period.

The duration of administration of the composition or pharmaceutical composition analogue, e.g., the period of time over which the composition or pharmaceutical composition is administered, can vary, depending on any of a variety of factors, including patient response. For example, the composition or pharmaceutical composition in combination or separately, can be administered over a period of time of about one day to one week, about one day to two weeks.

In embodiments, two or more biomolecules and/or antiviral agents, “agents”, can be administered sequentially, such as one before the other, or concurrently or simultaneously, such as at about the same time. The term “simultaneous administration”, as used herein, indicates that the first agent and the second agent in the therapeutic combination therapy are administered either less than about 15 minutes, e.g., less than about 10, 5, or 1 minute. When the first and second agents are administered simultaneously, the first and second treatments can be in the same composition (e.g., a composition comprising both the first and second therapeutic agents) or separately (e.g., the first therapeutic agent is contained in one composition and the second treatment is contained in another composition).

As used herein, the term “sequential administration” can indicate that the first agent and the second agent in combination therapy are greater than about 15 minutes, such as greater than about 20, 30, 40, 50, 60 minutes, or greater than 60 minutes. Either the first agent or the second agent can be administered first. The first and second agents are included in separate compositions, which can be included in the same or different packages or kits.

As used herein, the term “simultaneous administration” means that administration of a first therapeutic agent and a second therapeutic agent in a combination therapy overlap each other.

The therapeutic compositions of the disclosure provide methods and compositions for the administration of the active agent(s) to a subject using any available method and route suitable for drug delivery, including in vivo, in vitro and ex vivo methods, as well as systemic and localized routes of administration. Routes of administration include intranasal, intramuscular, intratracheal, subcutaneous, intra cerebroventricular, intradermal, topical application, intravenous, rectal, nasal, oral, and other enteral and parenteral routes of administration. Routes of administration can be combined, if desired, or adjusted depending upon the agent and/or the desired effect. An active agent can be administered in a single dose or in multiple doses.

Embodiments of the composition or pharmaceutical composition can be administered to a subject using available conventional methods and routes suitable for delivery of conventional drugs, including systemic or localized routes. Routes of administration can include, but are not limited to, enteral administration, parenteral administration, or inhalation.

Other compositions, compounds, methods, features, and advantages of the disclosure will be or become apparent to one having ordinary skill in the art upon examination of the following drawings, detailed description, and examples. It is intended that all such additional compositions, compounds, methods, features, and advantages be included within this description, and be within the scope of the disclosure.

Embodiments as described herein can comprise a step of administering to a subject in need thereof a composition or formulation as described herein. For example, Lipoxin A4, Resolvin D6, Resolving D6i, elovanoids, or ELV-N32 for example, can be administered in a subject to prevent or treat a viral infection, such as a coronavirus infection. The term “administering” can refer to providing a therapeutically effective amount of a formulation or pharmaceutical composition to a subject, using intravitreal, intranasal, intraocular, ocular, subretinal, intrathecal, intravenous, subcutaneous, transcutaneous, intracutaneous, intracranial, topical and the like administration. However, any route of administration, such as oral, intravenous, subcutaneous, peritoneal, intra-arterial, inhalation, vaginal, rectal, nasal, introduction into the cerebrospinal fluid, intravascular either veins or arteries, or instillation into body compartments can be used.

One advantageous route of administration is topical administration. As used herein, the term “topical administration” can refer to administration onto any accessible body surface of any human or animal species, preferably the human species, for example, such as to the surface of the eye. Accordingly, the term “topical pharmaceutical composition” can include those dosage forms in which the compound is administered externally by direct contact with a topical treatment site, for example, the eye or the skin. The term “topical ocular pharmaceutical composition” can refer to a pharmaceutical composition suitable for administration directly to the eye. The term “topical epidermal pharmaceutical composition” can refer to a pharmaceutical composition suitable for administration directed to the epidermal layer of the skin, for example, the eyelid, the eyebrow, the scalp or the body.

Pulmonary/respiratory drug delivery can be implemented by different approaches, including liquid nebulizers, aerosol-based metered dose inhalers (MDI's), sprayers, dry powder dispersion devices and the like. Such methods and compositions are well known to those of skill in the art, as indicated by U.S. Pat. Nos. 6,797,258, 6,794,357, 6,737,045, and 6,488,953, all of which are incorporated by reference. According to the invention, at least one pharmaceutical composition can be delivered by any of a variety of inhalation or nasal devices known in the art for administration of a therapeutic agent by inhalation. Other devices suitable for directing pulmonary or nasal administration are also known in the art. For example, for pulmonary administration, at least one pharmaceutical composition is delivered in a particle size effective for reaching the lower airways of the lung or sinuses. Non-limiting examples of commercially available inhalation devices suitable for the practice of this invention are Turbohaler™ (Astra), Rotahaler® (Glaxo), Diskus® (Glaxo), Spiros™ inhaler (Dura), devices marketed by Inhale Therapeutics, AERx™ (Aradigm), the Ultravent® nebulizer (Mallinckrodt), the Acorn II® nebulizer (Marquest Medical Products), the Ventolin® metered dose inhaler (Glaxo), the Spinhaler® powder inhaler (Fisons), or the like.

All such inhalation devices can be used for the administration of a pharmaceutical composition in an aerosol. Such aerosols can comprise either solutions (both aqueous and non aqueous) or solid particles. Metered dose inhalers can use a propellant gas and require actuation during inspiration. See, e.g., WO 98/35888; WO 94/16970. Dry powder inhalers use breath-actuation of a mixed powder. See U.S. Pat. Nos. 5,458,135; 4,668,218; PCT publications WO 97/25086; WO 94/08552; WO 94/06498; and European application EP 0237507, each of which is incorporated herein by reference in their entirety. Nebulizers produce aerosols from solutions, while metered dose inhalers, dry powder inhalers, and the like generate small particle aerosols. Suitable formulations for administration include, but are not limited to nasal spray or nasal drops, and can include aqueous or oily solutions of a therapeutic composition as described herein.

A spray comprising a pharmaceutical composition as described herein can be produced by forcing a suspension or solution of a composition through a nozzle under pressure. The nozzle size and configuration, the applied pressure, and the liquid feed rate can be chosen to achieve the desired output and particle size. An electrospray can be produced, for example, by an electric field in connection with a capillary or nozzle feed.

A pharmaceutical composition as described herein can be administered by a nebulizer such as a jet nebulizer or an ultrasonic nebulizer. For example, in a jet nebulizer, a compressed air source is used to create a high-velocity air jet through an orifice. As the gas expands beyond the nozzle, a low-pressure region is created, which draws a composition through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, for example, employing a piezoelectric transducer. This energy is transmitted to the composition creating an aerosol.

Different forms of the inventive formulation can be calibrated in order to adapt both to different individuals and to the different needs of a single individual. However, the formulation need not counter every cause in every individual. Rather, by countering the necessary causes, the formulation will restore the body to its normal function. Then the body will correct the remaining deficiencies.

The term “therapeutically effective amount” as used herein can refer to that amount of an embodiment of the composition or pharmaceutical composition being administered that will relieve to some extent one or more of the symptoms of the disease or condition being treated, and/or that amount that will prevent, to some extent, one or more of the symptoms of the condition or disease that the subject being treated has or is at risk of developing. As used interchangeably herein, “subject,” “individual,” or “patient,” can refer to a vertebrate, such as a mammal, for example a human. Mammals can include, but are not limited to, murines, simians, humans, farm animals, sport animals, and pets. The term “pet” can include a dog, cat, guinea pig, mouse, rat, rabbit, ferret, and the like. The term farm animal can include a horse, sheep, goat, chicken, pig, cow, donkey, llama, alpaca, turkey, and the like.

A therapeutically effective dose can depend upon a number of factors known to those of ordinary skill in the art. The dosage can vary depending upon known factors such as the pharmacodynamic characteristics of the active ingredient and its mode and route of administration; time of administration of active ingredient; identity, size, condition, age, sex, health and weight of the subject or sample being treated; nature and extent of symptoms; kind of concurrent treatment, frequency of treatment and the effect desired; and rate of excretion. These amounts can be readily determined by the skilled artisan.

A “pharmaceutically acceptable excipient,” “pharmaceutically acceptable diluent,” “pharmaceutically acceptable carrier,” or “pharmaceutically acceptable adjuvant” can refer to an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. “A pharmaceutically acceptable excipient, diluent, carrier and/or adjuvant” as used herein can include one and more such excipients, diluents, carriers, and adjuvants.

The phrase “pharmaceutical composition” or a “pharmaceutical formulation” can refer to a composition or pharmaceutical composition suitable for administration to a subject, such as a mammal, especially a human and that can refer to the combination of an active agent(s), or ingredient with a pharmaceutically acceptable carrier or excipient, making the composition suitable for diagnostic, therapeutic, or preventive use in vitro, in vivo, or ex vivo. A “pharmaceutical composition” can be sterile and can be free of contaminants that can elicit an undesirable response within the subject (e.g., the compound(s) in the pharmaceutical composition is pharmaceutical grade). Pharmaceutical compositions can be designed for administration to subjects or patients in need thereof via a number of different routes of administration including oral, intranasal, topical, intravenous, buccal, rectal, parenteral, intraperitoneal, intradermal, intracheal, intramuscular, subcutaneous, by stent-eluting devices, catheters-eluting devices, intravascular balloons, inhalational and the like.

In embodiments, the composition described herein comprises a biomolecule, such as Lipoxin A4, Resolvin D6, Resolvin D6i, elovanoids, isomers thereof, or variants thereof. In embodiments the terms “elovanoid-N32” and “elovanoid N-32” can be used interchangeably. In embodiments, the terms “elovanoid N-34” and “elovanoid-N34” can be used interchangeably. The term “biomolecule” can refer to any molecule of biological origin, composite, or fragmentary form thereof, isomer thereof, or derivative thereof. In embodiments, if a compound described herein (e.g. VLC-PUFA) can be used therapeutically, then compounds described herein (elovanoids, lipoxins, resolvins, isomers thereof, and derivatives thereof) can also be used therapeutically.

In embodiments, the pharmaceutical composition can comprise a therapeutically effective amount of an elovanoid, a VLC-PUFA, a Lipoxin, a Resolvin, and/or isomers thereof and a therapeutically effective amount of one or more additional active agents (such as one or more anti-oxidants, anti-allergenics, anti-inflammatory agents, anti-viral agents, pain relievers, or antipyretics). For example, the one or more anti-oxidants can be synthetic antioxidants, natural antioxidants, or a combination thereof. As used herein, the phrase “active agents” and “active ingredients” can be used interchangeably. As used herein, the phrase “active agent” can refer to a biologically active substance.

In aspects of the invention an anti-viral agent can be used in combination with a therapeutic composition described herein. Anti-viral agents can include, but are not limited to abacavir; acemannan; acyclovir; acyclovir sodium; adefovir; alovudine; alvircept sudotox; amantadine hydrochloride; amprenavir; aranotin; arildone; atevirdine mesylate; avridine; cidofovir; cipamfylline; cytarabine hydrochloride; delavirdine mesylate; desciclovir; didanosine; disoxaril; edoxudine; efavirenz; enviradene; enviroxime; famciclovir; famotine hydrochloride; fiacitabine; fialuridine; fosarilate; trisodium phosphonoformate; fosfonet sodium; ganciclovir; ganciclovir sodium; idoxuridine; indinavir; kethoxal; lamivudine; lobucavir; memotine hydrochloride; methisazone; nelfinavir; nevirapine; palivizumab; penciclovir; pirodavir; ribavirin; rimantadine hydrochloride; ritonavir; saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zidovudine; zinviroxime, interferon, cyclovir, alpha-interferon, and/or beta globulin. In certain aspects, other antibodies against viral proteins or cellular factors may be used in combination with a therapeutic composition described herein.

In embodiments, the anti-oxidants can protect the double bonds of the elovanoids and/or of VLC-PUFAs. As used herein, “elovanoid” or “of a VLC-PUFA” can be used interchangeably.

“Lipoxin” can refer to lipoxygenase interaction products, which are generally bioactive autacoid metabolites of arachidonic acid (AA). Lipoxins can be categorized as non-classic eicosanoids and members of the specialized pro-resolving mediator family of PUFA metabolites. Lipoxins include, for example, lipoxin A4 (LXA4), lipoxin B4 (LXB4) as well as epimers of the same (i.e., 15-epi-LXA4 and 15-epi-LXB4, respectively).Lipoxins are biosynthesized from arachidonic acid.

Lipoxins are potent mediators of the resolution phase of the inflammatory response and of dysfunctional immunity. See, Serhan C. N., et al. (1999) Adv. Exp. Med. Biol. 469:287-293; and Fiorucci S., et al. (2004) Proc. Natl. Acad. Sci. USA. 101: 15736-15741. Lipoxin A4 and its analogs, including lipoxin A4 epimer 15 (or 15-epi-lipoxin A4), are well known in the art. See, U.S. Pat. Nos. 6,831,186 and 6,645,978; LM. Fierro et al., Journal of Immunology, vol. 170, pp. 2688-2694 (2003); G. Bannenberg et al., Brit. J. Pharma. Vol. 143, pp. 43-52 (2004); and R. Scalia et al, Proc. Natl. Acad. Sci. USA. vol. 94, pp. 9967-9*972 (1997). Lipoxin A4 and docosahexaenoic acid-derived neuroprotectin D1 (NPD1) are lipid autacoids formed by 12/15 lipoxygenase (LOX) pathways that exhibit anti-inflammatory and neuroprotective properties. Mouse corneal epithelial cells were found to generate both endogenous lipoxin A4 and NPD1. See, K. Gronert et al, PNAS, vol. 280, pp. 15267-15278 (2005). Lipoxins have been reported to play a role in wound healing in the corneal of the eye. See, K. Gronert, Prostaglandins, Leukotrienes and Essential Fatty Acids, vol. 73, pp. 221-229 (2005). Lipoxin A4 was shown to be formed in the epithelium of healthy and injured corneas, and lipoxygenase (LOX) enzyme activity has been indicated in the cornea of rats and rabbits. In the mouse cornea, lipoxin A4 was found to be generated in the absence of inflammation. In other tissues, lipoxins are predominantly formed during the resolution phase of acute inflammation. (Gronert, 2005). Lipoxin A4 or LOX have not been reported from the cornea endothelial cells, or from any cells of the back of the eye, only from the corneal epithelial cells. See, also, Bazan, N. et al, Survey of Opth., Vol. 41, Supp.2, pp. S23-S34 (1997); Bazan, N. et ah, Int'l Opth., Vol. 14, pp. 335-344 (1990); and Bazan, N., The Ocular Effects of Prostaglandins and Other Eicosanoids, Pub. Alan R. Liss, Inc., pp. 15-37 (1989).

In embodiments, Lipoxin can comprise the following structure

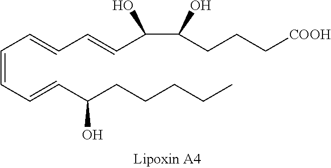 [SMILES: CCCCC[C@@H](O)/C=C/C=C\C=C\C=C\[C@@H](O)[C@@H](O)CCCC(=O)O]

“Resolvin” can refer to an autacoid that is a dihydroxy or trihydroxy metabolite of omega-3 fatty acids, including eicosapentaenoic acid (EPA), docosahexaenoic acid (DHA), docosapenaenoic acid (DPA) and clupanodonic acid. Resolvins are members of the specialized pro-resolving mediator class of PUFA metabolites. Resolvins include, for example, resolvin D1, resolvin D6 and resolvin E1.

In embodiments, resolvin D6 can comprise

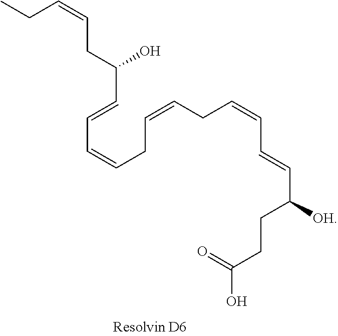 [SMILES: CC/C=C\C[C@H](O)/C=C/C=C\C/C=C\C/C=C\C=C\[C@@H](O)CCC(=O)O]

In embodiments, Resolvin D6 isomer can comprise

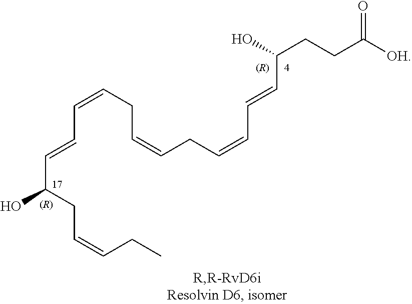

As used herein the terms “Resolvin D6 isomer”, “Resolvin D6i”, “RvD6i”, and “R,R-RvD6i” can be used interchangeably.

The term “isomer” can refer to stereoisomers and/or geometric isomers of the inventive polymers, e.g., cis- and trans-isomers, R- and S-enantiomers, diastereomers, (D)-isomers, (L)-isomers, the racemic mixtures thereof, as well as “head-to-tail” and “tail-to-tail” configurational isomers.

As used herein, the term “pro-homeostatic” can refer to the ability to promote or maintain homeostasis or a homeostatic state.

As used herein, the phrase “lipid mediator” can refer to a class of biologically active lipids which can be produced via biosynthesis in response to extracellular stimuli. See, for example, WO2016130522A1 and WO2018175288A1, each of which are incorporated by reference herein in their entireties. n-3 very-long-chain polyunsaturated fatty acids (“n-3 VLC-PUFA”, also “n3 VLC-PUFA”) can be converted in vivo to several types of VLC-PUFA hydroxylated derivatives named elovanoids (ELVs) that can protect and prevent the progressive damage to tissues and organs, whose functional integrity has been disrupted. Additionally, the biomolecules described herein can protect and prevent the progressive damage to tissues and organs, whose functional integrity has been disrupted.

In embodiments, the term “derivative” can refer to a structural analog. In embodiments, the term “derivative” can refer to a compound derived from a similar compound by a chemical reaction. ELVs have structures resembling docosanoids but with different physicochemical properties and alternatively-regulated biosynthetic pathways. In embodiments, for example, the elovanoids comprise 32- and/or 34-carbon elovanoids termed ELV-N32 and ELV-N34, or salts thereof. In embodiments, di-hydroxylated elovanoids can comprise 4 cis carbon-carbon double bonds starting at positions n-3, n-7, n-15 and n-18, and 2 trans carbon-carbon bonds starting at positions n-9, n-11. In embodiments, di-hydroxylated elovanoids can comprise 3 cis carbon-carbon double bonds starting at positions n-3, n-7, n-12 and n-15, and 2 trans carbon-carbon bonds starting at positions n-9, n-11. In embodiments, derivatives can comprise “phospholipid derivatives”. For example, an ester or ether bond of compounds described herein can be connected to a phospholipid.

The term “administration” can refer to introducing a composition described herein into a subject. Non-limiting examples of routes of administration of the composition comprise topical administration, oral administration, or intranasal administration. However, any route of administration, such as intravenous, subcutaneous, peritoneal, intra-arterial, inhalation, vaginal, rectal, introduction into the cerebrospinal fluid, intravascular either veins or arteries, or instillation into body compartments can be used.

As used herein, “treatment” and “treating” can refer to the management and care of a subject for the purpose of combating a condition, disease or disorder, such as a viral infection, viral disease, or viral induced inflammatory response, in any manner in which one or more of the symptoms of a disease or disorder are ameliorated or otherwise beneficially altered. The term can include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” can refer to the management and care of a patient for the purpose of hindering the development of the condition, disease or disorder, and includes the administration of the active compounds to prevent or reduce the risk of the onset of symptoms or complications.

As used herein, the term “prevention” or “preventing” can refer to stopping, or at least decreasing the probability of occurrence of an infection in a subject by a virus. By virtue of the administration of at least one composition described herein, the human or animal cells of said subject can become less permissive to the infection and can be more likely not to be infected with said coronavirus.

The phrase “alleviating a symptom of” can refer to ameliorating, reducing, or eliminating any condition or symptom associated with a viral infection, viral disease, or viral induced inflammatory response. Non-limiting examples of symptoms of viral infection, viral disease or viral induced inflammatory response comprise high viral loads, respiratory distress, and pulmonary damage correlated with high cytokine abundance. Cytokines coordinate the body's response to infection, trigger inflammation, and in COVID-19 (SARS-CoV-2) they can be generated in uncontrolled amounts. Generation of uncontrolled amounts of cytokines can be referred to as a “cytokine storm”. The term “cytokine storm” can refer to a series of events that result in a devastating and sometime fatal immune reaction that comprises a positive feedback loop between cytokines and immune cells that in turn leads to highly elevated levels of various cytokines. Cytokines that are induced during cytokine storm include, e.g., one or more of the following: IL4, IL2, IL12, TNF, IFNγ, IL6, IL8, and IL10. Cytokine storm can lead to multi-organ failure (heart, lung, kidneys) and lead to death. Non-limiting examples of symptoms of viral infections, for example SARS-CoV-2, include cough, shortness of breath, difficulty breathing, fever, chills, muscle pain, headache, exhaustion, sore throat, loss of taste or small, nausea, vomiting and/or diarrhea. Symptoms may appear 2, 5, 14, 28, or greater than 28 days after exposure to the virus.

As used herein, “coronavirus infection” can refer to a human or animal organism that has cells that have been infected by a coronavirus, such as SARS-CoV-2. The infection can be established by performing a detection and/or viral titration from respiratory samples, or by assaying blood-circulating CoV-specific antibodies. The detection in the individuals infected with the specific virus is made by conventional diagnostic methods, in particular of molecular biology (e.g. PCR), which are well known to those skilled in the art.

Still further, aspects of the invention are drawn to compositions and methods for ameliorating one or more symptoms of a virus infection. The term “amelioration” or “ameliorating” can refer to a lessening of severity of at least one symptom or indicator of a condition or disease, such as a viral infection. For example, in the context of a coronavirus infection, amelioration can include the reduction of inflammation. Embodiments herein can ameliorate one or more symptoms of coronavirus infection, including fever, cough, shortness of breath, fatigue, muscle or body aches, loss of taste or smell, or sore throat.

The patient to be treated can be a mammal, such as a human being. Treatment also encompasses any pharmaceutical use of the compositions herein, such as use for preventing, treating or alleviating a symptom of a disease, such as a viral infection, as provided herein. Treating an infection, such as a coronavirus infection, can refer to fighting the coronavirus infection in a subject. By virtue of the administration of at least one composition according to the invention, the viral infection rate (infectious titer) in the organism will decrease, and, in embodiments, the virus will completely disappear from the organism. The term “treatment” or “treating” can also refer to attenuating symptoms associated with the viral infection (respiratory syndrome, kidney failure, fever, for example).

In embodiments, the composition can further comprise one or more “nutritional components”. The term “nutritional component” as used herein can refer to such as protein, a carbohydrate, vitamins, minerals and other beneficial nutrients including functional ingredients of the disclosure, that is, ingredients that can produce specific benefits to a person consuming the food. The carbohydrate can be, but is not limited to, glucose, sucrose, fructose, dextrose, tagatose, lactose, maltose, galactose, xylose, xylitol, dextrose, polydextrose, cyclodextrins, trehalose, raffinose, stachyose, fructooligosaccharide, maltodextrins, starches, pectins, gums, carrageenan, inulin, cellulose based compounds, sugar alcohols, sorbitol, mannitol, maltitol, xylitol, lactitol, isomalt, erythritol, pectins, gums, carrageenan, inulin, hydrogenated indigestible dextrins, hydrogenated starch hydrolysates, highly branched maltodextrins, starch and cellulose.

Commercially available sources of nutritional proteins, carbohydrates, and the like and their specifications are known, or can be ascertained easily, by those of ordinary skill in the art of processed food formulation.

The compositions that include nutritional components can be food preparations that can be, but are not limited to, “snack sized”, or “bite sized” compositions that is, smaller than what might normally be considered to be a food bar. For instance, the food bar can be indented or perforated to allow the consumer to break off smaller portions for eating, or the food “bar” can be small pieces, rather than a long, bar-shaped product. The smaller pieces can be individually coated or enrobed. They can be packaged individually or in groups.

The food can include solid material that is not ground to a homogeneous mass, such as, without limitation. The food can be coated or enrobed, such as, and without limitation, with chocolate, including dark, light, milk or white chocolate, carob, yogurt, other confections, nuts or grains. The coating can be a compounded confectionary coating or a non-confectionary (e.g., sugar free) coating. The coating can be smooth or can contain solid particles or pieces.

Approaches to treat viral infections, such as SARS-CoV-2, include structure-based drug discoveries focused on proteases, the Spike (S) protein—ACE2 interactions, among others. Our approach is different and is drawn to fatty acids that disrupt synthesis and organization of the cell surface as well as of the endosomes. In addition, our approach is drawn to mediators that counter-regulate the expression of necessary molecules for viral attachment and entrance into cells as well as to counter-regulate the cytokine storm and other inflammatory/immune components activated by the virus in the lung and nasal mucosa. Referring to FIG. 22 for example, aspects of the invention are drawn to VLC-PUFAs (n-3) and elovanoids (ELVs) that can protect lungs and cells of other barrier organs (such as nasal mucosa, GI enterocytes) against virus infection, such as infection with SARS-CoV-2.

Aspects of this invention are drawn to compositions and methods for alleviating a symptom of, preventing, or treating viral infections, viral disease, or viral induced inflammatory responses. For example, the viral infection can be SARS-CoV-2.

Aspects of the invention are drawn towards compounds, compositions, and methods for the alleviation of a symptom of, the prevention of, and treatment of viral infections, viral disease, or viral inflammatory responses. This is based on new findings described herein regarding the key viral blocking and anti-inflammatory role of certain very long chain-polyunsaturated fatty acids (VLC-PUFA) and their related hydroxylated derivatives.

Long chain polyunsaturated fatty acids (LC-PUFAs) can include the omega-3 (n3) and omega-6 (n6) polyunsaturated fatty acids containing 18-22 carbons including: arachidonic acid (ARA, C20:4n6, i.e. 20 carbons, 4 double bonds, omega-6), eicosapentaenoic acid (EPA, C20:5n3, 20 carbons, 5 double bonds, omega-3), docosapentaenoic acid (DPA, C22:5n3, 22 carbons, 5 double bonds, omega-3), and docosahexaenoic acid (DHA, C22:6n3, 22 carbons, 6 double bonds, omega-3). LC-PUFAs are converted via lipoxygenase-type enzymes to biologically active hydroxylated PUFA derivatives that function as biologically active lipid mediators that play important roles in inflammation and related conditions. Most important among these are hydroxylated derivatives generated in certain inflammation-related cells via the action of a lipoxygenase (LO or LOX) enzyme (e.g. 15-LO, 12-LO), and result in the formation of mono-, di- or tri-hydroxylated PUFA derivatives with potent actions including anti-inflammatory, pro-resolving, neuroprotective or tissue-protective actions, among others. For example, neuroprotectin D1 (NPD1), a dihydroxy derivative from DHA formed in cells via the enzymatic action of 15-lipoxygenase (15-LO) was shown to have a defined R/S and Z/E stereochemical structure (10R,17S-dihydroxy-docosa-4Z,7Z,11E, 13E,15Z,19Z-hexaenoic acid) and a unique biological profile that includes stereoselective potent anti-inflammatory, homeostasis-restoring, pro-resolving, bioactivity. NPD1 has been shown to modulate neuroinflammatory signaling and proteostasis, and to promote nerve regeneration, neuroprotection, and cell survival.

Other important types of fatty acids are the n3 and n6 very-long-chain polyunsaturated fatty acids (n3 VLC-PUFA, n6 VLC-PUFA) that are produced in cells containing elongase enzymes that elongate n3 and n6 LC-PUFA to n3 and n6 VLC-PUFA containing from 24 to 42 carbons (C24-C42). The most important among these seem to be VLC-PUFA with 28-38 carbons (C28-C38). Representative types of VLC-PUFA include C32:6n3 (32 carbons, 6 double bonds, omega-3), C34:6n3, C32:5n3, and C34:5n3. These VLC-PUFA are biogenically-derived through the action of elongase enzymes, for example ELOVL4 (ELOngation of Very Long chain fatty acids 4). VLC-PUFA are also acylated in complex lipids including sphingolipids and phospholipids for example in certain molecular species of phosphatidyl choline. Referring to the examples, adding VLC-PUFAs to human bronchiole and alveoli cells in culture activates the synthesis of elovanoids (ELVs) 32 and 34. These two mediators counter-regulate the cytokine storm and other inflammatory components activated by a virus in the lung. The VLC-PUFA and compounds described herein curtail inflammation/cytokine storm by fostering the synthesis of protective bioactive mediators, the elovanoids. The VLC-PUFAs and compounds described herein target the damaging inflammatory response to a virus, for example SARS-CoV-2, on the immune system reflected in the cytokine storm. It is contained by activating pro-homeostatic pathways of ELVs synthesis in human bronchiole and alveoli.

See, for example, PCT/US2016/017112, PCT/US2018/023082, and U.S. Ser. No. 16/576,456, each of which are included herein by reference in their entireties. These VLC-PUFA and compounds described herein can display functions in membrane organization, and their significance to health is increasingly recognized.

The compounds, compositions and methods encompassed by the embodiments of the disclosure involve the use of n3 VLC-PUFA and compounds described herein for alleviating a symptom of, preventing, or treating a viral infection, viral disease, or viral induced inflammatory response.

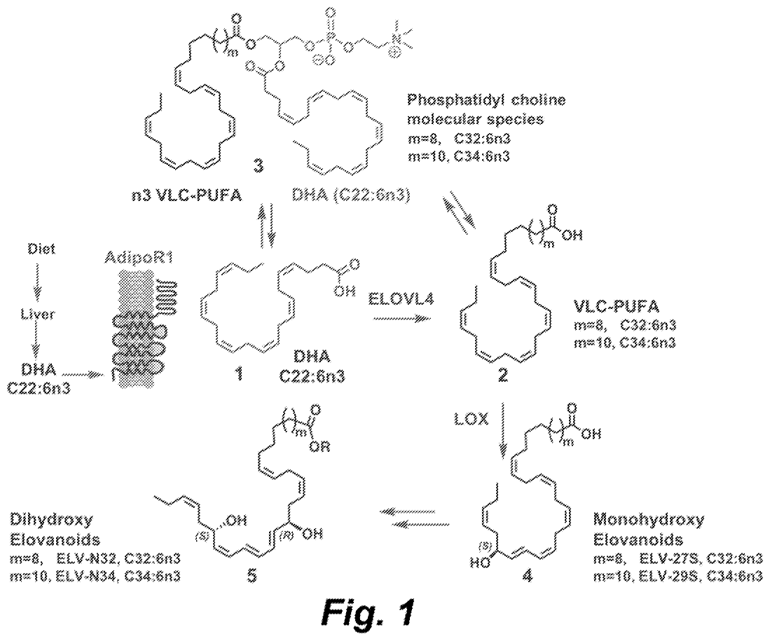

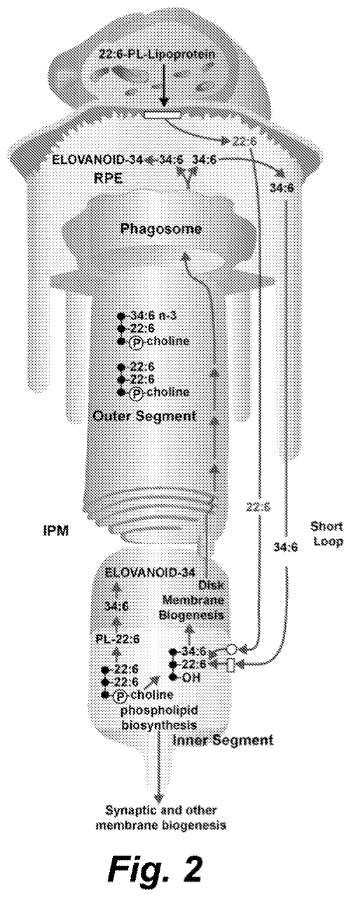

Biosynthetic pathways for n3 VLC-PUFA: The biosynthesis of n3 VLC-PUFA begins from lower-carbon PUFA that contain only an even number of carbons in their carbon chain, such as docosahexaenoic acid (DHA) that contains 22 carbons and 6 alternating C═C bonds (C22:6n3), and docosapentaenoic acid (DPA) that contains 22 carbons and 5 alternating C═C bonds (C22:5n3). The biosynthesis of n3 VLC-PUFA requires the availability of DHA or other shorter-chain PUFA as substrates, and the presence and actions of certain elongase enzymes, e.g. ELOVL4. As summarized in FIGS. 1 and 2, these 22-carbon omega-3 long-chain fatty acids (n3 LC-PUFA) are substrates to elongase enzymes, such as ELOVL4, which adds a 2-carbon CH₂CH₂ group at a time to the carboxylic end, forming n3 VLC-PUFA that contain carbon chains with at least 24 carbons of up to at least 42 carbons.

Docosahexaenoic acid (DHA, C22:6n3, 1 is incorporated at the 2-position of phosphatidyl choline molecular species (3) and is converted by elongase enzymes to longer-chain n3 VLC-PUFA. Elongation by the elongase enzyme ELOVL4 (ELOngation of Very Long chain fatty acids-4) leads to the formation of very long chain omega-3 polyunsaturated fatty acids (n3 VLC-PUFA, 2, including C32:6n3 and C34:6n3 that are then incorporated at the 1-position of phosphatidyl choline molecular species, 3. The presence of DHA at the 2-position and n3 VLC-PUFA at the 1-position offers redundant, complementary, and synergistic cytoprotective and neuroprotective actions that amplify the survival of neurons and other key cell types when challenged with pathological conditions.

Lipoxygenation of n3-VLC-PUFA, 3 leads to the formation of enzymatically-hydroxylated derivatives of n3-VLC-PUFA, termed elovanoids, which include monohydroxy compounds (e.g. ELV-27S and ELV-295, 4, and dihydroxy derivatives, e.g. ELV-N32 and ELV-N34, 5. Elovanoid ELV-N32 is the 20R,27S-dihydroxy 32:6 derivative (32-carbon, 6 double bond elovanoid with a neuroprotectin-like 20(R),27(S)-dihydroxy pattern). Elovanoid ELV-N34 is the 22R,29S-dihydroxy 34:6 derivative (34-carbon, 6 double bond elovanoid with a 22(R),29(S)-dihydroxy pattern).

FIG. 2, for example, illustrates the delivery of docosahexaenoic acid (DHA, C22:6n3) to photoreceptors, photoreceptor outer segment membrane renewal, and the synthesis of elovanoids. DHA or precursor C18:3n3 are obtained by diet, as is DHA itself (FIG. 1). The systemic circulation (mainly the portal system) brings them to the liver. Once within the liver, hepatocytes incorporate DHA into DHA-phospholipid (DHA-PL), which is then transported as lipoproteins to the choriocapillaries, neurovascular unit, and to the capillaries of other tissues.

DHA crosses Bruch's membrane from the choriocapillaries (FIG. 2) and is taken up by the retinal pigment epithelium (RPE) cells lining the back of the retina to be sent to the inner segment of photoreceptors. This targeted delivery route from the liver to the retina is referred to as the DHA long loop.

DHA then passes through the interphotoreceptor matrix (IPM) and to the photoreceptor inner segment, where it is incorporated into phospholipids for the photoreceptor outer segments, cell membrane and organelles. The majority is used in disk membrane biogenesis (outer segments). As new DHA-rich disks are synthesized at the base of the photoreceptor outer segment, older disks are pushed apically toward the RPE cells. Photoreceptor tips are phagocytized by the RPE cells each day, removing the oldest disks. The resulting phagosomes are degraded within the RPE cells, and DHA is recycled back to photoreceptor inner segments for new disk membrane biogenesis. This local recycling is referred to as the 22:6 short loop.

Elovanoids are formed from omega-3 very long chain polyunsaturated fatty acids (n3 VLC-PUFA) biosynthesized by ELOVL4 (ELOngation of Very Long chain fatty acids-4) in the photoreceptor inner segments. Thus, a phosphatidylcholine molecular species in the inner segment that contains VLC Omega-3 FA at C1 (C34:6n3 is depicted) and DHA (C22:6n3) at C2 is used for photoreceptor membrane biogenesis. This phospholipid has been found tightly associated to rhodopsin. Once the discs are phagocytized in RPE cells as a daily physiological process, upon homeostatic disturbances, a phospholipase A1 (PLA1) cleaves the acyl chain at sn-1, releasing C34:6n3 and leads to the formation of elovanoids (e.g. elovanoid-34, ELV-N34). VLC omega-3 fatty acids that are not used for elovanoid synthesis are recycled through the short loop.

Therefore, for biosynthetic reasons, the naturally occurring and biogenetically derived n3 VLC-PUFA contain only an even number of carbons, ranging from at least 24 carbons to at least 42 carbons (i.e. 24, 26, 28, 30, 32, 34, 36, 38, 40, 42 carbons). Thus, n3 VLC-PUFA that contain only an odd number of carbons ranging from at least 23 of up to at least 41 carbons (i.e. 23, 25, 27, 29, 31, 33, 35, 37, 39, 41 carbons) are not naturally occurring, but they can be synthesized and manufactured using synthetic chemical methods and strategies.

Stereocontrolled total synthesis and structural characterization of elovanoids ELV-N32 and ELV-N34 in the retina and the brain: As summarized in FIG. 3 and FIG. 4, for example, ELV-N32 (27S- and ELV-N34 were synthesized from three key intermediates (1, 2, and 3), each of which was prepared in stereochemically-pure form. The stereochemistry of intermediates 2 and 3 was pre-defined by using enantiomerically pure epoxide starting materials. Iterative couplings of intermediates 1, 2, and 3, led to ELV-N32 and ELV-N34 (4) that were isolated as the methyl esters (Me) or sodium salts (Na). The synthetic materials ELV-N32 and ELV-N34 were matched with endogenous elovanoids with the same number of carbons on their carbon chain, obtained from cultured human retinal pigment epithelial cells (RPE) (FIG. 3), and neuronal cell cultures (FIG. 4).

Experimental detection and characterization of the Elovanoids: Experimental evidence documents the biosynthetic formation of the elovanoids, which are mono-hydroxy and di-hydroxy n3 VLC-PUFA derivatives with molecular structures that are analogous to DHA-derived 17-hydroxy-DHA and the di-hydroxy compound NPD1 (10R,17S-dihydroxy-docosa-4Z,7Z,11E,13E,15Z,19Z-hexaenoic acid). The elovanoids are enzymatically generated hydroxylated derivatives of 32-carbon (ELV-N32) and 34-carbon (ELV-N34) n3 VLC-PUFA in that were first identified in cultures of primary human retinal pigment epithelial cells (RPE) (FIG. 3A-3K) and in neuronal cell cultures (FIG. 4A-4K).

The disclosure provides compounds having carbon chains related to n3 VLC-PUFA that in addition to having 6 or 5 C═C bonds, they also contain one, two or more hydroxyl groups. Considering that compounds of this type can be responsible for the protective and neuroprotective actions of n3 VLC-PUFA, we sought to identify their existence in human retinal pigment epithelial cells in culture, with added 32:6n3 and 34:6n3 VLC-PUFA fatty acids. Our results indicated mono-hydroxy- and di-hydroxy elovanoid derivatives from both 32:6n3 and 34:6n3 VLC-PUFA fatty acids. The structures of these elovanoids (ELV-N32, ELV-N34) were compared with standards prepared in stereochemical pure form via stereocontrolled total organic synthesis (FIG. 5A and FIG. 5B).

Beneficial Roles of n3 VLC-PUFA as Therapeutics: The data described herein provided support for the beneficial use of the provided n3 VLC-PUFA and/or elovanoid compounds, as therapeutics for the prevention and treatment of viral infection, viral disease, or viral inflammatory response.

The terms “inflammation” and “inflammatory response” can refer to the combined biological response of an individual's tissue to harmful stimuli such as pathogens, viruses, damaged cells, or irritants. Inflammation and inflammatory response can include secretion of cytokines, such as inflammatory cytokines (i.e., cytokines produced primarily by active immune cells such as microglia and involved in the amplification of inflammatory responses). Exemplary inflammatory cytokines include, but are not limited to, IL-1, IL-6, TNF-a, IL-17, IL21, IL23 and TGF-β. Exemplary inflammation includes acute inflammation and chronic inflammation. As used herein, the term “acute inflammation” can be characterized by the classic signs of inflammation (swelling, hyperemia, pain, high fever, loss of function) resulting from tissue infiltration by plasma and leukocytes. Acute inflammation occurs as long as harmful stimuli are present and stops once the stimuli are removed and degraded or surrounded by scars (fibrosis). As used herein, the term “chronic inflammation” can refer to a condition characterized by ongoing concurrent active inflammation, tissue destruction, and attempts at repair. Chronically inflamed tissues are characterized by infiltration, tissue destruction, and recovery of mononuclear immune cells (monocytes, macrophages, lymphocytes and plasma cells), angiogenesis and fibrosis including symptoms. Inflammation can be controlled as described herein by affecting, such as inhibiting, any of the events that form a complex biological response associated with an individual's inflammation. For example, in some embodiments, inflammation can be controlled by affecting, such as inhibiting, cytokine production, for example the production of inflammatory cytokines.

The phrase “viral inflammatory response” can refer to any mechanism by which inflammation is achieved and regulated. For example, the mechanism can comprise immune cell activation or migration and cytokine production. For example, the viral inflammatory response can refer to a cytokine storm. The phrase “cytokine storm” can refer to a series of events that result in an immune reaction that comprises a positive feedback loop between cytokines and immune cells that in turn leads to highly elevated levels of various cytokines.

The term “virus” can refer to a submicroscopic infectious agent that replicates inside of living cells of an organism. For example, the virus can refer to a coronavirus, such as severe acute respiratory syndrome coronavirus 2 (SARS-CoV-2). SARS-CoV-2 is a recently discovered human pathogen that is a member of the betacoronavirus genus. Infection with SARS-CoV-2 can result in disease and has led to a global pandemic. For example, the viral infection or viral disease can refer to coronavirus disease 19 (COVID-19). COVID-19 and SARS-CoV-2 can be used interchangeably. Infection with SARS-CoV-2 can lead to symptoms such as fever, severe respiratory illness, and pneumonia, some symptoms are so severe as to result in death (Wrapp et al. Science 13 Mar. 2020, 367(6483)₁₂₆₀-1263). As some of the most severe SARS-CoV-2 symptoms appear to affect the respiratory system, as well as several other organs, and with no currently known cure, a treatment for respiratory symptoms, and those resulting from other affected organs, induced by a virus or prevention of viral infection is desired.

Prevention of viral infection can include blocking the virus from binding to the cell surface of various organs (nasal mucosa, lung alveoli, gastrointestinal tract, etc.) and consequently entering the human body. For example, VLC-PUFAs induce lipidome remodeling, which without wishing to be bound by theory, disrupt tetraspanin-enriched membrane microdomains blocking SARS-CoV-2 binding and entrance in human bronchiole and alveoli. In another example, VLC-PUFAs that alter lipid biosynthesis that modify cell endosomal trafficking also halt viral replication.

The phrase “viral infection” or “viral disease” can be used interchangeably, and can refer to an infection, disease, or disorder caused by both RNA and DNA viruses and can refer to any stage of viral infection, including incubation phase, latent or dormant phase, acute phase, and development and maintenance of immunity to a virus. A “viral infection” or “viral disease” can be characterized by a strong correlation between exposure to a virus and the development of pathological changes, and that the pathological changes have an immune mechanism (i.e., a viral inflammatory response). For example, the immune mechanism can refer to leukocytes exhibit an immune response to viral stimulation. For example, the immune response can refer to increased production of pro-inflammatory cytokines and chemokines. For example, the immune response can refer to tissue inflammation. For example, the tissue can comprise ocular tissue, brain tissue, gastrointestinal tissue, skin tissue, heart tissue or another bodily tissue.

For example, an immune response can be indicated by increased production of pro-inflammatory cytokines, chemokines, or a combination thereof. For example, this can be measured clinically. For example, non-limiting examples of pro-inflammatory cytokines, chemokines, or a combination thereof can comprise IL-6, IL-1β, IL-8/CXCL8, CCL2/MCP-1, CXCL1/KC/GRO, VEGF, or ICAM1(CD54).

Origin of the compounds of the disclosure: The provided compounds were not isolated from tissues naturally occurring in nature, but from the result of an artificial experiment combining a human cell and a chemically synthesized n3-VLC-PUFA. The structures of the synthetic elovanoid compounds were matched using HPLC and mass spectrometry with compounds biosynthesized in human retinal pigment epithelial cells or detected in neuronal cell cultures. However, the natural occurrence of the provided mono- and di-hydroxylated elovanoids with C36 and C38 with specifically defined stereochemistry is not known at this time. Moreover, the provided compounds are not obtained from natural sources, but they are prepared by adapting stereocontrolled synthetic methods known in the art, starting with commercially available materials. The provided preparation methods were designed to be suitable to the unique hydrophobic properties of n3 VLC-PUFA, which differ significantly from compounds that have a total number of carbons of 22 carbons or less.

Embodiments described herein encompasses compounds that have stereochemically pure structures and are chemically synthesized and modified to have additional structural features and properties that allow them to exert pharmacological activity. The provided compounds are chemically modified pharmaceutically acceptable derivatives in the form of carboxylic esters or salts that enhance their chemical and biological stability and allow their use in therapeutic applications involving various forms of drug delivery.

The disclosure also provides pharmacologically effective compositions of the provided compounds that enhance their ability to be delivered to a subject in a manner that can reach the targeted cells and tissues.

The data described herein also provides support for the beneficial use of the provided n3 VLC-PUFA and/or elovanoid compounds and/or intermediaries, as therapeutics for the prevention and treatment of viral inflammatory/immune responses, such as by abrogating the production of pro-inflammatory cytokines and chemokines by a cell, such as an epithelial cell or a monocyte-derived macrophage.

Epithelium lines both the outside (skin) and the inside cavities and lumina of bodies. Epithelial cells are scutoidal shaped, tightly packed and form a continuous sheet. They have no intercellular spaces. All epithelia can be separated from underlying tissues by an extracellular fibrous basement membrane. The lining of the mouth, lung alveoli, nasal mucosa and kidney tubules are all made of epithelial cells. The lung epithelium acts as the initial protective barrier for the lungs. The lining of the blood and lymphatic vessels comprise specialized cells called endothelium.

The term “epithelial cell” can refer to cells that line the outside (skin), mucous membranes, and the inside cavities and lumina of the body. Most epithelial cells exhibit an apical-basal polarization of cellular components. Epithelial cells are classified by shape and by their specialization.

The epidermis (i.e., skin) consists of keratinized stratified squamous epithelium. Four cell types are present: keratinocytes produce keratin, a protein that hardens and waterproofs the skin. Mature keratinocytes at the skin surface are dead and filled almost entirely with keratin. Melanocytes produce melanin, a pigment that protects cells from ultraviolet radiation. Melanin from the melanocytes is transferred to the keratinocytes. Langerhans cells are phagocytic macrophages that interact with white blood cells during an immune response. Merkel cells occur deep in the epidermis at the epidermal-dermal boundary. They form Merkel discs, which, in association with nerve endings, serve a sensory function.

There are several layers making up the epidermis. “Thick skin,” found on the palms of the hands and soles of the feet, consists of five layers while “thin skin” consists of only four layers. The five layers include the stratum corneum contains many layers of dead, anucleate keratinocytes completely filled with keratin. The outermost layers are constantly shed. The stratum lucidum contains two to three layers of anucleate cells. This layer is found only in “thick skin” such as the palm of the hand and the sole of the foot. The stratum granulosum contains two to four layers of cells held together by desmosomes. These cells contain keratohyaline granules, which contribute to the formation of keratin in the upper layers of the epidermis. The stratum spinosum contains eight to ten layers of cells connected by desmosomes. These cells are moderately active in mitosis. The stratum basale (stratum germinativum) contains a single layer of columnar cells actively dividing by mitosis to produce cells that migrate into the upper epidermal layers and ultimately to the surface of the skin.

Nasal epithelial cells, for example, form the outermost protective layer against environmental factors, bacterial infection, and viral infection. They clean, humidify, and warm inhaled air. They produce mucus, which bind particles that are subsequently transported to the pharynx by cilia on the epithelial cells.

The corneal epithelium, for example, is made up of epithelial tissue and covers the front of the cornea. It acts as a barrier to protect the cornea, resisting the free flow of fluids from the tears, and prevents bacteria and viruses from entering the epithelium and corneal stroma.

Respiratory epithelium, or airway epithelium, is a type of ciliated columnar epithelium found lining most of the respiratory tract as respiratory mucosa. The cells in the respiratory epithelium are of four main types: a) ciliated cells, b) goblet cells, and c) club cells, and d) basal cells. The respiratory epithelium functions to moisten and protect the airways. It acts as a physical barrier to pathogens and foreign particles, as well as the removal of pathogens in the mechanism of mucociliary clearance, thus preventing infection and tissue injury by the secretion of mucus and the action of mucociliary clearance.

Non-limiting Exemplary Compounds

Described herein are compounds based on omega-3 very long chain polyunsaturated fatty acids and their hydroxylated derivatives, termed “elovanoids”.

The omega-3 very long chain polyunsaturated fatty acids have the structures of A or B, or derivatives thereof:

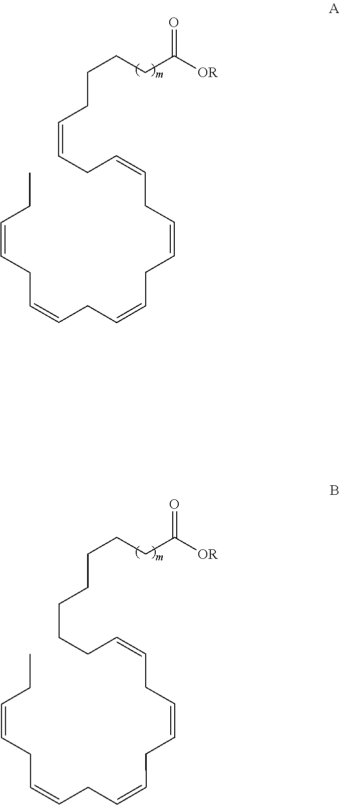

wherein: A contains a total from 23 to 42 carbon atoms in the carbon chain, and with 6 alternating cis carbon-carbon double bonds starting at positions n-3, n-6, n-9, n-12, n-15 and n-18, and wherein B contains a total from 23 to 42 carbon atoms in the carbon chain, and with 5 alternating cis carbon-carbon double bonds starting at positions n-3, n-6, n-9, n-12 and n-15. R can be hydrogen, methyl, ethyl, alkyl, or a cation such as an ammonium cation, an iminium cation, or a metal cation including, but not limited to, sodium, potassium, magnesium, zinc, or calcium cation, and wherein m is a number from 0 to 19.

The omega-3 very long chain polyunsaturated fatty acids of the disclosure can have a terminal carboxyl group “—COOR” wherein “R” can be a group covalently bonded to the carboxyl such as an alkyl group. In the alternative, the carboxyl group can further have a negative charge as “—COO” and R is a cation including a metal cation, an ammonium cation and the like.

In some omega-3 very long chain polyunsaturated fatty acids, m is a number selected from a group consisting of 0 to 15. Thus, can be a number selected from 1, 3, 5, 7, 9, 11, 13, or 15 where the fatty acid component contains a total of 24, 26, 28, 30, 32, 34, 36 or 38 carbon atoms in its carbon chain. In other omega-3 very long chain polyunsaturated fatty acids, m is a number selected from a group consisting of 0, 2, 4, 6, 8, 10, 12 or 14, where the fatty acid component contains a total of 23, 25, 27, 19, 31, 33, 35 or 37 carbon atoms in its carbon chain. In some omega-3 very long chain polyunsaturated fatty acids, m is a number selected from a group consisting of 5 to 15, where the fatty acid component contains a total of 28, 29, 30, 31, 32, 33, 34, 35, 36, 37 or 38 carbon atoms in its carbon chain. In some omega-3 very long chain polyunsaturated fatty acids, m is a number selected from a group consisting of 9 to 11, where the fatty acid component contains a total of 32 or 34 carbon atoms in its carbon chain.

In some embodiments the omega-3 very long chain polyunsaturated fatty acids is a carboxylic acid, i.e. R is hydrogen. In other embodiments the omega-3 very long chain polyunsaturated fatty acids is a carboxylic ester, wherein R is methyl, ethyl or alkyl. When the omega-3 very long chain polyunsaturated fatty acid is a carboxylic ester, R can be, but is not limited to, methyl or ethyl. In some embodiments the omega-3 very long chain polyunsaturated fatty acid is a carboxylic ester, wherein R is methyl.

In some embodiments the omega-3 very long chain polyunsaturated fatty acid can be a carboxylate salt, wherein R is an ammonium cation, iminium cation, or a metal cation selected from a group consisting of sodium, potassium, magnesium, zinc, or calcium cation. In some advantageous embodiments, R is ammonium cation or iminium cation. R can be a sodium cation or a potassium cation. In some embodiments, R is a sodium cation.

The omega-3 very long chain polyunsaturated fatty acid or derivative of the disclosure can have 32- or 34 carbons in its carbon chain and 6 alternating cis double bonds starting at the n-3 position, and have the formula A1 (14Z,17Z,20Z,23Z,26Z,29Z)-dotriaconta-14,17,20,23,26,29-hexaenoic acid) or formula A2 (16Z,19Z,22Z,25Z,28Z,31Z)-tetratriaconta-16,19,22,25,28,31-hexaenoic acid):

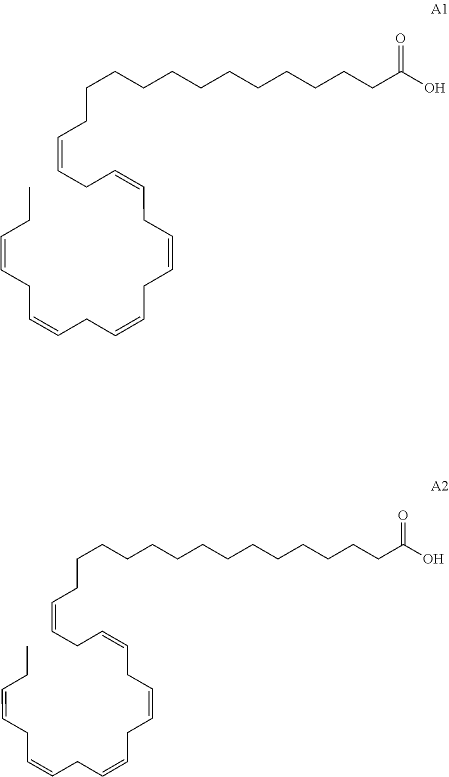 [SMILES: CC/C=C\C/C=C\C/C=C\C/C=C\C/C=C\C/C=C\CCCCCCCCCCCCC(=O)O ; CC/C=C\C/C=C\C/C=C\C/C=C\C/C=C\C/C=C\CCCCCCCCCCCCCCC(=O)O]

In some embodiments of the omega-3 very long chain polyunsaturated fatty acids, the carboxyl derivative is part of a glycerol-derived phospholipid, which can be readily prepared starting with the carboxylic acid form of the n3 VLC-PUFA of structure A or B, by utilizing methods known in the art, and represented by structures C, D, E, or F:

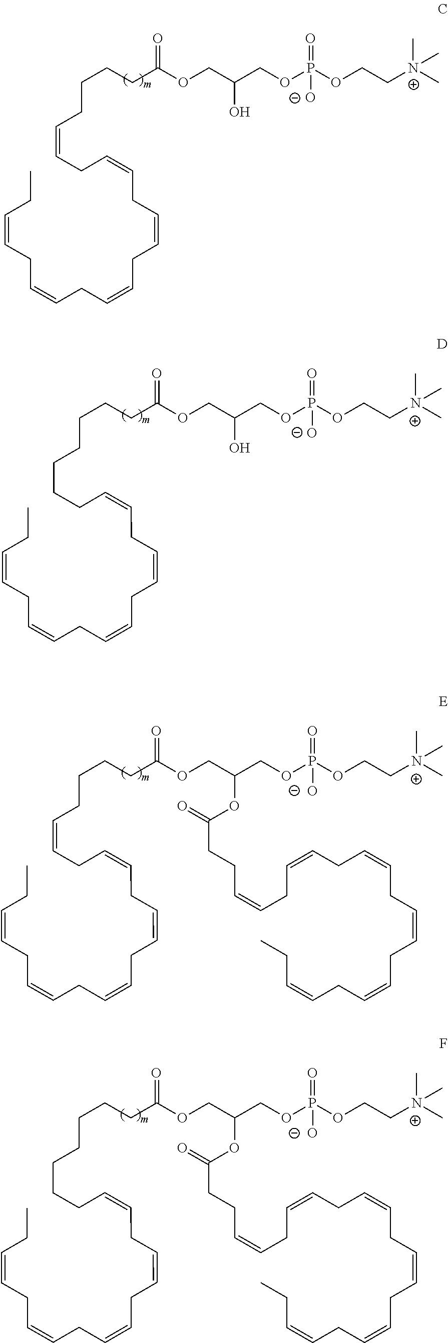

wherein C or E contains a total from 23 to 42 carbon atoms in the carbon chain, and with 6 alternating cis carbon-carbon double bonds starting at positions n-3, n-6, n-9, n-12, n-15 and n-18, and wherein D or E contains a total from 23 to 42 carbon atoms in the carbon chain, and with 5 alternating cis carbon-carbon double bonds starting at positions n-3, n-6, n-9, n-12 and n-15. In advantageous embodiments, m is a number selected from a group consisting of 0 to 15. In other embodiments, m is a number selected from 1, 3, 5, 7, 9, 11, 13, or 15 where the fatty acid component contains a total of 24, 26, 28, 30, 32, 34, 36 or 38 carbon atoms in its carbon chain. In additional advantageous embodiments, m is a number selected from a group consisting of 0, 2, 4, 6, 8, 10, 12 or 14, where the fatty acid component contains a total of 23, 27, 19, 31, 33, 35 or 37 carbon atoms in its carbon chain.

In some embodiments, m is a number selected from a group consisting of 5 to 15, where the fatty acid component contains a total of 28, 29, 30, 31, 32, 33, 34, 35, 36, 37 or 38 carbon atoms in its carbon chain. In some embodiments, m is a number selected from a group consisting of 5, 7, 9, 11, 13, or 15, where the fatty acid component contains a total of 28, 30, 32, 34, 36 or 38 carbon atoms in its carbon chain. In other embodiments, m is a number selected from a group consisting of 4, 6, 8, 10, 12 or 14, where the fatty acid component contains a total of 27, 29, 31, 33, 35 or 37 carbon atoms in its carbon chain. In advantageous embodiments, m is a number selected from a group consisting of 9 to 11, where the fatty acid component contains a total of 32 or 34 carbon atoms in its carbon chain.

The mono-hydroxylated elovanoids of the disclosure can have the structures of G, H, I or J:

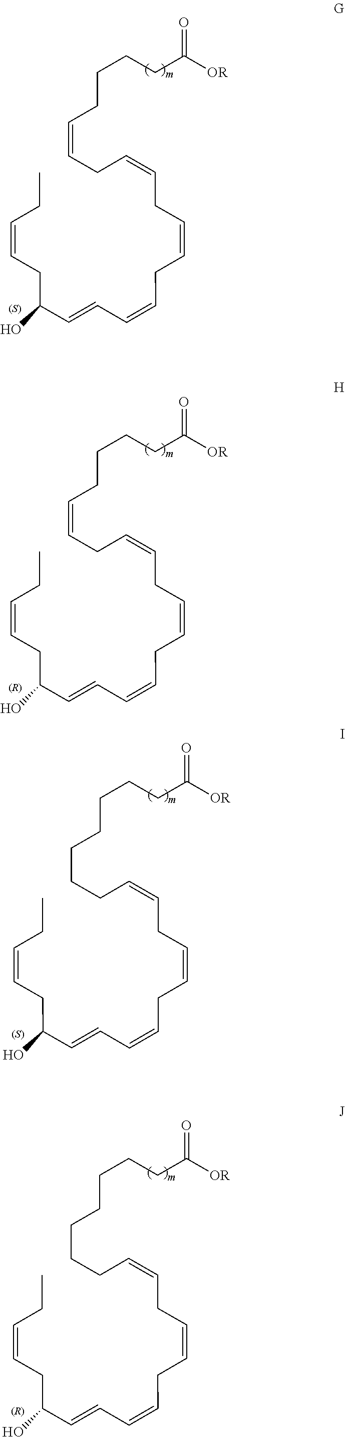

wherein compounds G and H have a total from 23 to 42 carbon atoms in the carbon chain, with cis carbon-carbon double bonds starting at positions n-3, n-9, n-12, n-15 and n-18 and a trans carbon-carbon double bond starting at positions n-7; and wherein compounds I and J have a total from 23 to 42 carbon atoms in the carbon chain, and with 4 cis carbon-carbon double bonds starting at positions n-3, n-9, n-12 and n-15, and a trans carbon-carbon double bond starting at positions n-7; wherein R is hydrogen, methyl, ethyl, alkyl, or a cation selected from a group consisting of: ammonium cation, iminium cation, or a metal cation selected from a group consisting of sodium, potassium, magnesium, zinc, or calcium cation, and wherein m is a number selected from a group consisting of 0 to 19; wherein compounds G and H can exist as an equimolar mixture; wherein compounds I and J can exist as an equimolar mixture; wherein, the provided compounds G and H are predominately one enantiomer with a defined (S) or (R) chirality at the carbon bearing the hydroxyl group; and wherein, the compounds G and H are predominately one enantiomer with a defined (S) or (R) chirality at the carbon bearing the hydroxyl group.

As used herein and in other structures of the present disclosure, the compounds of the disclosure are shown having a terminal carboxyl group “—COOR” the “R” can be a group covalently bonded to the carboxyl such as an alkyl group. In the alternative, the carboxyl group can further have a negative charge as “—COO” and R is a cation including a metal cation, an ammonium cation and the like.

In some embodiments of the mono-hydroxylated elovanoids of the disclosure, m is a number selected from a group consisting of 0 to 15. In other advantageous embodiments, m is a number selected from 1, 3, 5, 7, 9, 11, 13, or 15 where the fatty acid component contains a total of 24, 26, 28, 30, 32, 34, 36 or 38 carbon atoms in its carbon chain. In other embodiments, m is a number selected from a group consisting of 0, 2, 4, 6, 8, 10, 12 or 14, where the fatty acid component contains a total of 23, 25, 27, 19, 31, 33, 35 or 37 carbon atoms in its carbon chain.

In some embodiments, m is a number selected from a group consisting of 5 to 15, where the fatty acid component contains a total of 28, 29, 30, 31, 32, 33, 34, 35, 36, 37 or 38 carbon atoms in its carbon chain. In some embodiments, m is a number selected from a group consisting of 5, 7, 9, 11, 13, or 15, where the fatty acid component contains a total of 28, 30, 32, 34, 36 or 38 carbon atoms in its carbon chain. In other embodiments, m is a number selected from a group consisting of 4, 6, 8, 10, 12 or 14, where the fatty acid component contains a total of 27, 29, 31, 33, 35 or 37 carbon atoms in its carbon chain. In advantageous embodiments, m is a number selected from a group consisting of 9 to 11, where the fatty acid component contains a total of 32 or 34 carbon atoms in its carbon chain.

In some embodiments the mono-hydroxylated elovanoids of the disclosure are a carboxylic acid, i.e. R is hydrogen. In other embodiments the compound is a carboxylic ester, wherein R is methyl, ethyl or alkyl. In advantageous embodiments the compound is a carboxylic ester, wherein R is methyl or ethyl. In advantageous embodiments the compound is a carboxylic ester, wherein R is methyl. In other advantageous embodiments the compound is a carboxylate salt, wherein R is an ammonium cation, iminium cation, or a metal cation selected from a group consisting of sodium, potassium, magnesium, zinc, or calcium cation. In some advantageous embodiments, R is ammonium cation or iminium cation. In other advantageous embodiments, R is a sodium cation or a potassium cation. In advantageous embodiments, R is a sodium cation.

The di-hydroxylated elovanoids of the disclosure can have the structures K, L, M, or N

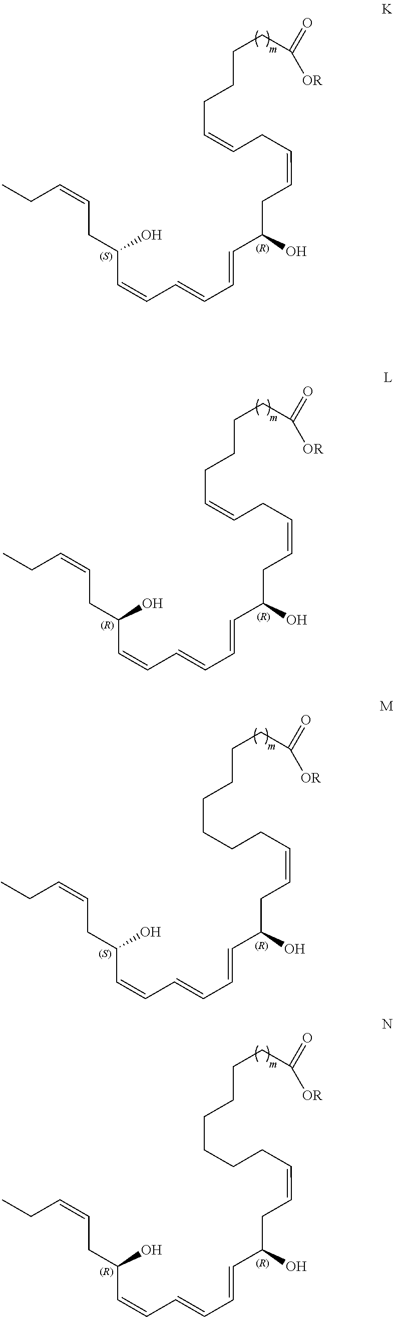

wherein compounds K and L have a total from 23 to 42 carbon atoms in the carbon chain, with 4 cis carbon-carbon double bonds starting at positions n-3, n-7, n-15 and n-18, and 2 trans carbon-carbon bonds starting at positions n-9, n-11; and wherein compounds M and N have a total from 23 to 42 carbon atoms in the carbon chain, with 3 cis carbon-carbon double bonds starting at positions n-3, n-7, n-12 and n-15, and 2 trans carbon-carbon bonds starting at positions n-9, n-11, wherein R is hydrogen, methyl, ethyl, alkyl, or a cation selected from a group consisting of: ammonium cation, iminium cation, or a metal cation selected from a group consisting of sodium, potassium, magnesium, zinc, or calcium cation, and wherein m is a number selected from a group consisting of 0 to 19; wherein compounds K and L can exist as an equimolar mixture; wherein compounds M and N can exist as an equimolar mixture, wherein the compounds K and L are predominately one enantiomer with a defined (S) or (R) chirality at the carbon bearing the hydroxyl group; and wherein, the provided compounds M and N are predominately one enantiomer with a defined (S) or (R) chirality at the carbon bearing the hydroxyl group. In embodiments K, L, M, and N can exist compounds with (R),(S) (wherein R is n-6 and S is n-13) and (S),(S) chiral centers at the carbon bearing hydroxyl group positions.

The di-hydroxylated elovanoids of the disclosure can have the following structures:

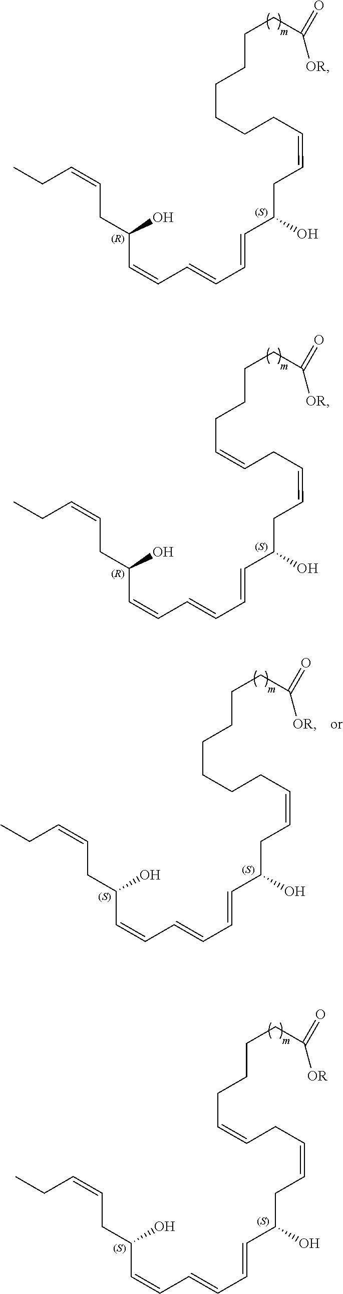

wherein, the compounds can have a total from 23 to 42 carbon atoms in the carbon chain, wherein if the compound has 6 double bonds there can be 4 cis carbon-carbon double bonds starting at positions n-3, n-7, n-15 and n-18, and 2 trans carbon-carbon bonds starting at positions n-9, n-11; and wherein if the compounds have 5 double bonds, there can be 3 cis carbon-carbon double bonds starting at positions n-3, n-7, n-12 and n-15, and 2 trans carbon-carbon bonds starting at positions n-9, n-11, wherein R is hydrogen, methyl, ethyl, alkyl, or a cation selected from a group consisting of: ammonium cation, iminium cation, or a metal cation selected from a group consisting of sodium, potassium, magnesium, zinc, or calcium cation, and wherein m is a number selected from a group consisting of 0 to 19; wherein the compounds can exist as an equimolar mixture or where predominately one enantiomer with a defined (S) or (R) chirality at any carbon bearing the hydroxyl group.

As used herein and in other structures of the present disclosure, the compounds of the disclosure are shown having a terminal carboxyl group “—COOR” the “R” can be a group covalently bonded to the carboxyl such as an alkyl group. In the alternative, the carboxyl group can further have a negative charge as “—COO” and R is a cation including a metal cation, an ammonium cation and the like.

In some embodiments of the di-hydroxylated elovanoids of the disclosure, m is a number selected from a group consisting of 5 to 15, where the fatty acid component contains a total of 28, 29, 30, 31, 32, 33, 34, 35, 36, 37 or 38 carbon atoms in its carbon chain. In advantageous embodiments, m is a number selected from a group consisting of 5, 7, 9, 11, 13, or 15, where the fatty acid component contains a total of 28, 30, 32, 34, 36 or 38 carbon atoms in its carbon chain. In other embodiments, m is a number selected from a group consisting of 4, 6, 8, 10, 12 or 14, where the fatty acid component contains a total of 27, 29, 31, 33, 35 or 37 carbon atoms in its carbon chain. In advantageous embodiments, m is a number selected from a group consisting of 9 to 11, where the fatty acid component contains a total of 32 or 34 carbon atoms in its carbon chain.

Some di-hydroxylated elovanoids of the disclosure are carboxylic acid, i.e. R is hydrogen. In other embodiments the di-hydroxylated elovanoid of the disclosure is a carboxylic ester, wherein R is methyl, ethyl or alkyl. In advantageous embodiments the compound is a carboxylic ester, wherein R is methyl or ethyl. In advantageous embodiments the compound is a carboxylic ester, wherein R is methyl.

In other embodiments the di-hydroxylated elovanoid of the disclosure is a carboxylate salt, wherein R is an ammonium cation, iminium cation, or a metal cation selected from a group consisting of sodium, potassium, magnesium, zinc, or calcium cation. In some advantageous embodiments, R is ammonium cation or iminium cation. In other advantageous embodiments, R is a sodium cation or a potassium cation. In advantageous embodiments, R is a sodium cation.

The alkynyl mono-hydroxylated elovanoids of the disclosure can have the structures of O, P, Q or R:

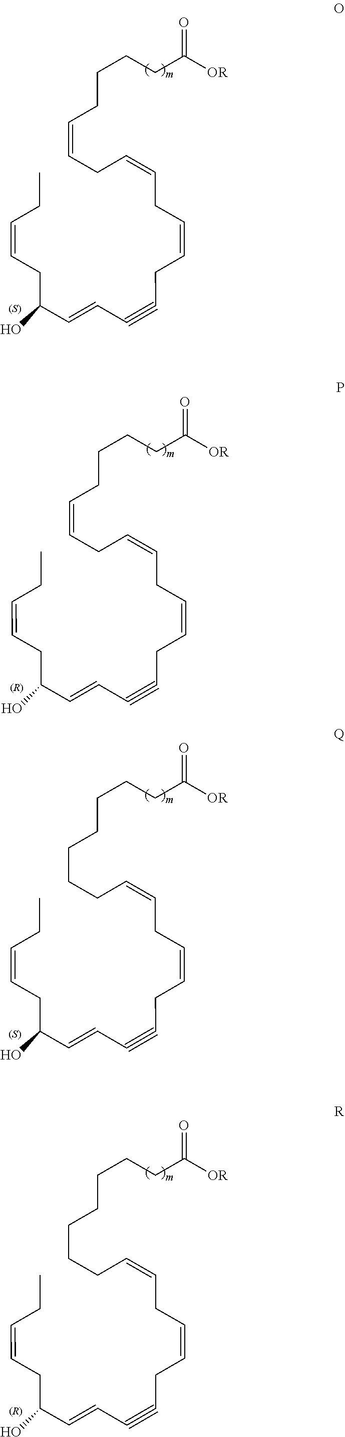

wherein compounds O and P have a total from 23 to 42 carbon atoms in the carbon chain, with 4 cis carbon-carbon double bonds starting at positions n-3, n-12, n-15 and n-18, a trans carbon-carbon bond starting at position n-7, and a carbon-carbon triple bond starting at position n-9; and wherein compounds I and J have a total from 23 to 42 carbon atoms in the carbon chain, with 3 cis carbon-carbon double bonds starting at positions n-3, n-12 and n-15, a trans carbon-carbon bond starting at position n-7, and a carbon-carbon triple bond starting at position n-9; wherein R is hydrogen, methyl, ethyl, alkyl, or a cation selected from a group consisting of: ammonium cation, iminium cation, or a metal cation selected from a group consisting of sodium, potassium, magnesium, zinc, or calcium cation, and wherein m is a number selected from a group consisting of 0 to 19; wherein compounds O and P can exist as an equimolar mixture; wherein compounds Q and R can exist as an equimolar mixture; wherein, the provided compounds O and P are predominately one enantiomer with a defined (S) or (R) chirality at the carbon bearing the hydroxyl group; and wherein, the provided compounds O and P are predominately one enantiomer with a defined (S) or (R) chirality at the carbon bearing the hydroxyl group.

As used herein and in other structures of the present invention, the alkynyl mono-hydroxylated elovanoids of the disclosure are shown having a terminal carboxyl group “—COOR” the “R” can be a group covalently bonded to the carboxyl such as an alkyl group. In the alternative, the carboxyl group can further have a negative charge as “—COO” and R is a cation including a metal cation, an ammonium cation and the like.

In some embodiments, m is a number selected from a group consisting of 0 to 15. In other embodiments, m is a number selected from 1, 3, 5, 7, 9, 11, 13, or 15 where the fatty acid component contains a total of 24, 26, 28, 30, 32, 34, 36 or 38 carbon atoms in its carbon chain.

In additional embodiments, m is a number selected from a group consisting of 0, 2, 4, 6, 8, 10, 12 or 14, where the fatty acid component contains a total of 23, 25, 27, 19, 31, 33, 35 or 37 carbon atoms in its carbon chain. In some embodiments, m is a number selected from a group consisting of 5 to 15, where the fatty acid component contains a total of 28, 29, 30, 31, 32, 33, 34, 35, 36, 37 or 38 carbon atoms in its carbon chain. In embodiments, m is a number selected from a group consisting of 5, 7, 9, 11, 13, or 15, where the fatty acid component contains a total of 28, 30, 32, 34, 36 or 38 carbon atoms in its carbon chain. In other embodiments, m is a number selected from a group consisting of 4, 6, 8, 10, 12 or 14, where the fatty acid component contains a total of 27, 29, 31, 33, 35 or 37 carbon atoms in its carbon chain. In some embodiments, m is a number selected from a group consisting of 9 to 11, where the fatty acid component contains a total of 32 or 34 carbon atoms in its carbon chain.

In some embodiments the alkynyl mono-hydroxylated elovanoids of the disclosure are carboxylic acids, i.e. R is hydrogen. In other embodiments the alkynyl mono-hydroxylated elovanoids of the disclosure are carboxylic esters, wherein R is methyl, ethyl or alkyl. In embodiments the alkynyl mono-hydroxylated elovanoids of the disclosure are carboxylic esters, wherein R is methyl or ethyl.

In some embodiments R is methyl. In other embodiments, alkynyl mono-hydroxylated elovanoids of the disclosure can be a carboxylate salt, wherein R is an ammonium cation, iminium cation, or a metal cation selected from a group consisting of sodium, potassium, magnesium, zinc, or calcium cation. In some embodiments, R is ammonium cation or iminium cation. In other embodiments, R is a sodium cation or a potassium cation. In embodiments, R is a sodium cation.

The alkynyl di-hydroxylated elovanoids can have the structures of S, T, U or V:

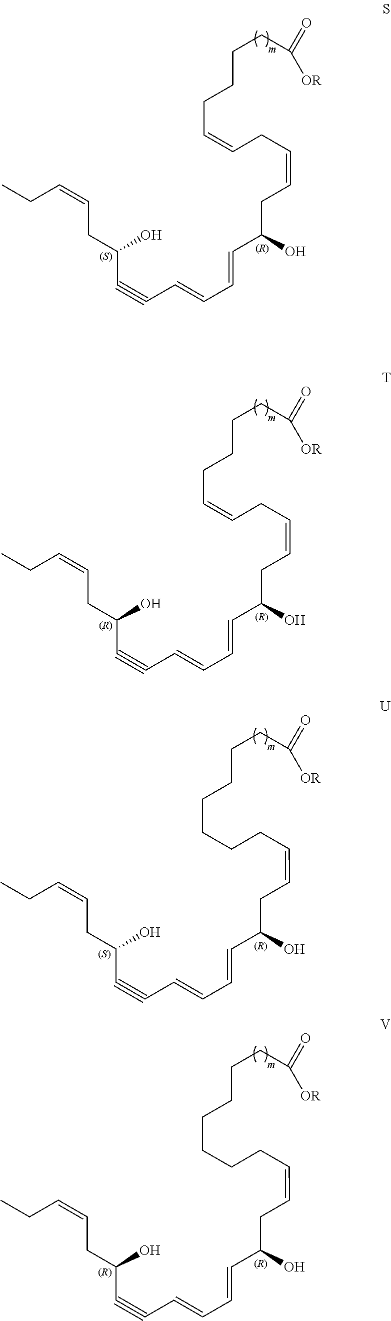

wherein compounds S and T have a total from 23 to 42 carbon atoms in the carbon chain, with 3 cis carbon-carbon double bonds starting at positions n-3, n-12, n-15 and n-18, with 2 trans carbon-carbon double bonds starting at positions n-9 and n-11, and a carbon-carbon triple bond starting at position n-7; and wherein compounds U and V have a total from 23 to 42 carbon atoms in the carbon chain, and with 2 cis carbon-carbon double bonds starting at positions n-3 and n-15, with 2 trans carbon-carbon double bonds starting at positions n-9 and n-11, and a carbon-carbon triple bond starting at position n-7; wherein R is hydrogen, methyl, ethyl, alkyl, or a cation selected from a group consisting of: ammonium cation, iminium cation, or a metal cation selected from a group consisting of sodium, potassium, magnesium, zinc, or calcium cation, and wherein m is a number selected from a group consisting of 0 to 19; wherein compounds S and T can exist as an equimolar mixture; wherein compounds U and V can exist as an equimolar mixture.

In some embodiments, the provided compounds S and T are predominately one enantiomer with a defined (S) or (R) chirality at the carbon bearing the hydroxyl group; and wherein, the provided compounds U and V are predominately one enantiomer with a defined (S) or (R) chirality at the carbon bearing the hydroxyl group. In embodiments, compounds S, T, U, and V can exist compounds with (R),(S) (wherein R is n-6 and S is n-13) and (S),(S) chiral centers at the carbon bearing hydroxyl group positions.

The alkynyl di-hydroxylated elovanoids can have the structures of

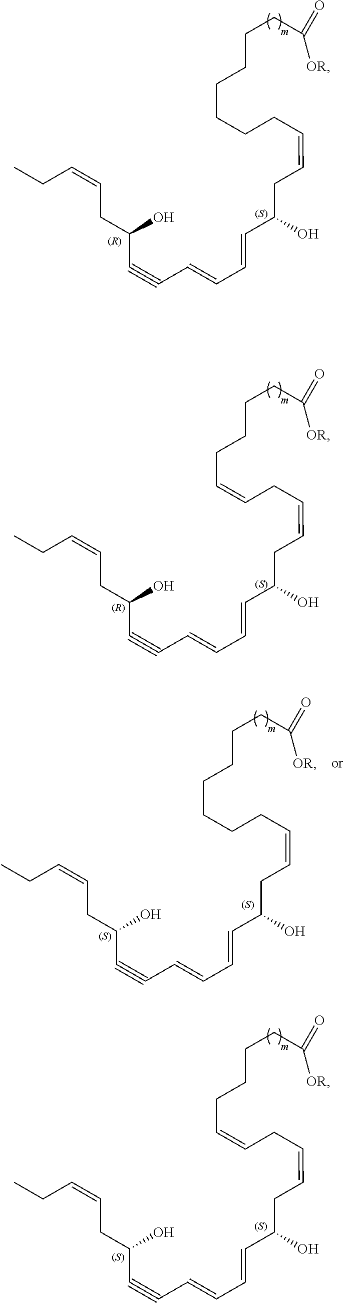

wherein, the compounds can have a total from 23 to 42 carbon atoms in the carbon chain, and wherein if there are 5 double bonds there can be 3 cis carbon-carbon double bonds starting at positions n-3, n-12, n-15 and n-18, with 2 trans carbon-carbon double bonds starting at positions n-9 and n-11, and a carbon-carbon triple bond starting at position n-7; and wherein if the compounds have 4 double bonds there can be 2 cis carbon-carbon double bonds starting at positions n-3 and n-15, with 2 trans carbon-carbon double bonds starting at positions n-9 and n-11, and a carbon-carbon triple bond starting at position n-7; wherein R is hydrogen, methyl, ethyl, alkyl, or a cation selected from a group consisting of: ammonium cation, iminium cation, or a metal cation selected from a group consisting of sodium, potassium, magnesium, zinc, or calcium cation, and wherein m is a number selected from a group consisting of 0 to 19; wherein the compounds can exist as an equimolar mixture or where predominately one enantiomer with a defined (S) or (R) chirality at any carbon bearing the hydroxyl group.

As used herein and in other structures described herein, the compounds of the invention are shown having a terminal carboxyl group “—COOR” the “R” can be a group covalently bonded to the carboxyl such as an alkyl group. In the alternative, the carboxyl group can further have a negative charge as “—COO” and R is a cation including a metal cation, an ammonium cation and the like.

In some embodiments, m is a number selected from a group consisting of 5 to 15, where the fatty acid component contains a total of 28, 29, 30, 31, 32, 33, 34, 35, 36, 37 or 38 carbon atoms in its carbon chain. In embodiments, m is a number selected from a group consisting of 5, 7, 9, 11, 13, or 15, where the fatty acid component contains a total of 28, 30, 32, 34, 36 or 38 carbon atoms in its carbon chain. In other embodiments, m is a number selected from a group consisting of 4, 6, 8, 10, 12 or 14, where the fatty acid component contains a total of 27, 29, 31, 33, 35 or 37 carbon atoms in its carbon chain. In embodiments, m is a number selected from a group consisting of 9 to 11, where the fatty acid component contains a total of 32 or 34 carbon atoms in its carbon chain.

In some embodiments the provided compound is a carboxylic acid, i.e. R is hydrogen.

In other embodiments the provided compound is a carboxylic ester, wherein R is methyl, ethyl or alkyl. In embodiments the provided compound is a carboxylic ester, wherein R is methyl or ethyl. In embodiments the provided compound is a carboxylic ester, wherein R is methyl. In other embodiments the provided compound is a carboxylate salt, wherein R is an ammonium cation, iminium cation, or a metal cation selected from a group consisting of sodium, potassium, magnesium, zinc, or calcium cation. In some embodiments, R is ammonium cation or iminium cation. In other embodiments, R is a sodium cation or a potassium cation. In embodiments, R is a sodium cation.

Embodiments described herein comprises a mono-hydroxylated 32-carbon methyl ester of formula G1, having the name: methyl (S,14Z,17Z,20Z,23Z,25E,29Z)-27-hydroxydotriaconta-14,17,20,23,25,29-hexaenoate; a mono-hydroxylated 32-carbon sodium salt of formula G2, having the name: sodium (S,14Z,17Z,20Z,23Z,25E,29Z)-27-hydroxydotriaconta-14,17,20,23,25,29-hexaenoate; a mono-hydroxylated 34-carbon methyl ester of formula G3, having the name: methyl (S,16Z,19Z,22Z,25Z,27E,31Z)-29-hydroxytetratriaconta-16,19,22,25,27,31-hexaenoate; or a mono-hydroxylated 34-carbon sodium salt of formula G4, having the name sodium (S,16Z,19Z,22Z,25Z,27E,31Z)-29-hydroxytetratriaconta-16,19,22,25,27,31-hexaenoate:

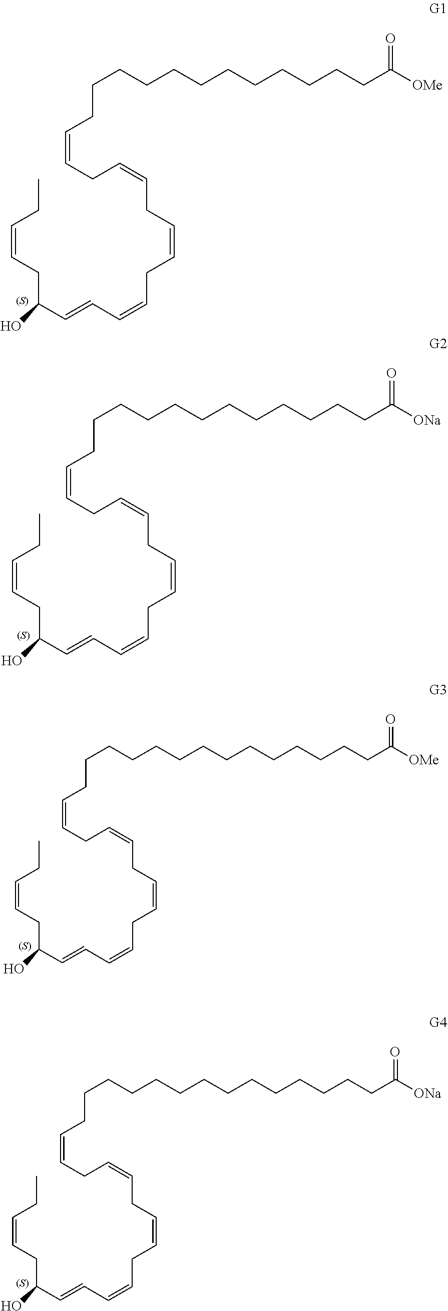

In embodiments, the compounds of structures G1, G2, G3, and G4 can exist with (R) stereochemistry at the chiral center bearing a hydroxyl group.

Embodiments described herein also comprises a di-hydroxylated 32-carbon methyl ester of formula K1, having the name: methyl (14Z,17Z,20R,21E,23E,25Z,27S,29Z)-20,27-dihydroxydotriaconta-14,17,21,23,25,29-hexaenoate; a di-hydroxylated 32-carbon sodium salt of formula K2, having the name: sodium (14Z,17Z,20R,21E,23E,25Z,27S,29Z)-20,27-dihydroxydotriaconta-14,17,21,23,25,29-hexaenoate; or a di-hydroxylated 34-carbon methyl ester of formula K3, having the name: methyl (16Z,19Z,22R,23E,25E,27Z,29S,31Z)-22,29-dihydroxytetratriaconta-16,19,23,25,27,31-hexaenoate; or a di-hydroxylated 34-carbon sodium salt of formula K4, having the name: sodium (16Z,19Z,22R,23E,25E,27Z,29S,31Z)-22,29-dihydroxytetratriaconta-16,19,23,25,27,31-hexaenoate:

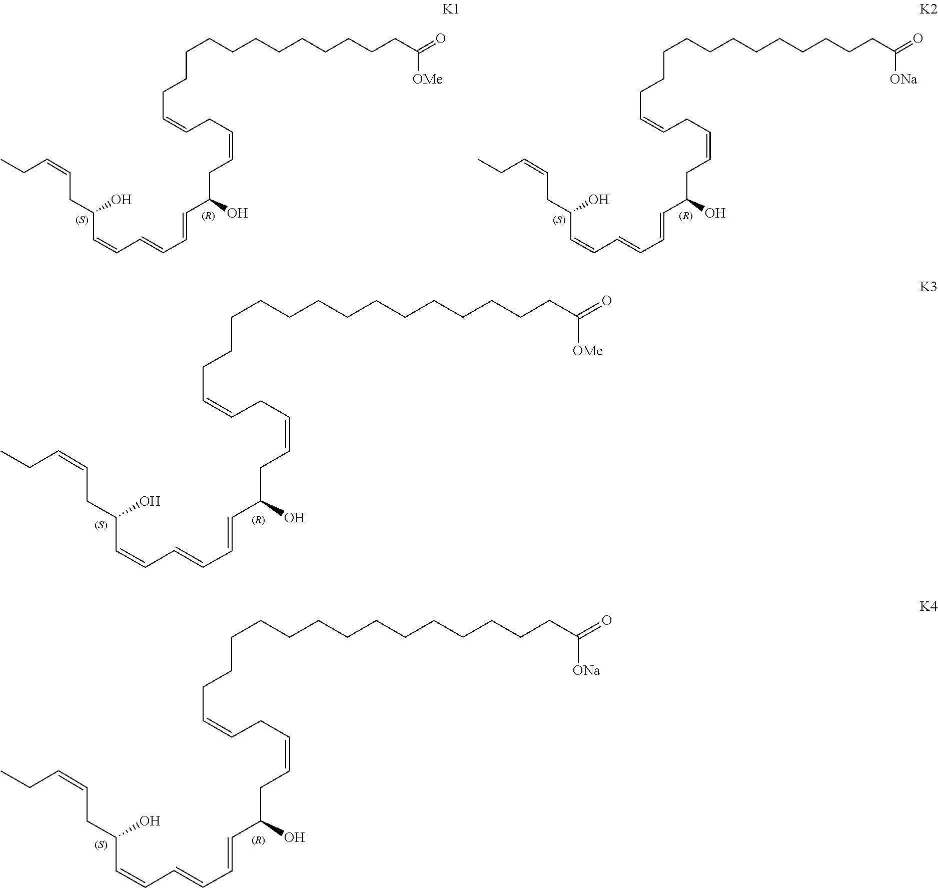

In embodiments, the compounds K1, K2, K3, and K4 can exist compounds (S),(R), (R), (R), (S), (5), and (R), (5) chiral centers at the carbon bearing hydroxyl group positions.

Other embodiments provide an alkynyl mono-hydroxylated 32-carbon methyl ester of formula O1, having the name: methyl (S,14Z,17Z,20Z,25E,29Z)-27-hydroxydotriaconta-14,17,20,25,29-pentaen-23-ynoate; an alkynyl mono-hydroxylated 32-carbon sodium salt of formula O2, having the name: sodium (S,17Z,20Z,25E,29Z)-27-hydroxydotriaconta-17,20,25,29-tetraen-23-ynoate; an alkynyl mono-hydroxylated 34-carbon methyl ester of formula O3, having the name: methyl (S,16Z,19Z,22Z,27E,31Z)-29-hydroxytetratriaconta-16,19,22,27,31-pentaen-25-ynoate; an alkynyl mono-hydroxylated 34-carbon sodium salt of formula O4, having the name: sodium (S,16Z,19Z,22Z,27E,31Z)-29-hydroxytetratriaconta-16,19,22,27,31-pentaen-25-ynoate:

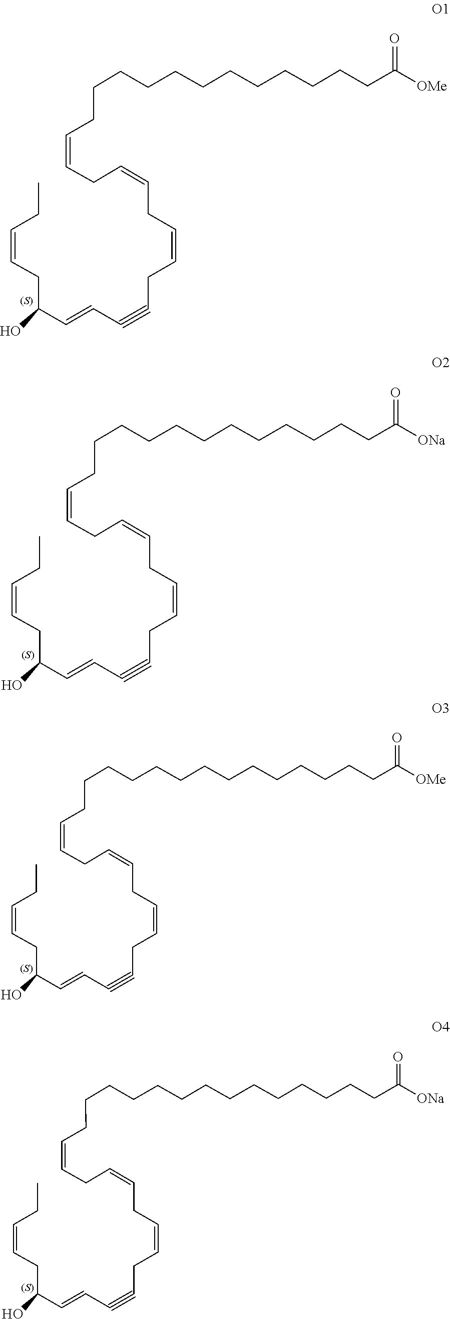

In embodiments, the compounds of structures 01, 02, 03, and 04 can exist with (R) stereochemistry at the chiral center bearing a hydroxyl group.

Still other embodiments provide an alkynyl di-hydroxylated 32-carbon methyl ester of formula S1, having the name: methyl (14Z,17Z,20R,21E,23E,27S,29Z)-20,27-dihydroxydotriaconta-14,17,21,23,29-pentaen-25-ynoate; an alkynyl di-hydroxylated 32-carbon sodium salt of formula S2, having the name: sodium (14Z,17Z,20R,21E,23E,27S,29Z)-20,27-dihydroxydotriaconta-14,17,21,23,29-pentaen-25-ynoate; or an alkynyl di-hydroxylated 34-carbon methyl ester of formula S3, having the name: methyl (16Z,19Z,22R,23E,25E,29S,31Z)-22,29-dihydroxytetratriaconta-16,19,23,25,31-pentaen-27-ynoate; or an alkynyl di-hydroxylated 34-carbon sodium salt of formula S4, having the name: sodium (16Z,19Z,22R,23E,25E,29S,31Z)-22,29-dihydroxytetratriaconta-16,19,23,25,31-pentaen-27-ynoate.

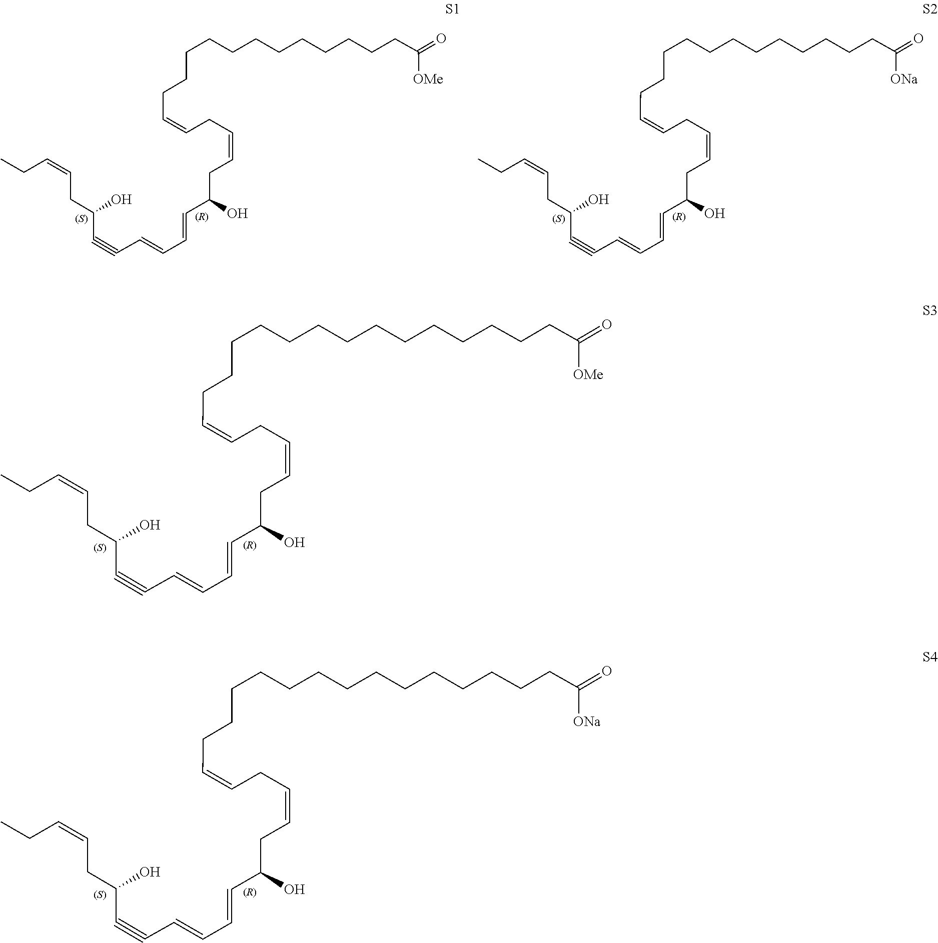

In embodiments, the compounds S1, S2, S3, and S4 can exist compounds (S), (R), (R), (R), (S), (5), and (R), (5) chiral centers at the carbon bearing hydroxyl group positions.

Methods of preparation and manufacturing of provided compounds: The compounds described herein can be readily prepared by adapting methods known in the art, starting with commercially available materials as summarized in Schemes 1-5 as shown in FIGS. 6-10.

Scheme 1 (FIG. 6) shows the detailed approach for the stereocontrolled total synthesis of compounds of type O, wherein n is 9, and the fatty acid chain contains a total of 32 carbon atoms, and the R group is methyl or sodium cation. For example, Scheme 1 shows the synthesis of compounds ELV-N32-Me and ELV-N32-Na, starting with methyl pentadec-14-ynoate (S1). By starting with heptadec-16-ynoate (T1), this process affords compounds ELV-N34-Me and ELV-N34-Na. The alkynyl precursors of ELV-N32-Me and ELV-N32-Na, namely 13a, 13b, and 15b are also among the provided compounds X and Z in this disclosure. Scheme 1 provides the reagents and conditions for the preparations of the provided compounds, by employing reaction conditions that are typical for this type of reactions.

Scheme 2 (FIG. 7) describes the total synthesis of the di-hydroxylated elovanoids K and L and their alkyne precursors S and T, by starting with intermediates 2, 5, and 7 that were also used in Scheme 1. The conversion of the protected (R) epoxide 4 to intermediate 15, and the coupling of 7 and 15 followed by conversion into intermediate 17 can be done according to literature procedures (Tetrahedron Lett. 2012; 53(14):1695-8).

Catalytic cross-coupling between intermediates 2 or 17 or between intermediates 5 or 17, followed by deprotection, leads to the formation of alkynyl compounds S and T, which are then selectively reduced to form di-hydroxylated elovanoids K and L. Hydrolysis and acidification affords the corresponding carboxylic acids, which can be converted into carboxylate salts with the addition of equivalent amounts of the corresponding base. Di-hydroxylated elovanoids of types K, L, S and T with at least 23 carbons and up to 42 carbons in their carbon chain, can be similarly prepared by varying the number of carbons in the alkyne starting material 7.

Scheme 3 (FIG. 8) describes the total synthesis of di-hydroxylated elovanoids with five unsaturated double bonds of types M and N, as well as their alkyne precursors U and V, by utilizing the same alkynyl intermediates 2 and 5, which were also used in Scheme 1. (Tetrahedron Lett. 2012; 53 (14): 1695-8).

The synthesis of the common intermediate 22 begins with the carboxylic acid 18, which is converted into orthoester 19, using known methodologies (Tetrahedron Lett. 1983, 24 (50), 5571-4). Reaction of the lithiated alkyne with epoxide 1 affords intermediate 21, which is converted into the iodide intermediate 22, similarly to the conversion of 16 to 17. Catalytic cross-coupling between intermediates 2 or 5 with 22, followed by deprotection, leads to the formation of alkynyl di-hydroxy elovanoids U and V, which are then selectively reduced to form di-hydroxylated elovanoids M and N.

Hydrolysis and acidification affords the corresponding carboxylic acids, which can be converted into carboxylate salts with the addition of equivalent amounts of the corresponding base. Di-hydroxylated elovanoids of types M, N, U and V with at least 23 carbons and up to 42 carbons in their carbon chain, can be similarly prepared by varying the number of carbons in the alkyne carboxylic acid 18.

Scheme 4 (FIG. 9) shows the stereocontrolled total synthesis of 32-carbon dihydroxylated elovanoids, starting with alkyne methyl ester 23, intermediate 15, and alkyne intermediate 2. For example, this scheme shows the total synthesis of the 32-carbon alkynyl elovanoid compound ELV-N32-Me-Acetylenic, and its conversion to elovanoid methyl ester ELV-N32-Me, the elovanoid carboxylic acid ELV-N32-H, and the elovanoid sodium salt ELV-N32-Na.

Scheme 5 (FIG. 10) shows the stereocontrolled total synthesis of 34-carbon dihydroxylated elovanoids, starting with alkyne methyl ester 30, and by employing the same sequence of reactions as in Scheme 4.

For example, this scheme shows the total synthesis of the 34-carbon alkynyl elovanoid compound ELV-N34-Me-Acetylenic, and its conversion to elovanoid methyl ester ELV-N34-Me, the elovanoid carboxylic acid ELV-N34-H, and the elovanoid sodium salt ELV-N34-Na.

The chemistry presented in Schemes 1-5 (FIGS. 6-10) can be also readily adapted for the total synthesis of additional mono-hydroxylated and di-hydroxylated elovanoids, having at least 23 carbons and up to 42 carbons in their carbon chain.

Pharmaceutical compositions for the treatment of diseases: In other embodiments, the present disclosure provides formulations of pharmaceutical compositions containing therapeutically effective amounts of one or more of compounds provided herein or their salts thereof in a pharmaceutically acceptable carrier.

The provided compositions contain one or more compounds provided herein or their salts thereof, and a pharmaceutically acceptable excipient, diluent, carrier and/or adjuvant. The compounds can be formulated into suitable pharmaceutical preparations such as solutions, suspensions, tablets, dispersible tablets, pills, capsules, powders, sustained release formulations or elixirs, for oral, buccal, intranasal, vaginal, rectal, ocular administration, sustained release from intravitreal implanted reservoirs or nano-devices or dendrimers, embedded in collagen or other materials on the eye surface, or in sterile solutions or suspensions for parenteral administration, dermal patches as well as transdermal patch preparation and dry powder inhalers. The provided formulations can be in the form of a drop, such as an eye drop, and the pharmaceutical formulation can further contain antioxidants and/or known agents for the treatment of eye diseases. The compounds described herein can be formulated into pharmaceutical compositions using techniques and procedures well known in the art (see, e.g., Ansel Introduction to Pharmaceutical Dosage Forms, Fourth Edition 1985, 126).

Embodiments of the disclosure provide pharmaceutical compositions containing various forms of the provided compounds, as the free carboxylic acids or their pharmaceutically acceptable salts, or as their corresponding esters or their phospholipid derivatives. In other advantageous embodiments, the disclosure provides pharmaceutical compositions containing one or more elovanoid that contains one or two hydroxyl groups at positions located between n-3 to n-18 of the very long chain polyunsaturated fatty acids, as the free carboxylic acids or their pharmaceutically acceptable salts, or as their corresponding esters.

In a further embodiment, the disclosure provides a pharmaceutical composition for alleviating the symptom of, treating, or preventing a viral inflammatory disease.

In the provided compositions, effective concentrations of one or more compounds or pharmaceutically acceptable derivatives is (are) mixed with a suitable pharmaceutical carrier or vehicle. The compounds can be derivatized as the corresponding salts, esters, enol ethers or esters, acids, bases, solvates, hydrates or prodrugs prior to formulation, as described above. The concentrations of the compounds in the compositions are effective for delivery of an amount, upon administration, that treats, prevents, or ameliorates one or more of the symptoms of a disease, disorder or condition.

As described herein, the compositions can be readily prepared by adapting methods known in the art. The compositions can be a component of a pharmaceutical formulation. The pharmaceutical formulation can further contain known agents for the treatment of inflammatory or degenerative diseases, including neurodegenerative diseases. The provided compositions can serve as pro-drug precursors of the fatty acids and can be converted to the free fatty acids upon localization to the site of the disease.

Embodiments described herein also provide packaged composition(s) or pharmaceutical composition(s) for prevention, restoration, or use in treating the disease or condition. Other packaged compositions or pharmaceutical compositions provided by the present disclosure further include indicia including at least one of: instructions for using the composition to treat the disease or condition. The kit can further include appropriate buffers and reagents known in the art for administering various combinations of the components listed above to the host.

Pharmaceutical formulations: Embodiments described herein can include a composition or pharmaceutical composition as identified herein and can be formulated with one or more pharmaceutically acceptable excipients, diluents, carriers, naturally occurring or synthetic antioxidants, and/or adjuvants. In addition, embodiments of the present disclosure include a composition or pharmaceutical composition formulated with one or more pharmaceutically acceptable auxiliary substances. For example, the composition or pharmaceutical composition can be formulated with one or more pharmaceutically acceptable excipients, diluents, carriers, and/or adjuvants to provide an embodiment of a composition of the present disclosure.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc. The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In an embodiment, the composition or pharmaceutical composition can be administered to the subject using any means which can result in the desired effect. Thus, the composition or pharmaceutical composition can be incorporated into a variety of formulations for therapeutic administration. For example, the composition or pharmaceutical composition can be formulated into pharmaceutical compositions by combination with appropriate, pharmaceutically acceptable carriers or diluents, and can be formulated into preparations in solid, semi-solid, liquid or gaseous forms, such as tablets, capsules, powders, granules, ointments, solutions, suppositories, injections, inhalants, creams, and aerosols. For example, the VLC-PUFAs may be deployed as an inhalable, lung surfactant to attenuate inflammation and disease onset and progression. The term “surfactant” can refer to synthetic and naturally occurring amphiphilic molecules that have hydrophobic portion(s) and hydrophilic portion(s) which due to their amphiphilic (amphipathic) nature, can reduce the surface tension at an interface. For example, the interface is between air and water. The term “lung surfactant” can refer to a surface-active agent that is found in the pulmonary system, especially the lining of alveoli, which protects the lungs from injury or infection. The term “inhalant” can refer to a therapeutic agent that is administered through inhalation.

Suitable excipient vehicles for the composition or pharmaceutical composition are, for example, water, saline, dextrose, glycerol, ethanol, or the like, and combinations thereof. In addition, the vehicle can contain minor amounts of auxiliary substances such as wetting or emulsifying agents, antioxidants or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pennsylvania, 17th edition, 1985. The composition or formulation to be administered will, in any event, contain a quantity of the composition or pharmaceutical composition adequate to achieve the desired state in the subject being treated.

Compositions described herein can include those that comprise a sustained release or controlled release matrix. In addition, embodiments of the present disclosure can be used in conjunction with other treatments that use sustained-release formulations. As used herein, a sustained-release matrix is a matrix made of materials, for example polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix. In another embodiment, the pharmaceutical composition of the present disclosure (as well as combination compositions) can be delivered in a controlled release system. For example, the composition or pharmaceutical composition can be administered using intravenous infusion, an implantable osmotic pump, a transdermal patch, liposomes, or other modes of administration. In one embodiment, a pump can be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions (as well as combination compositions separately or together) include those formed by impregnation of the composition or pharmaceutical composition described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

In another embodiment, the compositions or pharmaceutical compositions (as well as combination compositions separately or together) can be part of a delayed-release formulation. Delayed-release dosage formulations can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, M D, 2000, and “Pharmaceutical dosage forms and drug delivery systems”, 6th Edition, Ansel et al., (Media, PA: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

Embodiments of the composition or pharmaceutical composition can be administered to a subject in one or more doses. Those of skill will readily appreciate that dose levels can vary as a function of the specific the composition or pharmaceutical composition administered, the severity of the symptoms and the susceptibility of the subject to side effects. Dosages for a given compound are readily determinable by those of skill in the art by a variety of means.

In an embodiment, multiple doses of the composition or pharmaceutical composition are administered. The frequency of administration of the composition or pharmaceutical composition can vary depending on any of a variety of factors, e.g., severity of the symptoms, and the like. For example, in an embodiment, the composition or pharmaceutical composition can be administered once per month, twice per month, three times per month, every other week (qow), once per week (qw), twice per week (biw), three times per week (tiw), four times per week, five times per week, six times per week, every other day (qod), daily (qd), twice a day (qid), three times a day (tid), or four times a day. As discussed above, in an embodiment, the composition or pharmaceutical composition is administered 1 to 4 times a day over a 1 to 10-day time period.

The duration of administration of the composition or pharmaceutical composition analogue, e.g., the period of time over which the composition or pharmaceutical composition is administered, can vary, depending on any of a variety of factors, e.g., patient response, etc. For example, the composition or pharmaceutical composition in combination or separately, can be administered over a period of time of about one day to one week, about one day to two weeks.

The amount of the compositions and pharmaceutical compositions described herein can be effective in treating the condition or disease can be determined by standard clinical techniques. In addition, in vitro or in vivo assays can be employed to help identify optimal dosage ranges. The precise dose to be employed can also depend on the route of administration, and can be decided according to the judgment of the practitioner and each patient's circumstances.

Routes of Administration: Embodiments of the present disclosure provide methods and compositions for the administration of the active agent(s) to a subject (e.g., a human) using any available method and route suitable for drug delivery, including in vivo and ex vivo methods, as well as systemic and localized routes of administration. Routes of administration include intranasal, intramuscular, intratracheal, subcutaneous, intradermal, intravitreal, topical application, intravenous, rectal, nasal, oral, and other enteral and parenteral routes of administration. Routes of administration can be combined, or adjusted depending upon the agent and/or the desired effect. An active agent can be administered in a single dose or in multiple doses.

The n-3 VLC-PUFA and their biogenic derivatives can be formed in cells and are not a component of human diet. Routes of administration of the compounds provided herein comprise topical, oral, intranasal, and parenteral administration. For example, the provided formulations can be delivered in the form of a drop, such as an eye drop, or any other customary method for the treatment of a viral inflammatory disease of the eye. For example, the provided formulations can be delivered in the form of an intranasal spray or any other customary method for the treatment of a viral inflammatory disease of the nasal passage or lungs. For example, the provided formulations can be delivered in the form of a cream or gel or any other customary method for the treatment of a viral inflammatory disease of the skin.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to affect systemic or local delivery of the composition. Where systemic delivery is the goal, administration involves invasive or systemically absorbed topical or mucosal administration of pharmaceutical preparations. In an embodiment, the composition or pharmaceutical composition can also be delivered to the subject by enteral administration. Enteral routes of administration include, but are not limited to, oral and rectal (e.g., using a suppository) delivery.

Methods of administration of the composition or pharmaceutical composition through the skin or mucosa include, but are not limited to, topical application of a suitable pharmaceutical preparation, transdermal transmission, injection, inhalation, and epidermal administration. For transdermal transmission, absorption promoters or iontophoresis are suitable methods. Iontophoretic transmission can be accomplished using commercially available “patches” that deliver their product continuously via electric pulses through unbroken skin for periods of several days or more. For deployment via inhalation, the composition or pharmaceutical composition can also be administered as an inhalable lung surfactant. For example, the inhalable lung surfactant can be preventative, to attenuate inflammation, disease onset and progress in several scenarios. In further embodiments, the inhalable lung surfactant can be administered as an inhalable as a preventative mode and at disease onset in a higher concentration.

The compounds and compositions described herein can restore homeostasis and induce survival signaling in certain cells undergoing oxidative stress or other homeostatic disruptions. Aspects described herein are also drawn to methods of use of the compounds and compositions containing a hydroxylated derivative of very long chain polyunsaturated fatty acids, as the free carboxylic acids or their pharmaceutically acceptable salts, or as their corresponding esters or other prodrug derivatives. The provided compounds can be readily prepared by adapting methods known in the art, starting with commercially available materials.

The bioactivity of the compounds described herein, as exemplified by the elovanoid derivatives ELV-N32-Me, ELV-N32-Na, ELV-N34-Me and ELV-N34-Na, Resolvin D6, Lipoxin A4, and R,R-RvD6i, is attributed to their ability to reach the targeted human cells and exert their biological actions either by entering into the cell or/and by acting at a membrane bound receptor. Alternatively, the provided compounds can act via intracellular receptors (e.g. nuclear membrane), and thus they would work specifically by affecting key signaling events. Administration of a pharmaceutical composition, containing a provided compound and a pharmaceutically acceptable carrier, restores the homeostatic balance and promotes the survival of certain cells that are essential for maintaining normal function. The provided compounds, compositions, and methods can be used for the preventive and therapeutic treatment of inflammatory, degenerative, and neurodegenerative diseases. This disclosure targets critical steps of the initiation and early progression of these conditions by mimicking the specific biology of intrinsic cellular/organs responses to attain potency, selectivity, devoid of side effects and sustained bioactivity.

Accordingly, one aspect encompasses embodiments of a composition comprising at least one very long chain polyunsaturated fatty acid having at least 23 carbon atoms in its carbon chain.

In some embodiments, the composition can further comprise a pharmaceutically-acceptable carrier and formulated for delivery of an amount of the at least one very long chain polyunsaturated fatty acid effective in reducing a pathological condition of a tissue of a recipient subject or the onset of a pathological condition of a tissue of a recipient subject.

In some embodiments, the pathological condition can be a viral disease or viral inflammatory response or condition of a tissue of the recipient subject. For example, the virus can be SARS-CoV-2.

In some embodiments, the composition can be formulated for topical delivery of the biomolecules described herein to the tissue of the skin or eye of a recipient subject.

In some embodiments, the composition can be formulated for intranasal delivery of the at least one very long chain polyunsaturated fatty acid tissue to the nasal passage and/or lungs of a recipient subject.

In some embodiments, the composition can further comprise at least one nutritional component, and, for example, the composition can be formulated for the oral or parenteral delivery of the at least one very long chain polyunsaturated fatty acid, elovanoids, Resolvin, Resolvin-D6 isomer, or Lipoxin A4 to a recipient subject.

In some embodiments, the at least one very long chain polyunsaturated fatty acid can have from about 26 to about 42 carbon atoms in its carbon chain.

In some embodiments, the at least one very long chain polyunsaturated fatty acid can have 32 or 34 carbon atoms in its carbon chain.

In some embodiments, the very long chain polyunsaturated fatty acid can have in its carbon chain five or six double bonds with cis geometry.

In some embodiments, the very long chain polyunsaturated fatty acid is 14Z,17Z,20Z,23Z,26Z,29Z)-dotriaconta-14,17,20,23,26,29-hexaenoic acid or (16Z,19Z,22Z,25Z,28Z,31Z)-tetratriaconta-16,19,22,25,28,31-hexaenoic acid.

Another aspect encompasses embodiments of a composition comprising at least one elovanoid having at least 23 carbon atoms in its carbon chain.

In some embodiments, the composition can further comprise a pharmaceutically-acceptable carrier and can be formulated for delivery of an amount of the at least one elovanoid effective in reducing a pathological condition of a tissue of a recipient subject.

In some embodiments, the pathological condition can be a viral inflammatory disease.

In some embodiments, the at least one elovanoid can be selected from the group consisting of: a mono-hydroxylated elovanoid, a di-hydroxylated elovanoid, an alkynyl mono-hydroxylated elovanoid, and an alkynyl di-hydroxylated elovanoid, or any combination thereof.

In some embodiments, the at least one elovanoid can be a combination of elovanoids, wherein the combination is selected from the group consisting of: a mono-hydroxylated elovanoid and a di-hydroxylated elovanoid; a mono-hydroxylated elovanoid and an alkynyl mono-hydroxylated elovanoid; a mono-hydroxylated elovanoid and an alkynyl di-hydroxylated elovanoid; a di-hydroxylated elovanoid and an alkynyl mono-hydroxylated elovanoid; a di-hydroxylated elovanoid and an alkynyl di-hydroxylated elovanoid; a mono-hydroxylated elovanoid, a di-hydroxylated elovanoid, and an alkynyl mono-hydroxylated elovanoid; a mono-hydroxylated elovanoid, a di-hydroxylated elovanoid, and an alkynyl di-hydroxylated elovanoid; and a mono-hydroxylated elovanoid, a di-hydroxylated elovanoid, and an alkynyl mono-hydroxylated elovanoid an alkynyl di-hydroxylated elovanoid, wherein each elovanoid is independently a racemic mixture, an isolated enantiomer, or a combination of enantiomers wherein the amount of one enantiomer greater than the amount of another enantiomer; and wherein each di-hydroxylated elovanoid is independently a diastereomeric mixture, an isolated diastereomer, or a combination of diastereomers wherein the amount of one diastereomer is greater than the amount of another diastereomer.

In some embodiments, the composition can further comprise at least one very long-chain polyunsaturated fatty acid having at least 23 carbon atoms in its carbon chain.

In some embodiments, the at least one very long chain polyunsaturated fatty acid can have from about 26 to about 42 carbon atoms in its carbon chain.

In some embodiments, the at least one very long chain polyunsaturated fatty acid can have in its carbon chain five or six double bonds with cis geometry.

In some embodiments, the at least one very long chain polyunsaturated fatty acid can be 14Z,17Z,20Z,23Z,26Z,29Z)-dotriaconta-14,17,20,23,26,29-hexaenoic acid or (16Z,19Z,22Z,25Z,28Z,31Z)-tetratriaconta-16,19,22,25,28,31-hexaenoic acid.

In some embodiments, the mono-hydroxylated elovanoid can be selected from the group consisting of the formulas G, H, I or J:

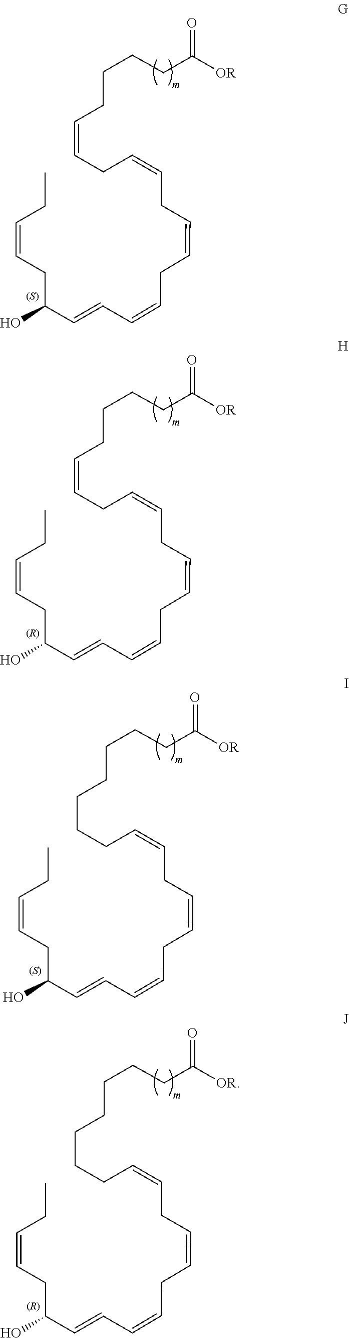

wherein: n can be 0 to 19 and —CO—OR can be a carboxylic acid group, or a salt or an ester thereof, and wherein: if —CO—OR can be a carboxylic acid group and the compound G, H, I or J can be a salt thereof, the cation of the salt can be a pharmaceutically acceptable cation, and if —CO—OR can be an ester, then R can be an alkyl group.

In some embodiments, the pharmaceutically acceptable cation can be an ammonium cation, an iminium cation, or a metal cation.

In some embodiments, the metal cation can be a sodium, potassium, magnesium, zinc, or calcium cation.

In some embodiments, the composition can comprise equimolar amounts of the enantiomers G and H wherein the enantiomers have (S) or (R) chirality at the carbon bearing the hydroxyl group.

In some embodiments, the composition can comprise amounts of the enantiomers I and J wherein the enantiomers have (S) or (R) chirality at the carbon bearing the hydroxyl group.

In some embodiments, the composition can comprise one of the enantiomers of G or H in an amount exceeding the amount of the other enantiomer of G or H.

In some embodiments, the composition can comprise one of the enantiomers of I or J in an amount exceeding the amount of the other enantiomer of I or J.

In some embodiments, the mono-hydroxylated elovanoid can be selected from a group consisting of: methyl (S,14Z,17Z,20Z,23Z,25E,29Z)-27-hydroxydotriaconta-14,17,20,23,25,29-hexaenoate (G1), sodium (S,14Z,17Z,20Z,23Z,25E,29Z)-27-hydroxydotriaconta-14,17,20,23,25,29-hexaenoate (G2), methyl (S,16Z,19Z,22Z,25Z,27E,31Z)-29-hydroxytetratriaconta-16,19,22,25,27,31-hexaenoate (G3); and sodium (S,16Z,19Z,22Z,25Z,27E,31Z)-29-hydroxytetratriaconta-16,19,22,25,27,31-hexaenoate (G4) having the formulas, respectively:

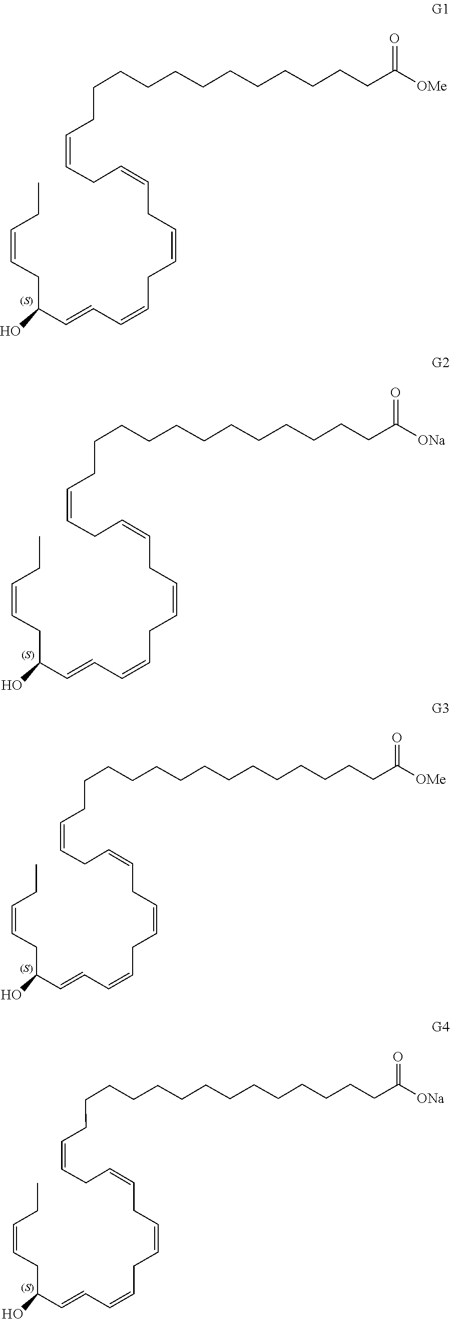

In some embodiments, the di-hydroxylated elovanoid can be selected from the group consisting of the formulas K, L, M, and N:

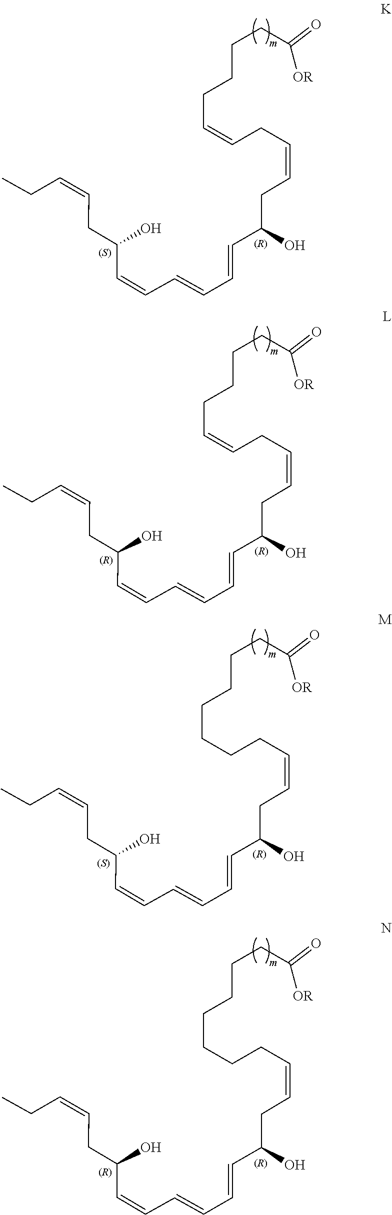

wherein: m can be 0 to 19 and —CO—OR can be a carboxylic acid group, or a salt or an ester thereof, and wherein: if —CO—OR can be a carboxylic acid group and the compound K, L, M, or N can be a salt thereof, the cation of the salt can be a pharmaceutically acceptable cation, and if —CO—OR can be an ester, then R can be an alkyl group.

In some embodiments, the pharmaceutically acceptable cation can be an ammonium cation, an iminium cation, or a metal cation.

In some embodiments, the metal cation can be a sodium, potassium, magnesium, zinc, or calcium cation.

In some embodiments, the composition can comprise equimolar amounts of the diastereomers K and L wherein the diastereomers have either (S) or (R) chirality at position n-6, and (R) chirality at position n-13.

In some embodiments, the composition can comprise equimolar amounts of the diastereomers M and N wherein the diastereomers have either (S) or (R) chirality at position n-6, and (R) chirality at position n-13.

In some embodiments, the composition can comprise one of the diastereomers of K or L in an amount exceeding the amount of the other diastereomer of K or L.

In some embodiments, the composition can comprise one of the diastereomers of M or N in an amount exceeding the amount of the other diastereomer of M or N.

In some embodiments, the di-hydroxylated elovanoid can be selected from the group consisting of: methyl (14Z,17Z,20R,21E,23E,25Z,27S,29Z)-20,27-dihydroxydotriaconta-14,17,21,23,25,29-hexaenoate (K1), sodium (14Z,17Z,20R,21E,23E,25Z,27S,29Z)-20,27-dihydroxydotriaconta-14,17,21,23,25,29-hexaenoate (K2), methyl (16Z,19Z,22R,23E,25E,27Z,29S,31Z)-22,29-dihydroxytetratriaconta-16,19,23,25,27,31-hexaenoate (K3), and sodium (16Z,19Z,22R,23E,25E,27Z,29S,31Z)-22,29-dihydroxytetratriaconta-16,19,23,25,27,31-hexaenoate (K4) having the formulas, respectively:

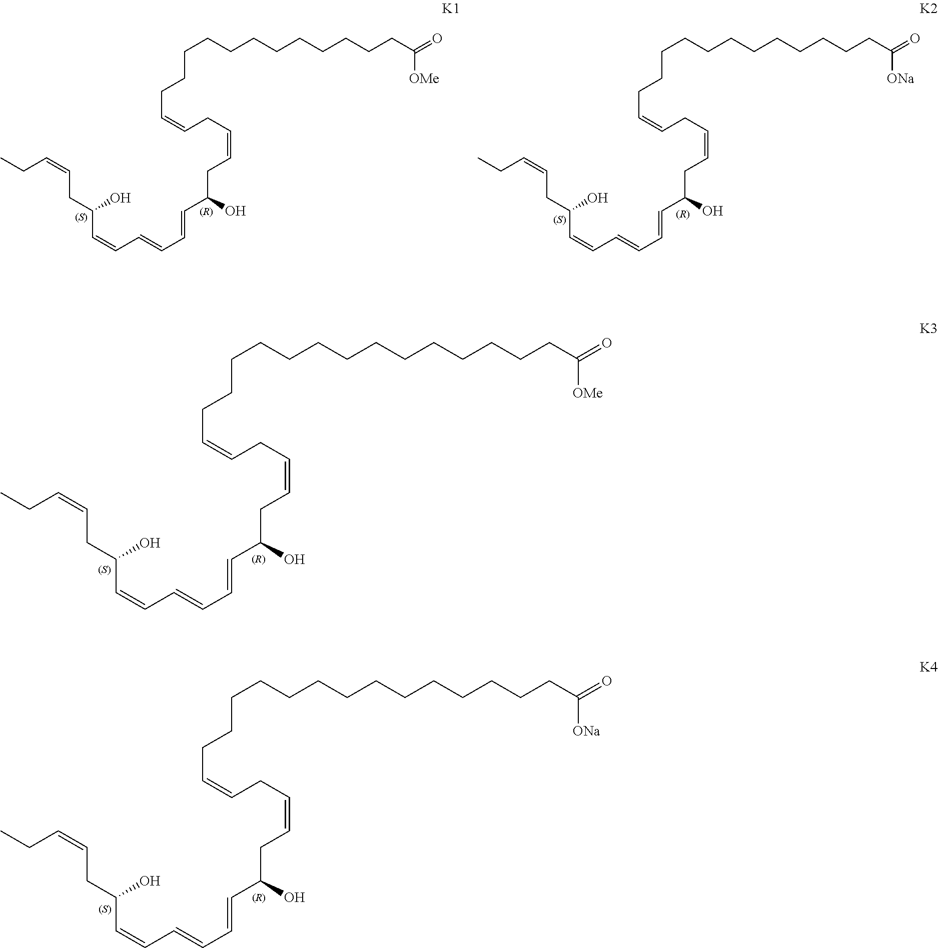

In some embodiments, the alkynyl mono-hydroxylated elovanoid can be selected from the group consisting of the formulas O, P, Q or R:

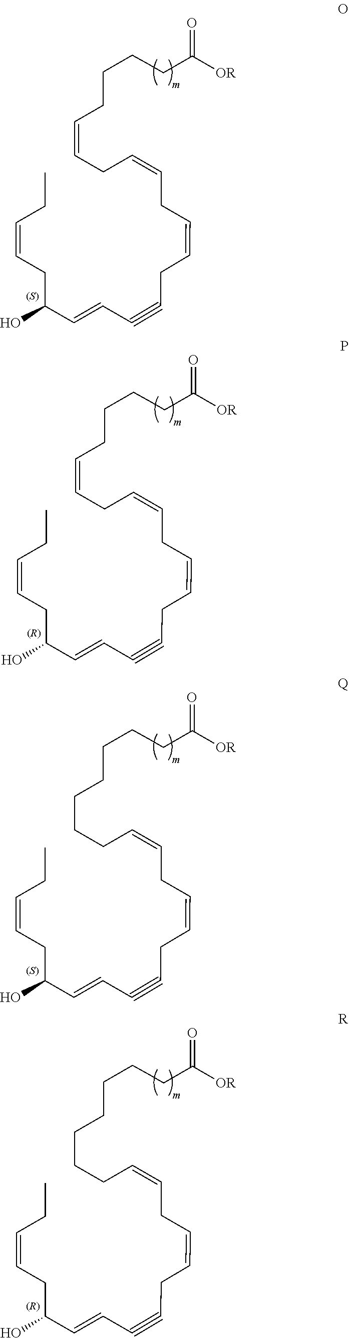

wherein: m can be 0 to 19 and —CO—OR can be a carboxylic acid group, or a salt or an ester thereof, and wherein: if —CO—OR can be a carboxylic acid group and the compound O, P, Q or R can be a salt thereof, the cation of the salt can be a pharmaceutically acceptable cation, and if —CO—OR can be an ester, then R can be an alkyl group, and wherein: compounds 0 and P each have a total from 23 to 42 carbon atoms in the carbon chain, with 4 cis carbon-carbon double bonds located at positions starting at n-3, n-12, n-15 and n-18; with a trans carbon-carbon double bond at position starting at n-7, and a carbon-carbon triple bond starting at position n-9; and compounds Q and R each have a total from 23 to 42 carbon atoms in the carbon chain, with 3 cis carbon-carbon double bond starting at positions n-3, n-12 and n-15, with a trans carbon-carbon double bond at position starting at n-7, and a carbon-carbon triple bond starting at position n-9.

In some embodiments, the alkynyl mono-hydroxylated elovanoid can be selected from the group consisting of: methyl (S,14Z,17Z,20Z,25E,29Z)-27-hydroxydotriaconta-14,17,20,25,29-pentaen-23-ynoate (01); sodium (S,17Z,20Z,25E,29Z)-27-hydroxydotriaconta-17,20,25,29-tetraen-23-ynoate (02); methyl (S,16Z,19Z,22Z,27E,31Z)-29-hydroxytetratriaconta-16,19,22,27,31-pentaen-25-ynoate (03); and sodium (S,16Z,19Z,22Z,27E,31Z)-29-hydroxytetratriaconta-16,19,22,27,31-pentaen-25-ynoate (04) and having the formulas, respectively:

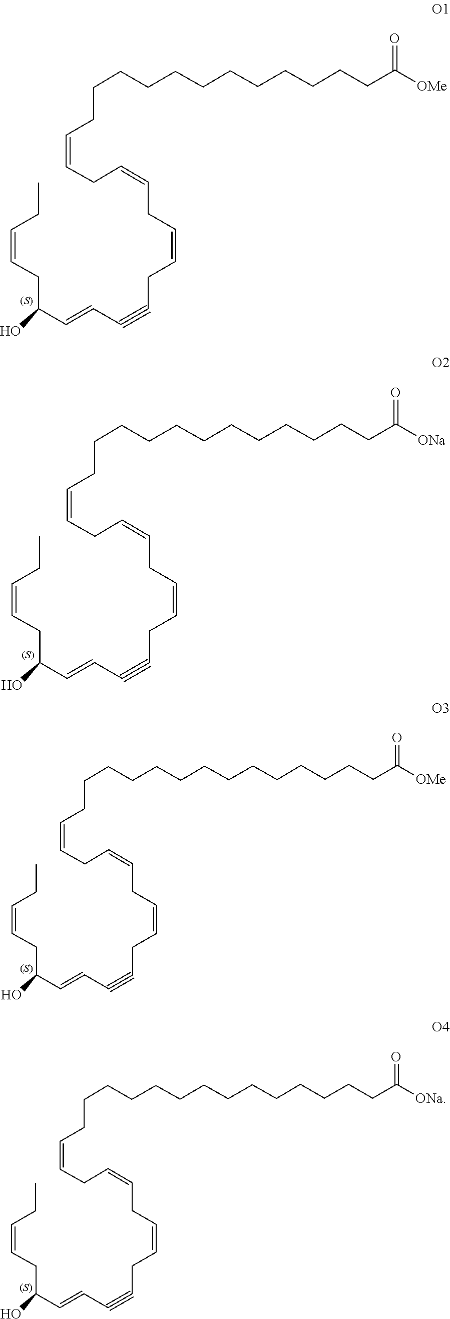

In some embodiments, the pharmaceutically acceptable cation can be an ammonium cation, an iminium cation, or a metal cation.

In some embodiments of this aspect of the disclosure, the metal cation can be a sodium, potassium, magnesium, zinc, or calcium cation.

In some embodiments, the composition can comprise equimolar amounts of the enantiomers O and P wherein the enantiomers have (S) or (R) chirality at the carbon bearing the hydroxyl group.

In some embodiments, the composition can comprise equimolar amounts of the enantiomers Q and R wherein the enantiomers have (S) or (R) chirality at the carbon bearing the hydroxyl group.

In some embodiments, the composition can comprise one of the enantiomers of 0 or P in an amount exceeding the amount of the other enantiomer of 0 or P.

In some embodiments, the composition can comprise one of the enantiomers of Q or R in an amount exceeding the amount of the other enantiomer of Q or R.

In some embodiments, the elovanoid can be an alkynyl di-hydroxylated elovanoid selected from the group consisting of the formulas S, T, U or V:

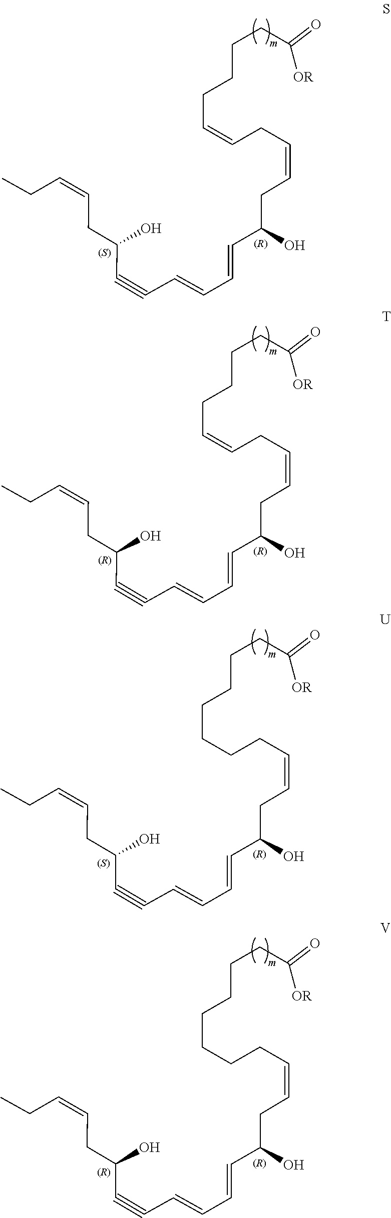

wherein: m can be 0 to 19 and —CO—OR can be a carboxylic acid group, or a salt or an ester thereof, and wherein: if —CO—OR can be a carboxylic acid group and the compound S, T, U or V can be a salt thereof, the cation of the salt can be a pharmaceutically acceptable cation, and if —CO—OR can be an ester, then R can be an alkyl group, and wherein: compounds S and T each have a total from 23 to 42 carbon atoms in the carbon chain, with 3 cis carbon-carbon double bonds starting at positions n-3, n-15 and n-18; 2 trans carbon-carbon double bonds starting at positions n-9, n-11; and a carbon-carbon triple bond starting at position n-7; and compounds U and V each have a total from 23 to 42 carbon atoms in the carbon chain, with 2 cis carbon-carbon double bond starting at positions n-3, and n-15; 2 trans carbon-carbon double bonds starting at positions n-9 and n-11; and a carbon-carbon triple bond starting at position n-7.

In some embodiments, the pharmaceutically acceptable cation is an ammonium cation, an iminium cation, or a metal cation.

In some embodiments, the metal cation is a sodium, potassium, magnesium, zinc, or calcium cation.

In some embodiments, the alkynyl mono-hydroxylated elovanoid can be selected from the group consisting of: methyl (14Z,17Z,20R,21E,23E,27S,29Z)-20,27-dihydroxydotriaconta-14,17,21,23,29-pentaen-25-ynoate (S1); sodium (14Z,17Z,20R,21E,23E,27S,29Z)-20,27-dihydroxydotriaconta-14,17,21,23,29-pentaen-25-ynoate (S2); methyl (16Z,19Z,22R,23E,25E,29S,31Z)-22,29-dihydroxytetratriaconta-16,19,23,25,31-pentaen-27-ynoate (S3); and sodium (16Z,19Z,22R,23E,25E,29S,31Z)-22,29-dihydroxytetratriaconta-16,19,23,25,31-pentaen-27-ynoate (S4), and having the formula, respectively:

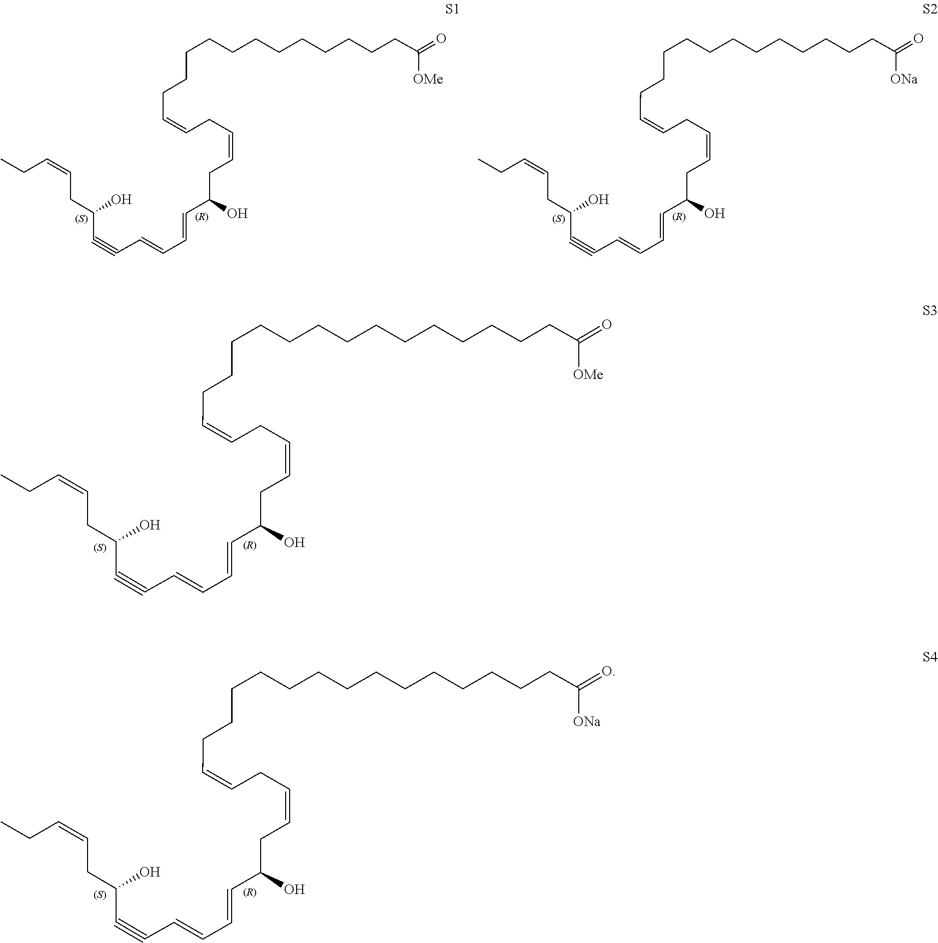

In some embodiments, the composition can comprise equimolar amounts of the diastereomers S and T wherein the diastereomers have (S) or (R) chirality at the carbons bearing the hydroxyl groups.

In some embodiments, the composition can comprise equimolar amounts of the diastereomers U and V wherein the diastereomers have either (S) or (R) chirality at position n-6, and (R) chirality at position n-13.

In some embodiments, the composition can comprise one of the diastereomers of S or T in an amount exceeding the amount of the other diastereomer of S or T.

In some embodiments, the composition can comprise one of the diastereomers of U or V in an amount exceeding the amount of the other diastereomer of U or V.

Other compositions, compounds, methods, features, and advantages of the present disclosure will be or become apparent to one having ordinary skill in the art upon examination of the following drawings, detailed descriptioinn, and examples. All such additional compositions, compounds, methods, features, and advantages can be included within this description, and be within the scope of the present disclosure.

### EXAMPLES

### Example 1

As shown herein, adding very long chain polyunsaturated fatty acids (VLC-PUFAs) to human bronchiole and alveoli cells in culture activates the synthesis of elovanoids (ELVs) 32 and 34. These two mediators counter-regulate the cytokine storm and other inflammatory components activated by the virus in the lung. Primary cell cultures of human bronchiole and alveoli, a mixture of ciliated cells, club cells, type-I pneumocytes and type-II pneumocytes (obtained from PromoCell, HSAEpC). The predominant cell is type II pneumocytes.

Without wishing to be bound by theory, the VLC-PUFA curtail inflammation and/or cytokine storm by fostering the synthesis of protective bioactive mediators, the elovanoids. The VLC-PUFAs target the damaging inflammatory response to SARS-CoV-2 on the immune system reflected in the cytokine storm. Without wishing to be bound by theory, it is contained by activating pro-homeostatic pathways of ELVs synthesis in human bronchiole and alveoli as we find now. These lipids were discovered (Bhattacharjee et al. Sci Adv 2017 and Do et al. PNAS 2019) and have since been shown to have potent pro-homeostatic properties (Bhattacharjee et al. Sci Adv 2017; Do et al. PNAS 2019; and Bazan et al. Mol Aspects Med. 2018).

32C and 34C VLC-PUFAs, n-3 (1 microM) added to primary cell cultures of human bronchiole and alveoli remarkably activates the synthesis of ELVs. See, for example, FIG. 11 and FIG. 12 which show fragmentation patterns of ELVs of 32C and 34C as well as of their stable precursors 27-hydroxy and 29-hydroxy respectively. This has not been seen before in cells. Human bronchiole and alveoli cells are very active in phospholipid synthesis, mainly of PC containing palmitic acid and oleic acid, principal components of the lung surfactant. This indicates the use of VLC-PUFAs that by stimulating endogenous elovanoids synthesis enhances the intrinsic anti-inflammatory ability of the lung against the virus.

FIG. 15, for example, shows that VLC-PUFAs induce lipidome remodeling, and without wishing to be bound by theory, this can disrupt tetraspanin-enriched membrane microdomains (they are not lipid rafts) blocking SARS-CoV-2 virus binding and entrance in human bronchiole and alveoli. FIG. 15 shows that the phosphatidylcholine composition was modified, resulting in a different membrane composition. In addition, the targeting of VLC-PUFAs that alters lipid biosynthesis that modifies cell endosomal trafficking would also halt viral replication, as viruses require host lipid membrane to assemble virions successfully.

The therapeutic use of VLC-PUFAs, elovanoids, lipoxins, resolvins, derivatives thereof, and isomers threof can be deployed in many formats, included as an inhalable, new lung surfactant. For example, such a composition can be utilized as a prophylactic, to attenuate disease-associated inflammation, and/or disease onset and progression. Embodiments described herein can be utilized in several scenarios, such as: a) in the elderly as a morning/afternoon inhalable form as a preventive mode, or b) at disease onset in a higher concentration.

Upon adding VLC-PUFAs (FA32:6 and FA34:6) to the incubation medium of cell cultures of human bronchiole and alveoli, 27-mono-hydroxy-32:6 and 29-mono-hydroxy-34:6, stable precursors of ELV32 (FIG. 11) and ELV34 (FIG. 12) were identified. Full fragmentation of these endogenous molecules shows good matches to their theoretical peaks. The inserts show the structures along with the product ions when they are cleaved at the given bonds.

ELV32 and ELV34 were synthesized from VLC-PUFAs (FA32:6 and FA34:6) in cultures of human bronchiole and alveoli. Full fragmentation patters from the mass spectrometry of endogenous ELV32 (FIG. 13) and ELV34 (FIG. 14) show good matches to their standards. The inserts show the structures along with the product ions when they are cleaved at given bonds.

The human lung cell cultures with VLC-PUFAs (FA32:6 and FA34:6) addition in the medium show that the VLC-PUFAs have been incorporated in the phosphatidylcholine molecular species (FIG. 15). They are paired with FA18:1 or FA18:0.

### REFERENCES CITED IN THIS EXAMPLE

1. Bhattacharjee S., Jun B., Belayev L., et al. Elovanoids are a novel class of homeostatic lipid mediators that protect neural cell integrity upon injury. Sci Adv. 2017; 3(9):e1700735.doi:10.1126/sciadv.1700735
2. Do K. V., Kautzmann M-Al, Jun B., et al. Elovanoids counteract oligomeric β-amyloid-induced gene expression and protect photoreceptors. PNAS. 2019; 116 (48): 24317-24325. Doi. 10.1073/pnas.1912959116
3. Bazan N. G., Docosanoids and elovanoids from omega-3 fatty acids are pro-homeostatic modulators of inflammatory responses, cell damage and neuroprotection. Mol Aspects Med. 2018; 64: 18-33. Doi: 10.1016/j.mam.2018.09.003

Our approach is different from current research that includes structure-based drug discovery focused on proteases; on the Spike (S) protein—ACE2 interactions; antivirals; further studies on hydroxychloroquine; target identification on COVID-19 viral genome; development of vaccines; use of steroids (suppression of the immune system is a drawback) and IL-6 inhibitors to reduce lungs macrophages flow. In addition, drugs repurposing and combination therapies, including Actemra (Tocilizumab), plus an antiviral drug; anakinra, which targets the spare IL-1 soluble receptor that reduces immune responses without interfering with the beneficial action of CD4 (initiation of immune response), and CD8 T-cells (antiviral cells).

### Example 2

We can use our lipids and other identified anti-inflammatory compounds to develop therapies that will block the binding, entrance, and replication of the SARS-CoV-2 in human bronchioles and alveoli. Without wishing to be bound by theory, these therapies would disrupt membrane microdomains that, in turn, would obstruct SARS-CoV-2 virus entrance and also impair formation of endosomes. Our compounds can also curtail inflammation and reduce the cytokine storm by fostering synthesis of protective bioactive mediators and downregulating ACE2 and TMPRSS2. These therapies could be deployed in many formats, including as a new oral inhalable lung surfactant to prevent or limit virus shedding/transmissibility, and in doing so, attenuates SARS-CoV-2 disease onset and prevents progression.

Our approach is different from present research that includes structure-based drug discovery of protease inhibitors and on the Spike (S) protein-ACE2 interactions, antivirals(Remdesivir), target identification in the SARS-CoV-2 viral genome, vaccine development, stem cells (for example, allogeneic marrow-derived or allogeneic mesenchymal stem cells (remestemcel-L) administered intravenously) that would reduce immune/inflammatory system hyperactivity, steroids (immune system suppression is a drawback), studies on hydroxychloroquine and IL-6 inhibitors to reduce lung macrophages flow. Also, our approach is different from drug repurposing and combination therapies, including Actemra (Tocilizumab), plus an antiviral and anakinra that targets the spare IL-1 soluble receptor that attenuates immune responses without interfering with the beneficial actions of CD4 T-cells (initiation of immune response), and CD8 T-cells (antiviral cells).

There is growing concern that repurposing SARS drugs for SARS-CoV-2 might be fruitless because, in spite of a high sequence similarity of the main viral protease, Mpro, its active site is very different than that of the same protein in SARS 1. Moreover, it is suspected that the S1 RBD might also vary and the structural stability of the protease Mpro, with respect to flexible loop mutations, indicated that SARS-CoV-2 mutability will create additional challenges to the rational design of small-molecule inhibitors. These concerns further support the uniqueness of our approach using our newly discovered lipids, related molecules and other new anti-inflammatory compounds.

Once SARS-CoV-2 enters a host cell, it reprograms the cell to foster conditions needed for viral replication, including rewiring immune/inflammatory responses, autophagy, and lipid metabolism. The studies described herein will test that Very Long-Chain-Poly Unsaturated Fatty Acids, n-3 (VLC-PUFAs) (≥C28) and innovative synthetic anti-inflammatory compounds will allow resistance to viral infection, attenuate viral transmission, and promote disease resolution. Without wishing to be bound by theory, our molecules will: a) disrupt lipidome remodeling of membrane microdomains that recruit tetraspanins (CD-9) and allow virus-cell surface binding; b) downregulate ACE2 availability to hinder virus binding to cell surface; c) downregulate type-II serine protease TMPRSS2 availability, the protease that mediates S protein activation, post-fusion, and initial viral cell entry; d) since host lipid membrane is required to assemble virions in endosomes, our molecules will perturb endosome formation/fate, and in so doing, thwart viral replication and limit virus shedding; and e) curtail inflammation/cytokine storm by fostering the synthesis of pro-homeostatic mediators, which will also allow for b) and c) described herein.

### Example 3

Very long chain polyunsaturated fatty acids (VLC-PUFAs, n-3) delivered to the human bronchiole and alveoli, and/or nasal mucosa will:

a) Curtail inflammation/cytokine storm by fostering the synthesis of elovanoids (ELVs). ELVs would counter-regulate the immune system alteration reflected in the cytokine storm and other overactivated inflammatory responses triggered by the virus in the lung or other organs. Human bronchiole and alveoli cells are very active in phospholipid synthesis, mainly of PC with palmitic acid and oleic acid, principal components of the lung surfactant.

b) Attenuate the systemic cytokine storm by stimulating endogenous ELVs synthesis that enhances intrinsic anti-inflammatory ability. See, for example, FIG. 22, which illustrates the arrival of VLC-PUFAs (such as after oral administration or nasal inhalation) and its uptake in cells of the bronchiole/alveoli or nasal mucosa where ELVs are biosynthesized using the VLC-PUFAs as a starting point. ELVs, would become paracrine of autocrine effectors′: as paracrine mediators attenuate the cytokine storm in the lung parenchyma (or nasal mucosa) as well as systemic cytokine storm, in addition they inhibit monocyte derived macrophage formation and inhibit T cell senescence. ELVs autocrine mediators through membrane receptors downregulate the overactivation of the immune/inflammatory response (that include inflammasome formation, Interleukin 6 synthesis, etc) and of senescence-triggered inflammation.

ELVs also modulates elements of the tetraspanin membrane microdomains (TEM) essential for the interaction between virus and host that include latching the receptor binding domain of the spike glycoprotein of SARS-CoV-2 to ACE2 for cell attachment. ELVs will modulate ACE2 expression, its shedding and counteract ensuing dysfunctions of the renin-angiotensin system. Dysfunctions of this system leads to induction of damaging inflammation in the lung. ELVs in addition modulate expression of host proteases (FURIN, TMPR5S2,DPP4) necessary for cleavage of the viral protein to allow a conformational change for fusion/entrance of the virus into the cell, ELVs also regulate expression of other molecules such as CD-9 and interferons.

c) Induce lipidome remodeling and disruption of TEM (tetraspanin-enriched membrane microdomains) blocking SARS-CoV-2 virus binding and entrance in human bronchiole and alveoli.

d) Alter lipid biosynthesis that modifies cell endosomal trafficking, halt viral replication, as viruses require host lipid membrane to assemble virions successfully. Will prevent the viral-induced hijacking of the lipid metabolism.

### Example 4

Our data further demonstrated that adding very long chain polyunsaturated fatty acids (VLC-PUFAs) to human bronchiole and alveoli cells in culture activates the synthesis of elovanoids (ELVs) 32 and 34 (FIG. 24-25),In addition we demonstrate now that adding C36:6 and C38:6 pathways are activated for the synthesis of new ELVs of 36:6 and 38:6 C are formed. Evidence for this is the identification of their full MS fragmentation as well as the isolation of 31:6 and 33:6 hydroxy-stable derivatives (FIGS. 24-25) of their corresponding short lived hydroperoxides precursors.

These mediators can downregulate expression of the ACE-2 receptor (FIG. 26) and counter-regulate the cytokine storm and other inflammatory components activated by the virus in the lung. We have used primary cell cultures of human bronchiole and alveoli, a mixture of ciliated cells, club cells, type-I pneumocytes and type-II pneumocytes. The predominant cell is type II pneumocytes.

Elovanoids 32 and 34 (ELVs) downregulate the expression of the ACE-2 receptor in human alveoli in culture.

FIG. 26 shows in the top that ELVs downregulate the expression of the ACE-2 receptor protein abundance analyzed by the Jess system. There are two bands that react with the antibody for ACE-2 receptor, upper and lower, and both show the same decreases by ELVs. This effect is dependent on addition of Interleukin-1 beta (IL-1b). The reason that we decided to include this cytokine is to stress the alveoli, as it happens in COVID-19 when the cytokine storm is activated.

ACE-2 Protein abundance assessed using Jess technology.

The Simple Protein platform Jess allows running a protein mixture in a capillary system that also serves as a probing matrix for the antibodies. This platform obviates the need to transfer the size-separated proteins onto a membrane with the consequent loss of sample. The sample amount that can be run in Jess is several times smaller than that required for Western blots. Therefore, high throughput is facilitated in cell cultures. The antibodies to this protein have been validated in our laboratory.

Elovanoid 32 (ELV-32) diminishes receptor-binding domain (RBD) of the SARS-CoV-2 spike entrance in human alveoli in culture.

The lower part of the FIG. 26 shows that in the presence of Interleukin1-beta ELV-32 diminishes receptor-binding domain (RBD) of the SARS-CoV-2 spike entrance in human alveoli in culture. The reason to include the cytokine is to stress the alveoli, as it happens in COVID-19 when the cytokine storm is activated (as in the ACE-2 expression).

Preparation of receptor binding domain (RBD) of the viral spike glycoprotein.

We have prepared and characterized the RBD by complexing it with the fluorescent Alexa Fluor 488 or 594 and by defining the conditions for binding and internalization of the labeled protein in our cell cultures of bronchioles and alveoli.

Assessment of the quantitative cell surface binding and internalization of the tag protein.

We assessed cell binding and internalization of a recombinant RBD domain from the Spike S1 protein from SARS-CoV-2, labeled with Alexa Fluor. We counterstained the nucleus and membrane, and we imaged the cells in z-stacks using confocal microscopy. The images were analyzed using the IMARIS Cell module that reconstructs a cell using the nuclear membrane and the protein signal. IMARIS can define positions in the Z-axis, which portion of the protein signal is located below, within, and above the membrane level that corresponds to the protein internalized, bound to the membrane and not being taken up by the cell. The intensity sum of all three fractions was standardized by the total signal giving a proportion of the protein that was internalized. The samples are processed automatically in an unbiased manner by the Batch Imaris module. The data are then processed automatically and reported in an Excel file with coded software used especially for this project.

Protocols

The experiments described herein will use the cell cultures of human bronchiole/alveoli or of human nasal epithelium. In several experiments, we will use Very Long-Chain-Poly Unsaturated Fatty Acids, n-3 (VLC-PUFAs) (≥C28).

a) Without wishing to be bound by theory, VLE-PUFAs will produce lipidome remodeling and disrupt tetraspanins-enriched membrane microdomains (they are not lipid rafts) blocking SARS-CoV-2 virus cell binding and entrance.

Approach: We will add VLC-PUFAS to cell cultures to modify the composition of membrane phospholipids resulting in microdomain perturbation. Protein palmitoylation mutations impair the assembly of tetraspanin membrane domains. In this experiment, we will use LC-MS/MS to analyze molecular species of phospholipids. in preliminary studies, we found that the major phospholipid of the lung exhibits altered molecular species of phosphatidylcholine under the experimental conditions. We will then compare protein abundance (ACE2, CD9, TMPRSS2, DPP4) in the cell cultures with and without disruption of microdomains. At the same time, we will determine if the changes in phospholipid composition have diminished spike protein binding and internalization in the cells.

b) Without wishing to be bound by theory, ELVs will down regulate ACE2 availability and hinder cell surface virus binding.

This is based on the regulation that ELVs exert on pro- and anti-homeostatic proteins in retinal pigment epithelial cells exposed to uncompensated oxidative stress. Alveoli confronted toSARS-CoV-2 are exposed to uncompensated oxidative stress We will also test dipeptidyl-peptidase 4 downregulation that SARS-CoV and MERS-CoV use to enter cells.

c) Without wishing to be bound by theory, ELVs downregulates type-II serine protease TMPRSS2 availability, the key host protease that mediates S protein activation and initial viral cell entry.

We will also target furin, a protease also involved in SARS-CoV2 pathogenesis. When alveolar cells sense the hostile environment created by the SARS-CoV-2 (as in prediction b), ELVs will downregulate expression of type-II serine protease. Because SARS-CoV-2 activates the proinflammatory environment, acute redox reprogramming of 15-lipoxygenase may rapidly generate the pro-ferroptotic signal 15-hydroperoxy-eicosa-tetra-enoyl-phosphatidylethanolamine. The nitroxygenation of eicosatetraenoyl (ETE)-PE intermediates and oxidatively truncated species by NO. donors and/or suppression of NO. production by iNOS inhibitors may reduce/prevent initial virus cell entry and present a complementary novel redox mechanism of pro-inflammation and viral pathology. We have found that ELVs block these events and protect cell function in epithelial cells.

d) Without wishing to be bound by theory, perturbation of endosome formation hinders virus replication and limits virus shedding.

Lipid metabolism perturbations will alter endoplasmic reticulum-derived membranes, that shelters viral RNA replication and sites of virion assembly. Therefore, we will target lipid biosynthesis to modify cell endosomal trafficking to halt viral replication, as viruses require host lipid membranes for assembly of infectious virions.

Approach: We will add acetylenic PUFAs, as well as structural analogs of fatty acids, to generate transient disturbances in the endosomal system. We will verify the biosynthesized molecular species of phospholipids by LC-MS/MS. We will also incubate cultured cells with viral mock particles from the entire SARS-CoV-2 recombinant protein. Without wishing to be bound by theory, this will block/slow down viral entry and replication.

e) Without wishing to be bound by theory, elovanoids and related compounds will curtail inflammation and prevent cytokine storm by fostering the synthesis of pro-homeostatic mediators. In turn, these events will also allow for b) and c) described herein.

Without wishing to be bound by theory, the damaging inflammatory response to the SARS-CoV-2 by the immune system reflected in the cytokine storm (in some patients) can be contained by activating pro-homeostatic pathways of ELVs synthesis. We discovered and characterized ELVs in 2017 and uncovered their potent pro-homeostatic properties.

Approach A: VLC-PUFAs, n-3 (1 μM, VLC-PUFAs) will be added to the cell cultures. Our data demonstrates that human bronchiole cells/alveolar cells incubated with VLC-PUFAs activates ELVs synthesis. FIGS. 27-28 depict fragmentation patterns of ELVs and of their stable precursors. This has not been seen before in these cells. Human bronchiole/alveolar cells actively synthesize phospholipids, mainly phosphatidylcholines containing palmitic acid and oleic acid, main components of the lung surfactant. Known lung lipids are from a different family of mediators (eicosanoids, from C20 arachidonic acid, n-6). Mediators derived from docosahexaenoic acid (n-3) have been identified that we call docosanoids (C-22), including Neuroprotectin D1 and other mediators. Without wishing to be bound by theory, inflammatory resolution mediators are key players in preventing/attenuating the cytokine storm. Human bronchioles/alveoli are targeted by SASP toxic actions induced by early virus exposure altering homeostasis and as a consequence, create an inflammatory milieu that facilitates virus entrance and propagation.

Approach B: Because we showed recently that ELVs counteracted amyloid f3 peptide-mediated senescence reprogramming and inflammaging in human retinal pigment epithelial cells, we will test the ability of ELVs to counteract inflammatory/immune/cytokine storm responses to SARS-CoV-2 to protect the integrity of human bronchioles/alveola cells. Thus, we will define ELVs down regulating: a) a senescence program reflected by enhanced gene expression of Cdkn2a, Mmpla, Trp53, Cdkn1a, (3Cdkn1b, Il-6, and SASP secretome. We have characterized these targets, and, which out wishing to be bound by theory, ELVs will blunt these events and protect the cells. b) P16INK4a protein abundance (as well as from other senescence regulatory proteins) will also be followed. Senescence events are present in the alveoli. Furthermore, we will validate whether ELVs restore expression of ECM remodeling matrix metalloproteinases which could uncover an additional disturbance in the lung intercellular matrix. The viral-mediated inflammation may be a low-grade, sterile, chronic proinflammatory inflammation linked to senescence of the immune system.

Screening of drugs.

The compounds belong to three classes of molecules that target inflammatory/immune responses and also modulate transcription/translation of key proteins necessary to sustain epithelial cell integrity when confronted with uncompensated oxidative stress. They are platelet-activating factor (PAF) synthetic antagonists, 5-lipoxygenase inhibitors, LC-PUFAs, n-3, synthetic elovanoids and other select lipid mediators.

### CONCLUSION

We will learn how to allow for prevention, viral infection resistance, disease attenuation/transmissibility, and remission of SARS-CoV-2 at the cellular level. This will facilitate our move to humans. These therapies can be deployed in many formats, including as an oral inhalable to prevent or limit virus shedding/transmissibility, and in doing so, attenuate COVID-19 disease onset and prevents progression.

### Example 5

Without wishing to be bound by theory, embodiments herein will meet the urgent demand for effective countermeasures against SARS-CoV-2 infectivity by defining the newly discovered elovanoids to attenuate viral cell entry and downregulate inflammation and cytokine storm. We will focus on the human alveoli and nasal mucosa. Embodiments herein will contribute new preventative and therapeutic avenues for COVID-19 onset and progression.

### Example 6

Research Strategy

A. SIGNIFICANCE. Coronavirus disease 2019 (COVID-19), caused by Severe Acute Respiratory Syndrome—coronavirus 2 (SARS-CoV-2), is highly transmissible from human to human and has spread rapidly across the globe. The first step of this virus's life cycle is to infect primarily type II alveolar cells (which explains severe lung damage), nasal cells, eye surface, gastrointestinal tract, and nervous system. Infection triggers a wide range of disease phenotypes. Therefore, there is a dire and immediate need for effective therapeutics.

SARS-CoV-2 spike glycoprotein (S Protein). The S protein is multifunctional and binds cell receptors and catalyzes fusion with cells followed by endocytosis of virions, allowing the virus genome to enter the cell. Trimers on the 51 domain of S protein contains the receptor-binding domain (RBD; aa 319 to 541) for the angiotensin-converting enzyme 2 (ACE2)′ and arginine-glycine-aspartic acid or “RGD” (Arg-Gly-Asp) motif. The RGD motif is the minimal peptide sequence required for binding integrins, which are receptors used by many human viruses 3. The conservation of the RGD motif and its localization in the RBD of the SARS-CoV-2 indicates that integrins can be alternative/complementary virus receptors.

The S protein is processed at the S2 site by a furin-like proprotein convertase type II serine protease and also by transmembrane serine protease 2 (TMPRSS2), and dipeptidyl peptidase 4 (DPP4) that mediate S protein activation and viral entry. A recent publication showed that there is a human-specific interferon-driven upregulation of ACE2 in airway epithelial cells and nasal cells 4. There is an apparent duality in the role of ACE2; it is a SARS-CoV-2 receptor, but it also protects from severe lung damage as a negative regulator of the proinflammatory renin-angiotensin system (RAS)⁵⁻⁷. ANG II, via activation of the AT1R, triggers vasoconstriction, reactive oxygen species formation, inflammation, and extracellular matrix remodeling⁵. ACE2 counter regulates damage induced by ANG II and AT1R via activation of AT2R⁵. Without wishing to be bound by theory, when ACE2 is taken over by the virus, an ANGII pro-inflammatory cascade is activated⁵.

Inflammatory and ‘cytokine storm’ in COVID-19. Dysregulated lung immune/inflammatory responses lead to pneumonia with ARDS caused by SARS-CoV-2. A subset of COVID-19 patients develops a cytokine storm, characterized by pro-inflammatory cytokines and alveoli infiltration of monocytes/macrophages. Here, we will validate the inflammatory response triggered by SARS-CoV-2 as cellular and molecular signaling events that occur initially to offset ensuing damage, but that often becomes an exaggerated reaction that can lead to injury of lung or other organs. We will study inflammaging, a low-grade, sterile, proinflammatory condition linked to senescence of the immune system^{8,9}. Without wishing to be bound by theory, the virus can activate senescence and inflammaging in the infected lung or nasal cells. Thus, altered homeostasis of the intercellular matrix creates an inflammatory milieu that contributes to ARDS and further virus susceptibility. In FIG. 40, we depict how ELVs can counter autocrine and paracrine inflammatory/immune dysregulation. ELVs restore expression of matrix metalloproteinases for extracellular matrix sustainment¹⁰ where integrins are located.

Senescence Associated Secretory Phenotype (SASP). SASP is a pro-inflammatory secretome that includes chemokines, metalloproteinases, proteases, cytokines (e.g., TNF-α, IL-6, and IL-8), and insulin-like growth factor binding proteins (vary in different tissues). The senescence genes validated here are p 16INK4a (Cdkn2a), p21CIP1 (Cdkn1A), p27KIP (Cdkn1B), p53 (Tp53 or TRP53), IL6, and MMP 1. We have recently found that ELVs reversed an injury-induced senescence program in human retinal epithelial cells¹⁰. Thus, without wishing to be bound by theory, ELVs can also coordinate a concerted inflammatory resolution in alveoli and nasal cells in response to SARS-CoV-2.

Very long chain polyunsaturated fatty acids,n-3 (VLC-PUFAs). ELOVL4 (elongation of very long chain fatty acids-4), catalyzes the biosynthesis of VLC-PUFAs (≥C28) from 26:6 fatty acids from DHA or eicosapentaenoic acid (EPA)^{11,12} VLC-PUFAs are then incorporated in phosphatidylcholine molecular species in retina and brain¹³. We have shown that adding these fatty acids to human alveolar cells in culture fosters the formation of atypical lung phospholipids (FIG. 40, Aim 2). On the other hand, lipoxygenation of VLC-PUFA leads to di-hydroxylated derivatives termed elovanoids (ELVs), ELV32 and ELV34 (FIG. 41). Because of the pro-homeostatic anti-inflammatory bioactivity of ELV mediators, they can down-regulate cytokine storm/inflammatory components activated by SARS-CoV-2 in the lung and likely, also in other organs.

Without wishing to be bound by theory, ELVs and VLCPUFAs can be harnessed as countermeasures against SARSCoV-2 attachment, entrance, endosome formation, inflammation, and cytokine storm (FIG. 40).

Our studies are a paradigm shift to understanding principles and molecular mechanisms of SARS-CoV-2 in humans, by focusing on fundamental processes underlying ELVs counteracting SARS-CoV-2 critical cell entrance. Our approach is different from current research that focuses primarily on structure-based drug discovery of protease inhibitors as well as S protein/ACE2 interactions, antivirals such as Remdesivir, target identification in the SARSCoV-2 viral genome, vaccine development, stem cells (e.g., allogeneic marrow-derived or allogeneic mesenchymal stem cells (remestemcel-L) that would reduce immune/inflammatory hyperactivity, convalescent plasma, studies on hydroxychloroquine and IL-6 inhibitors to reduce migration of macrophages to the lung, alpha interferon (IFN-α), lopinavir/ritonavir, ribavirin, chloroquine phosphate, and Arbidol. Our approach is also different from drug repurposing and combination therapies, including Actemra (Tocilizumab), plus an antiviral and anakinra that targets the spare IL-1 receptor that attenuates immune responses without interfering with the beneficial actions of CD4 T cells (initiation of the immune response), and CD8 T cells (antiviral cells). There is growing concern that repurposing SARS drugs for SARS-CoV-2 might be fruitless because, despite high sequence similarity of the viral protease Mpro, its active site is different than that of the SARS viral protease¹⁴. Moreover, it is suspected that the S1 RBD might also vary, and the structural stability of the protease Mpro, concerning flexible loop mutations, indicated that SARS-CoV-2 mutability would create additional challenges to the rational design of small-molecule inhibitors¹⁴. These concerns further support the uniqueness of our approach using our newly discovered lipids.

B. INNOVATION. This proposal incorporates new chemically synthesized ELVs, as well as methodological innovations, to define molecular principles to block SARS-CoV-2 cell binding and entry into alveoli and nasal mucosa. Also, it will bring the complex network of inflammation-resolving responses, including new pathways, to get a better understanding of lung protection mechanisms. Aspects of the invention are drawn to innovations comprising:

1) Disruption of lipidome remodeling of membrane microdomains that recruit tetraspanins can be harnessed to attenuate virus-cell surface binding.

2) Since host lipid membrane is required to assemble virions in endosomes, VLCPUFAsperturb endosome formation by triggering the biosynthesis of atypical lung phospholipids.

3) ELVs downregulate ACE2 expression and availability to hinder viral attachment.

4) ELVs downregulate the expression and availability of furin, type II serine protease TMPRSS2, and DPP4 proteases that mediate S protein activation, post-fusion, and initial viral cell entry

5) ELVs restore expression of matrix metalloproteinases for extracellular matrix sustainment, where integrins are located, to counteract viral infectivity

6) Upon virus infection, ELVs counteract senescence gene programming, SASP secretome release, and inflammaging.

C. Approach

Rigor and reproducibility: This project has been designed to ensure scientific rigor and the reproducibility of the results. The following is a listing of specific elements incorporated into this application that are consistent with published NIH guidelines: Data to support each of the three Specific Aims were analyzed for significance; experiments will use the same supplier of antibodies for experiments that were used in studies to generate the preliminary data and if other experiments were proposed the supplier and catalog number was noted (these antibodies were used in other projects by our lab); cell cultures will be randomly allocated to experimental groups and treatments; data acquisition and analysis, will be performed in a blinded manner; inclusion of multiple quantifiable endpoints relevant to each aspect of the project with previous determinations of needed sample sizes. Images will be taken (the same manner as performed with the preliminary data) automatically at random using the software FV31S-SW and using a ZDC module that allows the z-stacks to be taken on focus.

Statistical methods, sample sizes, and power analysis: Repeated measures analysis of variance (ANOVA), followed by Bonferroni procedures to correct for multiple comparisons, will be used for intergroup comparisons. Post-hoc comparisons between means will be conducted using t-tests with alpha level adjustment by a method of simulation based on the number of planned comparisons. Differences are considered significant at an alpha level of Power analyses of data indicate that, based on the differences and data variance, 12 cell culture wells/group will be required to achieve a power of 0.80 in experiments involving 73 or more pictures per condition data as outcomes. For the power analysis, using a pre-set alpha level of 0.05 and an expected power of 80%, effects as small as 50 cells/mm2 will be detectable using % of the signal found below the membrane as the outcome. Our extensive experience with analyses of cell culture studies indicates that all of these outcome variables can be dealt with under the assumption of asymptotic normality where sample sizes are adequate. For the case of small sample size and non-normality, related nonparametric methods will be used for analysis.

Outcome measures: Analyses will be targeted at defining restorative lipid ELVs and their biosynthesis.

1. Overall Experimental Design and Methods. To avoid repetition, below, we present the approaches to be used for experiments in the Aims (all these techniques are well established in our laboratories). In several experiments, we will use plasma membrane extracts of human kidney cells (HEK293T) overexpressing S protein fused to mCherry. The reason for the use of these cells is that the S protein embedded in the plasma membrane preserves the trimer configuration and thus yield more efficient ACE2 binding.

c.1.a. Primary cultures of human alveoli and human nasal cells. We will not use transformed lung cells because these cells do not respond to SARS-CoV-2 entrance the same way as primary lung cells¹⁵. Therefore, we have developed primary cultures of human alveoli (a mixture of type II pneumocytes, ciliated cells, club cells, and type I pneumocytes). These cells have been used to study the COVID-19 virus¹⁶, through PromoCell (HSAEpC). The predominant cell type in HSAEpC is type II pneumocytes. The proliferating cells arrive in P2 and go for >15 population doubling. We have characterized histology/immunocytochemistry of these primary cells (FIG. 42-44).

c.1.b. Protein abundance using Jess technology. The Simple Protein platform Jess allows running a protein mixture in a capillary electrophoretic system as a probing matrix for the antibodies (we refer to western blot as “Jess WB” here). This platform obviates the need to transfer the size-separated proteins onto a membrane with a potential loss of sample. The sample size run in Jess is several times smaller than that for classical WB. (400 ng protein/capillary column vs. 10 or more μg for classical WB). The proteins we assess will include ACE2, Furin, ADAM17, DPP4, TMPRSS2, protein surfactant D, and Cathepsin L. In addition, senescence and SASP proteins will also be analyzed. Antibodies to these proteins have been validated in our laboratory.

c.1.c. Droplet digital PCR (dd-PCR) for gene expression. This approach will be used to validate the expression of the genes encoding the proteins listed above. This technique can determine the absolute number of copies of a gene in a small sample (5 ng of total RNA) with errors of around 2% across wells, thus facilitating the high-throughput screening to evaluate gene expression.

c.1.d. Preparation and characterization of the RBD of the viral S glycoprotein. We have prepared and characterized the RBD of the S glycoprotein by complexing it with the fluorescent label Alexa-Fluor 546. Briefly, the RBD portion (25KDa) of the S protein was chemically labeled with Alexa-Fluor 546 using a commercial kit (Thermo Fisher cat #A10237), then we performed the purity, integrity, and efficiency controls of labeling via Nano drop, BCA, and WB assay (done once for each batch). Next, we defined the conditions for binding and internalization of the labeled protein in the primary culture of human type II pneumocytes by determining the amount of labeled protein required for visualization of internalization. For data, we used 500 ng of protein/well of a 48 well plate (FIG. 7,8,10).

c.1.e. Recombinant entire S protein from SARS-CoV-2 to produce mock viral particles. We have subcloned an ORF for the entire S protein into a carrier vector containing the mCHERRY tag. We acquired: a) SARS pcDNA 3.1(+)-P2A-eGFP expression plasmid containing the sequence P2A-eGFP (molecularcloud.org/plasmid/SARSpcDNA3.1-P2A-eGFP/MC-0101088.html); b) pUC57-2019-nCoV-S protein ORF (in Insect expression vector backbone) in the form of lyophilized plasmid (catalog ID SC1317: MC_0101084); c) pUC57-2019-nCoV-N nucleocapsid protein in the form of lyophilized plasmid (insect vector backbone) SC1317:MC_0101085); and d) pUC57-2019-nCoV-S(Original) in the form of lyophilized plasmid (SC1317: MC_01010800. This gives us access to the ORF of proteins S and N (nucleocapsid; an internal protein that packages the viral RNA in the virions). The ORFs have been subcloned in the mCherry tagged vector that will express the S or N protein fused to mCherry fluorescent protein at the N terminal, and a His tag will be incorporated at the end of the mCherry tag to facilitate the protein purification using a Co/Ni column. The N protein will be used as a negative control since it is not involved in the attachment of the viral particle. To reduce costs, the recombinant protein consisting of the whole SARS-CoV-2 protein will be produced in our laboratory and used in several proposed experiments, including to produce mock viral particles.

c.1.f. Quantification of cell surface binding and internalization of tag RBD of S protein or recombinant entire S protein from SARS-CoV-2. In studies, we assessed the binding and internalization of recombinant RBD from the S protein S1 labeled with Alexa Fluor (FIG. 46,47). We will also use the entire S protein and mock virus since certain drugs interact with the C-terminus of S1 in the RBD of the protein¹⁷. Likewise, we will follow the mCherry-tagged S protein containing S1 and S2 domains. We will counterstain the nucleus and membrane, image in z-stacks by confocal microscopy, and analyze images by IMARIS Cell module that reconstructs a cell using the nuclear membrane and the protein signal. IMARIS helps define the position in the Z-axis and which portion of the protein signal is located below, within, and above the membrane level corresponding to the protein that is internalized, bound to the membrane, and not taken up by the cell, respectively (FIG. 46,48). The intensity sum of all three fractions was standardized by the total signal providing the internalized protein proportion. The samples will be automatically processed in an unbiased manner by the Batch Imaris module, using a code written specifically for this task and reported in an Excel file.

Aim 1) ELVs downregulate availability of [a] ACE2 (and thus would hinder cell surface virus binding) and [b] key host proteases (that mediate S protein activation and viral cell entry).

Our data demonstrate that human alveolar cells display an endogenous synthesis of the prohomeostatic mediators ELVs when VLC-PUFAs are added (FIG. 41, 45) and that the addition of ELVs attenuated RBD cell entrance into the cells (FIG. 46) and ACE2 abundance (FIG. 47). ELV-induced RBD cell entrance is specific, as shown using the viral nucleocapsid N protein tagged with Alexa Fluor-594 (FIG. 49). Thus, we will define the mechanisms by which ELVs function during conditions that recapitulate virus infection and entry: proteolytic activation of the S protein for cell entry by assessing the host proteases TMPRSS2, Furin, and DPP4 that mediate protein activation and viral cell entry.

Rationale: The identification of compounds and their molecular mechanism(s) that attenuate/block SARS-CoV-2 cell entrance to limit onset and progressive lung (and other organs) damage is becoming increasingly relevant. Validating the importance of targeting SARS-CoV-2 cell entrance is the recent demonstration that the SARS-CoV-2 mutation D614 increases virus infectivity by 4-5 times due to an enhanced number of RBD sites¹⁸. Very recently, a newly recognized mutation G614 has been identified as the dominant pandemic variant (cov.lanl.gov) and to display an increased number of RBDs. The G614 mutation has rapidly increased worldwide in the last few months; this mutation confers increased infectivity and is associated with higher viral load¹⁹. We will work with the S protein containing G in position 614 (FIG. 48). Moreover, cryo-EM of the S protein RBD from SARS-CoV-2 was 10 to 20 times more likely to bind ACE2 on human cells than S from the SARS virus. Hence, it would enable SARS-CoV-2 to spread more readily from person to person. These findings underscore the importance of identifying attenuators/blockers of the RBD interaction with ACE2. We will also study the significance of RGD binding motifs in the RBD for specific integrins and of LDI motifs in the S protein²⁰. These motifs are important in SARS-CoV-2 cell recognition and infection and have a high affinity to integrins type αVβn. We identified these motifs and cloned these sites in the S protein (FIG. 48) and will use them to define integrins function in viral infectivity. We will determine whether integrins modify ELVs preventing S protein-ACE2 interaction. Central questions are how ELVs downregulate the availability of ACE2 and if ELVs downregulate proteases that mediate S protein activation and viral cell entry (FIG. 40). Also, we will investigate if ELVs impair endosome formation through decreasing or inactivating Cathepsin L, a protease involved in the intracellular processing of the viral particles. Moreover, we will study the effect of IFNα since type I IFNα-driven upregulation of ACE2 occurs in human airway epithelial and nasal cells⁴.

Design: We will define the timing for binding and internalization of RBD (complexed with Alexa 546) and of the recombinant entire S protein (mCherry-tagged) in cells (passage 4-incubated 1, 2, 4, 8, 16, and 32 h after treatment with either IL1-β/TNF-α or IFNγ or IFNα (100 ng/ml). Our data were obtained by incubating cells with the RBD for 24 h (FIG. 46,47). We will test different ELV structures to identify which is the most effective. For example, ELV acetylenic with triple bonds introduced between C25/C25 of ELVs is stable longer than the sodium salt and elicits sustained action based on the notion that the triple-bonded ELVs degrade slower than the naturally occurring molecule. ELVs 32 and 34, including R/R and S/S isomers will be tested (we have several additional structural analogs available). Once we have identified the most potent ELVs, we will analyze proteins by JESS WB to determine if ELVs would modify the availability of ACE2 and/or host proteases. Also, by dd-PCR, we will explore if ELVs modify ACE2 and host protease gene expression. We ask the below questions since we found that, when other epithelial cells are confronted with injury-oxidative stress, ELVs selectively modulate gene programming. In fact, they enhance expression (and abundance) of homeostatic, pro-cell survival proteins (sirtuin, Iduna/RNF146-PAR-binding dependent PARsylation-directed E3 ubiquitin ligase, cytoprotective prohibitin, and anti-apoptotic BCL-2 proteins) and selectively reduce the expression of cell-damaging, pro-inflammatory proteins (Bax-Bim, Bid)¹¹.

We will follow the internalization of the S protein approach, similar to the one shown in preliminary results (FIG. 46,47), except the entire recombinant S protein will be used. In FIG. 46,47, the RBD conjugated with fluorescent dyes was the mean used to trace the protein inside the cell. In these studies, we will take advantage of the complete S protein fused to mCherry tag to track its internalization and to define its cleavage in the alveolar cell surface interaction to delineate how ELVs might block. We will use nucleocapsid protein (N protein) from the original SARS-CoV-2 as the control (FIG. 49). We also use a cloned and mCherry-labeled ORF for N protein as a viral protein that is not in contact with ACE2 or other surface proteins and as a negative control to define if the effect(s) we observe with S protein are specific (FIG. 49). We will use primary cultures of human alveolar and nasal epithelial cells for all of these experiments:

1) We will test different ELV structures to select analog by analyzing proteins by JESS WB to determine the most effective ELVs that modify the availability of ACE2 and/or host proteases. Then cells will be exposed to the S protein fused to mCherry to tag the S2 portion that after cleavage would allow viral cell entrance. We will validate: a) binding and internalization of S purified protein (stand-alone protein) and b) plasma membrane extracts of human kidney cells (HEK293T) overexpressing S fused to mCherry. We will follow the fate of the protein over time by Jess WB of subcellular fractions and by immunocytochemistry using endosomal and lysosomal markers. We will perform mutagenesis on the S/mCherry original ORF (FIG. 48) to delete the RGD and LDI motifs and also to change the RGD to TGD (threonine-glycine-aspartic acid), TGE (threonine-glycine-glutamic acid), TAD (threonine-alanineaspartic acid), TAE (threonine-alanine-glutamic acid and RGS (arginine-glycine-serine)—changes that have been shown to reduce the in vitro infectivity of other viruses²¹.

2) In the S protein, the LDI motif is located downstream outside the ACE2 binding site in the RBD domain, but close to the cleavage site RRAR/VAS (FIG. 48). Does LDI influence cleavage of S into S1 and S2 and, by doing so, modify the efficiency of viral entry? To determine if cleavage occurs, we will use JESS WB to assess S protein cleavage upon interaction with ACE2 using antibodies against mCherry (fused to the Cterminal of S protein) and against S protein. Also, we will follow protein internalization after disrupting the LDI motif by replacing it with ADI (alanine-aspartic acid-Isoleucine), ADV (alanine-aspartic acid-isoleucine), LEI (leucine-glutamic acid-Isoleucine), LKI (leucine-lysine-Isoleucine) and AKV (alanine-lysine-valine).

3) Viruses with proteins containing RGD motifs infect cells via interaction with integrins such as αVβn³. We will use two approaches to validate binding and internalization of S protein when the RGD/integrins interaction is impaired: a) We will use neutralizing antibodies against integrins avb3 (LM609), avb5 (P1F6), a5 (P1D6), b1 (P4C10) and b2 (P4H₉-A11) from Merck Millipore, integrin a4 (HP2/1) from Immunotech and a6 (GoH3) from R&D Systems to block their interaction with S protein. b) We will also use LXW64, a cyclic octapeptide used to mimic RGD and deliver drugs into cells expressing αvβ3 integrin²². In this case, we will use the octapeptide to compete for the integrin with the RGD motif present in S and displace the binding, will follow internalization of S protein and determine the cleavage into S1 and S2 via Jess WB.

4) To determine if ELVs prevent and/or reduce S protein internalization, we will modify the proteins pool required for infectivity by treating cells with ELVs and exposing them to wild type or mutated (from Exp.1-2) recombinant S/mCherry protein. We will assess S protein internalization (FIG. 46, 47) and cleavage using Jess WB targeting mCherry tag. We will evaluate protein expression related to the recognition and incorporation of the S protein into the cell using Jess WB and dd-PCR at different time points (0, 6, 12, 18, 24 and 36 h) after treatment and will assess candidate genes and proteins: ACE2, Integrins (recognition) TMPRSS2, Furin (cleavage) and Cathepsin L (lysosomal processing). We will follow S protein in the cells by immunostaining using markers of early and late endosomal and lysosomal processing.

Conclusions: Without wishing to be bound by theory, the RGD conservative replacement of amino acids (Exp.1) will interrupt or decrease the protein binding to ACE2, and hence internalization of S protein will be reduced. Also, the replacement of the amino acids in the LDI motif will halt or decrease the cleavage of S into S1 and S2, and that will be reflected in the Jess WB. The recombinant S protein (original S plus mCherry tag) is 1509 amino acids and runs between the 160 to 180 KDa markers. The cleaved protein at the Furin cleavage site is S1=685 and S2 plus mCherry tag=824 amino acids. For the TMPRSS2 cleavage occurring between amino acid 808 and 820²³, we will see a small fragment of 689 aa encompassing the rest of the S2 plus the mCherry tag. Using an antibody against mCherry, we will observe a band of 160 to 180 KDa that is the uncleaved S protein and a band of 90 KDa corresponding to cleaved S2/mCherry or both bands if the cleavage is partial. So, the LDI interruption will retrieve a mix of the two bands or the higher band alone, indicating that the processing is totally abrogated. Under these conditions, internalization will also be impaired. Further, the cyclic octapeptide will block the interaction of RGD with integrins and prevent the binding of ACE2. By neutralizing each candidate with the corresponding antibody, we will determine which integrin is responsible for the interaction with ACE2 and Furin and/or TMPRSS2. By blocking the integrins, we will observesimilar results for Experiments 1 and 2, confirming that the integrins have a role in the binding, processing, and internalization of the S protein in SARS-CoV-2 infection. Based on our results (FIG. 46, 47), ELVs will reduce the internalization of the S protein. Also, ELVs will modulate the endosomal/lysosomal processing of the S protein and that this modulation will be reflected in immunostaining at different time points.

The techniques proposed have been used to generate our preliminary results, and they are routinely used in our laboratory. We cloned the S ORF in pEF1a-mCherry vector (TAKARA) that synthesizes the fusion protein S-mCherry, and after purification, the protein can be followed in the cells by confocal microscopy after counterstaining nuclei and membrane, as shown in FIG. 46, 47 or detected by JessWB of a cell lysate of human alveolar cells. We will use conventional directed mutagenesis techniques24. Alternatively, we will design the desired mutations in silico and have the sequence to produce the ORF synthesized commercially. If purified S protein does not interact with the cell-surface ACE2 receptor in the same way as SARS-CoV-2 because the context is different or if we detect a deficient internalization and/or cleavage of the protein, we will produce S proteins embedded in plasma membrane particles. We will obtain these particles by expressing the protein in HEK293T cells. Since the protein contains a signal peptide from amino acid 1 to 11, the protein will be inserted in the membrane of the host cell, and the purified plasma membrane will provide a structure similar to that of the viral particle.

Aim 2) VLC-PUFAs induce lipidome remodeling and disrupt tetraspanin-enriched membrane microdomains (that contribute to blocking virus-cell binding and entrance) and perturb endosome formation to hinder virus replication.

Rationale: Alveolar cells are very active in lipid metabolism, particularly in the synthesis of phosphatidylcholine as a component of lung surfactant. Unexpectedly the addition of VLC-PUFAs to cultured human alveolar cells leads to the synthesis of atypical phosphatidylcholines (FIG. 50). Therefore, we will determine if these atypical phospholipids perturb membrane function of alveolar cells and or nasal mucosa cells. Viral infectivity, at least in part, cluster transmembrane proteins (tetraspanins) in specific cell membrane domains²⁵. These microdomains are not lipid rafts since they luck glycosyl-phosphatidylinositol-linked proteins, caveolin, and Src-kinases²⁶. Thus, lipidome remodeling would disrupt tetraspanin-enriched membrane microdomains and thereby contribute to block viral attachment and entry. Moreover, phospholipid biosynthesis in the endoplasmic reticulum for endosome formation and virus trafficking would be perturbed due as well by the atypical phosphatidylcholines that would modify endosomal trafficking and arrest viral replication, since viruses require host lipid membranes for virion assembly (FIG. 40).

Design: We will determine if the content of phospholipids containing VLC-PUFAs correlates with decreased internalization and processing of S protein. Thus, we will treat alveolar or nasal cells with different concentrations of VLC-PUFA, isolate plasma membrane, microsomes and endosomes as a function of time +/−S protein. By LC-MS/MS, we will identify the fate of VLC-PUFA into phospholipids in each fraction. Phosphatidylcholines are the main lipids that take the VLC-PUFAs in the entire alveolar cells in culture (FIG. 50). At the same time, we will correlate with the abundance of internalized ACE2 and S protein. We will also analyze the lipids and ACE2 of endosomes, lysosomes, endoplasmic reticulum, and Golgi vesicles by immunocytochemistry and confocal microscopy along with the analysis of S protein cleavage by Jess WB. To assess different organelles and endosomes, the following markers will be used: Anti-LAMP2 (Abcam #ab25631, lysosomes), Rab4 (Abcam #ab109009 early sorted endosomes), CD63 (Abacam #ab1318, late endosomes/lysosomes) CD98 (Abcam #ab2528, plasma membrane), Cytochrome C oxydase (Abcam #ab198593, mitochondria), EEA1 (Abcam #ab2900, early endosomes), Anti-58K (Abcam #ab2704, Golgi), Calreticulin (Abacam #ab22683ER) and Anti-Nuclear Membrane Marker (Abcam #ab190725). We will use primary cultures of human alveolar and nasal epithelial cells for all of these experiments.

1. To follow deuterated (d6)-VLC-PUFAs fate. We will incubate cells for up to 32 h with VLC-PUFAs containing d6-32:6n-3, d6-34:6n-3 or together, at final concentrations of 2-10 μM. Then we will isolate plasma membrane, microsomes, and endosomes, extract and purify lipids to follow the fate of VLC-PUFA into phospholipids in each fraction by LC-MS/MS. Phosphatidylcholines are the main lipids that take the VLC-PUFAs in the entire alveolar cells in culture (FIG. 50). The outcome will be defining optimal conditions in so far as the concentration of the VLC-PUFAs and the time of incubation.

2. To validate the subcellular compartment modified by VLC-PUFAs that decreases internalized ACE2. Cells will be incubated with VLC-PUFAs (at optimal conditions defined in Exp.1) and then exposed to RBD/Alexa Fluor or the entire S protein/mCherry or plasma membranes from 293T expressing S protein. Then endosomes will be sorted following mCherry and Alexa via Fluorescence-activated organelle sorting²⁷. The endosomes containing the S protein will be subjected to lipidomic analysis by LC/MS-MS (to define the distribution of d6-VLC-PUFAs in lipid classes of endosomal membranes) and to determine the fate of S protein, ACE2, and proteases via Jess WB. In addition, similar measurements will be performed after treatment with either ILβ/TNF-α or IFNγ or IFNα (100 ng/ml). We will also analyze endosomes, lysosomes, endoplasmic reticulum and Golgi vesicles by immunocytochemistry and confocal microscopy along with S protein cleavage evaluation by Jess WB. We will carefully explore the plasma membrane fraction for enrichment in tetraspanin microdomains 28. N protein tagged with Alexa-Fluor or mCherry will be negative control (FIG. 49).

3. To validate whether the processing of the S protein is slowed down. A time course and dose-response will be performed to assess the processing of the S protein, following optimal conditions defined in Exp. 1. Endosomes and lysosomes will be assayed by immunocytochemistry to identify clathrin-coated, early or late endosome vesicles and lysosomes; ER and Golgi loaded with S protein will be traced by the mCherry tag. Dynasore (Tocris, Cat. No. 2897), is a non-competitive inhibitor of Dynamin and, thus, clathrin-mediated endocytosis²⁹, will be used to determine whether clathrin is involved in endocytosis of the S protein. Using antibodies against markers of different stages of the endosome (anti-S protein (targeting the S1 portion; Abcam ab273073) and anti-mCherry (Abcam ab213511), we will trace the fate and intracellular processing of the S1 and S2 parts of the protein,Images obtained by confocal microscopy Z-stacks and will be analyzed for signal position and intensity by Imaris software (FIG. 47,50).

Conclusions: Without wishing to be bound by theory, we will identify and follow the metabolic flow of d-6 VLCPUFAs in phospholipids of specific cellular compartments and define if they perturb the internalization of ACE2 and the S protein. Without wishing to be bound by theory, we will observe that 1) the phospholipids changes will follow a different time course in the plasma membrane (early) and endosomes(later) by the addition VLC-PUFAs and that, the early changes (plasma membrane) would interfere with ACE2/S1-RBD interaction and with ACE2 internalization. 2) They will impair or decrease interactions Furin/S2-S1 and/or TMPRSS2/S2-S1 attenuating S protein cleavage, and 3) perturbed endocytosis and subsequent S protein processing. We also will validate that it is possible to downregulate the endosomal load of S protein and its cleavage by activating the synthesis of atypical phospholipids. Thus, the increased content of atypical lung phospholipids containing VLC-PUFA will delay or decrease (or both) the binding and cleavage of S and alter the presence of the S protein in the endosomes. Also, VLC-PUFA will alter in quality as well as quantity endosomes and lysosomes carrying S protein fragments. The tracing using d-6 VLC-PUFA will uncover the timing of the flow and incorporation of the VLC-PUFA into phospholipids (within the timing of internalization of the S protein). Altogether, the addition of VLC-PUFA will delay and prevent the entrance and processing of the S protein. Nasal cells will behave similarly as the alveolar cells.

We will apply the sorting strategy for isolation of endosomes taking advantage of the fluorescent tag of the RBD or S protein. We have experience in cell sorting. As an alternative, we will use sucrose density gradient-based ultracentrifugation^{30,31} or immune-isolation of endosomes. For the latter, we will use antibodies against clathrin or AP2 (for early endosomes), Rab5 (early naked endosomes), Rab7 (late endosomes/lysosomes), and Rab4 (for sorted endosomes) coupled to magnetic beads^{32,33} to obtain the quantities required for analysis. Since the addition of VLC-PUFA decreases RBD internalization (FIG. 47A), we foresee a similar outcome with the entire S protein. As an alternative, we will produce an S protein ORF with the site RRAR/SVAS mutated (negative control for TMPRSS2 cleavage) and compare the outcomes. Alternatively, we will use an inhibitor of TMPRSS2 (camostat mesylate, Sigma Aldrich) as a negative control. We will use the same approach to block Furin (Hexa-D-arginine and SSM 3 trifluoroacetate, inhibitor of furin, Tocris Biosciences).

Aim 3) ELVs curtail inflammation and prevent cytokine storm in cultured human alveolar and nasal mucosa cells upon RBD binding or S protein entrance, such as occurs under SARS-CoV-2 attack.

Rationale: Without wishing to be bound by theory, VLC-PUFA curb inflammation/cytokine storm/cell damage in the alveoli and nasal mucosa as ELVs synthesis precursors (FIG. 40,51). We discovered ELVs in 2017^{11,34} and described that their bioactivity includes: a) pro-homeostatic regulation^{11,12,34} b)modulation of senescence gene programming, including SASP secretome release, c) attenuation of inflammaging¹⁰, and d) targeting of key protective events in the extracellular matrix between photoreceptors and the retinal pigment epithelial cells¹⁰.

Senescence gene programming is key in aging^{8,9}, lung diseases^{1,3,5,36}, and in COVID-19 pneumonia^{37,38}. In this aim, we focus on ELVs activities that would prevent/attenuate senescence programming, SASP secretome induction, inflammatory responses, including inflammaging and cytokine storm. ELVs would exert their functions by paracrine and autocrine activity, the latter mediated by GPCR receptors (FIG. 40). Without wishing to be bound by theory, alveoli and nasal mucosa cells display increased susceptibility to infection due, among other factors, to viral-induced SASP secretome that hijacks homeostasis. We envision this as a self-amplifying feedback loop linked to an evolving inflammatory milieu that facilitates virus entrance and propagation.

We will also study pneumocyte type II cell-specific secretion lipids that display immunoregulatory, anti-inflammatory, and antiviral properties^{37,39,40} phosphatidylglycerol (PhG) and phosphatidylinositol (PI). They are antagonists of Toll-like receptors 1, 2, 4, and 6 that prevent the formation of TNF-α, Il6 and 8, and other proinflammatory mediators⁴⁰. These lipids comprise about 10% of the pulmonary surfactant (complex of lipids-90%-and proteins) secreted by pneumocyte type II cells. The main function of the surfactant is to lower the surface tension at the air/liquid interface within the alveoli needed to lower the work of breathing and prevent alveolar collapse.

Design. We will assess if ELVs (selected in SA1 as the more potent and selective to downregulate ACE2 and proteases) would target senescence programming, SASP secretome and inflammatory responses in alveolar and nasal cells, and how they exert these protective effects. We will use: a) binding and internalization of either RBD or entire S purified protein and b) plasma membrane extracts of human kidney cells (HEK293T) overexpressing S fused to mCherry. We will validate whether (as in Exp.1,2) ELVs attenuate or block the following:

1. Senescence program induced during binding, cleavage, and processing of the S protein. We will use a model of age-related (replicative) senescence as in aging lungs^{41,42}. To induce senescence, we will culture our cells at high density for 2-4 days or use doxorubicin. For the nasal cells, we will use doxorubicin or mite dust to induce senescence (FIG. 52) and then ask if S protein will bind to the cells and/or be internalized more readily than in non-senescence cells. We will use dd-PCR and Jess WB to examine the senescence phenotype (expression of the genes Cdkn2a, Mmpla, Trp53, Cdkn1a, βCdkn1b, and Il-6) and SASP secretome, including ECM remodeling matrix metalloproteinases (as we did recently in human retinal epithelial cells¹⁰). In addition, we will perform the SASP β-gal assay that exploits the availability of a substrate that fluoresces when hydrolyzed by lysosomal galactosidase (FIG. 52A,B). We have experience detecting senescent cells¹⁰. We will then test S protein cell internalization as above. In other human epithelial cells¹⁰, we found that ELVs modify expression (and protein abundance) of p16INK4a (cyclin-dependent kinase inhibitor 2A, Cyclin-Dependent Kinase 4 Inhibitor A), a key in senescence programing protein¹⁰. We will follow the internalization of the S protein by immunocytochemistry by co-staining/co-localization for markers of early and late endosomes, lysosomes, Golgi and ER. We will target Furin, TMPRSS2, and Cathepsin L because these are relevant to the internal processing of S protein and SARS-CoV-2 cell entrance. The S1/S2 position cleavage by Furin and in the S2 position by TMPRSS2 will be monitored by Jess WB and antibodies specific for S and mCherry tag.

2. Inflammatory signaling activated upon RBD binding and/or S protein entrance. Without wishing to be bound by theory, S protein binding and its processing high jack cell metabolism, enhancing the inflammatory response. We will quantify expression of IL1α and β, TNF-α, IL8, IL6, TGFβ and HMGB1 alarmin in the incubation media of cells exposed to RBD, purified and trimeric S protein embedded in plasma membrane using Jess WB. We will follow S protein endocytosis along with cleavage by Furin under basal conditions for sorting endosomes at the TGN—trans-Golgi network^{23,24}. Thus, activation of NFκB to enhance the expression of cytokines and inflammatory mediators will ensue. We will measure the activity of p65, the major subunit of NFκB involved in pro-inflammatory gene expression via a) reporter gene expression as we have previously done²⁴ and b) translocation of p65 to120 min (range of time during which p65 is translocated into the nucleus after stimulation). We will also quantitate expression of the cytokines mentioned above after 4-6 h of exposure to either RBD, purified S protein, or S protein embedded in membranes as a trimer. We will treat the cells with the most effective ELVs (selected in SA1) to assess their effects on cytokine expression and p65 activation.

In addition, we will define the:

3. Mechanism(s) by which ELVs prevent or reduce S protein cell entry. We screened a pool of GPCRs using β-arrestin chimeras to detect receptors activation or deactivation (FIG. 51). Using the Genotype-Tissue Expression (GTEx) Project portal (Genotype Tissue Expression Portal https://www.gtexportal.org/home/), we find that, from the 11 candidate receptors that were activated or deactivated in our screening, 9 are expressed in the lung (FIG. 51): LTB4R, CNR2, GPR52, GPR132 GPR137, CCR1, GPR120, CHRM4, and BAI2.

Following knockdown of these GPCRs in human nasal and alveolar cells, we will challenge the cells with RBD, purified or trimeric S protein +/−ELVs using siRNA. The results will be compared with cells transfected with siRNA negative control challenged the same way as the GPCRs of silenced cells. We will determine the optimal concentration and time course for each of the siRNAs by monitoring expression via Jess WB and ddPCR. Validated siRNAs targeting the GPCRs are commercially available (Origene). To assess whether the GPCRs mediate the protection elicited by ELVs, we will silence or overexpress (using expression plasmids available from Origene) the GPCRs and determine the amount of protein for each by Jess WB. The cells will then be challenged with S protein with or without ELVs. To rule out non-specific interactions, we will use 12HETE, 15HETE, DHA and EPA, and other lipid mediators. In case the receptor has a cognate ligand, as is the case for LTB4 and CNR2, ligands of leukotriene B4 (LTB4) and 2-arachidonoylglycerol (2-AG) or CP55940, an agonist of CNR2 will be co-incubated with the lipid. A competition will be done when antagonism is suspected, for example, LTB4 Receptor/CNR2/ELV interaction. These experiments will determine S protein internalization using cytochemical staining, confocal microscopy, and Imaris analysis.

We will also explore if ELVs synergize with phosphatidylglycerol and phosphatidylinositol secreted by pneumocyte type II and known to be anti-inflammatory and antiviral in the lung^{44,45}. We will conduct a time course adding 200 μg/ml of each of the following lipids: dipalmitoylphosphatidylglycerol, dimyristoyl phosphatidylglycerol, and phosphatidyl inositol, +/−the most effective ELV concentrations (SA1). Then we will ask if they (+/−ELVs) modify the binding/internalization of S purified protein and plasma membrane extracts (human kidney cells, HEK293T) overexpressing S fused to mCherry. The fate of the protein over time will be documented by Jess WB of subcellular fractions and by immunocytochemistry using endosomal and lysosomal markers. Senescence programming and SAP secretome will be assessed as in Exp.1,2. Our cloned mCherry-labeled ORF for the N protein will be the controls since they are not in contact with ACE2 or other surface proteins (FIG. 50).

Conclusions: Without wishing to be bound by theory, senescent programming/SASP will converge with other inflammatory/immune system dysregulations to internalize S protein, and this can explain, at least in part, why older individuals are more susceptible to SARS-CoV-2. We will also contribute to defining cells and molecular targets that contribute to preventing and/or slowing down this and other related viral infections. Without wishing to be bound by theory, decreased internalization and processing of S protein when ELVs are added. Further, NFκB will be activated, cytokine, and HMGB1 expression will be enhanced by S protein internalization. ¹⁰ Also, ELVs will decrease cytokine expression. We will validate which GPCRs determine the protective activity of ELVs by measuring the internalization of S protein. Silencing GPCRs will abrogate the lipid messenger bioactivity in human alveolar and nasal cells. One receptor will be responsible for this activity, and the sets of GPCRs will be different for alveolar cells and nasal cells. Also, there can be a synergy between ELVs with phosphatidylglycerol and/or phosphatidylinositol on regulating S protein entry as well as innate immune processes above and beyond postulated immunoregulatory activities of these anionic lipids as decoys for TLRs and viral attachment 45. The parameters defined in Exp.1-2 will experimentally test +/−these anionic lipids.

FIG. 51 includes GPCRs that did not meet the cutoff % for Path Hunter assay (over 30% for agonist, over 35% for the antagonist) but were within the limit (28% for agonist, 34% for antagonists). We can test these other receptors. As an alternative, we will also test NPD1 (Neuroprotectin D1; derived from DHA^{46,47}). Additionally, we will silence RAMP1, 2, and 3 to assess the activity of the lipid mediators. RAMP proteins are single-transmembrane domain co-receptors that modulate GPCRs signaling⁴⁸. Finally, we have observed that when alveolar cells are cultured beyond 6-8 passages, they do not replicate and become senescent. We will try these alveolar cells as a way to mimic age-related replicative senescence (related to lung pathologies, which are risk factors in COVID-19). Alternatively, we can use doxorubicin to induce cell senescence and then find a condition to explore if ELVs exert protection to follow it as above.

The outcomes will validate the ability of VLC-PUFAs and ELVs as counter-regulators of SARS-CoV-2 cell entry and lung damage (and of other organs) and contribute to a mechanistic understanding of their actions on viral infection, leading to new avenues for disease-modifiable preventive and therapeutic approaches for COVID-19 as well as other viral infections.

### Example 7

Specific Aims

Coronavirus disease 2019 (COVID-19), caused by the Severe Acute Respiratory Syndrome—coronavirus 2 (SARS-CoV-2), is highly transmissible from human to human and has spread rapidly on a global scale. This virus infects alveolar lung cells (This explains the severe impairment in lung gas exchange.), nasal cells, the eye surface, gastrointestinal tract, and central nervous system. SARS-CoV-2 triggers a wide range of disease phenotypes, including severe acute respiratory distress syndrome (ARDS), which may result in death. Moreover, dysregulated immune/inflammatory lung responses in COVID-19 patients contribute to morbidity and mortality. A subset of COVID-19 patients develops a cytokine storm, characterized by increased pro-inflammatory cytokines and monocytes/macrophages that infiltrate the alveoli. Alveolar inflammation and damage lead to COVID-19 morbidity and mortality, so specific compounds/strategies that prevent/attenuate SARS-CoV-2 entrance and activity in the lungs (and other tissues) are needed.

In pursuit of specific mechanisms to control SARS-CoV-2 infectivity, we will test the overarching hypothesis that elovanoids (ELVs) could be harnessed to attenuate viral cell entry, downregulate inflammation and cytokine storm, and as a consequence, promote disease resolution. ELVs are stereospecific dehydroxylated derivatives from very long chain polyunsaturated fatty acids (≥C28, VLC-PUFAs,n-3). ELVs are pro-homeostatic mediators that, as shown in our preliminary results, attenuate the interaction of the receptor-binding domain (RBD) of the viral spike protein (S protein) with Angiotensin-converting enzyme 2 (ACE2) as well as reduce the availability of this receptor in human alveolar cells.

To accomplish our Specific Aims, we will use primary cultures of human alveolar and nasal mucosa cells as well as the RBD of the viral S protein and the recombinant entire viral S protein to test specific predictions of the hypothesis. Based on our recent publication showing that ELVs reversed an injury-induced senescence program in human retinal epithelial cells, we will also test whether the senescence program is critical to contain the binding, cleavage, and processing of the S protein. This will include using age-related (replicative) senescence as in aging lungs and SASP (senescence-associated secretory phenotype) a pro-inflammatory secretome that includes: chemokines, metalloproteinases, proteases, cytokines (e.g., TNF-α, IL-6, and IL-8), and insulin-like growth factor binding proteins as we have demonstrated recently in human retinal epithelial cells.

SA 1) ELVs downregulate the availability (by targeting gene expression) of ACE2, and thus hinder viral attachment. ELVs [also downregulate the host proteases furin, transmembrane serine protease 2 (TWIPRSS2)] and dipeptidyl peptidase 4 (DPP4) that mediate S protein activation and initial viral entry.

SA 2) VLC-PUFAs (n-3) induce lipidome remodeling and disrupt tetraspanin-enriched membrane microdomains (that contribute to blocking attachment and entry of SARS-CoV-2). VLC-PUFAs (n-3) also perturb endosome formation and hinder virus replication;

SA 3) ELVs curtail inflammation and prevent cytokine storm in cultured human alveolar and nasal mucosa cells upon RBD binding or S protein entrance, such as occurs under SARS-CoV-2 attack.

Scientific and Translational Impact: The results of our studies will validate the ability of VLCPUFAs and of ELVs to function as counter-regulators of SARS-CoV-2 cell entry and lung damage (and of other organs) as well as provide a mechanistic understanding of COVID-19 leading to new avenues for potential disease-modifiable therapeutic approaches, including prevention, for this infection as well as other viral infections.

### Example 8

Abstract

Coronavirus disease 2019 (COVID-19), caused by the Severe Acute Respiratory Syndrome—coronavirus 2 (SARS-CoV-2), is highly transmissible from human to human and has spread rapidly on a global scale. This virus infects lung type II alveolar cells (this explains the severe alveolar damage), nasal cells, the eye surface, gastrointestinal tract, and central nervous system and triggers a wide range of disease phenotypes, including severe acute respiratory distress syndrome. A subset of COVID-19 patients develops a cytokine storm, characterized by increased pro-inflammatory cytokines and monocytes/macrophages that infiltrate the alveoli and nasal mucosa. We will validate that ELVs counteract a senescence program and SASP (senescence-associated secretory phenotype), a pro-inflammatory secretome that includes: chemokines, metalloproteinases, proteases, cytokines (e.g., TNF-α, IL-6, and IL-8), and insulin-like growth factor-binding proteins, as we demonstrated recently in human retinal pigment epithelial (RPE) cells. Therefore, specific compounds/strategies that prevent/attenuate SARS-CoV-2 activity in the lungs (and also in other tissues) are needed. In pursuit of mechanisms that might limit virus infectivity, we will validate that elovanoids (ELVs) can be harnessed to attenuate viral cell entry, downregulate inflammation/cytokine storm, and as a consequence, promote disease resolution. To validate this, we will use cultures of primary human alveolar and nasal mucosa cells together with the receptor-binding domain (RBD) of the viral spike (S) protein as well as the entire recombinant S protein. In addition, we will test the prediction that very long chain polyunsaturated fatty acids (VLC-PUFAs,n-3; precursors of ELVs) disrupt membrane microdomains that, in turn, contribute to prevent viral cell attachment as well as impair the formation of endosomes needed for viral replication. Without wishing to be bound by theory, ELVs downregulate the availability (by targeting gene expression) of Angiotensin-converting enzyme 2 (ACE2) and of host proteases transmembrane serine protease 2 (TMPRSS2), furin, and dipeptidyl peptidase 4 (DPP4) that are involved in post-fusion, and viral entry. Moreover, ELVs will reduce inflammation and the ensuing cytokine storm. The Aims address the following: 1) ELVs downregulate availability of [a] ACE2 (and thus hinder cell surface virus binding) and [b] key host proteases (that mediate S protein activation and viral entry); 2) VLC-PUFAs (n-3) induce lipidome remodeling and disrupt tetraspanin-enriched membrane microdomains (that contribute to blocking SARS-CoV-2 virus-cell binding and entry) and also perturb endosome formation and hinder virus replication; and 3) ELVs curtail inflammation, and prevent cytokine storm. This project will validate ELVs as counter-regulators of SARS-CoV-2 cell entry and lung damage as well as provide new mechanistic understanding and avenues for potential therapeutic approaches for COVID-19.

### Example 9

Elovanoids Downregulate Canonical SARS-CoV-2 Cell-Entry Mediators and Enhance Protective Signaling in Human Alveolar Cells

Abstract

The pro-homeostatic lipid mediators elovanoids (ELVs) attenuate cell binding and entrance of the SARS-CoV-2 receptor-binding domain (RBD) in human primary alveoli cells in culture. We uncovered that very-long-chain polyunsaturated fatty acid precursors (VLC-PUFA,n-3) activate ELV biosynthesis in lung cells. Both ELVs and their precursors reduce the binding to RBD. ELVs downregulate angiotensin-converting enzyme 2 (ACE2) and enhance the expression of a set of protective proteins hindering cell surface virus binding and upregulating defensive proteins against lung damage. These findings open avenues for potential preventive and disease-modifiable therapeutic approaches for COVID-19.

The high transmissibility of Severe Acute Respiratory Syndrome—coronavirus 2 (SARS-CoV-2) is due, at least in part, to infectivity in lung type II alveolar cells1 SARS-CoV-2 triggers a wide range of disease phenotypes with severe acute respiratory distress syndrome (ARDS), including interstitial pneumonia² and viral sepsis³. Here, we tested if the pro-homeostatic lipid mediators, the elovanoids (ELVs)⁴⁻⁷, would block the entrance of the spike (S) protein receptor-binding domain (RBD) that would prompt a protective response against SARS-CoV-2 infection.

Lung alveoli viral attachment through the S protein RBD to ACE2 is followed by proteolytic activation for fusion and viral cell entry⁸⁻¹⁰. We use human alveolar primary cell cultures (FIG. 64 panel a, and FIG. 66). Most of the cells are type II (oil red, specific marker, FIG. 64 panel a, right panel, and FIG. 66, bottom panels) and positive to Foxj1, HT2-280 antigen, and β-tubulin IV (FIG. 64 panel a, right panel; FIG. 64 panels g,h and FIG. 66). Pneumocytes type II are also mobile, showing lamellae or filopodia positive to HT2-280, a specific type II (FIG. 64 panel b). We exposed these cells to RBD (from S protein)-Alexa 594 for 24 hours. In parallel, we used Nucleocapsid protein N as a specificity control of RBD internalization. In 3D reconstructions of Z-stack images (Imaris software, Bitplane, UK), the lipophilic staining (cell mask) shows a dense membrane above the nuclear zone that is localized close to oil red (FIG. 64 panels a,c i-viii). A below view (FIG. 64 panelc vi) shows that the RBD protein signal (red) passes through the membrane (white) to the intracellular space surrounding the nucleus (blue), and can also be seen in the above view (FIG. 64 panel c vii,viii). Herein, we demonstrate for the first time that RBD was shown to be internalized in SARS-CoV-2 since previous work have shown the same for SARS-CoV-11¹. When IL1β or TNFα was added, the internalized RBD signal was increased (FIG. 64 panel d). In addition, Nucleocapsid (N) protein, a structural viral protein not involved in ACE2 and SARS-CoV-2 interaction¹², is at the same level as the control with no protein added that accounted for autofluorescence. RBD was internalized at higher rates than N. In digitalized images, plotted vs. Z-axis in a Z-stack shows the differential position of the N protein versus the RBD with respect to the membrane level (FIG. 64 panel c i-v,ix-xi and FIG. 64 panel d). This observation documents that the N protein remains on the membrane and the extracellular side while the RBD spans intracellularly, passing through to the cytoplasm and demonstrating that RBD internalization is specific and dependent on IL1β and TNFα (FIG. 64 panels d,e). RBD-Alexa 594 internalization was decreased +/−IL1β+TNFα when ELV-N32 and ELV-N34 were added (FIG. 64 panel f, upper panel). In addition, acetylenic ELV-N32 or ELV-N34 (FIG. 67) showed a steep decrease in RBD protein internalization +/−IL1β (FIG. 64 panel f, lower panel). Moreover, the addition of the ELVs precursors 32:6 or 34:6 reduces RBD located below the membrane, suggesting that the pneumocytes convert these precursors into ELVs and thus prevent RBD internalization (FIG. 64 panel f, upper panel).

The reduction in RBD internalization is partially due to a decrease in ACE2 since acetylenic ELV-N32 or ELV-N34 decreases ACE2 in pneumocytes type II (FIG. 64 panel g, plot). In addition, ELV-N32 decreased TWIPRSS2 expression (FIG. 64 panel h) in the presence of IL1β (FIG. 64 panel h, plot).

Since ELVs stimulate protective proteins expression in cells confronted with uncompensated oxidative stress″, we next explored if these lipids under conditions that downregulate canonical SARS-CoV-2 cell-entry mediators in pneumocytes will also activate protective proteins synthesis. We found that ELV heightens the expression of Sirtuin 1 (FIG. 64 panel j), RNF146 (FIG. 68 panels a,b), PHB, Bcl-Xl, and Bcl2 (FIG. 68 panelsc-e). These proteins are involved in pro-homeostatic cellular functions. Sirtuin 1 (Silent information regulator factor 2-related enzyme 1) is a NAD(+)-dependent deacetylase of histone and non-histone proteins and transcription factors, and its regulatory functions target inflammation, aging, mitochondrial biogenesis, and cellular senescence¹³. RNF146 is an E3 ubiquitin-protein ligase that degrades parsylated proteins, thus protecting cells from Parthanatos cell death¹⁴. PHB (prohibitin type I) functions comprise scaffolding mitochondrial protein, adaptor in membrane signaling, transcriptional co-regulator, and neuroprotection⁶. Bcl-XL and Bcll2 downregulate apoptosis and inflammasome formation's. Our data indicates that, in addition to halting the entrance of the RBD, ELVs in the lung curb cell-damaging/apoptotic events and thus sustains homeostasis by counteracting inflammation over-activation by the formation of protective proteins.

To validate that alveolar cells in culture can synthesize ELVs, we incubated human alveolar cells with the precursors VLC-PUFAs (32:6 or 34:6) and then analyzed the products by LC-MS/MS. Interestingly, we found that ELVs are in fact, formed. ELV-N32 was synthesized where the precursor 32:6 was added and not in cells exposed to 34:6. Inversely, ELV-N34 was found in the cultures were 34:6 was added and not in cells exposed to 32:6 (FIG. 65 panels a,c). These results demonstrate that alveolar cells are endowed with pathways for the biosynthesis of ELV-N32 and ELV-N34 (FIG. 65 panel b). We show MS fragmentation for stable derivatives of intermediaries (FIG. 65 panel a-c) as well as of ELVs themselves (FIG. 65 panel a). Moreover, we uncovered that ELVs were actively released from cells to the incubation media, indicating that they act both as autocrine and paracrine mediators.

Our findings contribute to broadening our understanding of the duality of ACE2 in lung function and diseases. In health, ACE2 fosters lung homeostasis by generating Ang-(1-7) and enhancing host defense that would counteract ACE2 virus-induced downregulation of proinflammatory signaling. Herein, we show that ELVs uncover another participant when RBD of the S protein binds to ACE2 and enters alveolar cells in culture. Without wishing to be bound by theory, the ELVs are a part of a fast and coordinated pro-homeostatic inflammatory downregulatory response. We will validate that delayed ELV-mediated protective responses can lead to severe lung and systemic inflammation. So direct virus triggered cell damage is critical, but also the activation of the induction of protective proteins. Also, diet has been shown to affect ACE2 expression¹⁶ and the supply to build ELV precursors^{7,17}. This can contribute to explaining why some patients develop hyper-inflammatory/immune responses and severe disease, but others experience mild or even asymptomatic COVID-19. ELVs are the first protective mediators to be identified in the human alveoli confronted with the RBD of the S protein.

We will elucidate the molecular mechanisms of ACE2 downregulation. Also, the use of the entire S protein instead of the RBD, as in our present study, will provide the connection between cell attachment and cell entrance, as affected by ELVs and VLC-PUFAs, since proteases expression is correlated with ACE2 downregulation. Moreover, the use of the intact virus would offer a direct demonstration of the significance of ELVs. Since the SARS-CoV-2 affects nasal mucosa, GI, the eye, and the nervous system exploring the protective activity of ELVs in other cell types would further expand the scope of our observations beyond the lung. Our results offer specific mediators for interventions to modify disease risk, progression, and protection of the lung from COVID-19 or other pathologies.

Methods

Primary cultures of human alveolar cells and assessment of protein internalization. We have used primary cell cultures of human alveoli, which consist of a mixture of ciliated cells, club cells, type I pneumocytes, and type II pneumocytes (PromoCell, HSAEpC). We have characterized the histology and immunocytochemistry in these primary cultures (FIG. 64 panels a,b,g,h). We performed all the experiments in 48 wells with passage 4 cells seeded at 15000 cells per cm2 density. The cells were incubated to confluency and maintained in The proprietary Medium provided by Promocell with the addition of Pen/Strep and exposed to 0.5 ug of tagged protein per well for 24 hours in the presence or absence of 10 ng/ml IL1β (PeProTech Inc., Rocky Hill, NJ Cat #200-01B) and/or 10 ng/ml TNFα (Cell Sciences Inc., Newburyport MA. Cat #CRH520B). After this period, cells were incubated 10 min with 1/1000 cell mask (Thermo Scientific cat #C37608) medium and fixed using PFA 4%. After fixation, nuclei were stained with 10 ug/ml Hoechst 33342 (Thermo Scientific cat #H₃₅₇₀). To characterize the cell types in culture after fixation, we performed immunocytochemistry using the following primary antibodies: HT1-53, a marker of pneumocytes type 1, and HT2-280 (Terrace Biotech cat #HT1-53 and HT2-280); Foxj1 (Santa Cruz Biotech, sc-53139) and β-Tubulin IV marker of pneumocytes type 2 (Abcam cat #ab179509). ACE2 (Santa Cruz Biotech, sc-390851) and TMPRSS2 (Abcam cat #Ab109131) were used for Immunostain the two mentioned proteins in cell culture.

ACE2 Protein abundance using Jess technology. The western assay was performed using a Jess Protein Simple system (San Jose, CA, USA) following the manufacturer's protocol. Briefly, samples were lysed with RIPA buffer containing a protease inhibitor cocktail (Sigma, Cat. P8340). Soluble protein concentration was determined by BCA assay (Thermo Fisher Scientific, Cat. 23225) and 0.4 μg used/reaction. Samples were heated at 95° C./5 min, and 3 μL of each sample were loaded. The 12-230 kDa cartridge (Protein Simple—#SM-W004) was used. Primary antibodies were diluted in antibody diluent 2 buffer (Protein Simple, #042-203), and the working solution of secondary antibodies was provided by the company (Protein Simple, #042-206). For data analysis, the area of spectra that matched the molecular weight of the target protein was used (FIG. 64 panel j). We used the anti-ACE2 antibody from Abcam (cat #ab108252) in a concentration of 1 ug/ml. The standardization was performed using total protein stain and using an anti-GAPDH antibody from Santa Cruz Biotech (cat #Sc-25778).

Quantification of RNF-146. Western blot was performed from lysates obtained using RIPA buffer supplemented with protease inhibitor cocktail (Sigma, cat #P8340. St Louis MO). Total protein (30 mg) was mixed with Laemmli buffer containing DTT and loaded in Novex 4-12% precast gels and ran in X-Cell running system at 120V for 1.5 hours. The transference was performed using the Trans Blot Turbo dry transferring system (Bio-Rad, Hercules CA) on low fluorescent background PVDF membranes (GE Healthcare, Piscataway NJ). Membranes were incubated with the corresponding primary antibodies overnight. Primary antibodies used RNF-146 (UC Davis/NIH-Neuromab Lab Facility, cat #75-233) and GAPDH (Satnta Cruz Biotech Cat #sc-47724). After this period, the membranes were incubated with fluorescent-tagged secondary antibodies (GE healthcare, cat #PA45011) for 1 hour and imaged. Data was acquired using ChemiDoc 1VIP (Biorad). Densitometric analysis was performed using ImageLab 6.0.1. (Biorad).

Preparation of tagged RBD and N nucleocapside proteins. We have obtained the Recombinant SARS-CoV-2, S1 Subunit Protein (RBD), and Recombinant SARS-CoV-2 Nucleocapsid Protein from Raybiotech (cat #230-30162-1000 and 230-30164-500 correspondingly). The proteins were labeled using Alexa Fluor™ 546 Protein Labeling Kit from Thermo Scientific (cat #A10237) following manufacturer directions except for the RBD that was purified from the dye with Amicon-Ultra 10K cutoff filters (Merck, Millipore cat #UFC201024) instead of the column provided by the kit has a restrictive MW of 50KD (the recombinant RBD protein was 25KDa). The recovery of the protein and labeling efficiency was measured using nanodrop and was about 80% recovery and 0.02 dye molecules per aminoacid. We added 0.5 ug per well of protein.

Quantitation of cell surface binding and internalization of tag RBD of the viral S protein. Alexa 594-conjugated RBD domain belonging to the SARS-COVID-2 virus Spike protein (Raybiotech, Peachtree Corners GA. Cat. 230-30162-1000) was chemically accomplexed with the fluorophore Alexa-Fluor 594 using Alexa Fluor™ 594 Protein Labeling Kit (ThermoFisher, Waltham, MA. Cat. A10239). Briefly, 1 mg of protein was resuspended to a final concentration of 0.1 M bicarbonate and then incubated with the Alexa Fluor 594 dye-containing beads for one hour. The dye was washed away using an Amicon-Ultra centrifugal filter cutoff 10KDa (Merck, Millipore Carrigtwohill, CO. Cat. UFC201024). To assess the efficiency of the label, the protein was measured at 280 nm and 590 nm absorbance using NanoDrop One (Thermo Scientific). The final yield gave a ratio of 0.4 moles of dye/mole of protein and a recovery of about 80%.

Real-Time PCR using Taqman probes and SYBR green assay. cDNA was produced using lug of total RNA extracted by RNAeasy (Qiagen, Hilden Germany, cat #74104). The first strand of cDNA was produced using iScript™ Reverse Transcription Supermix for RT-qPCR (BioRad cat #1708840). The quantification of Sirt 1, RNF-146, Bcl2, BcL-xl was performed using SYBRgreen assay with primers designed in house (Table 1) using SsoAdvanced Universal SYBR Green Supermix (Biorad cat #1725270). The quantification of ACE2 and TMPRSS2 mRNA was performed using Taqman probes (Biorad cat #qHsaCEP0051563 & qHsaCIP0028919 respectively) labeled with FAM and standardized using PGK1 probe labeled with HEX (Biorad cat #qHsaCEP0050174).

### REFERENCES CITED IN THIS EXAMPLE

1 Tian S, Xiong Y, Liu H, Niu L, Guo J, Liao M, et al. Pathological study of the 2019 novel coronavirus disease (COVID-19) through postmortem core biopsies. Mod Pathol 2020; 33:1007-14. https://doi.org/10.1038/s41379-020-0536-x.
2 Raghu G, Wilson K C. COVID-19 interstitial pneumonia: monitoring the clinical course in survivors. The Lancet Respiratory Medicine 2020;0: https://doi.org/10.1016/S2213-2600(20)30349-0.
3 Li H, Liu L, Zhang D, Xu J, Dai H, Tang N, et al. SARS-CoV-2 and viral sepsis: observations and hypotheses. Lancet 2020; 395:1517-20. https://doi.org/10.1016/S0140-6736(20)30920-X.
4 Bhattacharjee S, Jun B, Belayev L, Heap J, Kautzmann M-A, Obenaus A, et al. Elovanoids are a novel class of homeostatic lipid mediators that protect neural cell integrity upon injury. Sci Adv 2017;3:e1700735. https://doi.org/10.1126/sciadv.1700735.
5 Bazan N G. Docosanoids and elovanoids from omega-3 fatty acids are pro-homeostatic modulators of inflammatory responses, cell damage and neuroprotection. Mol Aspects Med 2018; 64:18-33. https://doi.org/10.1016/j.mam.2018.09.003.
6 Jun B, Mukherjee P K, Asatryan A, Kautzmann M-A, Heap J, Gordon W C, et al. Elovanoids are novel cell-specific lipid mediators necessary for neuroprotective signaling for photoreceptor cell integrity. Sci Rep 2017; 7:5279. https://doi.org/10.1038/s41598-017-05433-7.
7 Do K V, Kautzmann M-AI, Jun B, Gordon W C, Nshimiyimana R, Yang R, et al. Elovanoids counteract oligomeric β-amyloid-induced gene expression and protect photoreceptors. Proc Natl Acad Sci USA 2019:201912959. https://doi.org/10.1073/pnas.1912959116.
8 Tang T, Bidon M, Jaimes J A, Whittaker G R, Daniel S. Coronavirus membrane fusion mechanism offers a potential target for antiviral development. Antiviral Res 2020; 178:104792. https://doi.org/10.1016/j.antivira1.2020.104792.
9 Andersen K G, Rambaut A, Lipkin W I, Holmes E C, Garry R F. The proximal origin of SARS-CoV-2. Nat Med 2020; 26:450-2. https://doi.org/10.1038/s41591-020-0820-9.
10 Hoffmann M, Kleine-Weber H, Schroeder S, Kruger N, Herrler T, Erichsen S, et al. SARS-CoV-2 Cell Entry Depends on ACE2 and T1VIPRSS2 and Is Blocked by a Clinically Proven Protease Inhibitor. Cell 2020; 181:271-280.e8. https://doi.org/10.1016/j.ce11.2020.02.052.
11 Wang S, Guo F, Liu K, Wang H, Rao S, Yang P, et al. Endocytosis of the receptor-binding domain of SARS-CoV spike protein together with virus receptor ACE2. Virus Res 2008; 136:8-15. https://doi.org/10.1016/j.virusres.2008.03.004.
12 Zeng W, Liu G, Ma H, Zhao D, Yang Y, Liu M, et al. Biochemical characterization of SARS-CoV-2 nucleocapsid protein. Biochem Biophys Res Commun 2020; 527:618-23. https://doi.org/10.1016/j.bbrc.2020.04.136.
13 Barnes P J, Baker J, Donnelly L E. Cellular Senescence as a Mechanism and Target in Chronic Lung Diseases. Am J Respir Crit Care Med 2019; 200:556-64. https://doi.org/10.1164/rccm.201810-1975TR.
14 Belayev L, Mukherjee P K, Balaszczuk V, Calandria J M, Obenaus A, Khoutorova L, et al. Neuroprotectin D1 upregulates Iduna expression and provides protection in cellular uncompensated oxidative stress and in experimental ischemic stroke. Cell Death Differ 2017; 24:1091-9. https://doi.org/10.1038/cdd.2017.55.
15 Bruey J-M, Bruey-Sedano N, Luciano F, Zhai D, Balpai R, Xu C, et al. Bcl-2 and Bcl-XL regulate proinflammatory caspase-1 activation by interaction with NALP1. Cell 2007; 129:45-56. https://doi.org/10.1016/j.ce11.2007.01.045.
16 Mukerjee S, Zhu Y, Zsombok A, Mauvais-Jarvis F, Zhao J, Lazartigues E. Perinatal exposure to Western diet programs autonomic dysfunction in the male offspring. Cell Mol Neurobiol 2018; 38:233-42. https://doi.org/10.1007/s10571-017-0502-4.
17 Bazan N G, Molina M F, Gordon W C. Docosahexaenoic acid signalolipidomics in nutrition: significance in aging, neuroinflammation, macular degeneration, Alzheimer's, and other neurodegenerative diseases. Annu Rev Nutr 2011; 31:321-51. http s://doi. org/10.1146/annurev. nutr. 012809.104635.

### Example 10

Aims to develop nasal/oral deliveries and topical application to the eye of therapeutics for prevention and treatment. Preventive oral inhalation: a) a preventative low dose treatment for elderly as a morning/afternoon (most susceptible individuals, immune weakened), and b) a treatment at disease onset/progression in higher concentrations. VLC-PUFAs (and related molecules) as an orally inhalable nebulizer reach the lung alveoli to prevent and/or slow down COVID-19 virus entrance and damaging consequences (other delivery forms are also included). They also curb inflammation, cytokine storm, and cell damage in the alveoli as ELV synthesis precursors. VLC-PUFAs for orally inhalable development are:
ELOVL4 (elongation of very long chain fatty acids-4) catalyzes the biosynthesis of VLC-PUFAs (≥C28) from 26:6 fatty acids from DHA or eicosapentaenoic acid (EPA).
VLC-PUFAs are then incorporated in phosphatidylcholine molecular species in retina and brain.
Adding these fatty acids to human alveolar cells in culture fosters the formation of atypical lung phospholipids. On the other hand, lipoxygenation of VLC-PUFA leads to di-hydroxylated derivatives termed elovanoids (ELVs), ELV32 and ELV34.
The pro-homeostatic anti-inflammatory bioactivity of ELV, they down-regulate cytokine storm/inflammation activated by SARS-CoV-2 in the lung and likely in other organs.

32:6n-3 and 34:6n-3 VLC-PUFA are incorporated in atypical lung phospholipids and lead to the formation of short-lived lipoxygenase metabolites, 27S-hydroperoxy-32:6 or 29S-hydroperoxy-34:6, respectively which in turn forms the stable 27S/OH-34:6 or 29S/OH-34:6. Elovanoid-32 and 34 are subsequently synthetized.

Description of Technology:
Oral inhalation targeting drugs locally to different regions of the respiratory tract or, alternatively, using the high surface area of the alveoli for systemic delivery.
Pulmozyme and the inhaled insulins are examples of the scope of pulmonary drug delivery of biopharmaceuticals.
Inhalation therapy is one of the oldest therapies to delivers drugs directly into the airways. The delivery of therapeutic aerosols dates back more than 2,000 years to Ayurvedic medicine in India, but the introduction of the first pressurized metered-dose inhaler (pMDI) in 1956 marked the beginning of the modern pharmaceutical aerosol industry. The pMDI portable and convenient inhaler effectively delivered drug to the lung. The Montreal Protocol in 1987 to reduce the use of CFCs as propellants for aerosols led to innovation that resulted in the diversification of inhaler technologies with enhanced delivery efficiency, including modern pMDIs, dry powder inhalers and nebulizer systems.
Tailoring particle size to deliver drugs to treat specific areas of the respiratory tract.

What are its advantages?
Oral inhalation is an alternative for the delivery of small molecules with difficult oral pharmacokinetics and/or extensive liver first-pass metabolism.
Advances in inhaler design and increased understanding of lung physiology makes oral inhalation of complex drugs a therapeutic option
Inhaled insulin was the poster child of orally inhaled complex drugs.
It worked very well (no other non-parenteral delivery route comes even close to the achieved bioavailability and rapidness of action).
Also, the platform technologies tackles the complex network of inflammation-resolving responses, including new pathways, for lung protection mechanisms. Thus, our strategy includes a number of product innovations:
Disruption of lipidome remodeling of membrane microdomains that recruit tetraspanins harnessed to attenuate virus-cell surface binding.
Since host lipid membrane is required to assemble virions in endosomes, we use VLC-PUFAs to perturb endosome formation by triggering the biosynthesis of atypical lung phospholipids
Downregulate ACE2 expression and availability to hinder viral attachment
Downregulate the expression and availability of furin, type II serine protease TMPRSS2, and DPP4 proteases that mediate S protein activation, post-fusion, and initial viral cell entry
Restore expression of matrix metalloproteinases for extracellular matrix sustainment, where integrins are located, to counteract viral infectivity
Upon virus infection, they counteract senescence gene programming, SASP secretome release and inflammaging
Complicated oral pharmacokinetics and/or extensive liver first-pass metabolism can be systemically delivered via the lungs.
Fast onset of drug action is desirable, for example, pain or migraine.
Several bio-drugs for topic delivery are at the horizon. Inhaled anti-sense oligonucleotides, therapeutic antibodies, A1AT and IFN-g are at various stages of clinical development.
Pulmozyme sales were >500 M CHF in2016, thus it is clearly possible to earn money with inhaled proteins.
A new generation of precision inhalers allow for exact dosing and delivery of complex drugs, and most importantly, these devices are designed to remove patient error.

### Example 11

SARS-CoV-2 enters lung epithelial and endothelial cells, triggering release of damage- or danger-associated molecular patterns (DAMPs, initiate/sustain an inflammatory response by innate immune system activation), and proinflammatory cytokines/chemokines release. Neutrophils and platelets are recruited/activated and initiate intravascular thrombin generation which promotes activation of endothelial cells, platelets and neutrophils in a feedback loop that propagates thrombin generation and thrombosis. In this cascade, complement activation also plays a prothrombotic role by recruiting neutrophils and amplifying platelet activation and enhancing endothelial dysfunction and proinflammatory milieu. Hypoxia enhances these processes. Complement activation leads to cytokine storm (associated with lung disease) and thrombophilia (accounting for multi-organ thrombotic microangiopathies). Thrombo-inflammation, hallmark of COVID-19 immunopathology and neutrophil-driven NETosis is a key disease-exacerbating mechanism cross-linked with all other pathogenic events. Complement activation trigger NETs generation, which amplifies complement activation, enhancing inflammation and thrombophilia. This thrombotic cascade leads to clinical manifestations of SARS-CoV-2 coagulopathy: deep vein thrombosis, pulmonary embolism, arterial thrombosis, microvascular thrombosis and ischemic stroke. Without wishing to be bound by theory, Elovanoids as a therapeutic intervention can ‘defuse’ this detrimental loop by interfering with the early pathogenic events of NETosis, preceding multiple organ damage associated with thrombo-inflammation. The following refers to a demonstration of neutrophil extracellular traps in COVID-19; J Exp Med 2020 Dec 7;217(12):e20201012, C Radermecker et al.

Compositions and methods as described herein for use to treat viral infections and pathologies associated therewith, such as COVID-19. For example, Elovanoids can block neutrophil-driven NETosis, a key COVID-19-exacerbating disease mechanism.

### Example 12

Elovanoid-N32 and RvD6-Isomer Decrease ACE2 and Binding of S Protein RBD after Injury or INFγ in the Eye

Abstract

The severe acute respiratory syndrome coronavirus 2 (SARS-CoV-2) infection that causes coronavirus disease 2019 (COVID-19) has resulted in a pandemic affecting the most vulnerable in society, triggering a public health crisis and economic tall around the world. Effective treatments to mitigate this virus infection are needed. Since the eye is a route of virus entrance, we use an in vivo rat model of corneal inflammation as well as human corneal epithelial cells in culture challenged with IFNγ to study this issue. We explore ways to block the receptor-binding domain (RBD) of SARS-CoV-2 spike (S) protein to angiotensin-converting enzyme 2 (ACE2). Elovanoid (ELV)-N32 or Resolvin D6-isomer (RvD6i), among the lipid mediators studied, consistently decreased the expression of the ACE2 receptor, furin, and integrins in damaged corneas or IFNγ stimulated human corneal epithelial cells (HCEC). There was also a concomitant decrease in the binding of spike RBD with the lipid treatments. Concurrently, we uncovered that the lipid mediators also attenuated the expression of cytokines that participate in the cytokine storm, hyper-inflammation and senescence programming. Thus, the bioactivity of these lipid mediators will contribute to opening therapeutic avenues for COVID-19 by counteracting virus attachment and entrance to the eye and other cells and the ensuing disruptions of homeostasis.

### INTRODUCTION

In December 2019, a new infectious respiratory disease (Coronavirus disease 2019, COVID-19¹) caused by severe acute respiratory syndrome coronavirus 2 (SARS-CoV-2) emerged^{2,3}, quickly becoming a pandemic and a global threat to public health. The virus has a single-stranded RNA with a 30 kb genome, which encodes the spike (S) protein that expresses a receptor-binding domain (RBD) for the angiotensin-converting enzyme 2 (ACE2) receptor⁴. In addition, S protein contains cleavage sites for cell proteases FURIN and transmembrane serine protease 2 (TMPRSS2) that allow viral cell entrance⁵. Cells from the alveoli, GI tract, and cornea epithelium, among others, co-expressed Ace2 and Tmprss2 genes^{6,7}.

The eye surface, such as the cornea, is a route of SARS-CoV-2 entrance^{6,7}. Also, the nasolacrimal duct could leak virus-containing tears into the upper respiratory tract. Several lipid mediators modulate inflammatory responses and have been hypothesized to counteract COVID-19 pathology^{8,9}. Lipid mediators facilitate debris clearance and antagonize pro-inflammatory cytokines by fostering inflammation resolution^{10,11}. Here, we study lipoxin A4 (LXA4) derived from the ω-6 arachidonic acid¹², the R,R stereoisomers Neuroprotectin D1 (NPD1)¹³ and Resolvin D6-isomer (RvD6i)¹⁴ called docosanoids since they are derived from ω-3 docosahexaenoic acid and Elovanoid (ELV)-N32 that belongs to a new lipid mediator class discovered in our laboratory—the elovanoids^{15,16}. These lipids are di-hydroxylated derivatives of very long chain polyunsaturated fatty acids (>28C, VLC-PUFAs) with pro-homeostatic and neuroprotective bioactivity 11,15,16. Here, we show that ELV-N32 and RvD6i selectively decrease ACE2 receptor expression and binding of RBD of the S protein in the cornea stroma in an in vivo rat model of cornea injury. We confirm that Ace2 is an interferon-stimulated gene in HCEC, a mechanism that would enhance SARS-CoV-2 infectivity¹⁷. Therefore, we use HCEC in culture challenged with IFNγ to demonstrate that ELV-N32 or RvD6i exert blockage of ACE2 receptor expression, binding of RBD, hyper-inflammation, senescence programming, and components of the cytokine storm.

Results

Lipid mediators decrease cornea injury-induced expression of ACE2 and binding of Alexa 594-RBD

Host proteases for S protein: FURIN5, TMPRSS2 and dipeptidyl peptidase 4 (DPP4)¹⁸ are expressed in the cornea (FIG. 53 panel a), indicating that it is a potential site for SARS-CoV-2 entrance, in agreement with clinical studies showing infected patients' epiphora, conjunctival congestion, or chemosis¹⁹. SARS-CoV-2 triggers lung injury and a systemic dysfunction of the inflammatory-immune system reflected in the cytokine storm^{20,21}. We found that our cornea injury model recapitulates inflammatory-immune system dysfuctions²², including ACE2 receptor expression upon injury. To identify mediators that modulate these responses and to understand consequent mechanisms, we tested the following lipid mediators: LXA4, ELV-N32, RvD6i, and NPD1 (FIG. 53 panels b), c). LXA4, ELV-N32, and RvD6i decrease ACE2 abundance and gene expression levels (FIG. 57) to non-injured tissue, while NPD1 had no effect (FIG. 53 panel d). Alexa 594-RBD displayed remarkable binding to injured cornea stroma, and LXA4, ELV-N32, and RvD6i counteracted these injury-induced effects. Again, NPD1 did not have an effect. Thus, there is a correlation between changes in the ACE2 receptor and RBD binding in the cornea after injury and lipid treatment. Interestingly, most of the RBD was detected in the stroma, and inflammatory cells labeled with CD68 showed co-localization with RBD (FIG. 58 panels a-b).

Lipid mediators disrupt the ACE2 upregulation, hyper-inflammation, senescence, and cytokine storm components in the injured cornea in vivo.

RNA-seq analysis 14 days after injury with and without treatment (FIG. 53 panel b) revealed well-clustered transcriptional profiles in each treated group (FIG. 54 panel a). In PCA plots, the transcriptomic profile of non-injured corneas, control (red), and injured corneas treated with vehicle (green) were well separated. Topical treatment with lipid mediators shows profiles closer to control corneas than to vehicle-treated corneas. ELV-N32 (pink) and RvD6i (cyan) were the nearest to the normal cornea. DEseq2 analysis allows comparison of all treated groups as well as control corneas to vehicle as a reference. Upregulated genes in vehicle-treated injury corneas revealed differences among treatment with lipid mediators, as depicted in Venn diagrams (FIG. 54 panel b). Since NPD1 failed to decrease the ACE2 expression and RBD binding upon injury (FIG. 53 panels d-h),h), we focused on the groups of shared genes between control-LXA4-ELV-N32-RvD6i (450 genes including Ace2) and control-ELV-N32-RvD6i (737 genes). KEGG pathway analysis of these two data sets revealed cytokines and senescence-related pathways (FIG. 54 panel c) with significant false discover rate (FDR) values. On the other hand, IPA analysis predicted several cytokines as upstream regulators of Ace2 increased expression after injury. Interestingly, in addition to cytokines, the CDKN2A (p16/INK4) and NFkB (complex) and its correlated genes were predicted as inducers of Ace2 (FIG. 54 panel d). The RNA-seq analysis of Cdkn2a gene (FIG. 54 panel e) and the IPA inhibition score and p-value of this gene (FIG. 54 panel f) and the NFκB complex (FIG. 54 panel g) confirm the prediction.

Lipid mediators counter-regulate cytokine storm components, NFkB/inflammation, and senescence-associated secretory phenotype after cornea injury.

Since Ace2 gene activation is caused by the action of cytokines, p16INK4a and NFkB, we targeted genes regulated by those inducers. Thus, we validated in the injured cornea: (i) activated cytokines found in the serum of SARS-CoV-2 patients20, (ii) senescence-associated secretory phenotype (SASP) genes²³, and (iii) NFkB/inflammation genes found in lung biopsies of SARS-CoV-224. The Venn diagram showed several shared genes by the three inducers (FIG. 55 panel a). Fifty-one injury-upregulated genes were counteracted by the lipid mediators (FIG. 55 panel b). The plot for each specific gene is provided in FIG. 59. Among those genes, Cxcl10, Hgf, and I11r1 (FIG. 59 panel c, 60 and 61) are related to SARS-CoV-2 load²⁵, while metalloproteinases related genes, such as Mmp9 (FIG. 60), Mmp3, Mmp12, and Timp 1 (FIG. 61) are increase after coronavirus infection and involved in degradation of the extracellular matrix, which facilitates hyperinflammation, leukocyte infiltration, and ECM remodeling and fibrosis^{26,27}. Further, transient receptor Trpc6 (FIG. 60) is a component of chronic obstructive pulmonary disease development²⁸.

Using the KEGG pathway analysis, we found similar pathways to those found in the entire transcriptome (FIG. 54 panel c and FIG. 59 panel a). Employing the EnrichR—Archs4 human analysis tissue database, the 51 genes are more abundant in the omentum and lung (bulk tissue) (FIG. 55 panel c). This indicates that genes detected in the injured cornea might recapitulate changes in gene expression that occur in lung injury. Three targeted cytokines Il1b, Il6, and Vegfa genes are plotted in FIG. 55 panel d. Our data showed that Il6 and Vegfa were upregulated by the injury, and the administration of LXA4, ELV-N32, or RvD6i reduced their expression (FIG. 55 panel d). We also focused on integrin genes since the spike protein contains an RGD motif in the RBD site that is recognized by some integrins as a potential receptor of SARS-CoV-2^{29,30}. Six integrins, which have the RGD binding domain in the heterodimer confirmation, are increased after injury and decreased by some of the lipid mediators (FIG. 55 panel e). Among these genes, Itga5 and Itgb 1 are of interest since their specific blocker ATN-161 greatly attenuates the SARS-CoV-2 infection in vitro³¹, and their expression is significantly decreased by ELV-N32 and RvD6i.

Lipid mediators attenuate IFNγ-specific induction of ACE2 expression, Alexa 594-RBD binding, and senescence programming in human corneal epithelial cells.

Based on the IPA prediction of upstream regulators of Ace2 targeted cytokines, we treated HCEC with IL1β, IL2, IL6, IL8, IFNγ, IFNα, IFNε or TNFα at 1, 10, and 100 ng/mL. IFNγ or 1FNα were the only cytokines to activate Ace2 expression, with IFNγ being the more potent of the two (FIG. 56 panel a and FIG. 62). We followed Ace2 expression by dd-PCR that provides absolute quantification. ELV-N32 or RvD6i markedly attenuated IFNγ-triggered Ace2 activation (FIG. 56 panel b). In addition, IFNγ stimulates the overexpression of senescence programming genes Cdkn2a (p16INK4a) and Mmp 1. ELV-N32, RvD6i, and NPD1 decrease Cdkn2a activation to control values, but LXA4 does not. IFNγ-stimulated Alexa 594-RBD binding (FIG. 56 panel c) correlates with increased ACE2 expression (FIG. 56 panel b). ELV-N32, RvD6, and NPD1 decrease IFNγ-stimulated RBD binding (FIG. 56 panel c). Therefore, our data show that following IFNγ-stimulated RBD binding to ACE2, induction of senescence programming genes Cdkn2a (p16INK4a) and Mmp 1 as well as SASP secretome activation takes place. These events are blocked by ELV-N32, RvD6i, and NPD1 but not by LXA4 (FIG. 56 panel d).

Discussion

Here, we discern bioactivity among a group of lipid mediators on critical targets related to SARS-CoV-2 entrance and deleterious consequences of this viral infection. We uncover that the lipid mediators ELV-N32 and RvD6i decrease ACE2 receptor expression, binding of RBD of the S protein, inflammatory responses, and senescence programming using the rat cornea in vivo model. In addition, we demonstrate using HCEC in culture challenged with IFNγ that ELV-N32 and RvD6i exert similar effects. ELV-N32 remarkably decreases Furin expression, a protease that cleaves the S1/S2 site required for SARS-CoV-2 entry in lung cells.

A key cytokine responding to viral infections is IFNγ32 that increases in the serum of severely affected COVID-19 patients^{20,33}. We found that IFNγ induces Ace2 expression in HCEC at a much lower dose than INFα. Moreover, IFNγ activates cellular senescence reflected in enhanced Cdkn2a expression and SASP secretome release. This observation could contribute to explain why aging populations are more susceptible to COVID-19³⁴. ELV-N32 does bear senolytic activity16, and both, ELV-N32 and RvD6i suppressed senescence genes and the SASP secretome in HCEC (FIG. 56 panel d). Therefore, S protein internalization can lead to IFNγ secretion, which would synergize with an integrin-rich environment amplifying the IFNγ effect³⁵ and stimulating Ace2 overexpression. As a result, the higher ACE2, the higher SARS-CoV-2 binding would be possible. ELV-N32 and RvD6i suppressed the IFNγ stimulation of Ace2 expression as well as the IFNγ-induced senescence, where many SASP components are pro-inflammatory cytokines. PEDF+DHA (the precursor of RvD6i) and RvD1 suppress type 1 pro-inflammatory macrophages (induced by IFNγ) while increasing the type 2 anti-inflammatory macrophage phenotype^{36,37}. Interestingly, ELV-N32, RvD6i, and NPD1 attenuated ACE2-RBD in the IFNγ-treated cells in culture (FIG. 56 panel c), while in the rat injured cornea, LXA4 displayed a significant effect on preventing ACE2-RBD interaction (FIG. 53 panels f-h).h). Of the lipid mediators studied, ELV-N32 and RvD6i consistently displayed protective bioactivity. RvD6i was recently identified in mouse tears as related to corneal nerve regeneration^{14,38}. ELV-N32 is a powerful neuroprotective and anti-inflammatory lipid mediator¹⁶.

ELV-N32 and RvD6i also decrease integrins expression. The S protein contains an RGD motif in the RBD site that recognizes integrins and stimulates virus internalization by activation PI-3K, a pathway predicted to increase along with ACE2 enhanced expression (FIG. 54 panel c)^{29,30}. Inhibition of integrin α5β1 by a non-RGD peptide derived from fibronectin, inhibit the binding of the S protein to ACE2 and decrease virus infection in in vitro³¹.

In conclusion, our data demonstrate that ELV-N32 or RvD6i diminish ACE2 expression and binding of the S protein RBD and, consequently, activate pro-homeostatic signaling and reduce tissue damage.

The application of these lipid mediators can be of therapeutic use alone or as a complement with current antiviral strategies for COVID-19. Without wishing to be bound by theory, the lipid mediators identified here work by similar mechanisms in other cell types and further expand the scope of their therapeutic applications beyond the eye.

Methods

Animals

Sprague-Dawley rats (8-week-old male) were obtained from Charles River Laboratories (Wilmington, MA, USA) and kept at the Animal Care of the Neuroscience Center of Excellence, Louisiana State University Health (LSUH; New Orleans, LA, USA). All animals were handled in compliance with the guidelines of the ARVO Statement for the Use of Animals in Ophthalmic and Vision Research, and the experimental protocol was approved by the Institutional Animal Care and Use Committee (IACUC) at LSUH.

Cornea Injury

The rats were anesthetized by intraperitoneal injection of Ketamine (50-100 mg/kg) plus xylazine (5-10 mg/kg). A 4 mm diameter filter paper soaked in 1 N NaOH was placed on the central cornea of the right eye for 45 seconds, and then the eye was thoroughly washed with mL of saline. After injury, the rats were randomly divided into five treatment groups: vehicle; lipoxin A4 (LXA4) from Cayman Chemical (Ann Arbor, MI, USA); R,R Resolvin D6 isomer (RvD6i), R,R neuroprotection D (NPD1), and elovanoid (ELV)-N32. All lipid solutions were prepared at the final concentration of 1011M using PBS with the minimal contamination of ethanol by evaporating the ethanol and immediately dissolve the lipids in PBS, then vortex well for 2 min. Topical administration (20 μl) was done 3×/day for 14 days. The experiments were double-blinded with the lipid mediators coded during the whole experiments. At the end of the study, when all data was collected, the code was opened.

Corneal RNA-Sequencing

Injured corneas (n=5/condition) were harvested and homogenized with TRIzol (Thermo Fisher Scientific) on ice with a glass Dounce homogenizer. RNA sequencing was performed as described¹⁴. Briefly, after mRNA extraction and determination of purity, 8 ng of total RNA was reverse transcribed, and total cDNAs were amplified using ISPCR primer, and the library was made with the Nextera XT DNA library preparation kit (Illumina, San Diego, CA, USA). The libraries were pooled with the same molarity and sequenced using the NextSeq 500/550 High Output Kit v2 (75 cycles, Illumina). After demultiplexing, RNA-seq data were aligned to the Rattus Norvegicus reference genome (.ensembl.org/pub/release-98/fasta/rattus_norvegicus/dna/) using the Subread package v2.0.1 alignment function39. The BAM files for sequencing data alignment were counted using featureCounts function of Subread tool⁴⁰ using the macOS Catalina. The raw count data were subjected to differential gene expression analysis using DESeq2 package for R⁴¹ with the vehicle group as reference. The adjusted p-values were named as the false discover rate (FDR). Significantly changed genes (FDR<0.05) between each treatment vs. vehicle were subjected to the enrichment analysis using EnrichR42 and NetworkAnalyst 3.043, and pathway analysis using the IPA (QIAGEN Inc., qiagenbioinformatics. com/products/ingenuity-pathway-analysi s).

Preparation of Alexa 594-Conjugated RBD Fragment of S Protein

RBD fragment of the Spike protein belonging to SARS-CoV-2 (Raybiotech, Peachtree Corners G A. Cat. 230-30162-1000) was labeled using Alexa Fluor™ 594 Protein Labeling Kit (ThermoFisher, Waltham, MA. Cat. A10239) following the manufacturer's directions. Briefly, 1 mg of protein was dissolved in 0.1 M bicarbonate and then incubated with the Alexa Fluor 594 dye for one hour. The dye was washed using an Amicon-Ultra centrifugal filter cutoff (Merck, Millipore Carrigtwohill, CO. Cat. UFC201024). To assess the efficiency of the label, the protein was measured at 280 nm and 590 nm absorbance using NanoDrop One (Thermo Scientific). There was a ratio of 0.4 moles of dye/mole of protein and a recovery of about 80%.

Human Corneal Epithelial Cells (HCEC) Culture

All experiments with human corneal epithelial cells were approved by the Institutional Review Board of LSUHNO and conducted in accordance with NIH guidelines. HCEC were kept frozen in the laboratory at passage 2544. Cells were maintained in keratinocyte growth (KGM) medium containing the keratinocyte basal medium (KBM) (Lonza: CC-3101) supplemented with bovine pituitary extract (BPE), hEGF, Insulin, Hydrocortisone and Gentamicin Sulfate-Amphotericin (GA-1000) (Lonza, Cat. CC-4131). For all experiments, cells were seeded at 30,000 cells/cm2.

For screening the stimulation of receptor ACE2 by cytokines, the HCEC were cultured with KGM until 50-60% confluence. Then, changed to KBM containing IL-1β, -2, -6 and 8, IFN-α, -ε, and -γ or TNFα at 1, 10 or 100 ng/ml. The cells were harvested after 6 hours and analyze for the gene expression of Ace2. In other experiments, HCEC were stimulated with IFNγ, and thereafter, lipid mediators were added. For the Alexa 594-conjugated RBD binding, IFNγ was used as a cytokine trigger. At 12 hours after cytokine exposure and lipid mediator treatments, 0.5 γg of labeled RBD was added to the medium. The evaluation of RBD binding was conducted 24 hours after.

Immunohistochemistry

Corneal tissue was fixed in Zamboni fixative (MasterTech Scientific, Lodi, CA USA) for 2 hours immediately after euthanasia. After thoroughly washing with PBS, the corneas were embedded in optimal cutting temperature compound, and serial 10-μm cryostat sections were obtained, dried at room temperature for 2 hours, and stored at −20° C. until use. For immunofluorescence, the sections were incubated with primary antibodies at the concentration described in Table 1 in a wet chamber at 4° C. overnight. The sections were washed 3×/5 min with PBS following by incubation for 1 hour at RT with Alexa Fluor-conjugated secondary antibodies (1:1000 dilution). All sections were counterstained with DAPI (ThermoFisher Scientific, Cat. D1306), and images of rat corneal samples were acquired with an Olympus IX71 fluorescent microscope.

TABLE 1
List of primary antibodies used in this study

| | | | Cat. | Immuno- | Western |
|---|---|---|---|---|---|
| No. | Name | Company | Number | fluorescence | Blot |
| 1 | Rabbit anti- | Abcam | Ab108252 | 1:1000 | 1:100 |
| | ACE2 | | | | |
| 2 | Rabbit anti- | Abcam | Ab129060 | 1:500 | 1:100 |
| | DPP4 | | | | |
| 3 | Rabbit anti- | Abcam | Ab183495 | 1:1000 | 1:100 |
| | FURIN | | | | |
| 4 | Rabbit anti- | Abcam | Ab109131 | 1:1500 | 1:100 |
| | TMPRSS2 | | | | |
| 5 | Rabbit anti- | Santa | Sc-25778 | | 1:1000 |
| | GAPDH | Cruz | | | |
| 6 | Anti-neutrophil | LSBio | LS-C348005 | 1:500 | |
| 7 | Mouse anti-rat | Bio-Rad | MCA341GA | 1:1000 | |
| | CD68 | | | | |

Unbiased Imaging-Based Evaluation of RBD Binding

Twenty-four hours after Alexa 594-RBD was added to the HCEC, the cells were washed with PBS (3×/5 min) and fixed with 4% paraformaldehyde for 30 minutes at RT. The HCEC were washed 2× with PBS and stained with Hoechst 33342 Solution (ThermoFisher Scientific, Cat. 62249) for 30 minutes at RT. Next, the HCEC were washed 2× with PBS before imaging. For unbiased data collection, 7 designated areas were defined in each well (FIG. 63) and captured with an Olympus FV3000 confocal laser scanning microscopy under “Multi Area Time Lapse” (MATL) mode. All images were acquired with the same parameters and Z-section range, converted and inputted in the Imaris software version 9.5.1. The threshold for the control images was defined by the HCEC without Alexa 594-conjugated RBD of S protein and using it as a threshold filter for the Imaris batch image processing function. The sum of total intensity for each image was used to evaluate the binding efficiency. The whole process was summarized in the FIG. 63 panels b,c.

Droplet Digital PCR (dd-PCR)

Total RNA was isolated using RNeasy Plus Mini Kit (Qiagen, Germany), and 1 μg of total RNA was reverse transcribed using an iScript cDNA Synthesis Kit (Bio-Rad, Cat. 170-8841). For ddPCR, 10 ng of cDNA was multiplexed with Ace2 and phosphoglycerate kinase 1 (Pgk1) probes (Bio-Rad, Cat. qHSACEP005-1563 and dHSACPE503-3809) using dd-PCR Supermix for Probes No dUTP (Bio-Rad, Cat. 1863024). Then, 20 μL of the reaction was mixed with 70 μL of Droplet Generation Oil (Bio-Rad Cat:1863005) to make the reaction droplets. The emulsified samples were carefully transferred to PCR plates (Bio-Rad, Cat. 12001925) and amplified using the cycling: 95° C. for 10 minutes, 40 cycles of a two-step cycling protocol (94° C. for 30 seconds and 60° C. for 1 minute), and 98° C. for 10 minutes. Next, the post-cycling plate was placed into the QX200 Droplet Reader with the FAM/HEX setting. The absolute quantity of DNA per sample (copies/μL) was processed using QuantaSoft Analysis Pro Software. For the data analysis, the ratio of quantified Ace2 to Pgk1 was used.

Capillary-based Western Blot

The capillary-based western assay was performed using a Jess Protein Simple system (San Jose, CA, USA) as manufacture suggested protocol. Briefly, samples were lysed with RIPA buffer containing a protease inhibitor cocktail (Sigma, Cat. P8340). Cell debris was removed after 10 min centrifugation at 16,000×g. Protein concentration was determined by BCA assay (Thermo Fisher Scientific, Cat. 23225) and 1 μg used/reaction. Fluorescent Master Mix was mixed with 40 mM DTT, and the mixture was added to each sample to provide a denaturing and reducing environment. Samples were heated at 95° C./5 min, and 3 μL of each sample were loaded. The 12-230 kDa cartridge (Protein Simple—#SM-W004) was used. Primary antibodies were diluted in antibody diluent 2 buffer (Protein Simple, #042-203) while the working solution of secondary antibodies was provided by the company (Protein Simple, #042-206). Then, the filled plate was spin-down for 10 min at 1,000×g to remove bubbles and plate, and capillaries were loaded into the Jess machine. For data analysis, the area of spectra that matched the molecular weight of the target protein was used. To reduce the coefficient variant, we analyzed the GAPDH for each capillary. The ratio of the targeted protein to GAPDH was used for statistical comparisons. For visualization, the artificial lanes generated from spectra volume was used.

High-throughput qPCR using Biomark™ HD

Quantitative PCR was performed with the Biomark HD system (Fluidigm, San Francisco, CA, USA). Briefly, 200 ng of RNA was reverse-transcribed using iScript Reverse Transcription Supermix (Bio-Rad), and the cDNA was pre-amplified using the PreAmp Master Mix (PN 100-5580; Fluidigm). The cDNA was then subjected to Exonuclease I treatment and diluted 5 times in TE Buffer. The qPCR reaction mixture and primer reaction mixture were made and loaded into the Biomark 96.96 IFC™ (Integrated Fluidic Circuit). The enzyme reaction was mixed using Juno™ Controller (Fluidigm) and run using the cycling program of (i) 70° C. for 40 minutes followed by 60° C. for 30 seconds, (ii) hot start for 1 minute at 95° C., (iii) 30 cycles of denaturation at 96° C. for 5 seconds, and annealing at 60° C. for 20 seconds, and (iv) melting curves between 60° C. and 95° C. with 1° C. increments/3 seconds. The Ct value of target genes was normalized to the house-keeping genes Gapdh, Hprt1, and Tfrc before normalized to the vehicle group. Relative fold changes from the ΔΔCT calculation was used to make the graph. Primer sequences are provided in Table 2.

TABLE 2
Primers for qPCR

| Gene | | |
|---|---|---|
| name | Forward | Reverse |
| Ace2 | CATTGGAGCAAGTGTTGG | GAGCTAATGCATGCCATTC |
| | ATCTT | TCA |
| | SEQ ID NO: 1 | SEQ ID NO: 2 |
| Cdkn2a | GGGGGCACCAGAGGCAGT | GGTTGTGGCGGGGGCAGTT |
| | SEQ ID NO: 3 | SEQ ID NO: 4 |
| Mmp1 | GGGCTTGAAGCTGCTTAC | CAGCATCGATATGCTTCAC |
| | GAATT | AGTTCT |
| | SEQ ID NO: 5 | SEQ ID NO: 6 |
| Gapdh | TGGACCTGACCTGCCGTC | CCCTGTTGCTGTAGCCAAA |
| | TA | TTC |
| | SEQ ID NO: 7 | SEQ ID NO: 8 |
| Tfrc | GGCTACTTGGGCTATTGT | CAGTTTCTCCGACAACTTT |
| | AAAGG | CTCT |
| | SEQ ID NO: 9 | SEQ ID NO: 10 |
| Hprt1 | GACCAGTCAACAGGGGAC | AACACTTCGTGGGGTCCTT |
| | AT | TTC |
| | SEQ ID NO: 11 | SEQ ID NO: 12 |

Statistical Analysis

Data are expressed as mean±SD. The data were analyzed by 1-way ANOVA followed by Dunnett's multiple comparisons post hoc test at 95% confidence level with the vehicle as reference. All graphs were made using GraphPad Prism 7 (GraphPad Software, La Jolla, CA, USA) with the mean±SD while all statistical analyses were done using built-in function of Prism 7.

### REFERENCES CITED IN THIS EXAMPLE

1. Naming the coronavirus disease (COVID-19) and the virus that causes it. who.int/emergencies/diseases/novel-coronavirus-2019/technical-guidance/naming-the-coronavirus-disease-(covid-2019)-and-the-virus-that-causes-it.
2. Zhu N. et al. A Novel Coronavirus from Patients with Pneumonia in China, 2019. Engl. J. Med. 382, 727-733 (2020).
3. Chen N. et al. Epidemiological and clinical characteristics of 99 cases of 2019 novel coronavirus pneumonia in Wuhan, China: a descriptive study. The Lancet 395, 507-513 (2020).
4. Hoffmann M. et al. SARS-CoV-2 Cell Entry Depends on ACE2 and TMPRSS2 and Is Blocked by a Clinically Proven Protease Inhibitor. Cell 181, 271-280.e8 (2020).
5. Hoffmann M., Kleine-Weber H. & Pohlmann S. A Multibasic Cleavage Site in the Spike Protein of SARS-CoV-2 Is Essential for Infection of Human Lung Cells. Cell 78, 779-784.e5 (2020).
6. Sungnak W. et al. SARS-CoV-2 entry factors are highly expressed in nasal epithelial cells together with innate immune genes. Med. 26, 681-687 (2020).
7. Zhou L. et al. ACE2 and TMPRSS2 are expressed on the human ocular surface, suggesting susceptibility to SARS-CoV-2 infection. Surf 18, 537-544 (2020).
8. Panigrahy D. et al. Inflammation resolution: a dual-pronged approach to averting cytokine storms in COVID-19? Cancer Metastasis Rev. (2020) doi:10.1007/s10555-020-09889-4.
9. Regidor P.-A. Covid-19 management with inflammation resolving mediators? Perspectives and potential. Hypotheses 142, 109813 (2020).
10. Serhan C. N. Pro-resolving lipid mediators are leads for resolution physiology. Nature 510, 92-101 (2014).
11. Bazan N. G. Docosanoids and elovanoids from omega-3 fatty acids are pro-homeostatic modulators of inflammatory responses, cell damage and neuroprotection. Aspects Med. 64, 18-33 (2018).
12. Serhan C. N., Hamberg M. & Samuelsson B. Lipoxins: novel series of biologically active compounds formed from arachidonic acid in human leukocytes. Natl. Acad. Sci. 81, 5335-5339 (1984).
13. Mukherjee P. K., Marcheselli V. L., Serhan C. N. & Bazan N. G. Neuroprotectin D1: a docosahexaenoic acid-derived docosatriene protects human retinal pigment epithelial cells from oxidative stress. Natl. Acad. Sci. U.S.A 101, 8491-8496 (2004).
14. Pham T. L. et al. Novel RvD6 stereoisomer induces corneal nerve regeneration and wound healing post-injury by modulating trigeminal transcriptomic signature. Rep. 10, 1-12 (2020).
15. Jun B. et al. Elovanoids are novel cell-specific lipid mediators necessary for neuroprotective signaling for photoreceptor cell integrity. Rep. 7, 5279 (2017).
16. Do K. V. et al. Elovanoids counteract oligomeric β-amyloid-induced gene expression and protect photoreceptors. Natl. Acad. Sci. U.S.A 116, 24317-24325 (2019).
17. Ziegler C. G. K. et al. SARS-CoV-2 Receptor ACE2 Is an Interferon-Stimulated Gene in Human Airway Epithelial Cells and Is Detected in Specific Cell Subsets across Tissues. Cell 181, 1016-1035.e19 (2020).
18. Li Y. et al. The MERS-CoV receptor DPP4 as a candidate binding target of the SARS-CoV-2 iScience (2020) doi:10.1016/j.isci.2020.101160.
19. Wu P. et al. Characteristics of Ocular Findings of Patients With Coronavirus Disease 2019 (COVID-19) in Hubei Province, China. JAMA Ophthalmol. (2020) doi:10.1001/j amaophthalmo1.2020.1291.
20. Huang C. et al. Clinical features of patients infected with 2019 novel coronavirus in Wuhan, Lancet Lond. Engl. 395, 497-506 (2020).
21. Pedersen S. F. & Ho Y.-C. SARS-CoV-2: a storm is raging. Clin. Invest. 130, 2202-2205 (2020).
22. Sotozono C. et al. Cytokine expression in the alkali-burned cornea. Eye Res. 16, 670-676 (1997).
23. Coppe J.-P., Desprez P.-Y., Krtolica A. & Campisi J. The Senescence-Associated Secretory Phenotype: The Dark Side of Tumor Suppression. Rev. Pathol. 5, 99-118 (2010).
24. Identification of SARS-CoV2-mediated suppression of NRF2 signaling reveals a potent antiviral and anti-inflammatory activity of 4-octyl-itaconate and dimethyl fumarate. (2020) doi:10.21203/rs.3.rs-31855/v1.
25. Yang Y. et al. Plasma IP-10 and MCP-3 levels are highly associated with disease severity and predict the progression of COVID-19. Allergy Clin. Immunol. 146, 119-127.e4 (2020).
26. Marten N. W. & Zhou J. The Role of Metalloproteinases in Corona Virus Infection. Models Mult. Scler. 839-848 (2005) doi:10.1007/0-387-25518-4_48.
27. Ueland T. et al. Distinct and early increase in circulating MMP-9 in COVID-19 patients with respiratory failure. Infect. (2020) doi:10.1016/j.jinf.2020.06.061.
28. Bose P., Bathri R., Kumar L., Vijayan V. K. & Maudar K. K. Role of oxidative stress & transient receptor potential in chronic obstructive pulmonary disease. Indian J. Med. Res. 142, 245-260 (2015).
29. Luan J., Lu Y., Gao S. & Zhang L. A potential inhibitory role for integrin in the receptor targeting of SARS-CoV-2. Infect. (2020) doi:10.1016/j.jinf.2020.03.046.
30. Sigrist C. J., Bridge A. & Le Mercier P. A potential role for integrins in host cell entry by SARS-CoV-2. Antiviral Res. 177, 104759 (2020).
31. Beddingfield B. et al. The Integrin Binding Peptide, ATN-161, as a Novel Therapy for SARS-CoV-2 Infection. bioRxiv(2020) doi:10.1101/2020.06.15.153387.
32. Samuel C. E. Antiviral Actions of Interferons. Microbiol. Rev. 14, 778-809 (2001).
33. Han H. et al. Profiling serum cytokines in COVID-19 patients reveals IL-6 and IL-10 are disease severity predictors. Microbes Infect. 9, 1123-1130 (2020).
34. Coronavirus Disease 2019 (COVID-19). Centers for Disease Control and Prevention cdc.gov/coronavirus/2019-ncov/need-extra-precautions/people-at-increased-risk.html (2020).
35. Krummel M. F. et al. Paracrine costimulation of IFN-γ signaling by integrins modulates CD8 T cell differentiation. Natl. Acad. Sci. U.S.A. 115, 11585-11590 (2018).
36. Schmid M., Gemperle C., Rimann N. & Hersberger M. Resolvin D1 Polarizes Primary Human Macrophages toward a Proresolution Phenotype through GPR32. Immunol. Baltim. Md 1950 196, 3429-3437 (2016).
37. He J., Pham T. L., Kakazu A. & Bazan H. E. P. Recovery of Corneal Sensitivity and Increase in Nerve Density and Wound Healing in Diabetic Mice After PEDF Plus DHA Treatment. Diabetes 66, 2511-2520 (2017).
38. Pham T. L. et al. Defining a mechanistic link between pigment epithelium-derived factor, docosahexaenoic acid, and corneal nerve regeneration. Biol. Chem. 292, 18486-18499 (2017).
39. Liao Y., Smyth G. K. & Shi W. The Subread aligner: fast, accurate and scalable read mapping by seed-and-vote. Nucleic Acids Res. 41, e108 (2013).
40. Liao Y., Smyth G. K. & Shi W. featureCounts: an efficient general purpose program for assigning sequence reads to genomic features. Oxf. Engl. 30, 923-930 (2014).
41. Love M. I., Huber W. & Anders S. Moderated estimation of fold change and dispersion for RNA-seq data with DESeq2. Genome Biol. 15, 550 (2014).
42. Kuleshov M. V. et al. Enrichr: a comprehensive gene set enrichment analysis web server 2016 update. Nucleic Acids Res. 44, W90-97 (2016).
43. Zhou G. et al. NetworkAnalyst 3.0: a visual analytics platform for comprehensive gene expression profiling and meta-analysis. Nucleic Acids Res. 47, W234-W241 (2019).
44. Sharma G. D., Kakazu A. & Bazan H. E. P. Protein kinase C alpha and epsilon differentially modulate hepatocyte growth factor-induced epithelial proliferation and migration. Eye Res. 85, 289-297 (2007).

### Example 14

Induction of NETosis and its Blockage Using Elovanoids
FIG. 71 shows a non-limiting exemplary Experimental Design for Induction of NETosis and its blockage using Elovanoids
FIG. 72 shows there is significant reduction in the amount of extracellular DNA in the cell culture supernatant with the lipids, and there is nice effect of the increasing dose of the lipids both for 34:6 Na and 32:6 Me. But 34:6 Na seems to be better. Human neutrophils were stressed with calcium ionophore A23187 [5 μM] for 4 hours and with Phorbol myristate acetate (PMA) [100 nM] for 5 hours induce Netosis.
FIG. 73 shows there is significant reduction in the amount of extracellular DNA in the cell culture supernatant with the lipids, and there is nice effect of the increasing dose of the lipids both for 34:6 Na and 32:6 Me. But 34:6 Na seems to be more effective. Human neutrophils were stressed with calcium ionophore A23187 [5 μM] for 4 hours and with Phorbol myristate acetate (PMA) [100 nM] for 5 hours to induce Netosis. Extracellular DNA was also measured with SYTOX green and was significantly reduced by addition of 34:6 Me and 32:6 Me-A.
FIG. 74 shows there is significant reduction in the amount of citrullination of Histone H₃ in the cell culture supernatant (from human polymorphonuclear leukocytes) with the lipids, and there is a nice effect of the increasing of dose of the lipids both for 34:6 Na and 32:6 Me. Human neutrophils were stressed with calcium ionophore A23187 [5 μM] for 4 hours and with Phorbol myristate acetate (PMA) [100 nM] for 5 hours to induce Netosis.
FIG. 75 shows there is significant reduction in the amount of extracellular DNA in the cell culture supernatant with the lipids, along with PMN elastase and human myeloperoxidase. Human neutrophils were stressed with calcium ionophore A23187 [5 μM] for 4 hours and with Phorbol myristate acetate (PMA) [100 nM] for 5 hours to induce Netosis.

### Example 15

Lipoxins as a Therapy for COVID-19

Cell entry of coronaviruses depends on binding of viral spike (S) proteins to cellular receptors as well as on S-protein priming by host cell proteases. ACE2 is a major receptor for SARS-CoV-2. TMPRSS2 and Furin are major proteases that facilitate virus entrance.

We discovered the effects of lipoxin A4 (LxA4) using the rat cornea after alkali burn as a model (FIG. 76).

LxA4 is a bioactive metabolite derived from arachidonic acid, then an eicosanoid, made in most cells. It acts to resolve inflammatory responses.

The RNA-seq data of the rat cornea uncovers SARS-CoV-2 risk in the ocular surface. Alkali burn stimulates the activation of a form of “cytokine storm” on the ocular surface enhancing the availability of proteins that facilitates the SARS-CoV-2-entrance. We found that (i) receptor ACE2, (ii) the proteases TMPRSS2, and Furin were increased after alkali burn. LxA4 anti-inflammatory lipid mediator remarkable blocked the availability of these key proteins. The protein amount of ACE2 and Furin are confirmed by JESS capillary-based Western Blot.

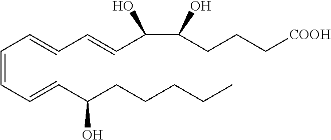 [SMILES: CCCCC[C@@H](O)/C=C/C=C\C=C\C=C\[C@@H](O)[C@@H](O)CCCC(=O)O]

In summary, Lipoxin A4 downregulates the gene expression and protein availability of the ACE2 receptor. In addition, it downregulates the proteins that act to facilitate the internalization of SARS-CoV-2 into cells. Without wishing to be bound by theory, Lipoxin A4 can be used therapeutically in the form of an inhalable formulation to arrive into the lungs and mucous to observe these effects.

The approach described herein is different from current research that includes structure-based drug discovery focused on proteases; on the Spike (S) protein—ACE2 interactions; antivirals; further studies on hydroxychloroquine; target identification on COVIDT-19 viral genome; development of vaccines; use of steroids (suppression of the immune system is a drawback) and IL-6 inhibitors to reduce lungs macrophages flow. In addition, drugs repurposing and combination therapies, including Actemra (Tocilizumab), plus an antiviral drug; anakinra, which targets the spare IL-1 soluble receptor that reduces immune responses without interfering with the beneficial action of CD4 (initiation of immune response), and CD8 T-cells (antiviral cells).

### Example 16

ELV-N32 or RvD6i Counteract SARS-CoV-2 Eye Entrance and Inflammatory Damaging Consequences

We have identified lipid mediators that attenuate/block entrance of the severe acute respiratory syndrome coronavirus 2 (SARS-CoV-2) that causes coronavirus disease 2019 (COVID-19) into the eye. This condition has resulted in a pandemic affecting the most vulnerable in society (elderly and disadvantaged minorities), triggering a public health crisis and economic fall around the world. Effective treatments to mitigate this viral infection are needed. Since the eye is a route of virus entrance, we use an in vivo rat model of corneal inflammation/injury as well as human corneal epithelial cells in culture challenged with IFNγ. We identified ways to block the binding of receptor-binding domain (RBD) of SARS-CoV-2 spike (S) protein to angiotensin-converting enzyme 2 (ACE2). Among the lipid mediators studied, Elovanoid-N32 (June et al., 2017) or Resolvin D6-isomer consistently decreased the expression of the ACE2 receptor, furin, and integrins in damaged corneas or IFNγ-stimulated human corneal epithelial cells. There was also a concomitant decrease in the binding of spike RBD upon the lipid treatments. We disclose here that the lipid mediators also attenuated the expression of cytokines that participate in the cytokine storm, hyper-inflammation, and senescence programming.

ELV-N32 or RvD6i, individually or combined, can be used topically on the eye surface to target the eye itself and the upper respiratory track. The nasolacrimal duct could deliver compounds into the upper respiratory tract. In addition, ELV-N32 or RvD6i, individually or combined, can be therapeutically formulated to counteract SARS-CoV-2 entrance and inflammatory consequences in the lungs, brain, heart, and other tissues.

In December 2019, a new infectious respiratory disease (Coronavirus disease 2019, COVID-19 (“Naming the coronavirus disease (COVID-19) and the virus that causes it,” n.d.)) caused by severe acute respiratory syndrome coronavirus 2 (SARS-CoV-2) emerged, quickly becoming a pandemic and a global threat to public health. The virus has a single-stranded RNA with a 30 kb genome, which encodes the spike (S) protein that expresses a receptor-binding domain (RBD) for the angiotensin-converting enzyme 2 (ACE2) receptor. In addition, S protein contains cleavage sites for cell proteases FURIN and transmembrane serine protease 2 (TMPRSS2) that allow viral cell entrance. Cells from the alveoli, GI tract, and cornea epithelium co-expressed Ace2 and Tmprss2 genes.

We targeted the lipid mediators disclosed here in the eye because the eye surface, particularly the cornea, is a route of SARS-CoV-2 entrance. Also, the nasolacrimal duct could leak virus-containing tears into the upper respiratory tract.

Several lipid mediators modulate the inflammatory response and can counteract COVID-19 pathology (Panigrahy et al., 2020; Regidor, 2020). Lipid mediators facilitate debris clearance and antagonize proinflammatory cytokines by fostering inflammation resolution. We have explored several mediators, including lipoxin A4 (LXA4) derived from the ω-6 arachidonic acid, Resolvin D6-isomer (RvD6i), Neuroprotectin D1 (NPD1), and Elovanoid-N32 (ELV-N32). The ELV lipids are di-hydroxylated derivatives of very long chain polyunsaturated fatty acids (>28C, VLC-PUFAs) with pro-homeostatic and neuroprotective bioactivity. Here, ELV-N32 and RvD6i selectively decrease ACE2 receptor expression and binding of RBD of the S protein in the cornea stroma in an in vivo rat model of cornea injury/inflammation. Ace2 is an interferon stimulated gene in human epithelial cells, mechanism that would enhance SARS-CoV-2 infectivity. Therefore, using human corneal epithelial cells in culture challenged with IFNγ, we disclose that ELV-N32 and RvD6i exert blockage of ACE2 receptor expression, binding of RBD, hyper-inflammation, senescence programing, and components of the cytokine storm.

### Example 17

Nebulizer Oral Delivery for COVID-19

Embodiments described herein comprise the development of nasal/oral deliveries and topical application, such as to the eye, of therapeutics for prevention and treatment of viral infection. Preventive oral inhalation comprises a) a preventative low dose treatment for elderly as a morning/afternoon (most susceptible individuals, immune weakened), and b) a treatment at disease onset/progression in higher concentrations. Embodiments as described herein as an orally inhalable nebulizer reach the lung alveoli to prevent and/or slow down COVID-19 virus entrance and damaging consequences (other delivery forms are also included).

Oral inhalation targeting drugs locally to different regions of the respiratory tract or, alternatively, using the high surface area of the alveoli for systemic delivery.

Pulmozyme and the inhaled insulins are examples of the scope of pulmonary drug delivery of biopharmaceuticals.

Inhalation therapy is one of the oldest therapies to deliver drugs directly into the airways. The delivery of therapeutic aerosols dates back more than 2,000 years to Ayurvedic medicine in India, but the introduction of the first pressurized metered-dose inhaler (pMDI) in 1956 marked the beginning of the modern pharmaceutical aerosol industry. The pMDI portable and convenient inhaler effectively delivered drug to the lung. The Montreal Protocol in 1987 to reduce the use of CFCs as propellants for aerosols led to innovation that resulted in the diversification of inhaler technologies with enhanced delivery efficiency, including modern pMDIs, dry powder inhalers and nebulizer systems.

Tailoring particle size to deliver drugs to treat specific areas of the respiratory tract.

A new wave of inhaled biomolecules has recently entered clinical trials.

Oral inhalation is an alternative for the delivery of small molecules with difficult oral pharmacokinetics and/or extensive liver first-pass metabolism.

Advances in inhaler design and increased understanding of lung physiology makes oral inhalation of complex drugs a therapeutic option.

Inhaled insulin was the poster child of orally inhaled complex drugs.

It worked very well (no other non-parenteral delivery route comes even close to the achieved bioavailability and rapidness of action).

Also, the platform technologies tackles the complex network of inflammation-resolving responses, including new pathways, for lung protection mechanisms.

Complicated oral pharmacokinetics and/or extensive liver first-pass metabolism can be systemically delivered via the lungs.

Fast onset of drug action is desirable, for example, pain or migraine.

Several bio-drugs for topic delivery are at the horizon. Inhaled anti-sense oligonucleotides, therapeutic antibodies, A1AT and IFN-g are at various stages of clinical development.

Pulmozyme sales were >500 M CHF in2016, thus it is clearly possible to earn money with inhaled proteins.

A new generation of precision inhalers allow for exact dosing and delivery of complex drugs, and most importantly, these devices are designed to remove patient error.

We claim:

1. A method of treating or preventing a viral infection or symptom thereof in a subject, the method comprising administering to the subject a therapeutically effective amount of a very long chain polyunsaturated fatty acid (VLC-PUFA), arachidonic acid, docosahexaenoic acid, or a derivative thereof.

2. The method of claim 1, wherein the treating or preventing comprises reducing an immune response.

3. The method of claim 2, wherein the immune response comprises tissue inflammation.

4. The method of claim 3, wherein the tissue comprises ocular tissue, brain tissue, gastrointestinal tissue, skin tissue, or heart tissue.

5. The method of claim 1, wherein the VLC-PUFA comprises a compound of A or B:

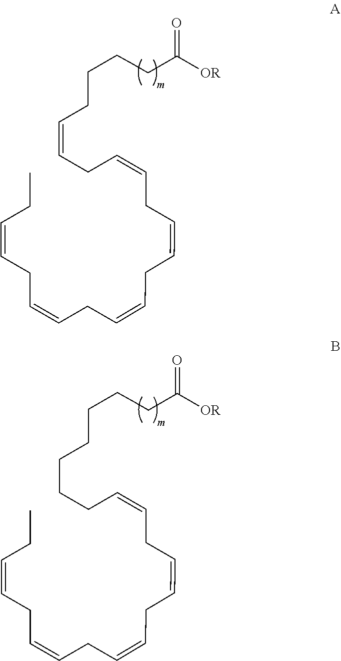

6. The method of claim 5, wherein R comprises —H, —OH, methyl, ethyl, propyl, or an alkyl group; wherein m comprises 0-19; or any combination thereof.

7. The method of claim 6, wherein the compound comprises (14Z,17Z,20Z,23Z,26Z,29Z)-dotriaconta-14,17,20,23,26,29-hexaenoic acid) or (16Z,19Z,22Z,25Z,28Z,31Z)-tetratriaconta-16,19,22,25,28,31-hexaenoic acid).

8. The method of claim 6, wherein the compound comprises a compound of A1 or B1:

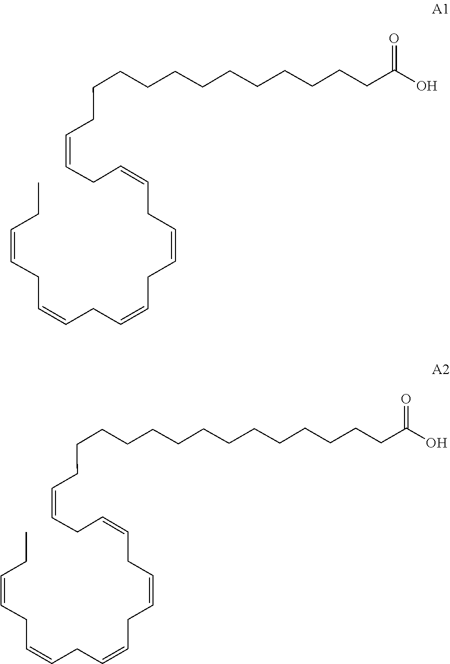 [SMILES: CC/C=C\C/C=C\C/C=C\C/C=C\C/C=C\C/C=C\CCCCCCCCCCCCC(=O)O ; CC/C=C\C/C=C\C/C=C\C/C=C\C/C=C\C/C=C\CCCCCCCCCCCCCCC(=O)O]

9. The method of claim 1, wherein the VLC-PUFA derivative comprises: 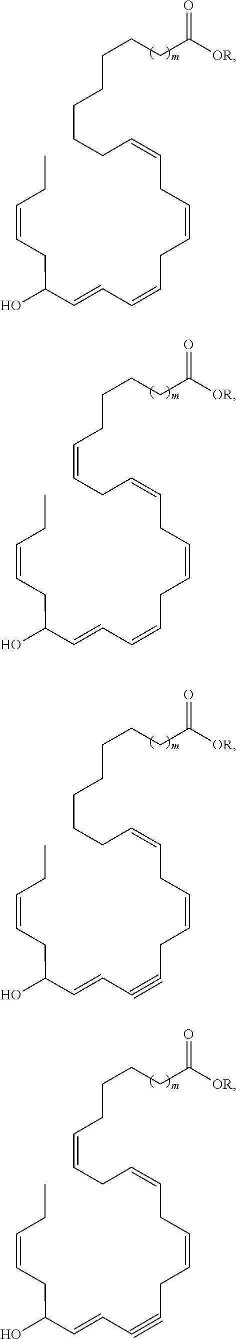 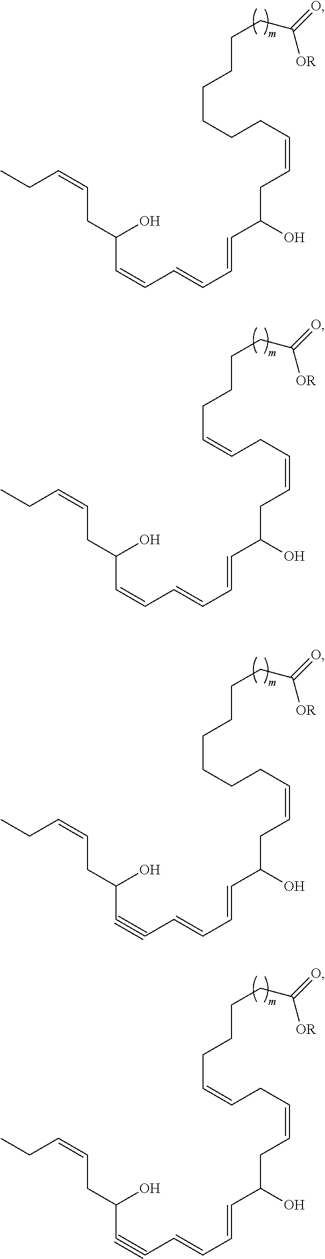 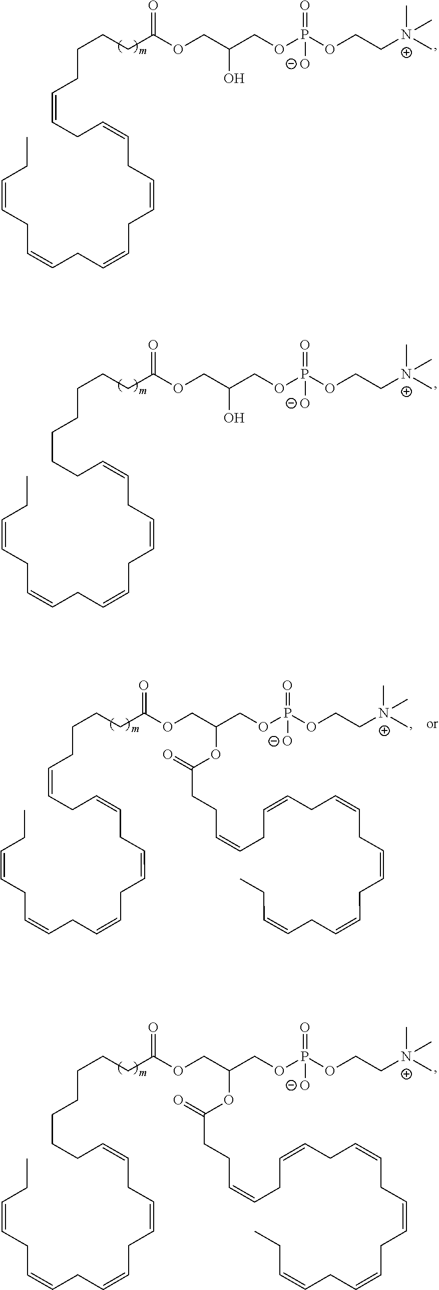
wherein m is selected from a group consisting of 0 to 19; and
wherein —COOR is a carboxylic acid group, a pharmaceutically acceptable carboxylic ester, or a pharmaceutically acceptable salt thereof.

10. The method of claim 9, wherein if —COOR is a carboxylic acid salt, the R group is a cation selected from a group consisting of an ammonium cation, an iminium cation, or a metal cation selected from a group consisting of sodium, potassium, magnesium, zinc, or calcium cation.

11. The method of claim 9, wherein if —COOR is a carboxylic ester, the R group is selected from a group consisting of methyl, ethyl, alkyl, a part of a phospholipid, or a derivative thereof.

12. The method of claim 1, wherein the arachidonic acid derivative comprises a lipoxin compound:

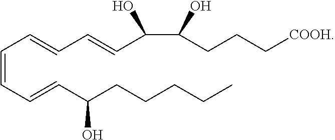 [SMILES: CCCCC[C@@H](O)/C=C/C=C\C=C\C=C\[C@@H](O)[C@@H](O)CCCC(=O)O]

13. The method of claim 1, wherein the docosahexaenoic acid derivative comprises a resolving compound:
Resolvin D6

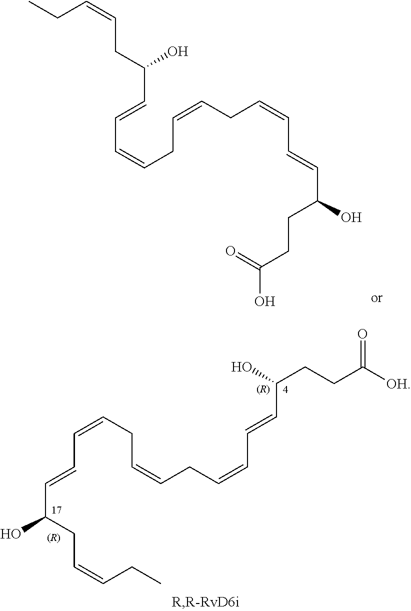

14. The method of claim 9, wherein the VLC-PUFA derivative comprises: 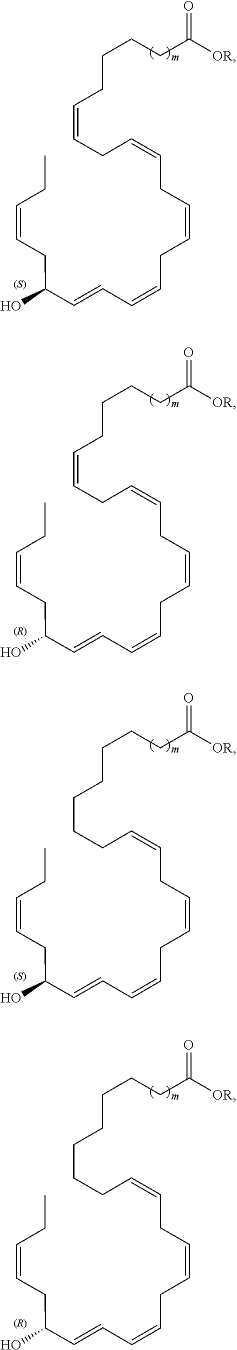 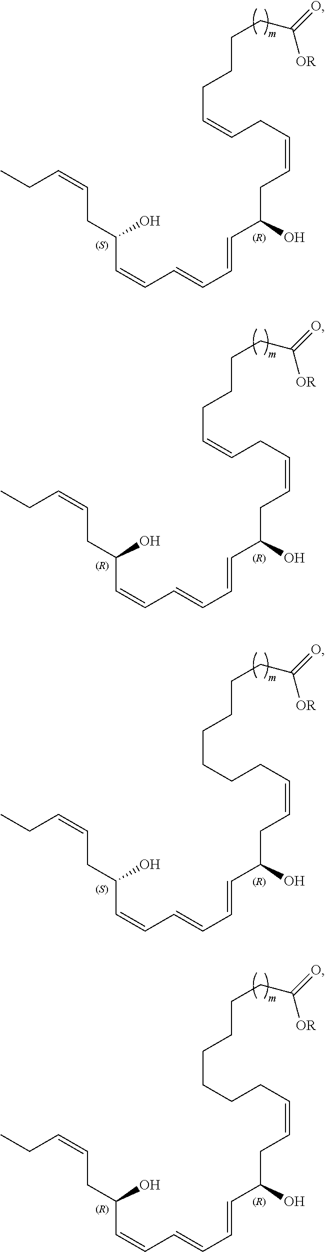 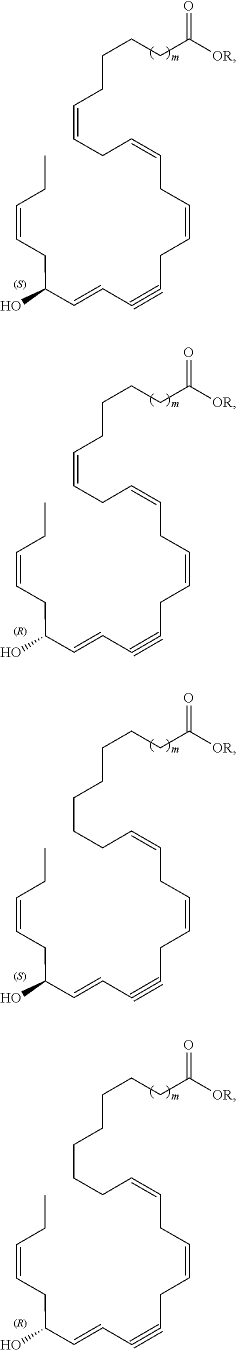 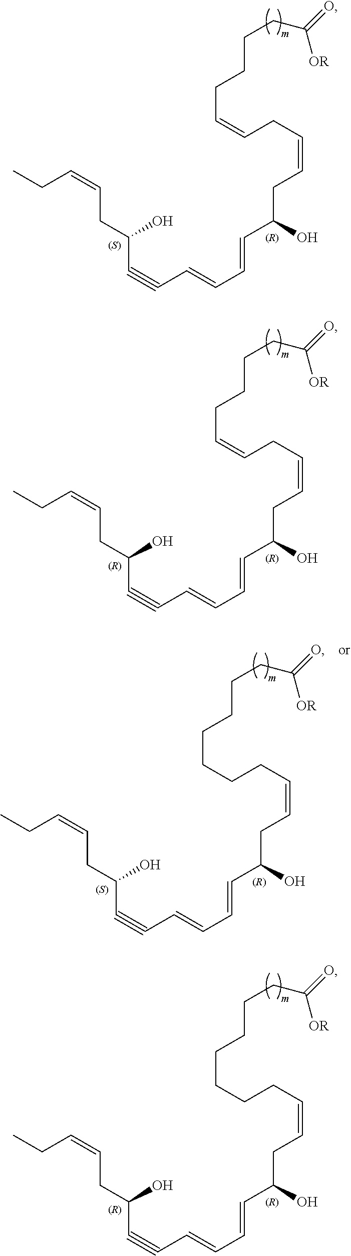
wherein m is selected from a group consisting of 0 to 19; and
wherein —COOR is a carboxylic acid group, a pharmaceutically acceptable carboxylic ester, or a pharmaceutically acceptable salt thereof.

15. The method of claim 14, wherein if —COOR is a carboxylic acid salt, the R group is a cation selected from a group consisting of an ammonium cation, an iminium cation, or a metal cation selected from a group consisting of sodium, potassium, magnesium, zinc, or calcium cation.

16. The method of claim 14, wherein if —COOR is a carboxylic ester, the R group is selected from a group consisting of methyl, ethyl, alkyl, a part of a phospholipid, or a derivative thereof.

17. The method of claim 14, wherein VLC-PUFA derivative comprises:

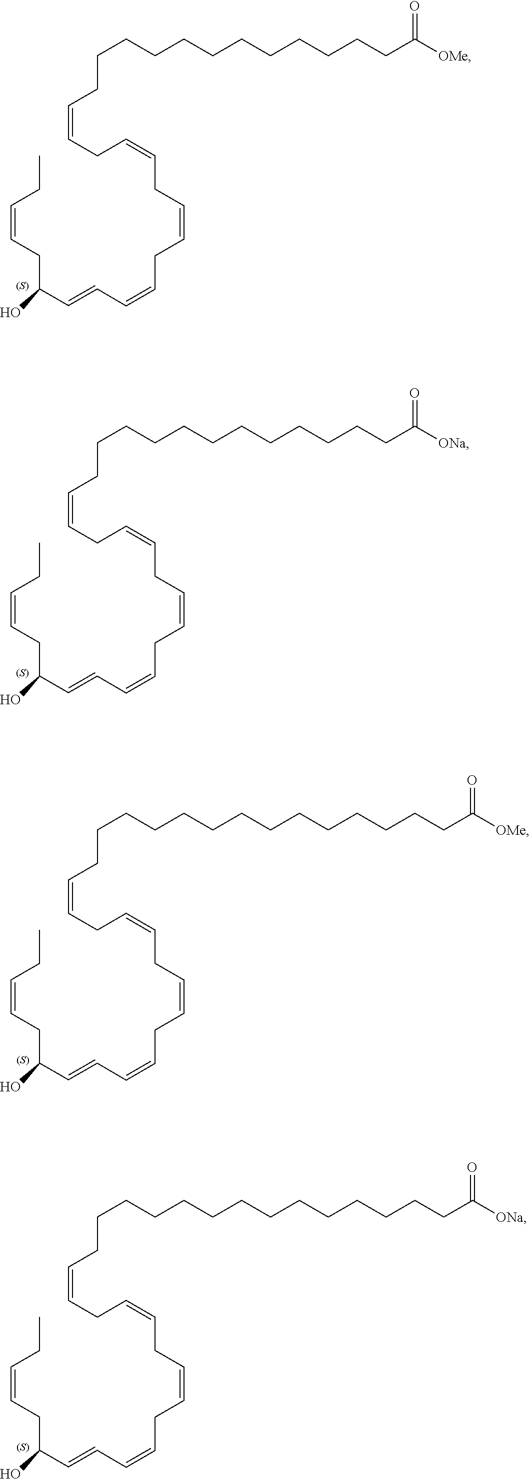

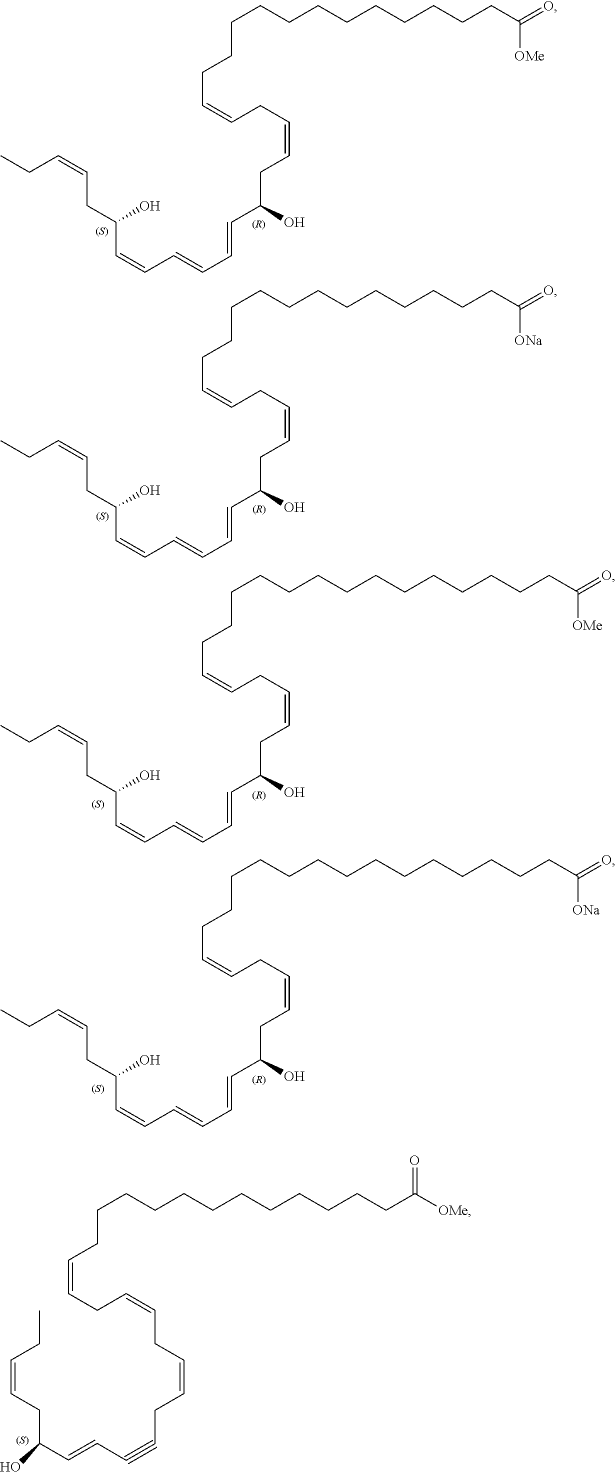

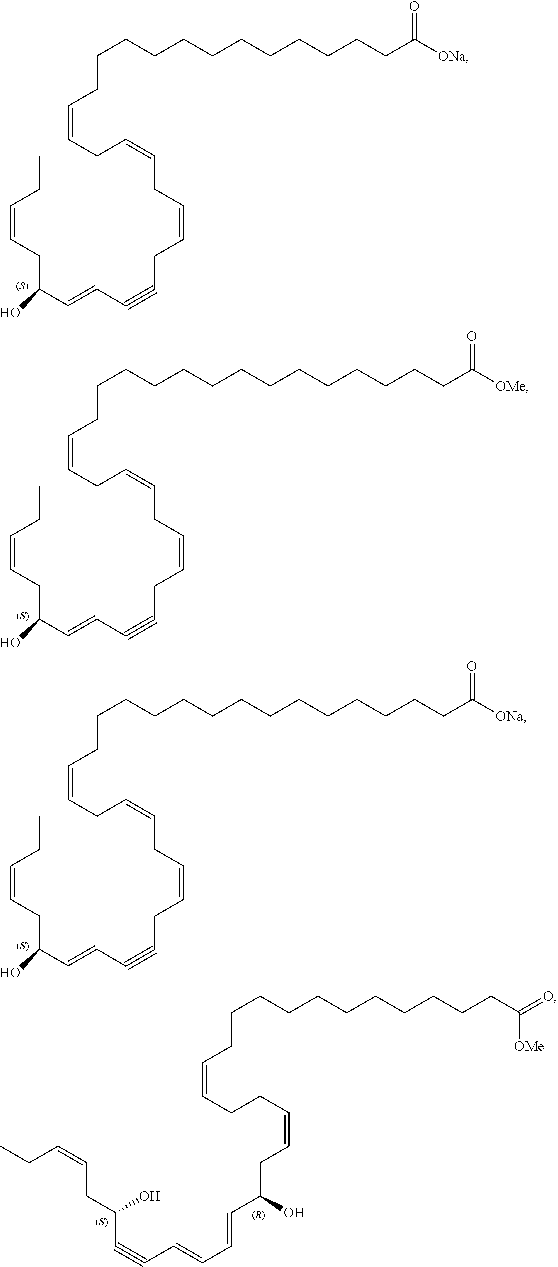

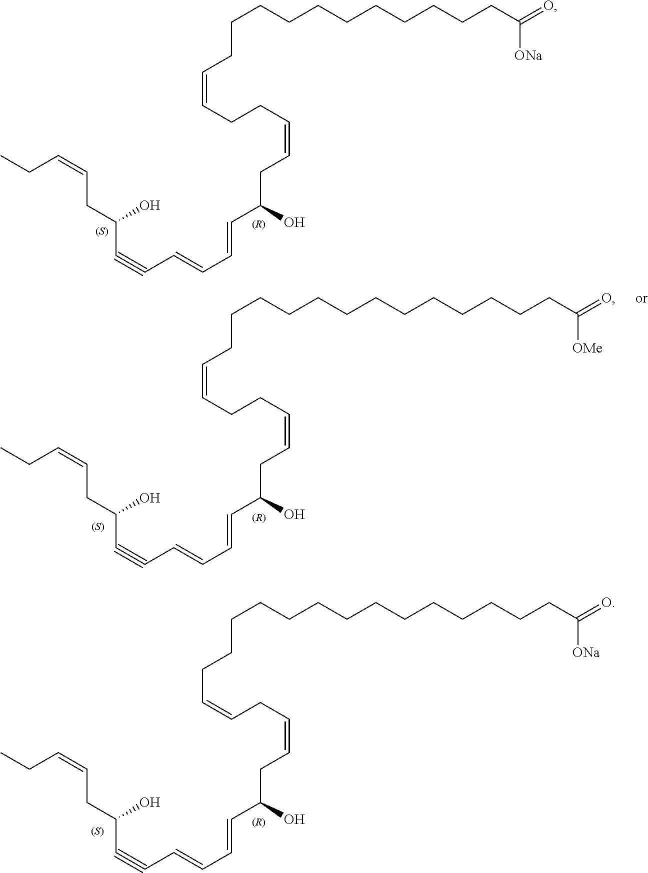

18. The method of claim 1, wherein the compound is a pharmaceutical composition.

19. The method of claim 18, wherein the pharmaceutical composition is administered topically, intranasally, orally, ocularly, parenterally, or nebulized.

20. The method of claim 19, wherein the nebulized pharmaceutical comprises an aerosol or spray.

21. The method of claim 19, wherein the pharmaceutical composition administered ocularly comprises an eye drop.

22. The method of claim 1, wherein the method further comprises administering to the subject one or more active agents.

23. The method of claim 22, wherein the agent comprises an anti-inflammatory, a pain reliever, an antioxidant, a PAF-receptor antagonist, or an antiviral.

24. The method of claim 1, wherein the wherein the VLC-PUFA, arachidonic acid, docosahexaenoic acid, or derivative thereof is administered prior to, subsequent to, or concurrently with onset of viral infection.

25. The method of claim 1, wherein the immune response is indicated by increased production of pro-inflammatory cytokines, chemokines, or a combination thereof.

26. The method of claim 29, wherein the pro-inflammatory cytokines and chemokines comprise IL-6, IL-1β, IL-8/CXCL8, CCL2/MCP-1, CXCL1/KC/GRO, VEGF, or ICAM1(CD54).

27. The method of claim 1, wherein the therapeutically effective amount comprises a concentration of about 500 nM to about 700 nM.

28. The method of claim 1, wherein the viral infection comprises a coronavirus infection, an influenza virus infection, or an adenovirus infection.

29. The method of claim 28, wherein the coronavirus comprises SARS-CoV-2.
